(19) United States
(12) Patent Application Publication
(10) Pub. No.: US 2015/0175530 A1
(43) Pub. Date: Jun. 25, 2015

(54) ANTIBACTERIAL AGENTS

(71) Applicant: Achaogen, Inc., South San Francisco, CA (US)

(72) Inventors: Brian D. Patterson, San Francisco, CA (US); Qing Lu, Foster City, CA (US); James Bradley Aggen, Boston, MA (US); Paola Dozzo, San Francisco, CA (US); Ramesh Annasaheb Kasar, Bellevue, WA (US); Martin Sheringham Linsell, San Mateo, CA (US); Timothy Robert Kane, Moss Beach, CA (US); Micah James Gliedt, Sunnyvale, CA (US); Darin James Hildebrandt, Cupertino, CA (US); Glenn A. McEnroe, San Mateo, CA (US); Frederick Cohen, San Francisco, CA (US); Heinz E. Moser, San Mateo, CA (US)

(21) Appl. No.: 14/536,286

(22) Filed: Nov. 7, 2014

Related U.S. Application Data
(63) Continuation of application No. PCT/US2013/040350, filed on May 9, 2013.
(60) Provisional application No. 61/644,659, filed on May 9, 2012.
(60) Provisional application No. 61/777,540, filed on Mar. 12, 2013.

(51) Int. Cl. C07C 235/66; C07D 233/64; C07D 261/08

(52) U.S. Cl. CPC C07C 235/66; C07D 261/08; C07D 233/64; C07C 2101/16

(57) ABSTRACT

Antibacterial compounds of formula (I) are provided:

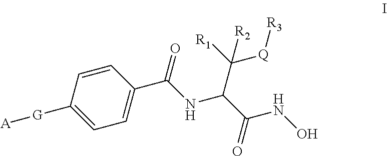

as well as stereoisomers and pharmaceutically acceptable salts thereof; pharmaceutical compositions comprising such compounds; methods of treating bacterial infections by the administration of such compounds; and processes for the preparation of such compounds.

### II. CROSS-REFERENCE TO RELATED APPLICATIONS

This application is a continuation of International PCT Application No. PCT/US2013/040350, which was filed on May 9, 2013, now pending, which claims the benefit under 35 U.S.C. §119(e) of U.S. Provisional Patent Application No. 61/644,659, filed May 9, 2012, and U.S. Provisional Patent Application No. 61/777,540, filed Mar. 12, 2013. The foregoing applications are incorporated herein by reference in their entireties.

### I. STATEMENT OF GOVERNMENT INTEREST

This invention was made with government support under Contract HDTRA1-07-C-0079 awarded by the United States Department of Defense. The government has certain rights in this invention.

### III. BACKGROUND

A. Field

This invention pertains generally to treating infections caused by gram-negative bacteria. More specifically, the invention described herein pertains to treating gram-negative infections by inhibiting activity of UDP-3-O—(R-3-hydroxydecanoyl)-N-acetylglucosamine deacetylase (LpxC). The present invention provides small molecule inhibitors of LpxC, pharmaceutical formulations containing such inhibitors, methods of treating patients with such pharmaceutical formulations, and methods of preparing such pharmaceutical formulations and inhibitors. The invention described herein pertains to treating gram-negative infections by administering compounds capable of inhibiting activity of UDP-3-O—(R-3-hydroxydecanoyl)-N-acetylglucosamine deacetylase (LpxC), either alone or in combination with administering a second antibacterial compound.

B. Introduction

Over the past several decades, the frequency of antimicrobial resistance and its association with serious infectious diseases have increased at alarming rates. The problem of antibacterial resistance is compounded by the existence of bacterial strains resistant to multiple antibacterials. Thus there is a need for new antibacterials, particularly antibacterials with novel mechanisms of action. A previously unexploited but highly conserved target, LpxC, provides a new opportunity for developing broad-spectrum antibacterial small molecules that comprise a new class of active bactericidal chemical entities that should encounter little, if any, naturally-occurring, target-related resistance. LpxC (the enzyme uridyldiphospho-3-O—(R-hydroxydecanoyl)-N-acetylglucosamine deacetylase) is present across all Gram-negative bacterial species of interest and is involved in the first committed step in outer membrane biosynthesis. Thus LpxC is essential for survival and presents an ideal target for antibiotic activity in Gram-negative bacterial species.

Researchers have identified some compounds with antibacterial activity that target lipid A biosynthesis. For example, Jackman et al. (J. Biol. Chem., 2000, 275(15), 11002-11009); Wyckoff et al. (Trends in Microbiology, 1998, 6(4), 154-159); U.S. Patent Application Publication No. 2001/0053555 (published 20 Dec. 2001, corresponding to International PCT Publication No. WO 98/18754, published 7 May 1998); International PCT Publication No. WO 00/61134 (published 19 Oct. 2000); U.S. Patent Application Publication No. 2004/0229955 (published 18 Nov. 2004); International PCT Publication No. WO 2008/027466 (published 6 Mar. 2008); International PCT Publication No. WO 2008/105515 (published 4 Sep. 2008); International PCT Publication No. WO 2008/154642 (published 18 Dec. 2008); International PCT Publication No. WO 2009/158369 (published 30 Dec. 2009); International PCT Publication No. WO 2010/017060 (published 11 Feb. 2010); International PCT Publication No. WO 2010/024356 (published 4 Mar. 2010); International PCT Publication No. WO 2010/031750 (published 25 Mar. 2010); International PCT Publication No. WO 2010/032147 (published 25 Mar. 2010); International PCT Publication No. WO 2010/100475 (published 10 Sep. 2010); International PCT Publication No. WO 2011/045703 (published 21 Apr. 2011); International PCT Publication No. WO 2011/073845 (published 23 Jun. 2011); and International PCT Publication No. WO 2011/132712 (published 27 Oct. 2011) all disclose compounds having antibacterial anti-LpxC activity. The commercial development of these LpxC inhibitors has been complicated by toxicity of these compounds in mammalian animals at concentrations at or near those required for antibacterial activity. The compounds presented herein are significantly better tolerated, more active and/or less protein-bound than other closely related compounds having anti-LpxC activity.

Although there have been advances in the field, there remains a need for LpxC inhibitors that have activity as bactericidal agents against gram-negative bacteria and have an acceptable efficacy and toxicity/tolerance profile. It is, accordingly, an object of this invention to provide compounds and combinations of such compounds for use in the preparation of non-toxic antibacterials and other pharmaceuticals capable of inhibiting gram-negative bacterial infections.

### IV. BRIEF SUMMARY OF THE INVENTION

The present invention provides novel compounds, pharmaceutical formulations including the compounds, methods of inhibiting UDP-3-O—(R-3-hydroxydecanoyl)-N-acetylglucosamine deacetylase (LpxC), and methods of treating gram-negative bacterial infections.

In one aspect, the invention provides compounds of formula I:

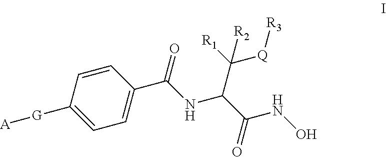

and stereoisomers, pharmaceutically acceptable salts, and esters thereof, wherein
A is selected from the group consisting of:
   (a) substituted C₁-C₂ alkyl, wherein at least one substituent is hydroxy;
   (b) substituted C₃-C₈ alkyl, wherein at least two substituents are hydroxy;
   (c) substituted cycloalkyl, wherein;
      (i) at least one substituent is dihydroxyalkyl; or
      (ii) at least two substituents independently are selected from hydroxy and hydroxyalkyl; and
   (d) substituted cycloalkylalkyl, wherein at least two substituents independently are selected from hydroxy and hydroxyalkyl and wherein each substitution independently is to either the cyclic portion or alkyl portion of the cycloalkylalkyl;
G is selected from the group consisting of —C≡C—, —CH═CH—C≡C—, —C≡C—CH═CH—, and —C≡C—C≡C—;
Q is O or NR, wherein R is hydrogen or an unsubstituted C₁-C₃ alkyl;
R¹ and R² independently are selected from the group consisting of hydrogen and substituted or unsubstituted C₁-C₃ alkyl, or R¹ and R², together with the carbon atom to which they are attached, form an unsubstituted C₃-C₆ cycloalkyl group or an unsubstituted 4-6 membered heterocyclic group; and
R³ is selected from the group consisting of hydrogen, substituted or unsubstituted C₁-C₆ alkyl, substituted or unsubstituted cycloalkyl, substituted or unsubstituted cycloalkylalkyl, substituted or unsubstituted aryl, substituted or unsubstituted arylalkyl, substituted or unsubstituted heterocyclyl, substituted or unsubstituted heterocyclylalkyl, substituted or unsubstituted heteroaryl, and substituted or unsubstituted heteroarylalkyl.

In certain implementations, R³ is hydrogen or a substituted or unsubstituted C₁-C₆ alkyl. In some implementations, G is —C≡C—C≡C—. In some implementations, Q is NR, wherein R hydrogen or unsubstituted C₁-C₃ alkyl, preferably wherein R is hydrogen. In some implementations both R¹ and R² are methyl. In some implementations, when A is substituted C₁-C₂ alkyl, wherein at least one substituent is hydroxy, A preferably is hydroxylmethyl or hydroxyethyl. In certain implementations, A is substituted C₃-C₈ alkyl, wherein at least two substituents are hydroxyl. In other implementations, A is substituted cycloalkyl, wherein at least two substituents independently are selected from hydroxy and hydroxyalkyl. In these implementations, the substituents preferably are selected from hydroxy and hydroxymethyl. In alternative implementations, A is substituted cycloalkyl, wherein at least one substituent is dihydroxyalkyl. In alternative implementations, A is substituted cycloalkylalkyl, wherein at least two substituents are independently selected from hydroxy and hydroxyalkyl, and wherein each substitution independently is to either the cyclic portion or alkyl portion of the cycloalkylalkyl. In these implementations, the substituents preferably are selected from hydroxy and hydroxymethyl.

One aspect of the invention provides compounds selected from the group consisting of:
(S)—N-(3-amino-1-(hydroxyamino)-3-methyl-1-oxobutan-2-yl)-4-(5-hydroxypenta-1,3-diynyl)benzamide (1);
(S)—N-(3-amino-1-(hydroxyamino)-3-methyl-1-oxobutan-2-yl)-4-(3-hydroxyprop-1-ynyl)benzamide (2);
(S,E)-N-(3-amino-1-(hydroxyamino)-3-methyl-1-oxobutan-2-yl)-4-(5-hydroxypent-3-en-1-ynyl)benzamide (3);
(S)—N-(3-hydroxy-1-(hydroxyamino)-3-methyl-1-oxobutan-2-yl)-4-(5-hydroxypenta-1,3-diynyl)benzamide (4);
(S)—N-(1-(hydroxyamino)-3-methyl-3-(methylamino)-1-oxobutan-2-yl)-4-(5-hydroxypenta-1,3-diynyl)benzamide (5);
(S)—N-(1-(hydroxyamino)-3-(2-hydroxyethylamino)-3-methyl-1-oxobutan-2-yl)-4-(5-hydroxypenta-1,3-diynyl)benzamide (6);
(S)—N-(3-amino-1-(hydroxyamino)-3-methyl-1-oxobutan-2-yl)-4-(6-hydroxyhexa-1,3-diynyl)benzamide (7);
(S)—N-(3-amino-1-(hydroxyamino)-3-methyl-1-oxobutan-2-yl)-4-(4-hydroxybut-1-ynyl)benzamide (8);
(S)—N-(1-(hydroxyamino)-3-methyl-3-(methylamino)-1-oxobutan-2-yl)-4-(6-hydroxyhexa-1,3-diynyl)benzamide (9);
(S)—N-(3-(ethylamino)-1-(hydroxyamino)-3-methyl-1-oxobutan-2-yl)-4-(6-hydroxyhexa-1,3-diynyl)benzamide (10);
(S)—N-(1-(hydroxyamino)-3-(2-hydroxyethylamino)-3-methyl-1-oxobutan-2-yl)-4-(6-hydroxyhexa-1,3-diynyl)benzamide (11);
(S)—N-(1-(hydroxyamino)-3-methyl-3-((5-methylisoxazol-3-yl)methylamino)-1-oxobutan-2-yl)-4-(6-hydroxyhexa-1,3-diynyl)benzamide (12);
(S)—N-(3-((1H-imidazol-4-yl)methylamino)-1-(hydroxyamino)-3-methyl-1-oxobutan-2-yl)-4-(6-hydroxyhexa-1,3-diynyl)benzamide (13);
N—((S)-3-amino-1-(hydroxyamino)-3-methyl-1-oxobutan-2-yl)-4-((S)-5-hydroxyhexa-1,3-diynyl)benzamide (14);
N—((S)-3-amino-1-(hydroxyamino)-3-methyl-1-oxobutan-2-yl)-4-((R)-5-hydroxyhexa-1,3-diynyl)benzamide (15);
N—((S)-1-(hydroxyamino)-3-methyl-3-(methylamino)-1-oxobutan-2-yl)-4-((S)-5-hydroxyhexa-1,3-diynyl)benzamide (16);
N—((S)-3-amino-1-(hydroxyamino)-3-methyl-1-oxobutan-2-yl)-4-(5,6-dihydroxyhexa-1,3-diynyl)benzamide (17);
(S)—N-(3-amino-1-(hydroxyamino)-3-methyl-1-oxobutan-2-yl)-4-(6-hydroxy-5-(hydroxymethyphexa-1,3-diynyl)benzamide (18);
N—((S)-3-amino-1-(hydroxyamino)-3-methyl-1-oxobutan-2-yl)-4-(6-chloro-5-hydroxy-5-(hydroxymethyl)hexa-1,3-diynyl)benzamide (19);
N—((S)-3-amino-1-(hydroxyamino)-3-methyl-1-oxobutan-2-yl)-4-((5S,6S)-6,7-dihydroxy-5-methylhepta-1,3-diynyl)benzamide (20A);
N—((S)-3-amino-1-(hydroxyamino)-3-methyl-1-oxobutan-2-yl)-4-((5S,6R)-6,7-dihydroxy-5-methylhepta-1,3-diynyl)benzamide (20B);
N—((S)-3-amino-1-(hydroxyamino)-3-methyl-1-oxobutan-2-yl)-4-(6,7-dihydroxyhepta-1,3-diynyl)benzamide (21);
4-(6,7-dihydroxyhepta-1,3-diynyl)-N—((S)-1-(hydroxyamino)-3-methyl-3-(methylamino)-1-oxobutan-2-yl)benzamide (22);
N—((S)-3-amino-1-(hydroxyamino)-3-methyl-1-oxobutan-2-yl)-4-(((1S,2R,3S)-2,3-bis(hydroxymethyl)cyclopropyl)buta-1,3-diynyl)benzamide (23A);
N—((S)-3-amino-1-(hydroxyamino)-3-methyl-1-oxobutan-2-yl)-4-(((1R,2R,3S)-2,3-bis(hydroxymethyl)cyclopropyl)buta-1,3-diynyl)benzamide (23B);
N—((S)-3-amino-1-(hydroxyamino)-3-methyl-1-oxobutan-2-yl)-4-(((R)-2,2-bis(hydroxymethyl)cyclopropyl)buta-1,3-diynyl)benzamide (24);
N—((S)-3-amino-1-(hydroxyamino)-3-methyl-1-oxobutan-2-yl)-4-((2-(1,2-dihydroxyethyl)cyclopropyl)buta-1,3-diynyl)benzamide (25);
N—((S)-3-amino-1-(hydroxyamino)-3-methyl-1-oxobutan-2-yl)-4-(5-hydroxy-5-(2-(hydroxymethyl)cyclopropyl)penta-1,3-diynyl)benzamide (26);
N—((S)-3-amino-1-(hydroxyamino)-3-methyl-1-oxobutan-2-yl)-4-(((1S,3R)-1-hydroxy-3-(hydroxymethyl)cyclobutyl)buta-1,3-diynyl)benzamide (27);
N—((S)-3-amino-1-(hydroxyamino)-3-methyl-1-oxobutan-2-yl)-4-(5-(1-hydroxy-3-(hydroxymethyl)cyclobutyl)penta-1,3-diynyl)benzamide (28);
N—((S)-3-amino-1-(hydroxyamino)-3-methyl-1-oxobutan-2-yl)-4-(((1R,3S)-3-hydroxy-3-(hydroxymethyl)cyclobutyl)buta-1,3-diynyl)benzamide (29);
N—((S)-3-amino-1-(hydroxyamino)-3-methyl-1-oxobutan-2-yl)-4-(((1S,2S,3S)-3-hydroxy-2-(hydroxymethyl)cyclobutyl)buta-1,3-diynyl)benzamide (30A);
N—((S)-3-amino-1-(hydroxyamino)-3-methyl-1-oxobutan-2-yl)-4-(((1S,2S,3R)-3-hydroxy-2-(hydroxymethyl)cyclobutyl)buta-1,3-diynyl)benzamide (30B);
N—((S)-3-amino-1-(hydroxyamino)-3-methyl-1-oxobutan-2-yl)-4-(((1S,2R,3R)-3-hydroxy-2-(hydroxymethyl)cyclobutyl)buta-1,3-diynyl)benzamide (31A);
N—((S)-3-amino-1-(hydroxyamino)-3-methyl-1-oxobutan-2-yl)-4-(((1S,2R,3S)-3-hydroxy-2-(hydroxymethyl)cyclobutyl)buta-1,3-diynyl)benzamide (31B);
N—((S)-3-amino-1-(hydroxyamino)-3-methyl-1-oxobutan-2-yl)-4-(((1S,3R,4S)-3,4-dihydroxycyclopentyl)buta-1,3-diynyl)benzamide (32);
N—((S)-3-amino-1-(hydroxyamino)-3-methyl-1-oxobutan-2-yl)-4-(((1S,4R)-4-hydroxy-4-(hydroxymethyl)cyclohexyl)buta-1,3-diynyl)benzamide (33A);
N—((S)-3-amino-1-(hydroxyamino)-3-methyl-1-oxobutan-2-yl)-4-(((1R,4S)-4-hydroxy-4-(hydroxymethyl)cyclohexyl)buta-1,3-diynyl)benzamide (33B);
(S)—N-(3-hydroxy-1-(hydroxyamino)-3-methyl-1-oxobutan-2-yl)-4-(6-hydroxyhexa-1,3-diyn-1-yl)benzamide (34);
N-((2S,3R)-3-amino-1-(hydroxyamino)-1-oxobutan-2-yl)-4-(6-hydroxyhexa-1,3-diyn-1-yl)benzamide (35);
N—((S)-3-amino-1-(hydroxyamino)-3-methyl-1-oxobutan-2-yl)-4-((S)-6,7-dihydroxyhepta-1,3-diyn-1-yl)benzamide (36);
N—((S)-3-amino-1-(hydroxyamino)-3-methyl-1-oxobutan-2-yl)-4-((R)-6,7-dihydroxyhepta-1,3-diyn-1-yl)benzamide (37);
N-((2S,3R)-3-hydroxy-1-(hydroxyamino)-1-oxobutan-2-yl)-4-(6-hydroxyhexa-1,3-diyn-1-yl)benzamide (38);
N—((S)-3-amino-1-(hydroxyamino)-3-methyl-1-oxobutan-2-yl)-4-(6-hydroxy-5-methylhexa-1,3-diyn-1-yl)benzamide (39);
N—((S)-3-hydroxy-1-(hydroxyamino)-3-methyl-1-oxobutan-2-yl)-4-((S)-5-hydroxyhexa-1,3-diyn-1-yl)benzamide (40);
N-((2S,3R)-3-amino-1-(hydroxyamino)-1-oxobutan-2-yl)-4-((S)-5-hydroxyhexa-1,3-diyn-1-yl)benzamide (41);
N-((2S,3R)-3-hydroxy-1-(hydroxyamino)-1-oxobutan-2-yl)-4-((S)-5-hydroxyhexa-1,3-diyn-1-yl)benzamide (42);
4-((S)-6,7-dihydroxyhepta-1,3-diyn-1-yl)-N—((S)-1-(hydroxyamino)-3-methyl-3-(methylamino)-1-oxobutan-2-yl)benzamide (43);
4-((R)-6,7-dihydroxyhepta-1,3-diyn-1-yl)-N—((S)-1-(hydroxyamino)-3-methyl-3-(methylamino)-1-oxobutan-2-yl)benzamide (44);
N-((2S,3R)-3-amino-1-(hydroxyamino)-1-oxobutan-2-yl)-4-(5,6-dihydroxyhexa-1,3-diyn-1-yl)benzamide (45);
N—((S)-3-amino-1-(hydroxyamino)-3-methyl-1-oxobutan-2-yl)-4-(5,7-dihydroxyhepta-1,3-diyn-1-yl)benzamide (46);
N—((S)-3-amino-1-(hydroxyamino)-3-methyl-1-oxobutan-2-yl)-4-(6-hydroxy-5-methoxyhexa-1,3-diyn-1-yl)benzamide (47);
N-((2S,3R)-1-(hydroxyamino)-3-(methylamino)-1-oxobutan-2-yl)-4-(6-hydroxyhexa-1,3-diyn-1-yl)benzamide (48);
N-((2S,3R)-1-(hydroxyamino)-3-(methylamino)-1-oxobutan-2-yl)-4-((S)-5-hydroxyhexa-1,3-diyn-1-yl)benzamide (49);
N—((S)-3-amino-1-(hydroxyamino)-3-methyl-1-oxobutan-2-yl)-4-(5,6-dihydroxy-5-methylhexa-1,3-diyn-1-yl)benzamide (50);
4-(5,6-dihydroxyhexa-1,3-diyn-1-yl)-N—((S)-1-(hydroxyamino)-3-methyl-3-(methylamino)-1-oxobutan-2-yl)benzamide (51);
N—((S)-3-amino-1-(hydroxyamino)-3-methyl-1-oxobutan-2-yl)-4-((E)-6,7-dihydroxyhept-3-en-1-yn-1-yl)benzamide (52);
N—((S)-3-amino-1-(hydroxyamino)-3-methyl-1-oxobutan-2-yl)-4-((5S,6S)-5,7-dihydroxy-6-methylhepta-1,3-diyn-1-yl)benzamide (53);
N—((S)-3-amino-1-(hydroxyamino)-3-methyl-1-oxobutan-2-yl)-4-((S)-5,7-dihydroxyhepta-1,3-diyn-1-yl)benzamide (54);
(S,E)-N-(3-amino-1-(hydroxyamino)-3-methyl-1-oxobutan-2-yl)-4-(6-hydroxyhex-3-en-1-yn-1-yl)benzamide (55);
N-((2S,3R)-1-(hydroxyamino)-3-(methylamino)-1-oxobutan-2-yl)-4-(5-hydroxypenta-1,3-diyn-1-yl)benzamide (56);
N—((S)-3-amino-1-(hydroxyamino)-3-methyl-1-oxobutan-2-yl)-4-((1-hydroxy-3-(hydroxymethyl)cyclopentyl)buta-1,3-diyn-1-yl)benzamide (57);
N—((S)-3-amino-1-(hydroxyamino)-3-methyl-1-oxobutan-2-yl)-4-((E)-5,6-dihydroxyhex-3-en-1-yn-1-yl)benzamide (58);
N-((2S,3R)-3-amino-4,4,4-trifluoro-1-(hydroxyamino)-1-oxobutan-2-yl)-4-((S)-5-hydroxyhexa-1,3-diyn-1-yl)benzamide (59);
4-(5,6-dihydroxyhexa-1,3-diyn-1-yl)-N—((S)-3-hydroxy-1-(hydroxyamino)-3-methyl-1-oxobutan-2-yl)benzamide (60);
N—((S)-3-amino-1-(hydroxyamino)-3-methyl-1-oxobutan-2-yl)-4-((S)-5,6-dihydroxyhexa-1,3-diyn-1-yl)benzamide (61); and
N—((S)-3-amino-1-(hydroxyamino)-3-methyl-1-oxobutan-2-yl)-4-((R)-5,6-dihydroxyhexa-1,3-diyn-1-yl)benzamide (62).

In another aspect, the present invention provides a pharmaceutical composition comprising a compound of Formula I, or a stereoisomer, pharmaceutically acceptable salt, or ester thereof, and a pharmaceutically acceptable carrier or diluent.

In another aspect, the present invention provides a pharmaceutical composition or formulation comprising an effective amount of an antibacterial compound of Formula I, or a stereoisomer, pharmaceutically acceptable salt, or ester thereof, and a pharmaceutically acceptable carrier or diluent.

In another aspect, the present invention provides a method of inhibiting a deacetylase enzyme in gram-negative bacteria, thereby affecting bacterial growth, comprising administering to a patient in need of such inhibition an LpxC-inhibitory compound of Formula I or a stereoisomer, pharmaceutically acceptable salt, or ester thereof.

In another aspect, the present invention provides a method of inhibiting LpxC, thereby modulating the virulence of a bacterial infection, comprising administering to a patient in need of such inhibition an LpxC-inhibitory compound of Formula I or a stereoisomer, pharmaceutically acceptable salt, or ester thereof.

In another aspect, the present invention provides a method for treating a patient having a bacterial infection comprising administering to the patient in need thereof an antibacterially effective amount of a compound of Formula I, or a stereoisomer, pharmaceutically acceptable salt, or ester thereof. In a more specific embodiment of the method of treatment, the bacterial infection is a gram-negative bacterial infection. In a further specific embodiment the patient is a mammal and in certain embodiments, a human.

In another aspect, the present invention provides a method of administering an antibacterially effective amount of a compound of Formula I or a stereoisomer, pharmaceutically acceptable salt, or ester thereof, to a patient infected with a fermentative or non-fermentative gram-negative bacteria. Examples of such bacteria include Pseudomonas aeruginosa, Stenotrophomonas maltophila, Burkholderia cepacia, Alcaligenes xylosoxidans, Enterobacteriaceae, Haemophilus, Franciscellaceae (e.g., Franciscella tularensis) and Neisseria species.

In another aspect, the present invention provides a method of administering an antibacterially effective amount of a compound of Formula I, or a stereoisomer, pharmaceutically acceptable salt, or ester thereof, to a patient infected with gram-negative bacteria. Examples of such bacteria include Enterobacteriaceae, such as Serratia, Proteus, Klebsiella, Enterobacter, Citrobacter, Salmonella, Providencia, Yersinia (e.g., Yersinia pestis), Morganella, Cedecea, Edwardsiella species and Escherichia coli.

These and other aspects of the invention will be evident upon reference to the following detailed description.

### V. DETAILED DESCRIPTION OF THE INVENTION

The present invention provides novel compounds, methods for inhibiting LpxC in gram-negative bacteria, and novel methods for treating bacterial infections. The compounds provided herein can be formulated into pharmaceutical formulations and medicaments that are useful in the methods of the invention. The invention also provides for the use of the compounds in preparing medicaments and pharmaceutical formulations, for use of the compounds in inhibiting LpxC, and for use of the compounds in treating bacterial infections in a patient. The invention further provides compositions and methods for treating gram-negative infections by administering compounds capable of inhibiting activity of UDP-3-O—(R-3-hydroxydecanoyl)-N-acetylglucosamine deacetylase (LpxC), either alone or in combination with administering a second antibacterial compound

A. Definitions

The following abbreviations and definitions are used throughout this application:

“LpxC” is an abbreviation that stands for UDP-3-O—(R-3-hydroxydecanoyl)-N-acetylglucosamine deacetylase.

As used herein, the following definitions shall apply unless otherwise indicated.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃C≡CH₂—).

“Alkoxy” refers to the group —O-alkyl, wherein alkyl is as defined herein. Alkoxy includes methoxy, ethoxy, n-propoxy, isopropoxy, n-butoxy, t-butoxy, sec-butoxy, n-pentoxy, and the like.

“Amino” refers to the group —NH₂.

“Alkenyl” refers to straight chain or branched hydrocarbyl groups having from 2 to 6 carbon atoms and preferably 2 to 4 carbon atoms and having at least 1 and preferably from 1 to 2 sites of vinyl (>C═C<) unsaturateduration. Such groups are exemplified by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic —C≡C— unsaturateduration. Examples of such alkynyl groups include acetylenyl (—C≡CH), and propargyl (—CH₂C≡CH).

“Carboxyl” or “carboxy” refers to —COOH or salts thereof.

“Cyano” or “nitrile” refers to the group —CN.

“Cycloalkyl” refers to cyclic alkyl groups of from 3 to 13 carbon atoms having single. Examples of cycloalkyl groups include cyclopropyl, cyclobutyl, cyclopentyl, cyclooctyl, and the like.

“Halo” or “halogen” refers to fluoro, chloro, bromo, and iodo and is typically fluoro or chloro.

“Hydroxy” or “hydroxyl” refers to the group —OH.

“Heterocycle,” “heterocyclic,” and “heterocyclyl” refer to a saturated or unsaturated group having a single ring, and having from 3 to 15 ring atoms, including 1 to 4 hetero atoms. These ring atoms are selected from the group consisting of nitrogen, sulfur, or oxygen. In one implementation, the nitrogen and/or sulfur atom(s) of the heterocyclic group are optionally oxidized to provide for the N-oxide, —S(O)—, or —SO₂— moieties.

“Nitro” refers to the group —NO₂.

“Oxo” refers to the atom (═O).

“Substituted” refers to a group having one or more hydrogens replaced with substituents selected from the group consisting of alkoxy, acyl, acylamino, acyloxy, amino, aminocarbonyl, aminocarbonylamino, aminothiocarbonylamino, aminocarbonyloxy, amidino, carboxyl ester, (carboxyl ester)amino, (carboxyl ester)oxy, cyano, halo, hydroxy, nitro, sulfonyl, thioacyl, and alkylthio, wherein said substituents are as defined herein. In certain substituted cyclic groups, “substituted” also refers to a group having one or more hydrogens replaced with an alkyl group or “substituted” refers a group having two hydrogens replaced with a single double bonded oxygen atom (an oxo group) or a single double bonded sulfur atom (thioxo). In some implementations, the substituted group has 1 to 3 of the aforementioned substituents. In other implementations, the substituted group has 1 to 2 of the aforementioned substituents

“Sulfonyl” refers to the group —SO₂-alkyl, —SO₂-substituted alkyl, —SO₂-alkenyl, —SO₂-substituted alkenyl, wherein alkyl, substituted alkyl, alkenyl, substituted alkenyl, alkynyl, and substituted alkynyl are as defined herein. Sulfonyl includes groups such as methyl-SO₂—.

“Thioacyl” refers to the groups H—C(S)—, alkyl-C(S)—, substituted alkyl-C(S)—, alkenyl-C(S)—, substituted alkenyl-C(S)—, alkynyl-C(S)—, and substituted alkynyl-C(S)—, wherein alkyl, substituted alkyl, alkenyl, substituted alkenyl, alkynyl, and substituted alkynyl are as defined herein.

“Thioxo” refers to the atom (═S).

“Alkylthio” refers to the group —S-alkyl, wherein alkyl is as defined herein. In other implementations, sulfur may be oxidized to —S(O)—. The sulfoxide may exist as one or more stereoisomers.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

Generally, reference to a certain element such as hydrogen or H is meant to include all isotopes of that element. For example, if a substituent group is defined to include hydrogen or H, it also includes deuterium and tritium.

The subject invention also includes isotopically-labeled compounds of the present invention, that are structurally identical to those disclosed herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, sulfur, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively. Compounds of the present invention, prodrugs thereof, and pharmaceutically acceptable salts of said compounds and of said prodrugs that contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, may afford certain therapeutic advantages resulting from greater metabolic stability, for example increased in vivo half-life or reduced dosage requirements and, hence, may be preferred in some circumstances. Isotopically labeled compounds of this invention and prodrugs thereof can generally be prepared by carrying out known or referenced procedures and by substituting a readily available isotopically labeled reagent for a non-isotopically labeled reagent.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

“Patient” refers to human and non-human animals, especially mammals.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, phosphate, sulfate and the like.

“Pharmaceutically effective amount” and “therapeutically effective amount” refer to an amount of a compound sufficient to treat a specified disorder or disease or one or more of its symptoms and/or to prevent the occurrence of the disease or disorder.

The term “antibacterial agent” refers to agents that have either bactericidal or bacteriostatic activity. The term “inhibiting the growth” indicates that the rate of increase in the numbers of a population of a particular bacterium is reduced. Thus, the term includes situations in which the bacterial population increases but at a reduced rate, as well as situations where the growth of the population is stopped, as well as situations where the numbers of the bacteria in the population are reduced or the population even eliminated. If an enzyme activity assay is used to screen for inhibitors, one can make modifications in uptake/efflux, solubility, half-life, etc. to compounds in order to correlate enzyme inhibition with growth inhibition. The activity of antibacterial agents is not necessarily limited to bacteria but may also encompass activity against parasites, virus, and fungi.

Unless the context requires otherwise, throughout the specification and claims which follow, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

B. Compounds, Compositions and Use Thereof

In one aspect, the present invention provides compounds of formula I

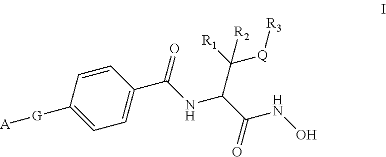

and stereoisomers, pharmaceutically acceptable salts, or esters thereof, wherein
A is selected from the group consisting of:
   (a) substituted C₁-C₂ alkyl, wherein at least one substituent is hydroxy;
   (b) substituted C₃-C₈ alkyl, wherein at least two substituents are hydroxy;
   (c) substituted cycloalkyl, wherein;
      (i) at least one substituent is dihydroxyalkyl; or
      (ii) at least two substituents independently are selected from hydroxy and hydroxyalkyl; and
   (d) substituted cycloalkylalkyl, wherein at least two substituents independently are selected from hydroxy and hydroxyalkyl and wherein each substitution independently is to either the cyclic portion or alkyl portion of the cycloalkylalkyl;
G is selected from the group consisting of —C≡C—, —CH═CH—C≡C—, —C≡C—CH═CH—, and —C≡C—C≡C—;
Q is O or NR, wherein R is hydrogen or an unsubstituted C₁-C₃ alkyl;
R¹ and R² independently are selected from the group consisting of hydrogen and substituted or unsubstituted C₁-C₃ alkyl, or R¹ and R², together with the carbon atom to which they are attached, form an unsubstituted C₃-C₆ cycloalkyl group or an unsubstituted 4-6 membered heterocyclic group; and
R³ is selected from the group consisting of hydrogen, substituted or unsubstituted C₁-C₆ alkyl, substituted or unsubstituted cycloalkyl, substituted or unsubstituted cycloalkylalkyl, substituted or unsubstituted aryl, substituted or unsubstituted arylalkyl, substituted or unsubstituted heterocyclyl, substituted or unsubstituted heterocyclylalkyl, substituted or unsubstituted heteroaryl, and substituted or unsubstituted heteroarylalkyl.

Compounds of the present invention can be readily synthesized using the methods described herein, or other methods, that are well known in the art. For example, the synthesis of hxdroxamic acids or similar scaffolds having a wide variety of substituents are comprehensively reviewed in Kline, T., et al., “Potent, novel in vitro inhibitors of the Pseudomonas aeruginosa deacetylase LpxC” J. Med Chem. 2002, 45(14), 3112-29; U.S. Pat. No. 5,925,659; Pirrung, M. C., et al., “A Convenient Procedure for the Preparation of Amino Acid Hydrokamates from Esters” J. Org. Chem. 1995, 60, 8084-8085; Nhu, K., et al., “A New and Efficient Solid Phase Synthesis of Hydroxamic Acids” J. Org. Chem. 1997, 62, 7088-7089; Internationa PCT Publication No. WO98/18754; Mellor, S. L., et al., “N-Fmoc-aminoxy-2-chlortrityl Polystyrene Resin: A Facile Solid-phase Methodology for the Synthesis of Hydroxamic Acids” Tetrahedron Lett. 1997, 38, 3311-3314; Khan, S. I., et al., “A Facile and Convenient Solid-phase Procedure for Synthesizing Nucleoside Hydroxamic Acids” Terahedron. Lett. 1998, 39, 8031-8034; Zhang, Y., et al., “Design, Combinatorial Chemical Synthesis, and in vitro Characterization of Novel Urea Based Gelatinase Inhibitors” Bioorg. Med. Chem. Lett. 1999, 9, 2823-2826; Ito, Y., et al., “Synthetic Reactions by Complex Catalysts. XXXI, A Novel and Versaturatedile Method of Heterocycle Synthesis” J. Am Chem. Soc. 1973, 95, 4447-4448; Ito, Y., et al., “Synthetic Reactions by Complex Catalysts XXXV” Syn. Commun. 1974, 4, 97-103; Witte, H., et al., “Cyclische Imidsaurester aus Nitrilen and Aminoalkoholen” Liebigs Ann. Chem. 1974, 996-1009; Pattenden, G., et al., “Naturally Occurring Linear Fused Thiazoline-Thiazole Containing Metabolites: Total Synthesis of (−) Didehydromirabazole A, a Cytotoxic Alkaloid from Blue-Green Algae” J. Chem. Soc. Perkin Trans 1993, 1, 1629-1636; Boyce, R. J., et al., “Total Synthesis of Thiangazole, A Novel Naturally Occurring HIV-1 Inhibitor from Polyangium sp.” Tetrahedron 1995, 51, 7321-7330; Galeotti, N., et al., “Synthesis of Peptidyl Aldehydes from Thiazolines” Tetrahedron. Lett. 1997, 38, 2459-2462; Charette, A. B., et al., “Mild Method for the Synthesis of Thiazolines from Secondary and Tertiary Amides” J. Org. Chem. 1998, 63, 908-909; Bergeron, R. J., et al., “Effects of C-4 Stereochemistry and C-4′ Hydroxylation on the Iron Clearing Efficiency and Toxicity of Desferrithiocin Analogues” J. Med. Chem. 1999, 42, 2432-2440; Raman, P., et al., “Titanium (IV)-mediated Tandem Deprotection-cyclodehydration of Protected Cysteine N-Amides: Biomimetic Synthesis of Thiazoline- and Thiazole-containing Heterocycles” Org. Lett. 2000, 2, 3289-3292; Fernandez, X., et al., “Novel Synthesis of 2-Thioazolines” Tetrahedron Lett. 2000, 41, 3381-3384; and Wipf, P., et al., “C. Thiolysis of Oxazolinenes: A New, Selective Method for the Direct Conversion of Peptide Oxazolines into Thiazolines” Tetrahedron Lett. 1995, 36, 6395-6398, which are incorporated herein by reference.

The compounds of Formula I are distinguished from previously known substances by a surprisingly low degree of binding to plasma proteins and can therefore provide a relatively high concentration of free, i.e. pharmacologically effective and available drug concentration. In order to achieve an antibacterial effect, the MIC for the target organism must be reached in vivo. Binding of the antibacterial agent to plasma proteins will decrease the available plasma concentration of the agent, making it more difficult to achieve a concentration at or above the MIC. The compounds disclosed herein demonstrate decreased protein binding as compared to previously known substances, and therefore can more easily achieve a therapeutic concentration in the patient.

In another aspect, the present invention provides a pharmaceutical composition comprising a compound of Formula I, or a stereoisomer, pharmaceutically acceptable salt, or ester thereof, and a pharmaceutically acceptable carrier or diluent.

In another aspect, the invention provides a method of inhibiting a deacetylase enzyme in a gram-negative bacteria, thereby affecting bacterial growth, comprising administering to a patient in need of such inhibition a compound of Formula I or a stereoisomer, pharmaceutically acceptable salt, or ester thereof.

In another aspect, the invention provides a method of inhibiting LpxC, thereby modulating the virulence of a bacterial infection, comprising administering to a patient in need of such inhibition a compound of Formula or a stereoisomer, pharmaceutically acceptable salt, or ester thereof. In certain embodiments of the method of inhibiting LpxC using a compound of the present invention, the IC₅₀ value of the compound is less than or equal to 10 μM with respect to LpxC. In other embodiments, the IC₅₀ value is less than or equal to 1 μM, is less than or equal to 0.1 μM, is less than or equal to 0.050 μM, is less than or equal to 0.030 μM, is less than or equal to 0.025 μM, or is less than or equal to 0.010 μM.

In another aspect, the invention provides a method for treating a patient having a gram-negative bacterial infection comprising administering to the patient in need thereof an antibacterially effective amount of a compound of Formula I or a stereoisomer, pharmaceutically acceptable salt, or ester thereof.

In another aspect, the invention provides a method of administering a therapeutically effective amount of a compound of Formula I or a stereoisomer, pharmaceutically acceptable salt, or ester thereof, to a patient infected with a fermentative or non-fermentative gram-negative bacteria. Examples of fermentative or non-fermentative gram-negative bacteria include Pseudomonas aeruginosa, Stenotrophomonas maltophila, Burkholderia cepacia, Alcaligenes xylosoxidans, Enterobacteriaceae, Haemophilus, Franciscellaceae (e.g., Franciscella tularensis) and Neisseria species.

In another aspect, the invention provides a method of administering an inhibitory amount of a compound described herein to gram-negative bacteria, such as Enterobacteriaceae which is selected from the group consisting of organisms such as Serratia, Proteus, Klebsiella, Enterobacter, Citrobacter, Salmonella, Providencia, Yersinia (e.g., Yersinia pestis), Morganella, Cedecea, Edwardsiella species and Escherichia coli.

In certain embodiments, the patient may be a mammal, and in some embodiments, a human.

Bacterial infections susceptible to treatment according to the present invention include primary infections and co-infections caused by a species of bacteria and one or more additional infectious agents such as, for example, bacteria, virus, parasite and fungus.

Compounds of the invention can be used for treating conditions caused by the bacterial production of endotoxin and, in particular, by gram-negative bacteria and bacteria that use LpxC in the biosynthesis of lipopolysaccharide (LPS) or endotoxin.

Compounds of the invention also are useful in treating conditions that are caused or exacerbated by the bacterial production of lipid A and LPS or endotoxin, such as sepsis, septic shock, systemic inflammation, localized inflammation, chronic obstructive pulmonary disease (COPD) and acute exacerbations of chronic bronchitis (AECB). For these conditions, treatment includes the administration of a compound of the invention, or a combination of compounds of the invention, optionally with a second agent wherein the second agent is a second antibacterial agent or a non-antibacterial agent.

For sepsis, septic shock, systemic inflammation, localized inflammation, chronic obstructive pulmonary disease (COPD) and acute exacerbations of chronic bronchitis (AECB), representative non-antibacterial agents include antiendotoxins including endotoxin receptor-binding antibodies, endotoxin-binding antibodies, anti-CD14-binding protein antibodies, antilipopolysaccharide-binding protein antibodies and tyrosine kinase inhibitors.

In treatment of serious or chronic respiratory tract infections, compounds of the present invention may also be used with non-antibacterial agents administered via inhalation. Representative non-antibacterial agents used in this treatment include anti-inflammatory steroids, non-steroidal anti-inflammatory agents, bronchiodilators, mucolytics, anti-asthma therapeutics and lung fluid surfactants. In particular, the non-antibacterial agent may be albuterol, salbuterol, budesonide, beclomethasone, dexamethasone, nedocromil, beclomethasone, fluticasone, flunisolide, triamcinolone, ibuprofin, rofecoxib, naproxen, celecoxib, nedocromil, ipratropium, metaproterenol, pirbuterol, salmeterol, formoterol, indacaterol, bronchiodilators, mucolytics, calfactant, beractant, poractant alfa, surfaxin or pulmozyme (also called domase alfa).

Compounds of the invention can be used alone or in combination with a second antibacterial agent for the treatment of a serious or chronic respiratory tract infection including serious lung and nosocomial infections such as those caused by Enterobacter aerogenes, Enterobacter cloacae, Escherichia coli, Klebsiella pneumoniae, Klebsiella oxytoca, Proteus mirabilis, Serratia marcescens, Stenotrophomonas maltophilia, Pseudomonas aeruginosa, Burkholderia cepacia, Alcaligenes xylosoxidans, Flavobacterium meningosepticum, Providencia stuartii and Citrobacter freundi, community lung infections such as those caused by Haemophilus Influenzae, Legionella species, Moraxella catarrhalis, Branhamella catarrhalis, Enterobacter species, Klebsiella species, and Proteus species, infections caused by other bacterial species such as Neisseria species, Shigella species, Salmonella species, Helicobacter pylori, Vibrionaceae and Bordetella species, as well as infections caused by a Brucella species, Francisella tularensis and/or Yersinia Pestis.

When used for treating patients infected with gram-negative bacterial infections, compounds of the present invention can be used to sensitize gram-negative bacteria to the effects of a second agent.

The present invention provides novel combinations of compounds including a compound of Formula I or a stereoisomer, pharmaceutically acceptable salt, or ester thereof, as well as methods for treating patients infected with gram-negative bacteria. The novel combinations provided herein can be formulated into pharmaceutical formulations and medicaments that are useful in the methods of the invention. The invention also provides for the use of the novel combinations in preparing medicaments and pharmaceutical formulations, for use of the combinations in treating bacterial infections in a patient.

In one embodiment, a second antibacterial agent is used in combination with a compound of Formula I, or stereoisomer or pharmaceutically acceptable salt thereof. Examples of suitable second antibactieral agents include, but are not limited to, vancomycin, linezolid, azithromycin, imipenem, teicoplanin, daptomycin, clindamycin, rifampin, cefotaxime, gentamicin, novobiocin or telavancin. In one such embodiment, the antibacterial agent is vancomycin, teicoplanin, rifampin, azithromycin, telavancin or novobiocin. Most preferably, the antibacterial agent is vancomycin or rifampin. In some embodiments of the invention, the antibacterial agent and/or the compound of Formula I, or stereoisomer or pharmaceutically acceptable salt thereof, is administered at a sub-therapeutic dose, wherein a subtherapeutic dose is a dose that would be insufficient to treat bacterial infections, if administered alone.

Pharmaceutical compositions of the present invention comprise a therapeutically effective amount of a compound of Formula I, or a stereoisomer or pharmaceutically acceptable salt thereof, formulated together with one or more pharmaceutically acceptable carriers or diluents. As used herein, the term “pharmaceutically acceptable carrier” means a non-toxic, inert solid, semi-solid or liquid filler, diluent, encapsulating material or formulation auxiliary of any type. Some examples of materials that can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and other animals orally, rectally, parenterally (as by intravenous, intramuscular or subcutaneous injection), intracisternally, intravaginally, intraperitoneally, topically (as by powders, ointments, or drops), bucally, or as an oral or nasal spray, or a liquid aerosol or dry powder formulation for inhalation.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, 1% lidocaine, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions that can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution that, in turn, may depend upon crystal size and crystalline form. Alternatively, delayed absorption of a parenterally administered drug form may be accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions that are compatible with body tissues.

Compositions for rectal or vaginal administration are preferably suppositories that can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Solid compositions of a similar type may also be employed as fillers in soft and hard-filled gelatin capsules using such excipients as lactose or milk sugar as well as high molecular weight polyethylene glycols and the like.

The antibacterial compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like arealso contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compositions of the invention may also be formulated for delivery as a liquid aerosol or inhalable dry powder. Liquid aerosol formulations may be nebulized predominantly into particle sizes that can be delivered to the terminal and respiratory bronchioles where bacteria reside in patients with bronchial infections, such as chronic bronchitis and pneumonia. Pathogenic bacteria are commonly present throughout airways down to bronchi, bronchioli and lung parenchema, particularly in terminal and respiratory bronchioles. During exacerbation of infection, bacteria can also be present in alveoli. Liquid aerosol and inhalable dry powder formulations are preferably delivered throughout the endobronchial tree to the terminal bronchioles and eventually to the parenchymal tissue.

Aerosolized formulations of the invention may be delivered using an aerosol forming device, such as a jet, vibrating porous plate or μLtrasonic nebulizer, preferably selected to allow the formation of a aerosol particles having with a mass medium average diameter predominantly between 1 to 5 μm. Further, the formulation preferably has balanced osmolarity ionic strength and chloride concentration, and the smallest aerosolizable volume able to deliver effective dose of the compounds of the invention to the site of the infection. Additionally, the aerosolized formulation preferably does not impair negatively the functionality of the airways and does not cause undesirable side effects.

Aerosolization devices suitable for administration of aerosol formulations of the invention include, for example, jet, vibrating porous plate, μLtrasonic nebulizers and energized dry powder inhalers, that are able to nebulize the formulation of the invention into aerosol particle size predominantly in the size range from 1-5 pm. Predominantly in this application means that at least 70% but preferably more than 90% of all generated aerosol particles are 1 to 5 μm range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An μLtrasonic nebulizer works by a piezoelectric crystal that shears a liquid into small aerosol droplets. A variety of suitable devices are available, including, for example, AeroNeb and AeroDose vibrating porous plate nebulizers (AeroGen, Inc., Sunnyvale, Calif.), Sidestream7 nebulizers (Medic-Aid Ltd., West Sussex, England), Pad LC7 and Pari LC Star7 jet nebulizers (Pari Respiratory Equipment, Inc., Richmond, Va.), and Aerosonic (DeVilbiss Medizinische Produkte (Deutschland) GmbH, Heiden, Germany) and pLtraAire7 (Omron Healthcare, Inc., Vernon Hills, Ill.) μLtrasonic nebulizers.

Compounds of the invention may also be formulated for use as topical powders and sprays that can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present invention, bacterial infections are treated or prevented in a patient such as a human or lower mammal by administering to the patient a therapeutically effective amount of a compound of Formula I, or a stereoisomer or pharmaceutically acceptable salt thereof, in such amounts and for such time as is necessary to achieve the desired result. By a “therapeutically effective amount” of a compound of the invention is meant a sufficient amount of the compound to treat bacterial infections, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The total daily dose of the compounds of this invention administered to a human or other mammal in single or in divided doses can be in amounts, for example, from 0.01 to 200 mg/kg body weight or more usually from 0.1 to 50 mg/kg body weight. In certain embodiments, the total daily dose administered to a human or other mammal is from 1.0 to 100 mg/kg body weight or from 5.0 to 25 mg/kg body weight. Single dose compositions may contain such amounts or submultiples thereof to make up the daily dose. In general, treatment regimens according to the present invention comprise administration to a patient in need of such treatment from about 10 mg to about 15 g of the compound(s) of this invention per day in single or multiple doses, more usually, from 100 mg to 5 g, and even more usually from 250 mg to 1 g per day in single or multiple doses.

Methods of formulation are well known in the art and are disclosed, for example, in Remington: The Science and Practice of Pharmacy, Mack Publishing Company, Easton, Pa., 19th Edition (1995). Pharmaceutical compositions for use in the present invention can be in the form of sterile, non-pyrogenic liquid solutions or suspensions, coated capsules, suppositories, lyophilized powders, transdermal patches or other forms known in the art.

A “kit” as used in the instant application includes a container for containing the pharmaceutical compositions and may also include divided containers such as a divided bottle or a divided foil packet. The container can be in any conventional shape or form as known in the art that is made of a pharmaceutically acceptable material, for example a paper or cardboard box, a glass or plastic bottle or jar, a resealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle that is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil that is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It maybe desirable to provide a written memory aid, where the written memory aid is of the type containing information and/or instructions for the physician, pharmacist or other health care provider, or patient, e.g., in the form of numbers next to the tablets or capsules whereby the numbers correspond with the days of the regimen that the tablets or capsules so specified should be ingested or a card that contains the same type of information. Another example of such a memory aid is a calendar printed on the card e.g., as follows “First Week, Monday, Tuesday,” . . . etc. . . . “Second Week, Monday, Tuesday, . . . ” etc. Other variations of memory aids will be readily apparent. A “daily dose” can be a single tablet or capsule or several tablets or capsules to be taken on a given day. When the kit contains separate compositions, a daily dose of one or more compositions of the kit can consist of one tablet or capsule while a daily dose of another one or more compositions of the kit can consist of several tablets or capsules.

Another specific embodiment of a kit is a dispenser designed to dispense the daily doses one at a time in the order of their intended use. Preferably, the dispenser is equipped with a memory-aid, so as to further facilitate compliance with the regimen. An example of such a memory-aid is a mechanical counter, that indicates the number of daily doses that has been dispensed. Another example of such a memory-aid is a battery-powered micro-chip memory coupled with a liquid crystal readout, or audible reminder signal that, for example, reads out the date that the last daily dose has been taken and/or reminds one when the next dose is to be taken.

The kits of the present invention may also include, in addition to a compound of the present invention, one or more additional pharmaceutically active compounds. For example, the additional compound second antibacterial. The additional compounds may be administered in the same dosage form as the compound of the present invention or in a different dosage form. Likewise, the additional compounds can be administered at the same time as the compound of the present invention or at different times.

Compositions of the present compounds may also be used in combination with other known antibacterial agents of similar spectrum to (1) enhance treatment of severe gram-negative infections covered by the spectrum of this compound or (2) add coverage in severe infections in which multiple organisms are suspected in which another agent of a different spectrum may be required in addition to this compound. Potential agents include members of the aminoglycosides, penicillins, cephalosporins, fluoroquinolones, macrolides, glycopeptides, lipopeptides and oxazolidinones. The treatment can involve administering a composition having both a compound of the present invention and a second antibacterial compound or administration of a compound of the present inventive compounds followed by or preceded by administration of a second antibacterial agent.

The foregoing may be better understood by reference to the following examples, that are presented for illustration and not to limit the scope of the inventive concepts.

### VI. EXAMPLES

A. Compound synthesis

The following are abbreviations used in the examples:
ACN: Acetonitrile
AcOH: Acetic acid
aq: Aqueous
BOC: tert-butoxycarbonyl
DCM: Dichloromethane
DIBAL-H: Diisobutylaluminium hydride
DIPEA: Diisopropylethylamine
DMAP: 4-Dimethylaminopyridine
DMF: N,N-Dimethylformamide
DMSO: Dimethyl sulfoxide
EA: Ethyl acetate
Et₂O: Diethylether
HATU: 2-(1H-7-Azabenzotriazol-1-yl)-1,1,3,3-tetramethyl uronium hexafluorophosphate Methanaminium
HOBt: N-Hydroxybenzotriazole
IPA: Isopropyl alcohol
m-CPBA: meta-Chloroperoxybenzoic acid
MTBE: Methyl tert-butyl ether
NBS N-Bromosuccinimide
NMO: N-methylmorpholine N-oxide
PCC: Pyridinium Chlorochromate
PE: Petroleum Ether
PE:EA: Petroleum Ether:Ethyl acetate
RP HPLC Reverse phase high performance liquid chromatography
rt: Room temperature
TBAF: Tetra-n-butylammonium fluoride
TEA: Triethylamine
TFA: Trifluoroacetic acid
THF: Tetrahydrofuran
TLC: Thin layer chromatography

Referring to the examples that follow, compounds of the present invention were characterized by high performance liquid chromatography (HPLC) using a Waters Millenium chromatography system with a 2690 Separation Module (Milford, Mass.) or an Agilent 1100 series chromatography system (Santa Clara, Calif.). The analytical columns were Phenomenex Luna C18(2) reversed phase, 10 μm, 100 Å, axia packed, 2.0×50 mm and the preparative columns were Phenomenex Luna C18(2) reversed phase, 10 μm, 100 Å, axia packed, 21.2×250 or 50×250 mm. A gradient elution was used, typically starting with 100% water and progressing to 100% acetonitrile over a varying lengths of time All solvents contained 0.1% acetic acid (AcOH). Compounds were detected by μLtraviolet light (UV) absorption at either 220 or 254 nm. In some instances, purity was assessed by thin layer chromatography (TLC) using glass or plastic backed silica gel plates, such as, for example, Baker-Flex Silica Gel 1 B2-F flexible sheets. TLC results were readily detected visually under μLtraviolet light, or by employing well known iodine vapor and other various staining techniques Mass spectrometric analysis was performed on one of three LCMS instruments: a Waters System. (Alliance HT HPLC and a Micromass ZQ mass spectrometer; Column: Eclipse XDB-C-18, 2.1×50 mm; solvent system: 5-95% (or 35-95%, or 65-95% or 95-95%) acetonitrile in water with 0.05% TFA; flow rate 0.8 mL/min; molecular weight range 500-1500; cone Voltage 20 V; column temperature 40° C.) or a Hewlett Packard System (Series 1100 HPLC; Column: Eclipse XDB-C18, 2.1×50 mm; solvent system: 1-95% acetonitrile in water with 0.05% TFA; flow rate 0.4 mL/min; molecular weight range 150-850; cone Voltage 50 V; column temperature 30° C.). or an Agilent System (Series 1100 HPLC; Column: Waters Sunfire C18 reversed phase, 2.5 μm, 100 Å, 2.1×50 mm; solvent system: 1-95% acetonitrile in water with 0.1% TFA; flow rate 0.5 mL/min; molecular weight range 150-1500; cone Voltage 70 V; column temperature 35° C.).

GCMS analysis was performed on a Hewlett Packard instrument (HP6890 Series gas chromatograph with a Mass Selective Detector 5973; injector volume: 1 μL; initial column temperature: 50° C.; final column temperature: 250 C; ramp time: 20 minutes; gas flow rate: 1 mL/min; column: 5% phenyl methyl siloxane, Model #HP 190915-443, dimensions: 30.0 m×25 m×0.25 m).

Nuclear magnetic resonance (NMR) analysis was performed with a Varian 300 MHz NMR (Palo Alto, Calif.). and a Varian Unity Enova 400 MHz NMR spectrometer (Palo Alto, Calif.). The spectral reference was either TMS or the known chemical shift of the solvent. Some compound samples were run at elevated temperatures (e.g. 75° C.) to promote increased sample solubility.

Procedure 1 (C—C Coupling Reaction Using CuCl-Cadiot):

Hydroxylamine hydrochloride (0.235 mmol, 0.06 eq) and CuCl (0.08, 0.02 eq) were dissolved in 23% aqueous n-butylamine (1 mL) and the resulting solution was cooled to 0° C. A solution of the alkyne (4.3 mmol, 1.1 eq) in 23% aqueous butylamine (2 mL) was then added. The bromo-alkyne (3.92 mmol) and hydroxylamine hydrochloride (0.235 mmol, 0.06 eq) were dissolved in 23% aqueous butylamine (2 mL) and THF (3 mL), and they were slowly added to the reaction mixture. The reaction was stirred for 1 hr, followed by quenching with EtOAc and water. The organic layer was separated and washed with brine, dried over Na₂SO₄, filtered and concentrated under reduced pressure to yield the desired coupled product.

Procedure 2 (Boc Deprotection Using TFA):

To the Boc-protected compound (3.39 mmol) at 0° C. was added a TFA:DCM solution (9 mL, 2:1) and the reaction was stirred for 1 hour. The reaction was concentrated under reduced pressure to yield a crude residue, which was azeotroped with IPA twice to yield the desired deprotected product.

Procedure 3 (Hydroxamate Formation):

To a stirring solution of the ester (3.38 mmol) in IPA (4 mL) at 0° C. was slowly added 50% aqueous NH₂OH (40 eq), and the reaction was stirred overnight. The reaction was quenched with AcOH (0.121 mol, 20 eq) or until the pH is 6. The volatiles were removed under reduced pressure, and the resulting solution was purified by RP HPLC.

Procedure 4a (Formation of Imine in Reductive Amination to NHMe):

To a stirred solution of the amine (2.37 g, 7.20 mmol) in DMF (14.39 mL) was added DIPEA (1.885 mL, 10.79 mmol) followed by formaldehyde (37% in water) (1.071 mL, 14.39 mmol) and the reaction was stirred for 2 hours. The excess aldehyde was quenched with n-butylamine (30% in water) (2.63 g, 10.79 mmol) and stirred for one hour. The reaction mixture was diluted with water, and lyophilized to yield the desired imine.

Procedure 4B (Reduction to Amine in Reductive Amination to NHMe):

To a stirring solution of the imine (3.96 g, 11.60 mmol) in THF (23.17 mL) and MeOH (2.439 mL) was added acetic acid (1.328 mL, 23.20 mmol) followed by sodium cyanoborohydride (10.94 g, 174 mmol) and the reaction was stirred for 1 hour. The reaction mixture was diluted with water (7 mL) and concentrated under reduced pressure to yield the crude amine.

### (S)-methyl 2-(4-(bromoethynyl)benzamido)-3-((tert-butoxycarbonyl)amino)-3-methylbutanoate (INT-1)

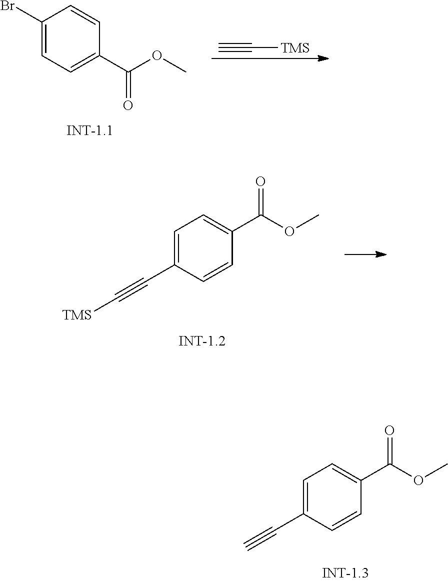

Ethynyltrimethylsilane (82.4 g, 0.84 mol, 1.2 equiv) was added dropwise over 10 min under a nitrogen atmosphere to a solution of methyl 4-bromobenzoate (150 g, 0.7 mol 1.0 equiv), PdCl₂ (PPh₃)₂ (15 g, 0.021 mol, 0.03 equiv) and CuI (13 g, 0.068 mol, 0.1 equiv) in TEA (1.5 L). The reaction was stirred at 90° C. for 30 minutes, whereupon LCMS showed complete consumption of methyl 4-bromobenzoate. Then, the reaction mixture was filtered and the filter cake was washed with EtOAc (5×500 mL). The filtrate was concentrated under reduced pressure to give a residue, which was distilled under reduced pressure to yield methyl 4-((trimethylsilyl)ethynyl)benzoate (INT-1.2) as an off-white solid (156 g, 96%).

To a solution of methyl 4-((trimethylsilyl)ethynyl)benzoate (156 g, 0.67 mol, 1.0 equiv) in methanol (800 mL) was added dropwise KOH/methanol (18 g/250 mL) keeping the temperature below 10° C., the mixture was allowed to warm to room temperature for 5 min. The reaction mixture was neutralized with 2M HCl. The reaction suspension was filtered to collect methyl 4-ethynylbenzoate (INT-1.3) as a white solid (97 g, 90%). MS: m/z calcd for C₁₀H₈O₂160.0. found [M+H]⁺ 161.

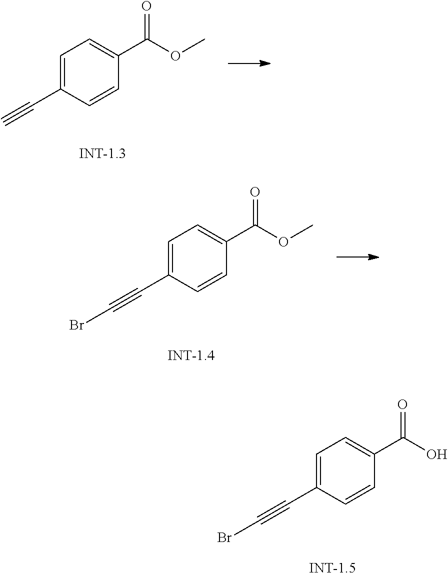

To a solution of methyl 4-ethynylbenzoate (50 g, 0.3125 mol, 1.0 equiv) in acetone (750 mL) was added AgNO₃ (5 g, 29.7 mmol 0.095 equiv) and the reaction mixture was stirred for 1 hr. NBS (61.2 g, 0.344 mol, 1.1 equiv) was added and the reaction mixture was stirred at room temperature for 20 hr, filtered and concentrated under reduced pressure. The residue was diluted in EA, and washed with iced 20% H₂SO₄. The EA layer was washed with water and brine, dried (Na₂SO₄), and filtered. The filtrate was concentrated under reduced pressure to give a residue, which was recrystallized from MeOH (1 mL/4 g) to yield methyl 4-(bromoethynyl)benzoate (INT-1.4) as an off-yellow solid (67 g, 90%). ¹H NMR (400 MHz, CDCl₃) δ 7.983 (d, J=8.8 Hz, 2H), 7.506 (d, J=8.8 Hz, 2H), 3.918 (s, 3H).

To a solution of methyl 4-(bromoethynyl)benzoate (67 g, 280 mmol, 1.0 equiv) in CH₃OH/THF/H₂O=5/5/1 (1100 mL) was added NaOH (44.84 g, 4.0 equiv) and the reaction mixture was stirred at 25° C. for 3 hr. The volatiles were removed under reduced pressure and the resulting solution was neutralized with 1 N HCl to pH 3-5. 4-(bromoethynyl)benzoic acid (INT-1.5) was collected by filtration. The filter cake was washed with water, and dried in an oven at 50° C. for 5 hr (61 g, 96%).

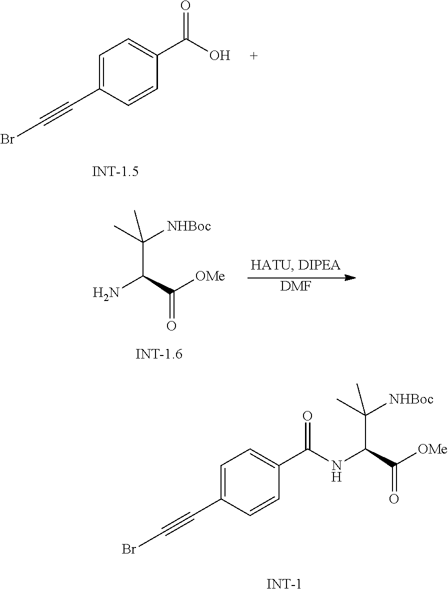

(S)-methyl 2-amino-3-(tert-butoxycarbonylamino)-3-methylbutanoate oxalate (9.34 g, 27.8 mmol) (synthesized as described in WO 2008/154642 at pages 240-6) was suspended in ethyl acetate (80 mL) and water (80 mL). While cooling in an ice bath, potassium carbonate (7.67 g, 55.5 mmol) was added and the reaction was stirred for 10 min. The aqueous layer was separated and extracted with ethyl acetate (2×75 mL) and the combined organic extracts were dried over sodium sulfate, and concentrated under reduced pressure to give a clear oil. To a stirring solution of 4-(bromoethynyl)benzoic acid (5.68 g, 25.2 mmol) in DMF (45 mL) at 0° C. was added a solution of (S)-methyl 2-amino-3-(tert-butoxycarbonylamino)-3-methylbutanoate in DMF (40 mL), followed by HATU (11.52 g, 30.3 mmol) and N-ethyl-N-isopropylpropan-2-amine (13.19 mL, 76 mmol) and the reaction was stirred for two hours with warming to room temperature. The reaction mixture was poured into water (300 mL) and was extracted with ethyl acetate (3×200 mL). The combined extracts were washed with saturated NaCl, dried over sodium sulfate and concentrated under reduced pressure to yield a crude, which was purified by flash chromatography (silica gel/20-60% EtOAc/Hexanes) to yield the desired product, (S)-methyl 2-(4-(bromoethynyl)benzamido)-3-((tert-butoxycarbonyl)amino)-3-methylbutanoate (INT-1), (9.24 g) as a white foam: TLC ethyl acetate/hexanes 1:1 R_{f} 0.5; MS: m/z calcd for C₂₀H₂₅BrN₂O₅ 452.09. found [M-Boc+H]⁺ 353.1.

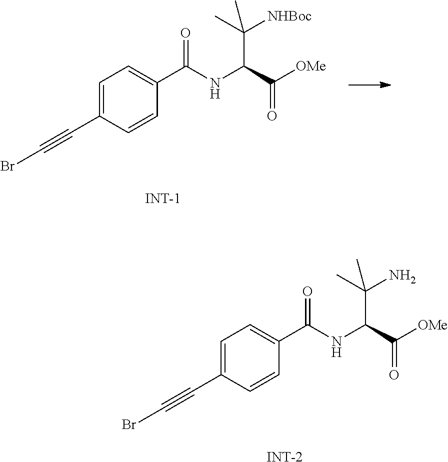 [SMILES: COC(=O)[C@@H](NC(=O)c1ccc(C#CBr)cc1)C(C)(C)NC(=O)OC(C)(C)C ; "INT-1" ; COC(=O)[C@@H](NC(=O)c1ccc(C#CBr)cc1)C(C)(C)N ; "INT-2"]

INT-2 was generated from INT-1 according to Procedure 2.

### (S)-methyl 3-((tert-butoxycarbonyl)amino)-2-(4-iodobenzamido)-3-methylbutanoate (INT-3)

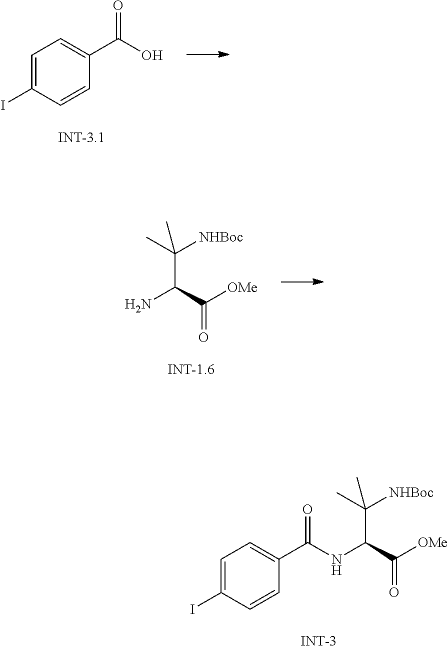

| | | | | Sam- | | Den- |
|---|---|---|---|---|---|---|
| | | | | ple | | sity |
| | | | Moles | Mass | Vol | (g/ |
| Reactant | MW | Eq | (mmol) | (g) | (ml) | ml) |
| 4-iodobenzoic acid | 248.018 | 1.000 | 34.8 | 8.62 | | |
| (S)-methyl 2- | 246.303 | 1.000 | 34.8 | 11.69 | | |
| amino-3-((tert- | | | | | | |
| butoxycarbonyl)amino)- | | | | | | |
| 3-methylbutanoate, | | | | | | |
| Oxalic acid | | | | | | |
| HATU | 235.265 | 2 | 69.5 | 26.4 | | |
| TEA | 101.190 | 1 | 34.8 | 3.52 | 4.84 | 0.726 |

To a stirring solution of iodobenzoic acid (8.62 g, 34.8 mmol) in acetonitrile (69.5 mL) was added HATU (26.4 g, 69.5 mmol), followed by TEA (4.84 mL, 34.8 mmol). INT-1.6 (11.69 g, 34.8 mmol) was then added and the reaction was stirred for 1 hr. Additional TEA (1 eq) was added and the reaction was stirred for 2.5 hr. The mixture was concentrated under reduced pressure to a crude, which was purified by flash chromatography (silica gel/10-50% EtOAc in hexanes) to yield INT-3 (11.7 g, 70.7%).

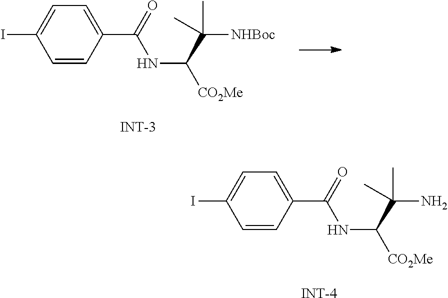 [SMILES: COC(=O)[C@@H](NC(=O)c1ccc(I)cc1)C(C)(C)NC(=O)OC(C)(C)C ; "INT-3" ; COC(=O)[C@@H](NC(=O)c1ccc(I)cc1)C(C)(C)N ; "INT-4"]

(S)-methyl 3-((tert-butoxycarbonyl)amino)-2-(4-iodobenzamido)-3-methylbutanoate (INT-3) (11.1 g, 23.3 mmol) was dissolved in dichloromethane (50 mL) and treated with trifluoroacetoic acid (50 mL) at ambient temperature for 5 minutes. The solvent was concentrated under reduced pressure, and the resulting crude was re-dissolved in DCM and concentrated again. The residue was then partitioned between MTBE and saturated sodium bicarbonate. The organic layer was washed with water, semi-saturated sodium bicarbonate then saturated sodium bicarbonate, dried over magnesium sulfate, and concentrated under reduced pressure to yield INT-4 (2 g, 5.3 mmol, 23%).

### 1. (S)—N-(3-amino-1-(hydroxyamino)-3-methyl-1-oxobutan-2-yl)-4-(5-hydroxypenta-1,3-diynyl)benzamide (1)

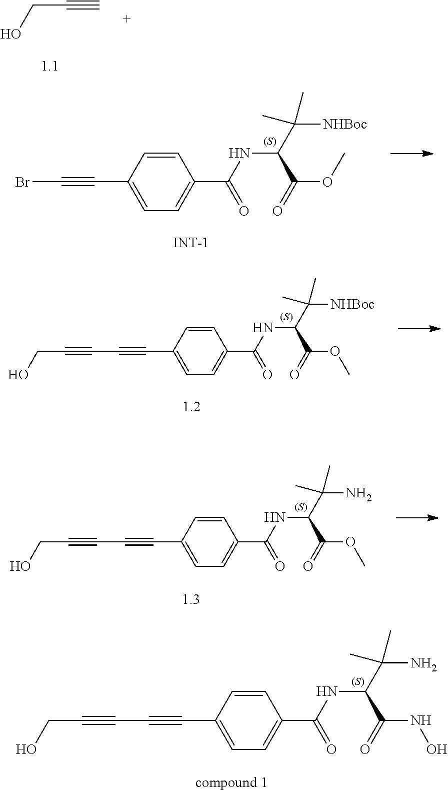

Prop-2-yn-1-ol was coupled with (S)-methyl 2-(4-(bromoethynyl)benzamido)-3-((tert-butoxycarbonyl)amino)-3-methylbutanoate (INT-1) using Procedure 1. Subsequent deprotection of the BOC group using Procedure 2 (TFA 20 mL) and hydroxamate formation using Procedure 3 (NH₂OH 10 mL) afforded, after RP HPLC purification, (S)—N-(3-amino-1-(hydroxyamino)-3-methyl-1-oxobutan-2-yl)-4-(5-hydroxypenta-1,3-diynyl)benzamide (1) (114 mgs): MS: m/z calcd for C₁₇H₁₉N₃O₄ 329.14. found [M+H]⁺ 330.0.

| | | | | Sample |
|---|---|---|---|---|
| | | | Moles | Mass |
| Reactant | MW | Eq | (mmol) | (g) |
| butan-1-amine | 73.137 | 1.000 | 178 | 13.05 |
| hydroxylamine hydrochloride | 33.030 | 0.06 | 10.70 | 0.744 |
| copper(I) chloride | 98.999 | 0.02 | 3.57 | 0.353 |
| prop-2-yn-1-ol | 56.063 | 1.000 | 178 | 10 |
| (S)-methyl 2-(4- | 453.327 | 0.025 | 4.41 | 2 |
| (bromoethynyl)benzamido)-3-((tert- | | | | |
| butoxycarbonyl)amino)-3- | | | | |
| methylbutanoate (INT-1) | | | | |

### 2. (S)—N-(3-amino-1-(hydroxyamino)-3-methyl-1-oxobutan-2-yl)-4-(3-hydroxyprop-1-ynyl)benzamide (2)

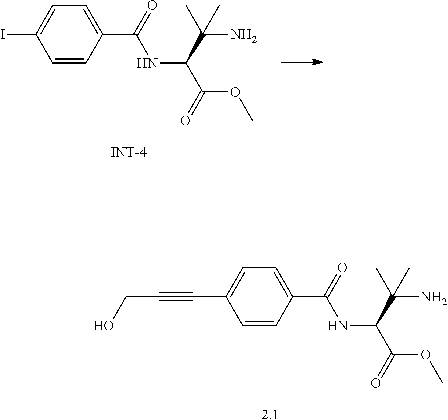 [SMILES: COC(=O)[C@@H](NC(=O)c1ccc(I)cc1)C(C)(C)N ; "INT-4" ; COC(=O)[C@@H](NC(=O)c1ccc(C#CCO)cc1)C(C)(C)N ; "2.1"]

To a stirring solution of compound INT-4 (0.5 g, 1.329 mmol) in THF (3.12 mL) were added PdCl₂(PPh₃)₂ (0.023 g, 0.033 mmol) and copper(I) iodide (0.013 g, 0.066 mmol), followed by propargyl alcohol (0.119 mL, 1.994 mmol) and TEA (1.042 mL), and the reaction was stirred at room temperature for 6 hours. The reaction was diluted with ethyl acetate, and the solids were removed by filtration. The organic layer was then concentrated under reduced pressure to yield compound 2.1 (0.527 g, 01.732 mmol) MS: m/z calcd for C₁₆H₂₀N₂O₄ 304.14. found [M+H]⁺ 305.2.

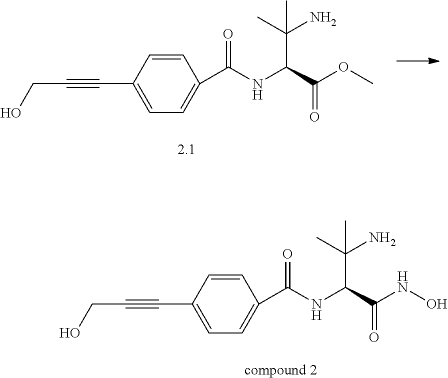

To a stirring solution of hydroxylamine hydrochloride (0.602 g, 8.66 mmol) in MeOH (3.19 mL) was added dropwise sodium methoxide (25% in methanol) (2.99 g, 13.85 mmol) and the reaction was stirred for one hour. A solution of compound 2.1 (0.527 g, 1.732 mmol) in MeOH (3.19 mL) was added to the above solution and the reaction mixture was placed in the freezer (−15° C.) for 18 hours. The reaction was then concentrated under reduced pressure to give a brown oil, which was purified by RP HPLC to yield (S)—N-(3-amino-1-(hydroxyamino)-3-methyl-1-oxobutan-2-yl)-4-(3-hydroxyprop-1-ynyl)benzamide (2, 0.025 g, 0.081 mmol, 5%). MS: m/z calcd for C₁₅H₁₉N₃O₄ 305.14. found [M+H]⁺ 306.2.

### 3. (S,E)-N-(3-amino-1-(hydroxyamino)-3-methyl-1-oxobutan-2-yl)-4-(5-hydroxypent-3-en-1-ynyl)benzamide (3)

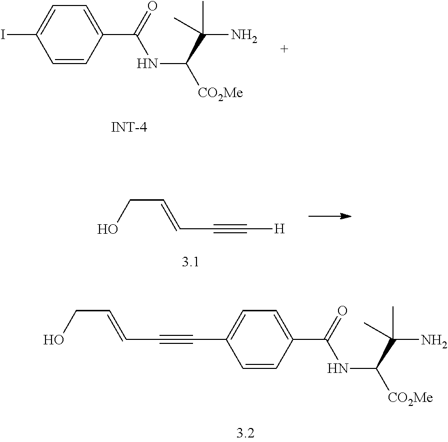 [SMILES: COC(=O)[C@@H](NC(=O)c1ccc(I)cc1)C(C)(C)N ; "INT-4" ; C#C/C=C/CO ; COC(=O)[C@@H](NC(=O)c1ccc(C#C/C=C/CO)cc1)C(C)(C)N]

(S)-methyl 3-amino-2-(4-iodobenzamido)-3-methylbutanoate (INT-4) (2.0 g, 5.32 mmol) was dissolved in THF (15 mL) and treated with (E)-pent-2-en-4-yn-1-ol (3.1) (436 mg, 5.32 mmol), copper(I) iodide (41 mg, 0.21 mmol), palladium(II) bis(triphenylphosphine)dichloride (75 mg, 0.11 mmol) followed by triethylamine (5.0 mL) at room temperature for 40 hours. The crude reaction was concentrated under reduced pressure, acidified with acetic acid and purified by RP HPLC (2″ column, 0.1% AcOH in water/ACN) to yield (S,E)-methyl 3-amino-2-(4-(5-hydroxypent-3-en-1-yn-1-yl)benzamido)-3-methylbutanoate (3.2) (900 mg, 2.7 mmol, 51%).

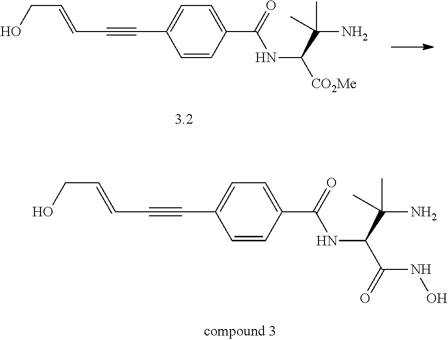 [SMILES: COC(=O)[C@@H](NC(=O)c1ccc(C#C/C=C/CO)cc1)C(C)(C)N ; CC(C)(N)[C@H](NC(=O)c1ccc(C#C/C=C/CO)cc1)C(=O)NO]

(S,E)-methyl 3-amino-2-(4-(5-hydroxypent-3-en-1-yn-1-yl)benzamido)-3-methylbutanoate (3.2) (900 mg, 2.7 mmol) was dissolved in IPA/50% aqueous hydroxylamine (1:1, 6 mL) and kept at 4° C. for 20 hours. The reaction mixture was acidified with acetic acid and purified by RP HPLC (2″ column, 0.1% AcOH in water/ACN) to yield (S,E)-N-(3-amino-1-(hydroxyamino)-3-methyl-1-oxobutan-2-yl)-4-(5-hydroxypent-3-en-1-yn-1-yl)benzamide as its acetate salt (3, 341 mg, 0.87 mmol, 32%): MS: m/z calcd for C₁₇H₂₁N₃O₄ 331.15. found [M+H]⁺ 332.1.

### 4. (S)—N-(3-hydroxy-1-(hydroxyamino)-3-methyl-1-oxobutan-2-yl)-4-(5-hydroxypenta-1,3-diynyl)benzamide (4)

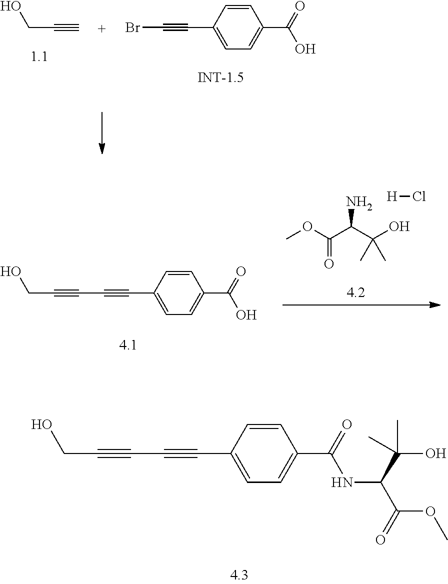

Compound 4.1 was synthesized from compounds 1.1 and INT-1.5 according to Procedure 1. 4-(5-Hydroxypenta-1,3-diyn-1-yl)benzoic acid (4.1) (280 mg, 1.4 mmol), (S)-methyl 2-amino-3-hydroxy-3-methylbutanoate hydrochloride (4.2, synthesized as described in WO 2008/154642 at page 247 et seq) (308 mg, 1.7 mmol) and HATU (638 mg, 1.7 mmol) were dissolved in DMF (3 mL). DIPEA (586 μL, 3.4 mmol) was added and the mixture was stirred for 100 minutes at ambient temperature. The reaction was partitioned between ethyl acetate and water. The organics were washed with 1M citric acid, saturated sodium bicarconate, saturated sodium chloride, dried over magnesium sulfate and concentrated under reduced pressure to give (S)-methyl 3-hydroxy-2-(4-(5-hydroxypenta-1,3-diyn-1-yl)benzamido)-3-methylbutanoate (4.3) (490 mg, 1.5 mmol), which was used without further purification.

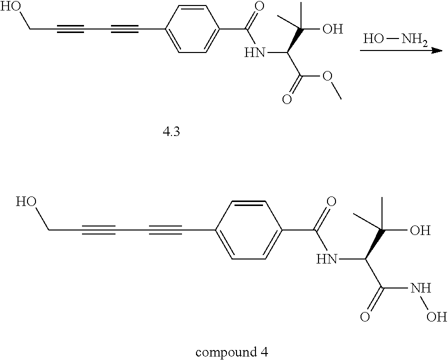

(S)-Methyl 3-hydroxy-2-(4-(5-hydroxypenta-1,3-diyn-1-yl)benzamido)-3-methylbutanoate (4.3) (490 mg, 1.5 mmol) was dissolved in IPA (5 mL) and 50% aqueous hydroxylamine (5 mL) was added. The mixture was kept at 4° C. for three days then concentrated under reduced pressure to give a residue, which was acidified with acetic acid (˜3 mL), diluted with water/ACN and purified by RP HPLC (2″ column, 0.1% TFA in water/ACN) to yield (S)—N-(3-hydroxy-1-(hydroxyamino)-3-methyl-1-oxobutan-2-yl)-4-(5-hydroxypenta-1,3-diyn-1-yl)benzamide (4, 182 mg, 0.55 mmol, 39% over 2 steps). MS: m/z calcd for C₁₇H₁₈N₂O₅ 330.12. found [M+H]+331.1. ¹H NMR (400 MHz, DMSO-d₆) δ 10.58 (s, 1H), 8.89 (br s, 1H), 8.09 (d, 1H, J=9.2 Hz), 7.86 (d, 2H, J=8.4 Hz), 7.63 (d, 2H, J=8.4 Hz), 5.47 (br s, 1H), 4.76 (br s, 1H), 4.35 (d, 1H, J=9.2 Hz), 4.24 (s, 2H), 1.16 (s, 3H), 1.12 (s, 3H).

### 5. (S)—N-(1-(hydroxyamino)-3-methyl-3-(methylamino)-1-oxobutan-2-yl)-4-(5-hydroxypenta-1,3-diynyl)benzamide (5)

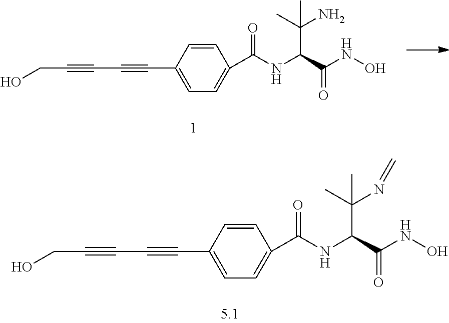 [SMILES: CC(C)(N)[C@H](NC(=O)c1ccc(C#CC#CCO)cc1)C(=O)NO ; C=NC(C)(C)[C@H](NC(=O)c1ccc(C#CC#CCO)cc1)C(=O)NO]

With (S)—N-(3-amino-1-(hydroxyamino)-3-methyl-1-oxobutan-2-yl)-4-(5-hydroxypenta-1,3-diyn-1-yl)benzamide (1) as the incoming amine, the reaction was carried out according to Procedure 4A to yield compound 5.1 (3.96 g), which was carried through to the next step without further purification. MS: m/z calcd for C₁₈H₁₉N₃O₄ 341.14. found [M+H]⁺ 342.2.

| | | | | | Sam- | | |
|---|---|---|---|---|---|---|---|
| | | | | | ple | | % |
| | | | Density | Moles | Mass | Vol | Wt |
| Reactant | MW | Eq | (g/mL) | (mmol) | (g) | (mL) | (%) |
| (S)-N-(3-amino-1- | 329.35 | 1.000 | | 7.20 | 2.37 | | |
| (hydroxyamino)- | | | | | | | |
| 3-methyl-1- | | | | | | | |
| oxobutan-2-yl)-4- | | | | | | | |
| (5-hydroxypenta- | | | | | | | |
| 1,3-diyn-1- | | | | | | | |
| yl)benzamide (1) | | | | | | | |
| Hunig'sBase | 129.24 | 1.5 | 0.74 | 10.79 | 1.395 | 1.885 | |
| formaldehyde | 30.03 | 2 | 1.09 | 14.39 | 1.168 | 1.071 | 37 |
| (37% in water) | | | | | | | |
| Butylamine | 73.14 | 1.5 | | 10.79 | 2.63 | | 30 |
| (30% in water) | | | | | | | |

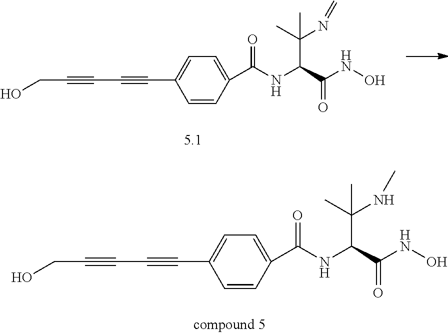 [SMILES: C=NC(C)(C)[C@H](NC(=O)c1ccc(C#CC#CCO)cc1)C(=O)NO ; CNC(C)(C)[C@H](NC(=O)c1ccc(C#CC#CCO)cc1)C(=O)NO]

The next step was carried out according to Procedure 4B to yield the crude amine, which was purified on a 2-inch RP HPLC to yield (S)—N-(1-(hydroxyamino)-3-methyl-3-(methylamino)-1-oxobutan-2-yl)-4-(5-hydroxypenta-1,3-diynyl)benzamide (5, 0.264 g, 0.687 mmol, 6%). MS: m/z calcd for C₁₈H₂₁N₃O₄ 343.15. found [M+H]⁺ 344.2.

| | | | Den- | | Sample | |
|---|---|---|---|---|---|---|
| | | | sity | Moles | Mass | Vol |
| Reactant | MW | Eq | (g/mL) | (mmol) | (g) | (mL) |
| (S)-N-(1- | 341.36 | 1.000 | | 11.60 | 3.96 | |
| (hydroxyamino)- | | | | | | |
| 3-methyl-3- | | | | | | |
| (methyleneamino)- | | | | | | |
| 1-oxobutan-2-yl)-4- | | | | | | |
| (5-hydroxypenta- | | | | | | |
| 1,3-diyn-1- | | | | | | |
| yl)benzamide | | | | | | |
| acetic acid | 60.05 | 2 | 1.05 | 23.20 | 1.393 | 1.328 |
| sodium | 39.85 | 15 | | 174 | 10.94 | |
| cyanoborohydride | | | | | | |

### 6. (S)—N-(1-(hydroxyamino)-3-(2-hydroxyethylamino)-3-methyl-1-oxobutan-2-yl)-4-(5-hydroxypenta-1,3-diynyl)benzamide (6)

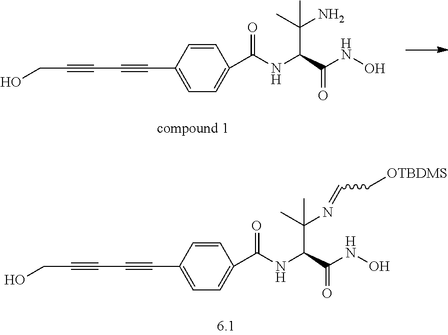 [SMILES: CC(C)(N)[C@H](NC(=O)c1ccc(C#CC#CCO)cc1)C(=O)NO ; CC(C)(N=CCO[Si](C)(C)C(C)(C)C)[C@H](NC(=O)c1ccc(C#CC#CCO)cc1)C(=O)NO]

With (S)—N-(3-amino-1-(hydroxyamino)-3-methyl-1-oxobutan-2-yl)-4-(5-hydroxypenta-1,3-diyn-1-yl)benzamide (1) as the incoming amine, the reaction was carried out according to Procedure 4A to yield compound 6.1 (5.13 g), which was carried through to the next step without further purification. MS: m/z calcd for C₂₅H₃₅N₃O₅Si 485.23. found [M+H]⁺ 486.3.

| | | | Density | Moles | Sample | Vol | % Wt |
|---|---|---|---|---|---|---|---|
| Reactant | MW | Eq | (g/mL) | (mmol) | Mass (g) | (mL) | (%) |
| (S)-N-(3-amino-1- | 329.35 | 1.000 | | 7.83 | 2.58 | | |
| (hydroxyamino)-3- | | | | | | | |
| methyl-1-oxobutan-2-yl)-4-(5- | | | | | | | |
| hydroxypenta-1,3-diyn- | | | | | | | |
| 1-yl)benzamide (1) | | | | | | | |
| Hunig'sBase | 129.24 | 1.5 | 0.74 | 11.75 | 1.519 | 2.052 | |
| (tert- | 174.31 | 2 | 0.91 | 15.67 | 2.73 | 3.00 | |
| butyldimethylsilyloxy)acetaldehyde | | | | | | | |
| Butylamine (30% in water) | 73.14 | 2 | | 15.67 | 3.82 | | 30 |

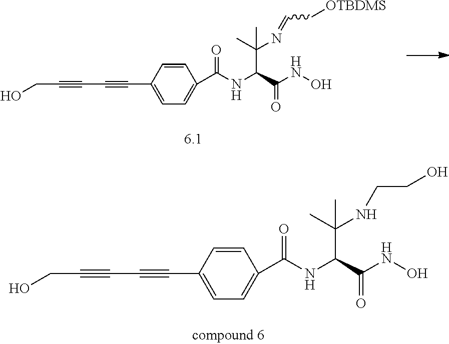 [SMILES: CC(C)(N=CCO[Si](C)(C)C(C)(C)C)[C@H](NC(=O)c1ccc(C#CC#CCO)cc1)C(=O)NO ; CC(C)(NCCO)[C@H](NC(=O)c1ccc(C#CC#CCO)cc1)C(=O)NO]

To a stirring solution of 6.1 (5.13 g, 10.56 mmol) in THF (21.10 mL) and MeOH (2.221 mL) was added acetic acid (1.814 mL, 31.7 mmol) followed by sodium cyanoborohydride (6.64 g, 106 mmol) and the reaction was stirred for 1 hour. Next TFA (81 mL, 1056 mmol) was added and the reaction was stirred for 1 hour. The reaction mixture was concentrated under reduced pressure to give an orange oil, which was purified by RP HPLC to yield (S)—N-(1-(hydroxyamino)-3-(2-hydroxyethylamino)-3-methyl-1-oxobutan-2-yl)-4-(5-hydroxypenta-1,3-diynyl)benzamide (6) (0.058 g, 0.151 mmol, 1.4%). MS: m/z calcd for C₁₉H₂₃N₃O₅ 373.16. found [M+H]⁺ 374.1.

### 7. (S)—N-(3-amino-1-(hydroxyamino)-3-methyl-1-oxobutan-2-yl)-4-(6-hydroxyhexa-1,3-diynyl)benzamide (7)

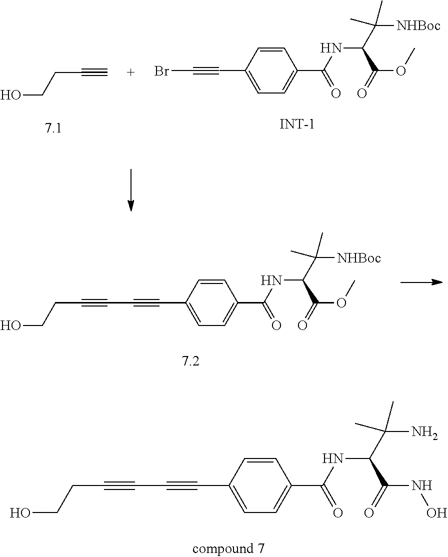

But-3-yn-1-ol (7.1) was coupled with (S)-methyl 2-(4-(bromoethynyl)benzamido)-3-((tert-butoxycarbonyl)amino)-3-methylbutanoate (INT-1) using Procedure 1. Subsequent deprotection of BOC group, using Procedure 2, and hydroxamate formation, using Procedure 3, yielded after RP-HPLC (S)—N-(3-amino-1-(hydroxyamino)-3-methyl-1-oxobutan-2-yl)-4-(6-hydroxyhexa-1,3-diyn-1-yl)benzamide (7, 3.2 g): MS: m/z calcd for C₁₈H₂₁N₃O₄ 343.15. found [M+H]⁺ found 344.1.

| | | | Moles | Sample | | % Wt |
|---|---|---|---|---|---|---|
| Reactant | MW | Eq | (mmol) | Mass (g) | Vol (mL) | (%) |
| TFA | 114.023 | 5 | 463 | 52.8 | 35.7 | |
| hydroxylamine hydrochloride | 33.030 | 0.1 | 9.26 | 0.644 | | |
| hydroxylamine | 33.030 | 20 | 1853 | 122 | | 50 |
| copper(I) chloride | 98.999 | 0.02 | 1.853 | 0.183 | | |
| but-3-yn-1-o (7.1)I | 70.090 | 1.000 | 93 | 6.49 | | |
| (S)-methyl | 453.327 | 1.000 | 93 | 42 | | |
| 2-(4-(bromoethynyl)benzamido)- | | | | | | |
| 3-((tert-butoxycarbonyl)amino)-3- | | | | | | |
| methylbutanoate (INT-1) | | | | | | |

### 8. (S)—N-(3-amino-1-(hydroxyamino)-3-methyl-1-oxobutan-2-yl)-4-(4-hydroxybut-1-ynyl)benzamide (8)

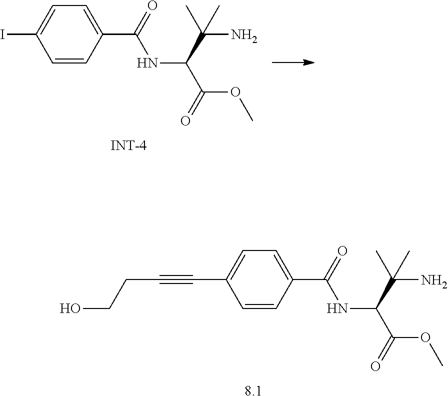 [SMILES: COC(=O)[C@@H](NC(=O)c1ccc(I)cc1)C(C)(C)N ; "INT-4" ; COC(=O)[C@@H](NC(=O)c1ccc(C#CCCO)cc1)C(C)(C)N ; "8.1"]

To a stirring solution of INT-4 (0.5 g, 1.329 mmol) in THF (3.12 mL) were added PdCl₂(PPh₃)₂ (0.023 g, 0.033 mmol) and copper(I) iodide (0.013 g, 0.066 mmol), followed by 3-butyn-1-ol (0.151 mL, 1.994 mmol) and TEA (1.042 mL), and the reaction was stirred at room temperature for 1 hour. The reaction was diluted with ethyl acetate (8 mL), the solids were removed by filtration, and the filtrate was concentrated under reduced pressure to yield compound 8.1 (0.423 g, 1.33 mmol, 100%). MS: m/z calcd for C₁₇H₂₂N₂O₄ 318.16. found [M+H]⁺ 319.2.

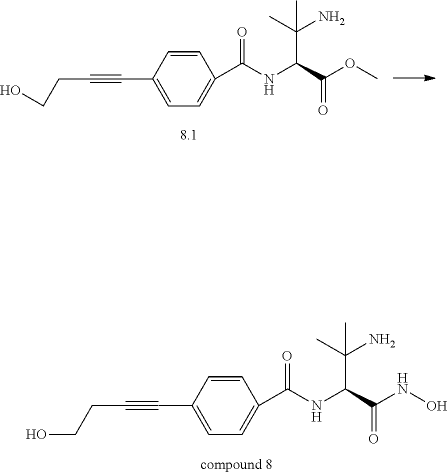

To a stirring solution of compound 8.1 (0.588 g, 1.847 mmol) in IPA (1 mL) was added dropwise 50% hydroxylamine (4.9 g, 35.4 mmol), and the reaction was stirred for 3 hours. The reaction was extracted with methyl-THF (2×50 mL), and the combined organic layers were concentrated under reduced pressure to yield a crude, which was purified by RP HPLC to yield (S)—N-(3-amino-1-(hydroxyamino)-3-methyl-1-oxobutan-2-yl)-4-(4-hydroxybut-1-ynyl)benzamide (8, 0.23 g, 0.718 mmol, 39%). MS: m/z calcd for C₁₆H₂₁N₃O₄ 319.15. found [M+H]⁺ 320.2.

### 9. (S)—N-(1-(hydroxyamino)-3-methyl-3-(methylamino)-1-oxobutan-2-yl)-4-(6-hydroxyhexa-1,3-diynyl)benzamide (9)

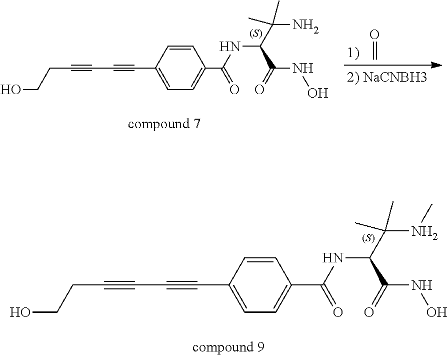

(S)—N-(3-amino-1-(hydroxyamino)-3-methyl-1-oxobutan-2-yl)-4-(6-hydroxyhexa-1,3-diyn-1-yl)benzamide (7) was treated with formaldehyde using Procedure 4A and the resulting imine was reduced using Procedure 4B, followed by purification by RP HPLC to yield (S)—N-(1-(hydroxyamino)-3-methyl-3-(methylamino)-1-oxobutan-2-yl)-4-(6-hydroxyhexa-1,3-diyn-1-yl)benzamide (9, 0.994 g): MS: m/z calcd for C₁₉H₂₃N₃O₄ 357.17. found [M+H]⁺ 358.2.

| | | | | Sample |
|---|---|---|---|---|
| | | | Moles | Mass |
| Reactant | MW | Eq | (mmol) | (g) |
| TEA | 101.19 | 1.000 | 11.65 | 1.179 |
| sodium cyanoborohydride | 39.85 | 1.000 | 11.65 | 0.732 |
| (S)-N-(3-amino-1-(hydroxyamino)- | 343.38 | 1.000 | 11.65 | 4 |
| 3-methyl-1-oxobutan-2-yl)-4-(6- | | | | |
| hydroxyhexa-1,3-diynyl)benzamide | | | | |
| (7) | | | | |
| formaldehyde | 30.03 | 1.000 | 11.65 | 0.350 |

### 10. (S)—N-(3-(ethylamino)-1-(hydroxyamino)-3-methyl-1-oxobutan-2-yl)-4-(6-hydroxyhexa-1,3-diynyl)benzamide (10)

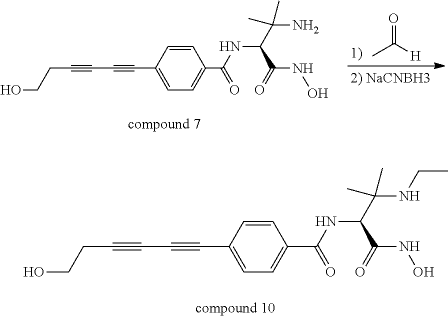

(S)—N-(3-amino-1-(hydroxyamino)-3-methyl-1-oxobutan-2-yl)-4-(6-hydroxyhexa-1,3-diyn-1-yl)benzamide (7) (72 mgs) was treated with acetaldehyde using Procedure 4A and the resulting imine was reduced using Procedure 4B. Purification by RP HPLC afforded (S)—N-(3-(ethylamino)-1-(hydroxyamino)-3-methyl-1-oxobutan-2-yl)-4-(6-hydroxyhexa-1,3-diyn-1-yl)benzamide (10, 0.015 g). MS: m/z calcd for C₂₀H₂₅N₃O₄ 371.18. found [M+H]⁺ 372.2.

| | | | Sample |
|---|---|---|---|
| | | Moles | Mass |
| Reactant | MW | (mmol) | (mg) |
| sodium cyanoborohydride | 39.853 | 3.15 | 198 |
| acetaldehyde | 44.053 | 0.210 | 9.24 |
| (S)-N-(3-amino-1-(hydroxyamino)-3-methyl- | 343.377 | 0.210 | 72 |
| 1-oxobutan-2-yl)-4-(6-hydroxyhexa-1,3- | | | |
| diyn-1-yl)benzamide (7) | | | |

### 11. (S)—N-(1-(hydroxyamino)-3-(2-hydroxyethylamino)-3-methyl-1-oxobutan-2-yl)-4-(6-hydroxyhexa-1,3-diynyl)benzamide (11)

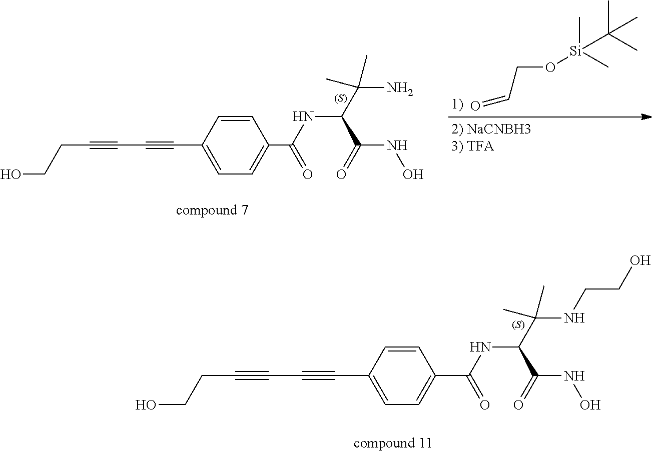

(S)—N-(3-amino-1-(hydroxyamino)-3-methyl-1-oxobutan-2-yl)-4-(6-hydroxyhexa-1,3-diyn-1-yl)benzamide (7) was treated with 2-((tert-butyldimethylsilyl)oxy)acetaldehyde using Procedure 4A and the resulting imine was reduced using Procedure 4B, deprotection with TFA, and purification by RP HPLC afforded (S)—N-(1-(hydroxyamino)-3-((2-hydroxyethyl)amino)-3-methyl-1-oxobutan-2-yl)-4-(6-hydroxyhexa-1,3-diyn-1-yl)benzamide (11, 1.25 g): MS: m/z calcd for C₂₀H₂₅N₃O₅ 387.18. found [M+H]⁺ 388.2.

| | | | | Sample |
|---|---|---|---|---|
| | | | Moles | Mass |
| Reactant | MW | Eq | (mmol) | (g) |
| TEA | 101.19 | 1.000 | 11.07 | 1.120 |
| sodium cyanoborohydride | 39.85 | 1.000 | 11.07 | 0.695 |
| (S)-N-(3-amino-1-(hydroxyamino)-3- | 343.38 | 1.000 | 11.07 | 3.8 |
| methyl-1-oxobutan-2-yl)-4-(6- | | | | |
| hydroxyhexa-1,3- | | | | |
| diynyl)benzamide (7) | | | | |
| 2-(tert- | 174.31 | 1.000 | 11.07 | 1.929 |
| butyldimethylsilyloxy)acetaldehyde | | | | |

### 12. (S)—N-(1-(hydroxyamino)-3-methyl-3-((5-methylisoxazol-3-yl)methylamino)-1-oxobutan-2-yl)-4-(6-hydroxyhexa-1,3-diynyl)benzamide (12)

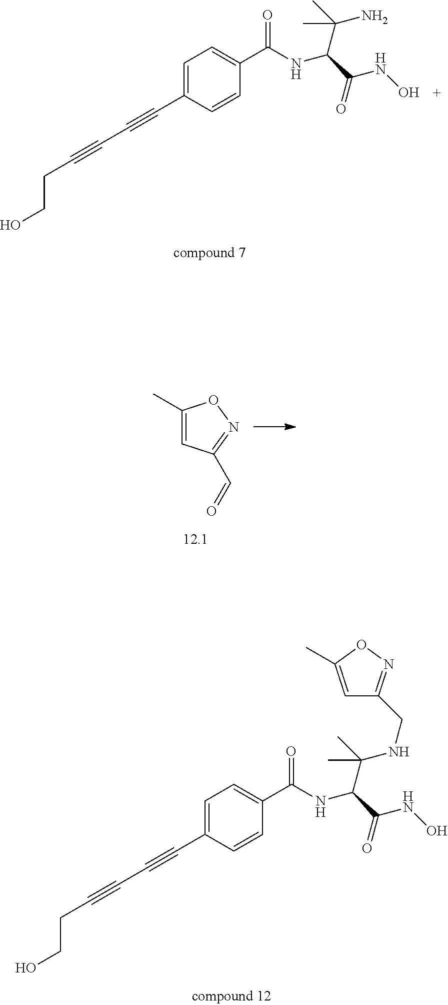

(S)—N-(3-amino-1-(hydroxyamino)-3-methyl-1-oxobutan-2-yl)-4-(6-hydroxyhexa-1,3-diyn-1-yl)benzamide (7) (25 mg, 0.062 mmol) was dissolved in DMF (0.2 mL) and then DIPEA (0.022 mL, 0.124 mmol) and 5-methylisoxazole-3-carbaldehyde (12.1) (68.8 mg, 0.620 mmol) were added at room temperature. After 2.5 h, NaCNBH₃ (38.9 mg, 0.620 mmol), methanol (0.200 mL) and acetic acid (0.032 mL, 0.558 mmol) were added. After 45 min, TFA (0.043 mL, 0.558 mmol) was added and the reaction was complete after 20 min. The crude material was purified (RP-HPLC, 0.1% TFA in water/ACN) to give (S)—N-(1-(hydroxyamino)-3-methyl-3-(((5-methylisoxazol-3-yl)methyl)amino)-1-oxobutan-2-yl)-4-(6-hydroxyhexa-1,3-diyn-1-yl)benzamide (12) (5 mg, 9.05 μmol, 14.60% yield). MS: m/z calcd for C₂₃H₂₅N₄O₅ 438.19. found [M+H]⁺ 339.3.

### 13. (S)—N-(3-(1H-imidazol-4-yl)methylamino)-1-(hydroxyamino)-3-methyl-1-oxobutan-2-yl)-4-(6-hydroxyhexa-1,3-diynyl)benzamide (13)

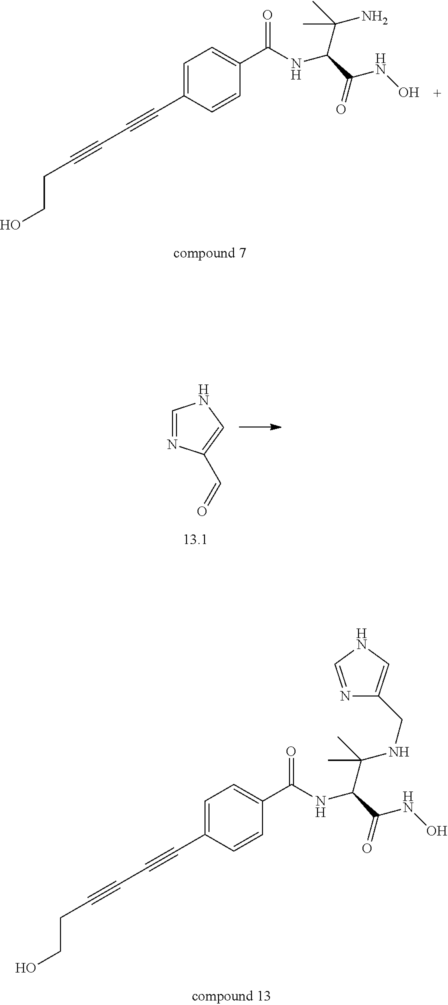 [SMILES: CC(C)(N)[C@H](NC(=O)c1ccc(C#CC#CCCO)cc1)C(=O)NO ; O=Cc1c[nH]cn1 ; CC(C)(NCc1c[nH]cn1)[C@H](NC(=O)c1ccc(C#CC#CCCO)cc1)C(=O)NO]

(S)—N-(3-amino-1-(hydroxyamino)-3-methyl-1-oxobutan-2-yl)-4-(6-hydroxyhexa-1,3-diyn-1-yl)benzamide (7) (25 mg, 0.062 mmol) was dissolved in DMF (0.2 mL) and then DIPEA (0.022 mL, 0.124 mmol) and 1H-imidazole-4-carbaldehyde (13.1) (59.5 mg, 0.620 mmol) were added at room temperature. After 2.5 h, NaCNBH₃ (38.9 mg, 0.620 mmol), methanol (0.200 mL) and acetic acid (0.032 mL, 0.558 mmol) were added. After 1 hr, TFA (0.043 mL, 0.558 mmol) was added and the reaction was complete after 10 min. The crude material was purified (RP HPLC, 0.1% TFA in water/ACN) to give (S)—N-(3-(((1H-imidazol-4-yl)methyl)amino)-1-(hydroxyamino)-3-methyl-1-oxobutan-2-yl)-4-(6-hydroxyhexa-1,3-diyn-1-yl)benzamide (13) (5 mg, 0.012 mmol, 19.05% yield). MS: m/z calcd for C₂₂H₂₅N₅O₄ 423.19. found [M+H]⁺ 424.4.

### 14. N—((S)-3-amino-1-(hydroxyamino)-3-methyl-1-oxobutan-2-yl)-4-((S)-5-hydroxyhexa-1,3-diynyl)benzamide (14)

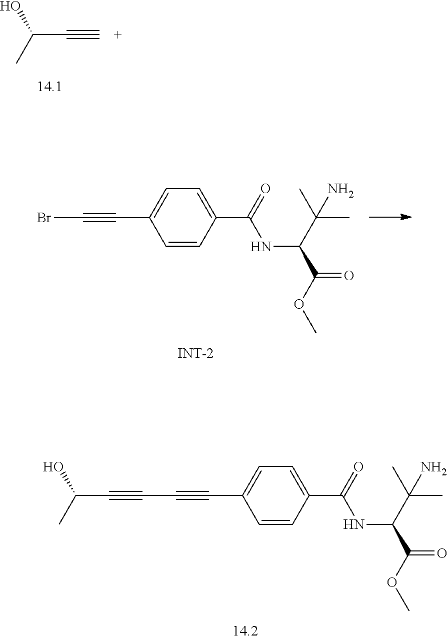

A solution of hydroxylamine hydrochloride (0.186 g, 2.68 mmol) and copper(I) chloride (0.088 g, 0.893 mmol) in 23% n-butylamine/water (40 mL) was cooled in an ice bath. To this was added a 23% n-butylamine/water solution (100 mL) containing (S)-but-3-yn-2-ol (14.1) (3.87 mL, 49.1 mmol) while maintaining the temperature at 0-5° C. A yellow precipitate formed. Next, a solution of (S)-methyl 3-amino-2-(4-(bromoethynyl)benzamido)-3-methylbutanoate hydrochloride (INT-2) (17.4 g, 44.7 mmol), methanol (42 mL) and hydroxylamine hydrochloride (0.186 g, 2.68 mmol) in 23% aqueous n-butylamine solution (100 mL) was added via addition funnel over 30 min. The reaction mixture was stirred for 1.5 hours in the ice bath. The reaction was extracted with ethyl acetate (300 mL) and the organic layer was washed with 1:1 water/brine (200 mL) and dried with sodium sulfate, filtered and concentrated under reduced pressure to provide (S)-methyl 3-amino-2-(4-((S)-5-hydroxyhexa-1,3-diynyl)benzamido)-3-methylbutanoate (14.2) (12.6 g, 36.8 mmol, 82%). MS: m/z calcd for C₁₉H₂₂N₂O₄ 342.16. found [M+H]⁺ 343.2.

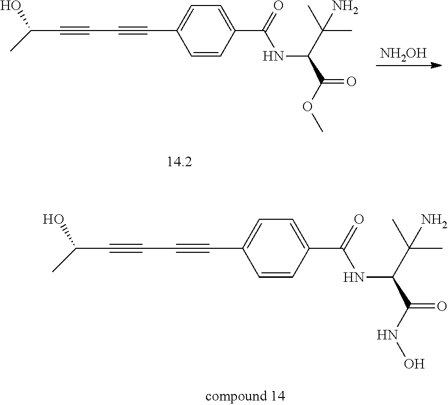

(S)-methyl 3-amino-2-(4-((S)-5-hydroxyhexa-1,3-diynyl)benzamido)-3-methylbutanoate (14.2) (12.6 g, 36.8 mmol) was dissolved in IPA (25 mL) and 50% hydroxylamine in water (49 mL) and kept at 4° C. overnight. The reaction was concentrated under reduced pressure, and the resulting solution was acidified with AcOH (60 mL), purified by RP-HPLC (water/MeOH with 0.1% AcOH) to give N—((S)-3-amino-1-(hydroxyamino)-3-methyl-1-oxobutan-2-yl)-4-((S)-5-hydroxyhexa-1,3-diynyl)benzamide (14) (2.99 g, 7.41 mmol, 20.1%). MS: m/z calcd for C₁₈H₂₁N₃O₄ 343.15. found [M+H]⁺ 344.2; ¹H NMR (400 MHz, DMSO-d₆) δ 7.86 (d, 2H), 7.63 (d, 2H), 4.53 (q, 1H), 4.29 (s, 1H), 1.86 (s, 2.3H*), 1.33 (d, 3H), 1.09 (s, 3H), 1.01 (s, 3H). *Acetate signal. The compound contains a substoichiometric amount of acetate.

### 15. N—((S)-3-amino-1-(hydroxyamino)-3-methyl-1-oxobutan-2-yl)-4-((R)-5-hydroxyhexa-1,3-diynyl)benzamide (15)

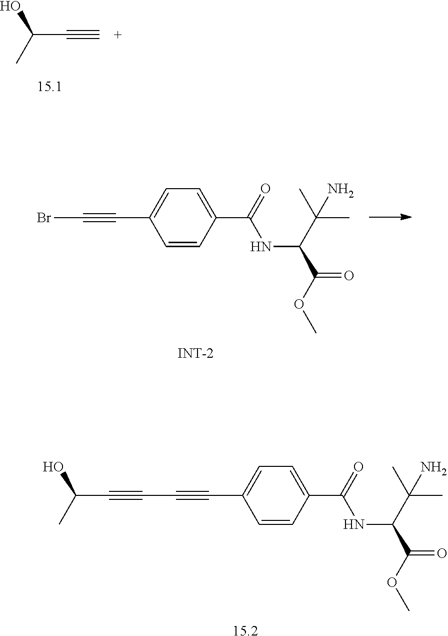

A solution of hydroxylamine hydrochloride (0.117 g, 1.678 mmol) and copper(I) chloride (0.055 g, 0.559 mmol) in 23% n-butylamine/water (25 mL) was cooled in an ice bath. To this was added a 23% n-butylamine/water solution (62 mL) containing (R)-but-3-yn-2-ol (15.1) (2.423 mL, 30.8 mmol) while keeping the temperature at 0-5° C. A yellow precipitate formed. A solution of, (S)-methyl 3-amino-2-(4-(bromoethynyl)benzamido)-3-methylbutanoate hydrochloride (INT-2) (10.9 g, 28.0 mmol), methanol (26 mL) and hydroxylamine hydrochloride (0.117 g, 1.678 mmol) in 23% n-butylamine/water solution (62 mL) was added via addition funnel over 30 min. The reaction mixture was stirred for 1 hour in the ice bath. The reaction was extracted with ethyl acetate (300 mL) and the organic layer was washed with 1:1 water/brine (200 mL) and dried with sodium sulfate, filtered and concentrated under reduced pressure to provide (S)-methyl 3-amino-2-(4-((R)-5-hydroxyhexa-1,3-diynyl)benzamido)-3-methylbutanoate (15.2) (7.68 g, 22.43 mmol, 80%). MS: m/z calcd for C₁₉H₂₂N₂O₄ 342.16. found [M+H]⁺ 343.2.

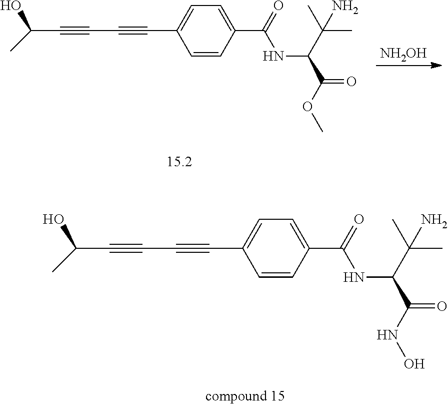

(S)-methyl 3-amino-2-(4-((R)-5-hydroxyhexa-1,3-diynyl)benzamido)-3-methylbutanoate (15.2) (7.68 g, 22.43 mmol) was dissolved in IPA (15 mL), then 50% hydroxylamine in water (30 mL, 29.6 g, 449 mmol) was added. The slightly cloudy mixture was stirred at room temperature. After 5 hours, the reaction was diluted with brine (15 mL) and extracted with 20% MeOH/MeTHF (2×50 mL). The combined organic layers were dried over sodium sulfate, filtered and concentrated under reduced pressure to give a crude, which was purified (RP-HPLC, with 0.1% acetic acid in water (A) and 0.1% acetic acid in methanol) to provide N—((S)-3-amino-1-(hydroxyamino)-3-methyl-1-oxobutan-2-yl)-4-((R)-5-hydroxyhexa-1,3-diynyl)benzamide (15) (1.17 g, 2.91 mmol, 13.0%). MS: m/z calcd for C₁₈H₂₁N₃O₄ 343.15. found [M+H]⁺ 344.2; ¹H NMR (400 MHz, DMSO-d₆) δ 7.87 (d, 2H), 7.64 (d, 2H), 4.53 (q, 1H), 4.35 (s, 1H), 1.88 (s, 1.9H*), 1.33 (d, 3H), 1.12 (s, 3H), 1.04 (s, 3H). *Acetate signal. The compound contains a substoichiometric amount of acetate.

### 16. N—((S)-1-(hydroxyamino)-3-methyl-3-(methylamino)-1-oxobutan-2-yl)-4-((S)-5-hydroxyhexa-1,3-diynyl)benzamide (16)

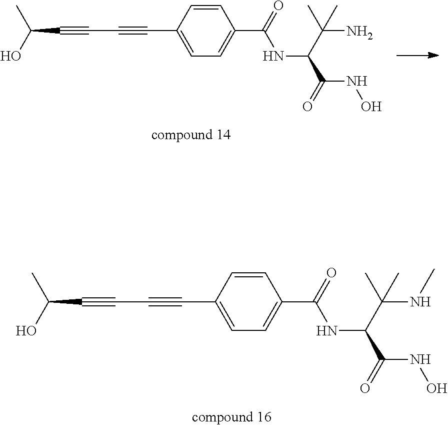 [SMILES: C[C@H](O)C#CC#Cc1ccc(C(=O)N[C@H](C(=O)NO)C(C)(C)N)cc1 ; CNC(C)(C)[C@H](NC(=O)c1ccc(C#CC#C[C@H](C)O)cc1)C(=O)NO]

To a stirring solution of N—((S)-3-amino-1-(hydroxyamino)-3-methyl-1-oxobutan-2-yl)-4-((S)-5-hydroxyhexa-1,3-diyn-1-yl)benzamide (14) (400 mg, 1.17 mmol) in THF (1.7 mL) was added formaldehyde (1.6 mL, 17.47 mmol, 37% in water). The reaction was stirred for 15 minutes before volatiles were removed under reduced pressure. The residue was re-slurried in THF (1.7 mL) and treated with n-butylamine (3.5 mL, 34.9 mmol) for 15 minutes. The volatiles were again removed under reduced pressure and the upper oily layer was discarded. The residue was re-slurried in THF (1.7 mL), treated with AcOH (1 mL) and NaCNBH₃ (150 mg, 2.33 mmol) for 1 hr. The reaction mixture was concentrated under reduced pressure, dissolved in water (5 mL) and purified by RP HPLC (0-30% ACN in water) to provide N—((S)-1-(hydroxyamino)-3-methyl-3-(methylamino)-1-oxobutan-2-yl)-4-((S)-5-hydroxyhexa-1,3-diynyl)benzamide (16) (152 mg, 0.364 mmol, 31%). MS m/z calcd for C₁₉H₂₃N₃O₄ 357.17. found [M+H]⁺358.2; ¹H NMR (400 MHz, DMSO-d₆) δ 11.4 (s, 1H), 9.23 (s, 1H), 8.66 (d, 1H), 7.95 (d, 2H), 7.67 (d, 2H), 5.65 (s, 1H), 4.84 (d, 1H), 4.52 (q, 1H), 3.35 (s, 1H), 2.53 (s, 3H), 1.36 (s, 3H), 1.33 (d, 3H), 1.27 (s, 3H).

### 17. N—((S)-3-amino-1-(hydroxyamino)-3-methyl-1-oxobutan-2-yl)-4-(5,6-dihydroxyhexa-1,3-diynyl)benzamide (17)

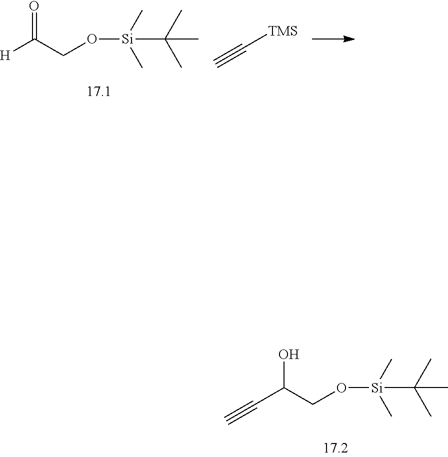

In a 500 mL round-bottomed flask was charged ethynyltrimethylsilane (8.45 g, 86 mmol) and N¹,N¹,N²,N²-tetramethylethane-1,2-diamine (10.00 g, 86 mmol) in dry THF (100 mL) to give a colorless solution. n-Butyllithium (60 mL, 95 mmol) was added at −78° C. over 10 min and stirring was continued for 30 min. 2-((tert-butyldimethylsilyl)oxy)acetaldehyde (17.1) (15 g, 86 mmol) in THF (15 mL) was added and stirring was continued for an additional 3 hr. The reaction mixture was quenched with saturated aq NH₄Cl (30 mL) at −78° C., and the product was extracted with ethyl acetate (2×250 mL), dried over Na₂SO₄ and concentrated under reduced pressure. The crude product (18.7 g) was treated with aqueous ammonia (25 mL) in methanol (25 mL) for 18 h. The resulting product was extracted with EtOAc (2×200 mL) and concentrated under reduced pressure to give 1-((tert-butyldimethylsilyl)oxy)but-3-yn-2-ol (17.2) (14.2 g), which was used in the next step without any further purification.

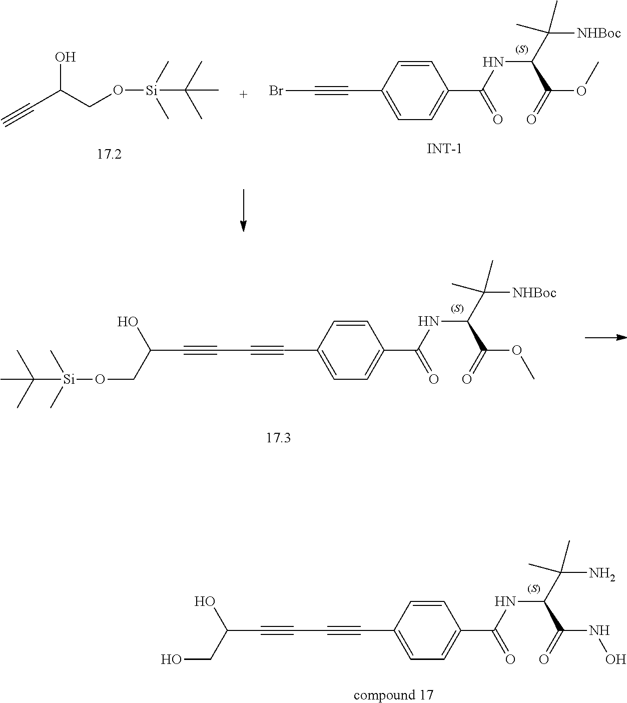

1-((tert-butyldimethylsilyl)oxy)but-3-yn-2-ol (17.2) was coupled with (S)-methyl 2-(4-(bromoethynyl)benzamido)-3-((tert-butoxycarbonyl)amino)-3-methylbutanoate (INT-1) using Procedure 1 to give (2S)-methyl 3-((tert-butoxycarbonyl)amino)-2-(4-(6-((tert-butyldimethylsilyl)oxy)-5-hydroxyhexa-1,3-diyn-1-yl)benzamido)-3-methylbutanoate (17.3). Subsequent deprotection of BOC using Procedure 2 (TFA 20 mL) and hydroxamate formation using Procedure 3 gave after RP HPLC purification N—((S)-3-amino-1-(hydroxyamino)-3-methyl-1-oxobutan-2-yl)-4-(5,6-dihydroxyhexa-1,3-diyn-1-yl)benzamide (17) (2.76 g): MS: m/z calcd for C₁₈H₂₁N₃O₅ 359.15. found [M+H]⁺ 360.2.

| | | | | Sample |
|---|---|---|---|---|
| | | | Moles | Mass |
| Reactant | MW | Eq | (mmol) | (g) |
| hydroxylamine hydrochloride | 33.030 | 1.000 | 32.4 | 2.255 |
| CuCl | 98.999 | 0.02 | 0.649 | 0.064 |
| 1-((tert- | 200.350 | 1 | 32.4 | 6.5 |
| butyldimethylsilyl)oxy)but- | | | | |
| 3-yn-2-ol | | | | |
| (S)-methyl 2-(4- | 453.327 | 1.256 | 40.7 | 18.47 |
| (bromoethynyl)benzamido)-3- | | | | |
| ((tert-butoxycarbonyl)amino)-3- | | | | |
| methylbutanoate (INT-1) | | | | |
| hydroxylamine | 33.030 | 40 | 1298 | 86 |

### 18. (S)—N-(3-amino-1-(hydroxyamino)-3-methyl-1-oxobutan-2-yl)-4-(6-hydroxy-5-(hydroxymethyl)hexa-1,3-diynyl)benzamide (18)

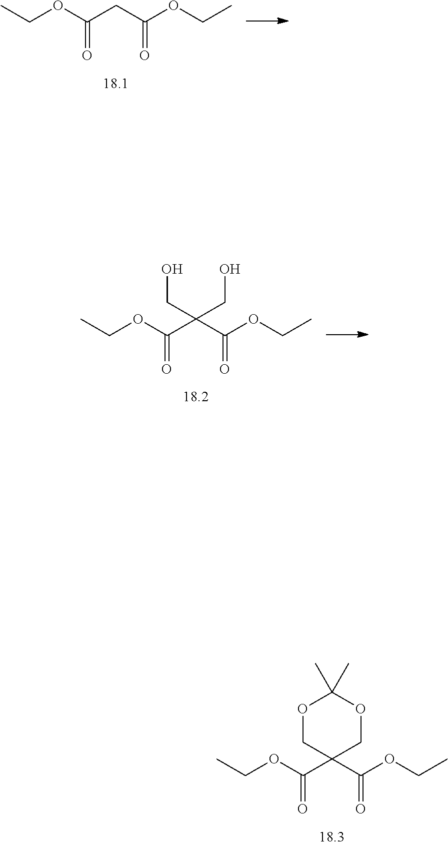 [SMILES: CCOC(=O)CC(=O)OCC ; CCOC(=O)C(CO)(CO)C(=O)OCC ; CCOC(=O)C1(C(=O)OCC)COC(C)(C)OC1]

To a solution of diethyl malonate (18.1) (48.3 g, 0.3 mol) in 37% HCHO (164.8 g), was added Na₂CO₃ (2.0 g) and the mixture was stirred at rt for 10 hours. The progress of the reaction was monitored by GCMS. The excess HCHO was removed under reduced pressure, and the residue was extracted with DCM (3×200 mL). The combined organic layers were dried and concentrated under reduced pressure to give a yellow oil, which was purified by flash chromatography (silica gel/PE:EA=5:1 to 2:1) to yield 18.2 (36.0 g, 55%). ¹H NMR (400 MHz, DMSO-d₆) δ 4.86 (t, J=5.2 Hz, 2H), 4.06 (m, 4H), 3.84 (m, 4H), 1.14 (t, J=0.6 Hz, 6H).

To a stirring solution of 18.2 (33.0 g, 150 mmoL) in 2,2-dimethoxypropane (200 mL), was added p-TsOH (2.58 g, 15 mmoL) and the reaction mixture was kept stirring at 80° C. for 6 hours. The solvent was removed under reduced pressure to yield 18.3 (32.0 g) as a yellow oil (82%).

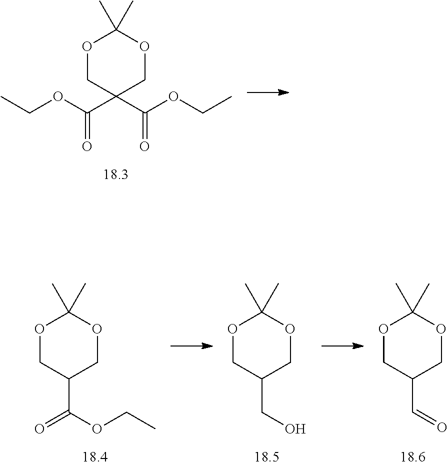

To a stirring solution of 18.3 (32.0 g, 123.0 mmoL) in DMSO (350 mL) was added NaCl (7.20 g, 123.0 mmoL), and H₂O (4.43 g, 246.0 mmoL) and the reaction mixture was heated to 180° C. for 48 hour. The mixture was cooled to r.t, diluted with DCM (500 mL), and washed with water (3×500 mL). The organic layer was dried over Na₂SO₄ and concentrated under reduced pressure to give 18.4 (12.0 g, 56%) as a red oil.

To a suspension of LiAlH₄ (0.50 g, 13.2 mmol) in Et₂O (30 mL) was added a solution of 18.4 (2.07 g, 11 mmoL) in Et₂O (30 mL) dropwise under argon. The reaction mixture was stirred at r.t for 3 hr. The reaction was quenched with water (0.9 mL), filtered, and the filtrate was washed with Et₂O. The combined organic layers were dried and concentrated under reduced pressure to give 18.5 (1.56 g, 96%) as a colorless oil. ¹H NMR (400 MHz, DMSO-d₆) δ 4.52 (t, J=5.2 Hz, 1H), 3.81 (q, 2H), 3.60 (q, 2H), 3.38 (q, 2H), 1.70 (m, 1H), 1.29 (s, 6H).

To a stirring solution of 18.5 (731 mg, 5 mmoL) in DCM (20 mL) was added PCC (2.15 g, 10 mmoL) and the reaction mixture was stirred at r.t for 8 hours. The reaction was filtered, and the filtrate was washed with DCM. The combined organic layers were dried over Na₂SO₄ and concentrated under reduced pressure to give a crude, which was purified by flash chromatography (silica gel/Et₂O) to give 18.6 (450 mg, 60%) as a colorless oil.

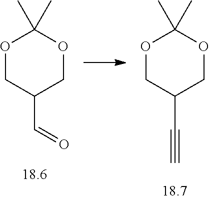

To a stirring solution of 18.6 (432.5 mg, 3 mmoL) in CH₃OH/Et₂O (15 mL, 2:1) were added Bestmann reagent (1.15 g, 6 mmoL) and K₂CO₃ (1.66 g, 12 mmoL) and the reaction was stirred at r.t for 5 hours. The reaction mixture was diluted with water (20 mL), extracted with PE (3×20 mL), and the combined organic layers were dried and concentrated under reduced pressure to give a crude, which was purified by flash chromatography (silica gel/PE:Et₂O=5:1-1:1) to give 18.7 (100 mg, 19%) as a colorless oil.

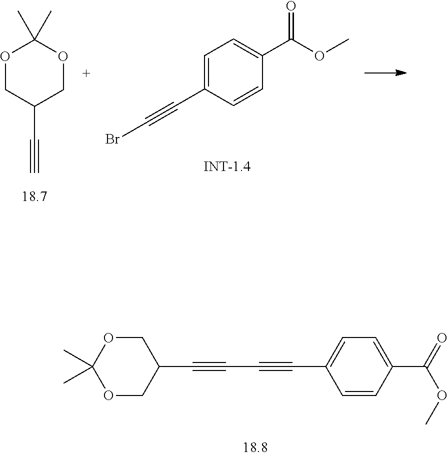

A stirring solution 18.7 (84 mg, 0.6 mmol), Pd(PPh₃)₂Cl₂ (21 m g, 0.03 mmol), CuI (12 mg, 0.06 mmol), i-Pr₂NH (182 mg, 1.8 mmol) in THF (10 mL), was treated with methyl 4-bromoethynylbenzoate (INT-1.4) (157 mg, 0.66 mmol) under argon for 5 hours. The solvent was removed under reduced pressure, and the residue was diluted with water (20 mL), extracted with DCM (2×20 mL), the organic layer was dried over Na₂SO₄ and concentrated under reduced pressure to a crude red oil, which was purified by flash chromatography (silica gel/PE:EA=50:1-30:1) to yield 18.8 (120 mg, 53.6%): MS: m/z calcd for C₁₈H₁₈O₄ 298.12. found [M+H]⁺ 299.1.

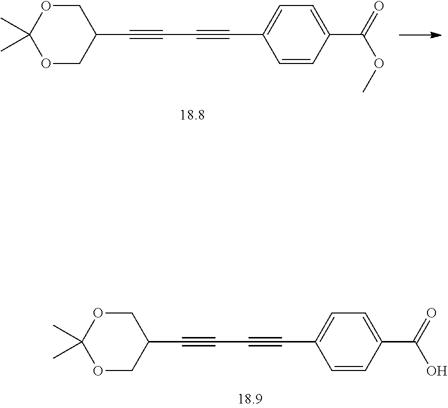 [SMILES: COC(=O)c1ccc(C#CC#CC2COC(C)(C)OC2)cc1 ; CC1(C)OCC(C#CC#Cc2ccc(C(=O)O)cc2)CO1]

To a stirring solution of 18.8 (75 mg, 0.25 mmol) in THF (10 mL) was added 1M LiOH (1 mL) and the reaction was stirred at r.t for 8 hours. The solvent was removed under reduced pressure, and the residue was diluted with water (20 mL), extracted with DCM (2×20 mL), the organic layer was dried over Na₂SO₄ and concentrated under reduced pressure to give 18.9 (100 mg, 95%) as a yellow solid: MS: m/z calcd for C₁₇H₁₆O₄ 284.10. found [M+H]⁺ 285.1.

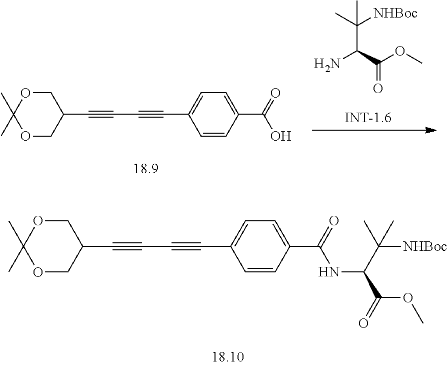

To a stirring solution of 18.9 (71 mg, 0.25 mmol), HOBt (40.5 mg, 0.30 mmol), and EDC (72.6 mg, 0.38 mmol) in DMF (10 mL) was added INT-1.6 (synthesized as described in WO 2008/154642 at pages 240-6), said publication incorporated herein by reference in its entirety (73.8 mg, 0.30 mmoL) and DIPEA (162 mg, 1.25 mmoL) and the reaction was stirred at rt overnight. The reaction was then diluted with DCM (20 mL), washed with 5% LiCl (2×20 mL), the organic layer was dried over Na₂SO₄ and concentrated under reduced pressure to give a yellow oil, which was purified by flash chromatography (silica gel/PE:EA=10:1-5:1) to yield 18.10 (100 mg, 78%) as a colorless oil. MS: m/z calcd for C₁₈H₂₆N₂O₇ 512.25. found [M+H]⁺513.3.

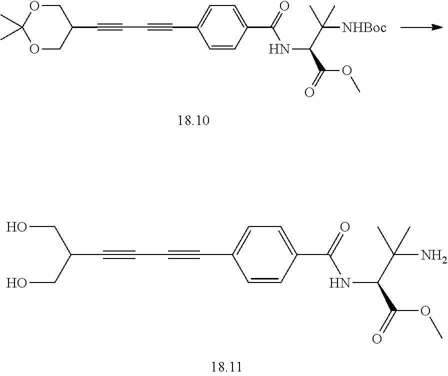

Compound 18.10 (82 mg, 0.16 mmoL) was dissolved in CH₃OH (10 mL) and treated with dry HCl gas for 10 min. The solvent was removed under reduced pressure to yield 18.11 (60 mg, 92%) as a yellow solid: MS: m/z calcd for C₂₀H₂₄N₂O₅ 372.17. found [M+H]⁺ 373.2. ¹H NMR (400 MHz, DMSO-d₆) δ 9.00 (d, J=8.0 Hz, 1H), 8.29 (s, 3H), 7.96 (d, J=8.0 Hz, 2H), 7.65 (d, J=8.0 Hz, 2H), 4.88 (d, J=8.4 Hz, 1H), 3.71 (s, 3H), 3.54 (m, 4H), 2.73 (t, J=5.6 Hz, 1H), 1.38 (s, 6H), 1.22 (s, 1H), 1.04 (t, J=6.8 Hz, 1H).

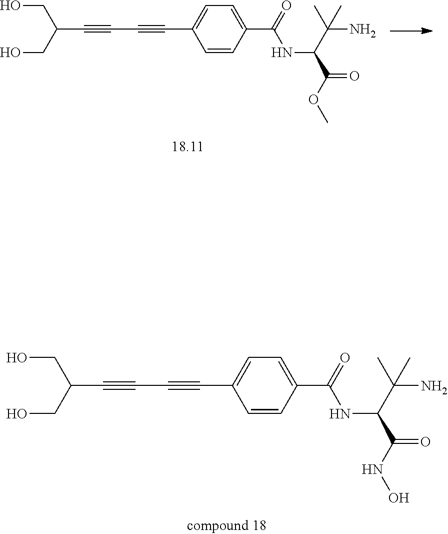 [SMILES: COC(=O)[C@@H](NC(=O)c1ccc(C#CC#CC(CO)CO)cc1)C(C)(C)N ; CC(C)(N)[C@H](NC(=O)c1ccc(C#CC#CC(CO)CO)cc1)C(=O)NO]

Compound 18.11 (8.3 g, 22.29 mmol) was dissolved in THF:MeOH:H₂O (16 mL:16 mL:16: mL) and treated with NH₂OH (50% aq., 1.55 mL) overnight. The reaction was concentrated under reduced pressure and purified by RP HPLC to give (S)—N-(3-amino-1-(hydroxyamino)-3-methyl-1-oxobutan-2-yl)-4-(6-hydroxy-5-(hydroxymethyl)hexa-1,3-diynyl)benzamide (18). MS: m/z calcd for C₁₉H₂₃N₃O₅ 373.16. found [M+H]⁺ 374.2.

### 19. N—((S)-3-amino-1-(hydroxyamino)-3-methyl-1-oxobutan-2-yl)-4-(6-chloro-5-hydroxy-5-(hydroxymethyl)hexa-1,3-diynyl)benzamide (19)

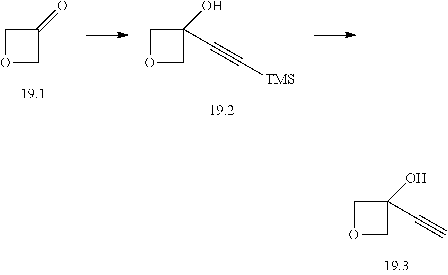

Ethynyltrimethylsilane (19.61 mL, 139 mmol) and N,N,N,N-tetramethylethane-1,2-diamine (20.81 mL, 139 mmol) were dissolved in anhydrous tetrahydrofuran (150 mL), and cooled to −78° C. under a nitrogen atmosphere. n-Butyllithium (87 mL, 139 mmol, 1.6M hexane) was added dropwise and the reaction was stirred for 1 hour at −78° C. A solution of oxetan-3-one (19.1) (5 g, 69.4 mmol) in anhydrous tetrahydrofuran (30 mL) was added dropwise to the reaction mixture over 30 min, and the stirring was continued for 3 hours. The reaction was quenched with water and saturated ammonium chloride, the product was extracted with diethyl ether, and the organic extracts were washed with saturated NaCl, dried over sodium sulfate, and concentrated under reduced pressure to yield a yellow oil (11.48 g, TLC 1:1 EtOAc/hex Rf: 0.33), which was purified by flash chromatography (silica gel/15 to 55% EtOAc/hexanes) to yield compound 19.2 (9.24 g): ¹H-NMR (400 MHz, DMSO-d₆) δ 6.49 (s, 1H), 4.61 (d, J=6.4 Hz, 2H), 4.48 (d, J=6.8 Hz, 2H), 0.17 (s, 9H).

3-((trimethylsilyl)ethynyl)oxetan-3-ol (19.2) (6.29 g, 36.9 mmol) was dissolved in tetrahydrofuran (50 mL), and cooled in an ice bath. Tetrabutylammonium fluoride (1M in THF, 44.3 mL, 44.3 mmol) was added dropwise and the reaction was stirred for 1 hr. Diethyl ether (100 mL) was then added, followed by water (50 mL). The aqueous layer was separated and extracted with diethyl ether, the combined organic extracts were dried over sodium sulfate, and concentrated under reduced pressure to yield 19.3: TLC EtOAc/hex 1:1 Rf 0.45. ¹HNMR (400 MHz, DMSO-d₆) δ 6.47 (s, 1H), 4.63 (m, 2H), 4.49 (m, 2H), 3.61 (s, 1H).

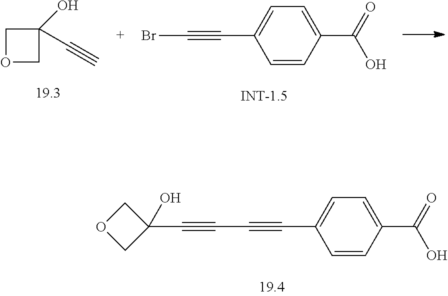

To a stirring solution of copper (I) chloride (0.073 g, 0.738 mmol) in 30% aqueous n-butylamine (32 mL) at 0° C. was added a solution of 3-ethynyloxetan-3-ol (19.3) (3.62 g, 36.9 mmol) in 30% n-butylamine/water (80 mL), followed by the addition of a solution of 4-(bromoethynyl)benzoic acid (INT-1.5) (4 g, 17.77 mmol) and hydroxylamine hydrochloride (0.154 g, 2.214 mmol) in 30% n-butylamine/water (40 mL), and the reaction was stirred for 2 hr at 0° C. Ethyl acetate (500 mL) was added and the mixture was acidified to pH 2. MeOH (100 mL) was added and the aqueous layer was separated. The combined extracts were dried over sodium sulfate and concentrated under reduced pressure to yield 19.4 (3.11 g) as a tan solid, which was carried through to the next step without further purification.

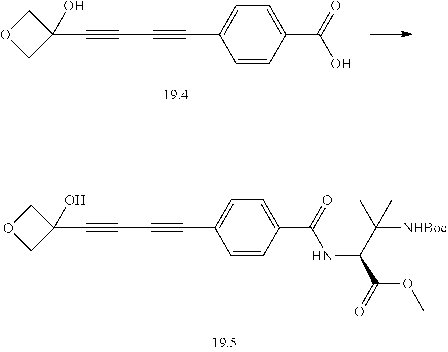 [SMILES: O=C(O)c1ccc(C#CC#CC2(O)COC2)cc1 ; COC(=O)[C@@H](NC(=O)c1ccc(C#CC#CC2(O)COC2)cc1)C(C)(C)NC(=O)OC(C)(C)C]

To a stirring solution of (S)-methyl 2-amino-3-(tert-butoxycarbonylamino)-3-methylbutanoate oxalate (4.75 g, 14.12 mmol) in ethyl acetate (50 mL) and water (50 mL) at 0° C. was added potassium carbonate (3.90 g, 28.2 mmol) and the reaction was stirred for 10 min. The aqueous layer was separated and extracted with ethyl acetate (2×50 mL). The combined extracts were dried over sodium sulfate, and concentrated under reduced pressure to give (S)-methyl 2-amino-3-(tert-butoxycarbonylamino)-3-methylbutanoate, which was dissolved in DMF (30 mL) and added to a stirring solution of 4-((3-hydroxyoxetan-3-yl)buta-1,3-diynyl)benzoic acid (19.4) (3.11 g, 12.84 mmol) in anhydrous dimethylformamide (45 mL) at 0° C. HATU (5.86 g, 15.41 mmol) was added, followed by N-ethyl-N-isopropylpropan-2-amine (6.71 mL, 38.5 mmol) and the reaction was stirred overnight at room temperature. The reaction mixture was poured into water (300 mL), extracted with ethyl acetate (3×200 mL) and the combined extracts were washed with saturated NaCl, dried over sodium sulfate and concentrated under reduced pressure to yield a crude, which was purified by flash chromatography (silica gel/20 to 60% ethylacetate/hexanes) to yield the desired compound 19.5 (4.2 g) as a yellow oil. MS: m/z calcd for C₂₅H₃₀N₂O₇ 470.21. found [M-Boc+H]⁺ 371.2.

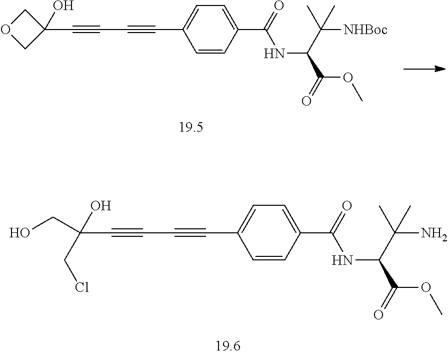 [SMILES: COC(=O)[C@@H](NC(=O)c1ccc(C#CC#CC2(O)COC2)cc1)C(C)(C)NC(=O)OC(C)(C)C ; COC(=O)[C@@H](NC(=O)c1ccc(C#CC#CC(O)(CO)CCl)cc1)C(C)(C)N]

To a stirring solution of (S)-methyl 3-(tert-butoxycarbonylamino)-2-(4-((3-hydroxyoxetan-3-yl)buta-1,3-diynyl)benzamido)-3-methylbutanoate (19.5) (4.20 g, 8.93 mmol) in methanol (20 mL) at 0° C. was added hydrogen chloride (13.39 mL, 53.6 mmol, 4M in dioxane) and the reaction was stirred at room temperature for 5 hr. Solvent removal under reduced pressure yielded 19.6 (3.91 g). MS: m/z calcd for C₂₀H₂₃ClN₂O₅ 406.13. found [M+H]⁺ found 407.1.

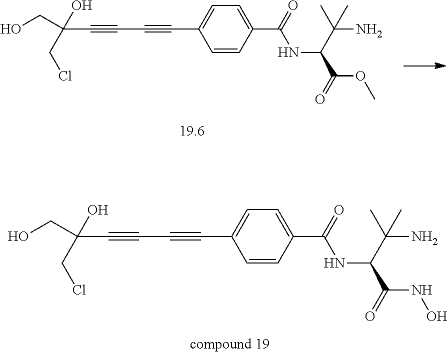 [SMILES: COC(=O)[C@@H](NC(=O)c1ccc(C#CC#CC(O)(CO)CCl)cc1)C(C)(C)N ; CC(C)(N)[C@H](NC(=O)c1ccc(C#CC#CC(O)(CO)CCl)cc1)C(=O)NO]

To a stirring solution of (2S)-methyl 3-amino-2-(4-(6-chloro-5-hydroxy-5-(hydroxymethyl)hexa-1,3-diynyl)benzamido)-3-methylbutanoate hydrochloride (19.6) (3.91 g, 8.82 mmol) in isopropanol (30 mL) at 0° C. was slowly added 50% hydroxylamine/water (11.65 mL, 176 mmol) and the reaction was placed in the freezer (−15° C.) for 72 hr. The IPA was removed under reduced pressure, and the resulting solution was acidified to pH 4 with acetic acid, and purified by RP HPLC to yield N—((S)-3-amino-1-(hydroxyamino)-3-methyl-1-oxobutan-2-yl)-4-(6-chloro-5-hydroxy-5-(hydroxymethyl)hexa-1,3-diyn-1-yl)benzamide (19) (1.407 g) as a white solid as the acetate salt: MS: m/z calcd for C₁₉H₂₂ClN₃O₅ 407.12. found [M+H]⁺ 408.1. ¹H NMR (400 MHz, DMSO-d₆) δ 8.3 (br s, 1H), 7.89 (d, J=7.2 Hz, 2H), 7.68 (d, J=6.8 Hz, 2H), 4.33 (s, 1H), 3.76 (d, J=10.8 Hz, 1H), 3.67 (d, J=10.8 Hz, 1H), 3.52 (m, 2H), 1.88 (s, 3H acetate), 1.11 (s, 3H), 1.03 (s, 3H).

### 20. N—((S)-3-amino-1-(hydroxyamino)-3-methyl-1-oxobutan-2-yl)-4-((5S,6S)-6,7-dihydroxy-5-methylhepta-1,3-diynyl)benzamide (20A) and N—((S)-3-amino-1-(hydroxyamino)-3-methyl-1-oxobutan-2-yl)-4-((5S,6R)-6,7-dihydroxy-5-methylhepta-1,3-diynyl)benzamide (20B)

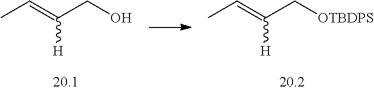

To a stirring solution of alcohol 20.1 (10 mL, 116 mmol) in DCM (80 mL) at 0° C. was added triethylamine (35.7 mL, 256 mmol), followed by tert-butylchlorodiphenylsilane (38.8 mL, 151 mmol) and the reaction was stirred for 4 hr. The reaction mixture was washed with NH₄Cl, NaHCO₃, brine, dried over Na₂SO₄, filtered and concentrated under reduced pressure to yield the desired product 20.2, as a mixture of isomers: ¹H NMR (400 MHz, DMSO-d₆) δ 7.3-7.7 (m, 20H), 6.56 (s, 1H), 5.75 (s, 3H), 4.1 (d, 4H), 1.6 (d, 6H), 0.95 and 0.98 (2 s, 18H).

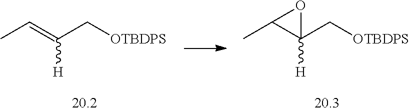

To a stirring solution of 20.2 (2 g, 6.44 mmol) in DCM (20 mL) at 0° C. was added m-CPBA (1.44 g, 8.4 mmol) and the reaction was stirred overnight. The reaction was washed with NaHCO₃, 10% thiosulfate, brine, dried over Na₂SO₄, filtered and concentrated under reduced pressure to yield the desired epoxide 20.3 as a mixture of isomers, which were carried through to the next step without further purification. ¹H NMR (400 MHz, DMSO-d₆) δ 7.3-7.7 (m, 20H), 3.81 (dd, 2H), 3.6 (dd, 2H), 2.80-2.9 (m, 4H), 1.18 (d, 6H), 0.95 and 0.98 (2 s, 18H).

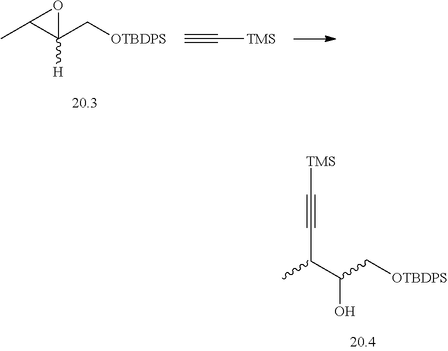

To a stirring solution of ethynyltrimethylsilane (2.71 mL, 19.20 mmol) in toluene (45 mL) at −78° C. was added n-Buli (7 mL, 17.46 mmol) and the reaction was stirred for 20 min. The reaction was warmed to 0° C. and stirred for 10 min. Dimethylaluminum chloride (17.5 mL, 17.46 mmol) was added and the reaction was stirred for 20 min. Compound 20.3 (1.9 g, 5.82 mmol) was then added and the reaction was stirred overnight with warming to room temperature. The reaction was cooled to 0° C. and quenched with 1 M HCl (40 mL), the aqueous layer was separated and extracted with EtOAc (3×40 mL), the combined organic layers were washed with saturated aqueous NaHCO₃, brine, dried over Na₂SO₄, filtered and concentrated under reduced pressure to yield the desired product 20.4, which was carried through to the next step without further purification.

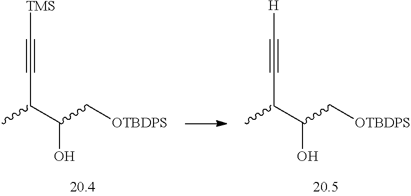

To a stirring solution of alcohol 20.4 (2.2 g, 5.18 mmol) in MeOH (20 mL) was added K₂CO₃ (1.4 g, 10.36 mmol) and the reaction was allowed to stir for 1 hr. Water (10 mL) was added, followed by EtOAc (20 mL) and the aqueous phase was separated and extracted with EtOAc (2×). The combined organic layers were washed with NH₄Cl, brine, dried over Na₂SO₄, filtered and concentrated under reduced pressure to yield 20.5, which was carried through to the next step without further purification.

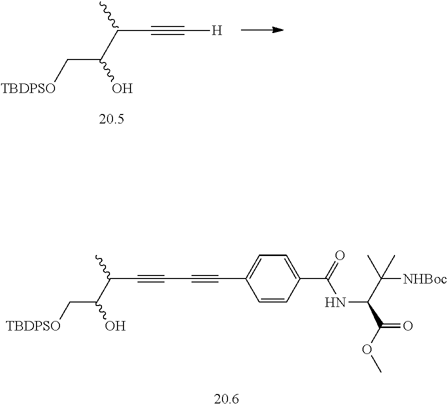

The reaction was carried out according to Procedure 1 to yield a crude, which was purified by flash chromatography (silica gel/EtOAc 30-100%/hexanes) to yield the desired product 20.6 (138 mg, 0.190 mmol, 11.3%) and starting material 20.5 (1 g, 2.20 mmol, 57% recovered). MS: m/z calcd for C₄₂H₅₂N₂O₇ 724.35. found [M-Boc+H]⁺ 625.4.

| | | | Moles | Sample |
|---|---|---|---|---|
| Reactant | MW | Eq | (mmol) | Mass |
| copper(I) chloride | 98.999 | 0.02 | 0.077 | 7.66 mg |
| 1-((tert-butyldiphenylsilyl)oxy)- | 352.542 | 1.1 | 4.25 | 1.5 g |
| 3-methylpent-4-yn-2-ol (20.5) | | | | |
| hydroxylamine hydrochloride | 33.030 | 0.06 | 0.232 | 0.016 g |
| butan-1-amine | 73.137 | 23 | 89 | 6.51 g |
| (S)-methyl 2-(4- | 453.327 | 1.000 | 3.87 | 1.753 g |
| (bromoethynyl)benzamido)- | | | | |
| 3-((tert-butoxycarbonyl)amino)- | | | | |
| 3-methylbutanoate (INT-1) | | | | |

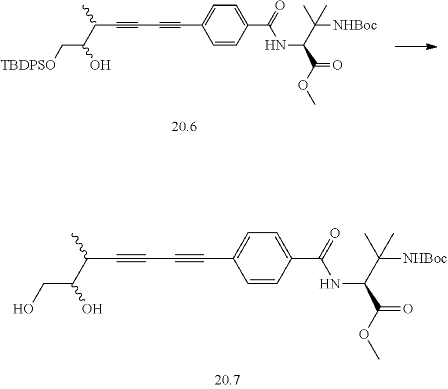

To a stirring solution of 20.6 (0.138 mg, 0.190 mmol) in THF (2 mL) was added TBAF (0.6 mL, 0.57 mmol) and the reaction was stirred overnight. The reaction was diluted with EtOAc (3 mL), washed with saturated aqueous NH₄Cl, NaHCO₃, brine, dried over Na₂SO₄, filtered and concentrated under reduced pressure to yield diol 20.7, which was carried through to the next step without further purification. MS: m/z calcd for C₂₆H₃₄N₂O₇ 486.24. found [M-Boc+H]⁺ 387.1.

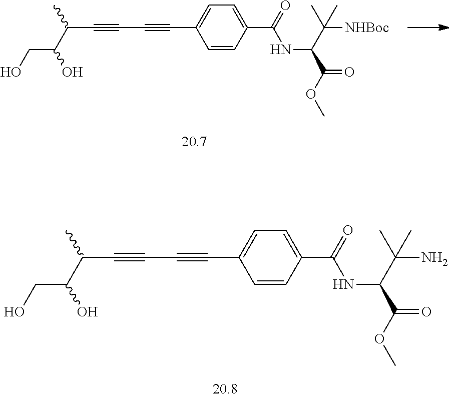

The Boc deprotection of compound 20.7 (93 mg, 0.191 mmol) was carried out according to Procedure 2 to yield compound 20.8, which was carried through to the next step without further purification. MS: m/z calcd for C₂₁H₂₆N₂O₅ 386.18. found [M+H]⁺ 387.4.

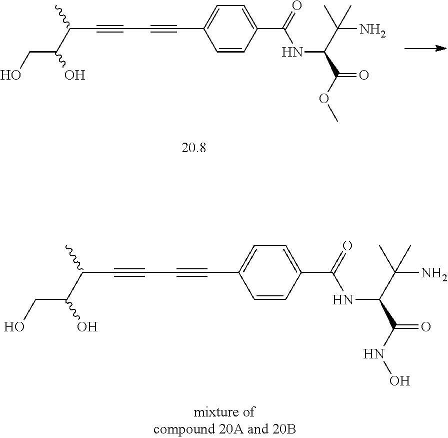

The hydroxamate formation of compound 20.8 was carried out according to Procedure 3 to yield a crude, which was purified on a 1-inch RP HPLC to yield N—((S)-3-amino-1-(hydroxyamino)-3-methyl-1-oxobutan-2-yl)-4-((5S,6S)-6,7-dihydroxy-5-methylhepta-1,3-diynyl)benzamide (20A) and N—((S)-3-amino-1-(hydroxyamino)-3-methyl-1-oxobutan-2-yl)-4-((5S,6R)-6,7-dihydroxy-5-methylhepta-1,3-diynyl)benzamide (20B). MS: m/z calcd for C₂₀H₂₅N₃O₅ 387.18. found [M+H]⁺388.3.

| | | | | Sample | | % |
|---|---|---|---|---|---|---|
| | | | Moles | Mass | Vol | Wt |
| Reactant | MW | Eq | (mmol) | (g) | (mL) | (%) |
| hydroxylamine | 33.030 | 40 | 7.66 | 0.506 | 0.5 | 50 |
| (2S)-methyl 3-amino- | 386.442 | 1.000 | 0.191 | 0.074 | | |
| 2-(4-(6,7-dihydroxy- | | | | | | |
| 5-methylhepta-1,3- | | | | | | |
| diyn-1-yl)benzamido)- | | | | | | |
| 3-methylbutanoate | | | | | | |
| (20.8) | | | | | | |

### 21. N—((S)-3-amino-1-(hydroxyamino)-3-methyl-1-oxobutan-2-yl)-4-(6,7-dihydroxyhepta-1,3-diynyl)benzamide (21)

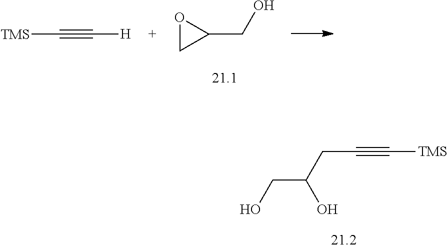

To a stirring solution of ethynyltrimethylsilane (1.908 mL, 13.50 mmol) in THF (15 mL) at −78° C. was added n-butyllithium (8.44 mL, 1.6M, 13.50 mmol) and the reaction was stirred for 20 min. (Diethyloxonio)trifluoroborate (BF₃.OEt₂, 1.71 mL, 13.5 mmol) was then added and the reaction was stirred for 20 min. Oxiran-2-ylmethanol (21.1) (0.9 mL, 13.5 mmol) was then added and the reaction was stirred for 1 hour. The reaction was warmed to 0° C. and stirred for 20 min. The reaction was quenched with NaHCO₃, and the organic layer was separated and concentrated under reduced pressure. The residue was extracted with EtOAc (3×), washed with brine, dried over Na₂SO₄, filtered and concentrated under reduced pressure to yield a crude, which was purified by flash chromatography (silica gel, 2-50% EtOAc/hexanes) to yield the desired diol (21.2) (0.546 g, 3.17 mmol, 23.5%). ¹H NMR (400 MHz, DMSO-d₆) δ 3.65-3.78 (m, 1H), 3.55-3.64 (m, 1H), 3.40-3.50 (m, 1H), 2.2-2.43 (m, 2H), 1.95-2.1 (bs, 1H), 1.5-1.62 (bs, 1H), 0.0 (s, 9H). (Tetrahedron 2011, 67:429-445).

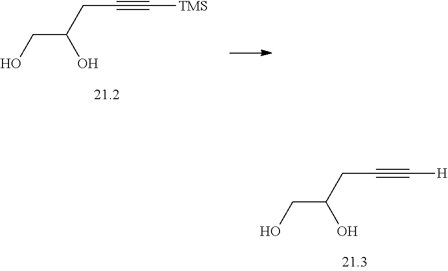 [SMILES: C[Si](C)(C)C#CCC(O)CO ; C#CCC(O)CO]

To a stirring solution of compound 21.2 (0.546 g, 3.17 mmol) in MeOH (5 mL) was added conc. NH₄OH (1.9 mL) and the reaction was stirred overnight. The solvent was removed under reduced pressure to yield compound 21.3, which was used in the next step without further purification.

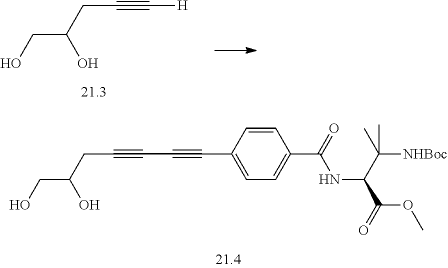 [SMILES: C#CCC(O)CO ; COC(=O)[C@@H](NC(=O)c1ccc(C#CC#CCC(O)CO)cc1)C(C)(C)NC(=O)OC(C)(C)C]

The coupling reaction of 21.3 was carried out according to Procedure 1 to yield compound 21.4, which was carried through to the next step without further purification. MS: m/z calcd for C₂₅H₃₂N₂O₇ 472.22. found [M-Boc+H]⁺ 373.1, [M+Na]⁺495.2.

| | | | Moles | Sample |
|---|---|---|---|---|
| Reactant | MW | Eq | (mmol) | Mass |
| copper(I) chloride | 99.00 | 0.02 | 0.078 | >7.76 mg |
| hydroxylamine hydrochloride x2 | 33.03 | 0.06 | 0.235 | 0.016 g |
| pent-4-yne-1,2-diol | 100.12 | 1.1 | 4.31 | 0.431 g |
| butan-1-amine | 73.14 | 23 | 90 | 6.59 g |
| (S)-methyl 2-(4- | 453.33 | 1.000 | 3.92 | 1.776 g |
| (bromoethynyl)benzamido)- | | | | |
| 3-(tert-butoxycarbonylamino)- | | | | |
| 3-methylbutanoate (INT-1) | | | | |

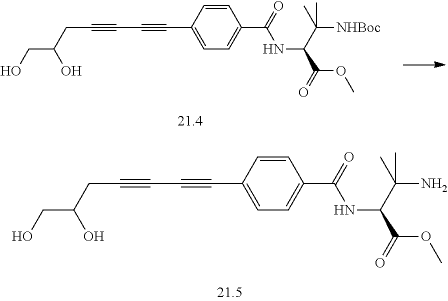 [SMILES: COC(=O)[C@@H](NC(=O)c1ccc(C#CC#CCC(O)CO)cc1)C(C)(C)NC(=O)OC(C)(C)C ; COC(=O)[C@@H](NC(=O)c1ccc(C#CC#CCC(O)CO)cc1)C(C)(C)N]

The Boc deprotection of 21.4 was carried out according to Procedure 2 to yield compound 21.5, which was carried through to the next step without further purification. MS: m/z calcd for C₂₀H₂₄N₂O₅ 372.17. found [M+H]⁺ 373.1.

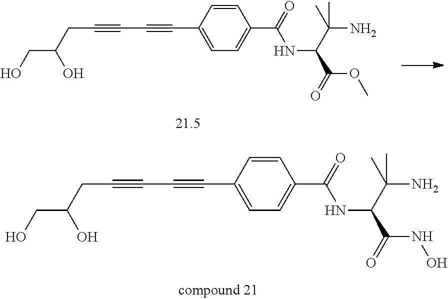 [SMILES: COC(=O)[C@@H](NC(=O)c1ccc(C#CC#CCC(O)CO)cc1)C(C)(C)N ; CC(C)(N)[C@H](NC(=O)c1ccc(C#CC#CCC(O)CO)cc1)C(=O)NO]

The hydroxamate formation of 21.5 was carried out according to Procedure 3 to yield a crude, which was purified on a 2-inch RP HPLC to yield N—((S)-3-amino-1-(hydroxyamino)-3-methyl-1-oxobutan-2-yl)-4-(6,7-dihydroxyhepta-1,3-diynyl)benzamide (21, 230 mg): MS: m/z calcd for C₁₉H₂₃N₃O₅ 373.16. found [M+H]⁺374.1.

| | | | | Sam- | | |
|---|---|---|---|---|---|---|
| | | | | ple | | % |
| | | | Moles | Mass | Vol | Wt |
| Reactant | MW | Eq | (mmol) | (g) | (mL) | (%) |
| hydroxylamine | 33.030 | 40 | 135.2 | 4.47 | 5 mL | 50 |
| (2S)-methyl 3-amino-2- | 372.415 | 1.000 | 3.38 | 1.26 | | |
| (4-(6,7-dihydroxyhepta- | | | | | | |
| 1,3-diyn-1-yl)benzamido)- | | | | | | |
| 3-methylbutanoate | | | | | | |

### 22. 4-(6,7-dihydroxyhepta-1,3-diynyl)-N—((S)-1-(hydroxyamino)-3-methyl-3-(methylamino)-1-oxobutan-2-yl)benzamide (22)

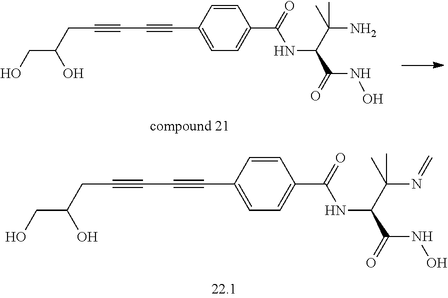 [SMILES: CC(C)(N)[C@H](NC(=O)c1ccc(C#CC#CCC(O)CO)cc1)C(=O)NO ; C=NC(C)(C)[C@H](NC(=O)c1ccc(C#CC#CCC(O)CO)cc1)C(=O)NO]

Starting with N-(3-amino-1-(hydroxyamino)-3-methyl-1-oxobutan-2-yl)-4-(6,7-dihydroxyhepta-1,3-diyn-1-yl)benzamide (21), the reaction was carried out according to Procedure 4A to yield compound 22.1 (0.062 g), which was carried through to the next step without further purification. MS: m/z calcd for C₂₀H₂₃N₃O₅ 385.16. found [M+H]⁺ 386.2.

| | | | Den- | | Sam- | | |
|---|---|---|---|---|---|---|---|
| | | | sity | | ple | | % |
| | | | (g/ | Moles | Mass | Vol | Wt |
| Reactant | MW | Eq | mL) | (mmol) | (g) | (mL) | (%) |
| N-(3-amino-1- | 373.403 | 1.000 | | 0.161 | .06 | | |
| (hydroxyamino)- | | | | | | | |
| 3-methyl-1- | | | | | | | |
| oxobutan-2- | | | | | | | |
| yl)-4-(6,7- | | | | | | | |
| dihydroxyhepta- | | | | | | | |
| 1,3-diyn-1- | | | | | | | |
| yl)benzamide | | | | | | | |
| (21) | | | | | | | |
| DIPEA | 129.243 | 1.5 | 0.74 | 0.241 | 0.031 | 0.042 | |
| formaldehyde | 30.026 | 25 | 1.09 | 4.02 | 0.326 | 0.299 | 37 |
| (37% in water) | | | | | | | |
| Butylamine | 73.14 | 25 | | 4.02 | 0.979 | | 30 |
| (30% in water) | | | | | | | |

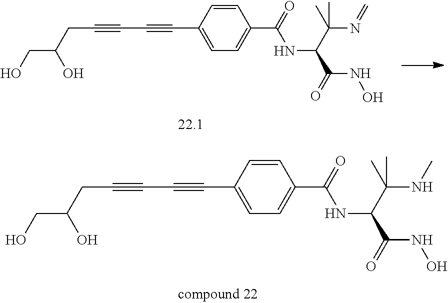 [SMILES: C=NC(C)(C)[C@H](NC(=O)c1ccc(C#CC#CCC(O)CO)cc1)C(=O)NO ; CNC(C)(C)[C@H](NC(=O)c1ccc(C#CC#CCC(O)CO)cc1)C(=O)NO]

The reaction was carried out according to Procedure 4B to yield the crude amine, which was purified on a 2-inch RP HPLC to yield 4-(6,7-dihydroxyhepta-1,3-diynyl)-N—((S)-1-(hydroxyamino)-3-methyl-3-(methylamino)-1-oxobutan-2-yl)benzamide (22) (0.0025 g, 0.00613 mmol, 2.3%). MS: m/z calcd for C₂₀H₂₅N₃O₅ 387.18. found [M+H]⁺ 388.2.

| | | | | | Sam- | |
|---|---|---|---|---|---|---|
| | | | Den- | | ple | |
| | | | sity | Moles | Mass | Vol |
| Reactant | MW | Eq | (g/mL) | (mmol) | (g) | (mL) |
| 4-(6,7-dihydroxy- | 385.414 | 1.000 | | 0.259 | .1 | |
| hepta-1,3- | | | | | | |
| diyn-1-yl)-N-(1- | | | | | | |
| (hydroxyamino)- | | | | | | |
| 3-methyl-3- | | | | | | |
| (methyleneamino)- | | | | | | |
| 1-oxobutan-2- | | | | | | |
| yl)benzamide | | | | | | |
| (22.1) | | | | | | |
| acetic acid | 60.052 | 30 | 1.049 | 7.78 | 0.467 | 0.446 |
| sodium | 39.853 | 20 | | 5.19 | 0.326 | |
| cyanoborohydride | | | | | | |

### 23. N—((S)-3-amino-1-(hydroxyamino)-3-methyl-1-oxobutan-2-yl)-4-(((1S,2R,3S)-2,3-bis(hydroxymethyl)cyclopropyl)buta-1,3-diynyl)benzamide (23A) and N—((S)-3-amino-1-(hydroxyamino)-3-methyl-1-oxobutan-2-yl)-4-(((1R,2R,3S)-2,3-bis(hydroxymethyl)cyclopropyl)buta-1,3-diynyl)benzamide (23B)

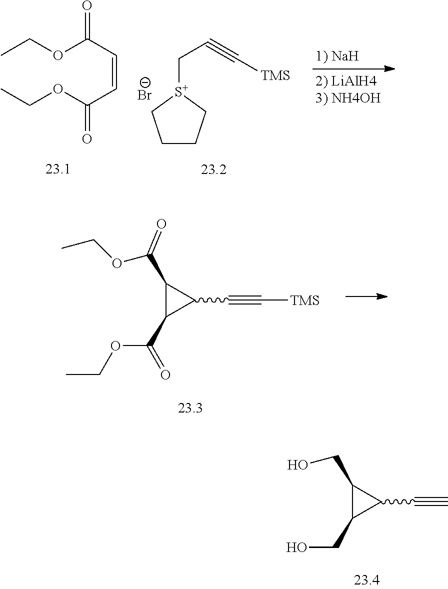

To a stirring solution of compound 23.2 (20.12 g, 72.0 mmol) and diethyl maleate (23.1) (12.40 g, 72.0 mmol) in THF (200 mL) was added NaH (1.729 g, 72.0 mmol), and the reaction mixture was stirred for 18 h. The reaction was quenched with saturated ammonium chloride (10 mL), and the product was extracted with ethyl acetate (2×200 mL), dried (Na₂SO₄) and concentrated under reduced pressure to give 23.3, which was re-dissolved in THF (50 mL) and treated with LiAlH₄ (6.84 g, 180 mmol, 2.5 eq) for 2 hr. Excess LiAlH₄ was quenched with ethyl acetate (25 mL) and saturated sodium sulfate and the reaction was extracted with ethyl acetate (2×200 mL), dried, concentrated under reduced pressure to afford the corresponding alcohol (9.2 g), which was treated with ammonium hydroxide (28 mL) in MeOH (20 mL). The product was extracted with ethyl acetate (2×100 mL), dried and concentrated under reduced pressure to afford (3-ethynylcyclopropane-1,2-diyl)dimethanol (23.4) (6.8 g), which was used in the next step without further purification.

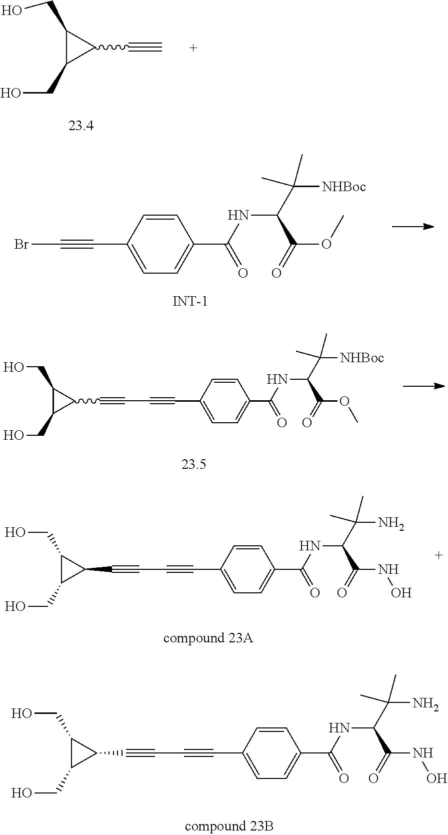

(3-ethynylcyclopropane-1,2-diyl)dimethanol (23.4) was coupled with (S)-methyl 2-(4-(bromoethynyl)benzamido)-3-((tert-butoxycarbonyl)amino)-3-methylbutanoate (INT-1) using Procedure 1 to give (2S)-methyl 2-(4-((2,3-bis(hydroxymethyl)cyclopropyl)buta-1,3-diyn-1-yl)benzamido)-3-((tert-butoxycarbonyl)amino)-3-methylbutanoate (23.5). Subsequent Boc deprotection using Procedure 2 and hydroxamate formation using Procedure 3 gave, after RP-HPLC purification, N—((S)-3-amino-1-(hydroxyamino)-3-methyl-1-oxobutan-2-yl)-4-(((1s,2R,3S)-2,3-bis(hydroxymethyl)cyclopropyl)buta-1,3-diyn-1-yl)benzamide (23A, 445 mg), and N—((S)-3-amino-1-(hydroxyamino)-3-methyl-1-oxobutan-2-yl)-4-(((1r,2R,3S)-2,3-bis(hydroxymethyl)cyclopropyl)buta-1,3-diyn-1-yl)benzamide (23B, 54 mg). MS: m/z calcd for C₂₁H₂₅N₃O₅ 399.18. found [M+H]⁺ 400.2, 400.3.

| | | | | Sample | |
|---|---|---|---|---|---|
| | | | Moles | Mass | % Wt |
| Reactant | MW | Eq | (mmol) | (g) | (%) |
| TFA | 114.023 | 5 | 55.0 | 6.27 | |
| hydroxylamine | 33.030 | 0.06 | 0.660 | 0.046 | |
| hydrochloride | | | | | |
| copper(I) chloride | 98.999 | 0.02 | 0.220 | 0.022 | |
| (3-ethynylcyclopropane- | 126.153 | 01 | 11 | 1.388 | |
| 1,2-diyl)dimethanol | | | | | |
| (S)-methyl 2-(4- | 453.327 | 1.003 | 11.03 | 5 | |
| (bromoethynyl)benzamido)- | | | | | |
| 3-((tert- | | | | | |
| butoxycarbonyl)amino)-3- | | | | | |
| methylbutanoate (INT-1) | | | | | |
| hydroxylamine | 33.030 | 40 | 440 | 29.1 | 50 |

### 24. N—((S)-3-amino-1-(hydroxyamino)-3-methyl-1-oxobutan-2-yl)-4-(((R)-2,2-bis(hydroxymethyl)cyclopropyl)buta-1,3-diynyl)benzamide (24)

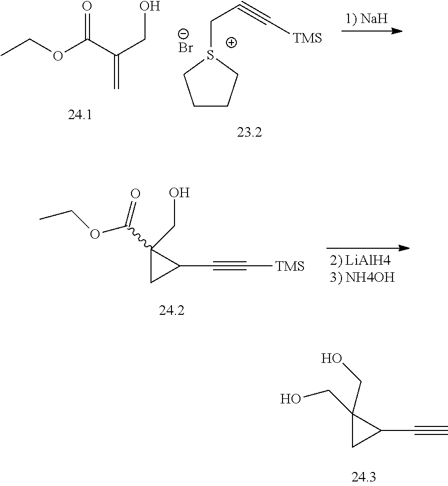

To a stirring solution of ethyl 2-(hydroxymethyl)acrylate (24.1) (4.66 g, 35.8 mmol) and compound 23.2 (10 g, 35.8 mmol) in THF (50 mL) was added sodium hydride (1.718 g, 71.6 mmol) and the reaction mixture was stirred for 18 hr. The reaction was quenched with saturated ammonium chloride (10 mL), and the product was extracted with ethyl acetate (2×200 mL), dried (Na₂SO₄) and concentrated under reduced pressure to give ethyl 1-(hydroxymethyl)-2-((trimethylsilyl)ethynyl)cyclopropanecarboxylate (24.2, 6 g). Compound 24.2 was dissolved in THF (50 mL) and was treated with LiAlH₄ (36 mL, 1M LiAlH₄, 37.5 mmmol) and stirring was continued for 2 hr. Excess LiAlH₄ was quenched with ethyl acetate (25 mL) and saturated sodium sulfate, and the reaction was extracted with ethyl acetate (2×200 mL), dried, and concentrated under reduced pressure to afford the reduced alcohol (4.9 g), which was treated with ammonium hydroxide (15 mL) in methanol (20 mL). The product was extracted with ethyl acetate (2×100 mL), dried and concentrated under reduced pressure to afford (2-ethynylcyclopropane-1,1-diyl)dimethanol (24.3, 4.2 g), which was used in the next step without further purification.

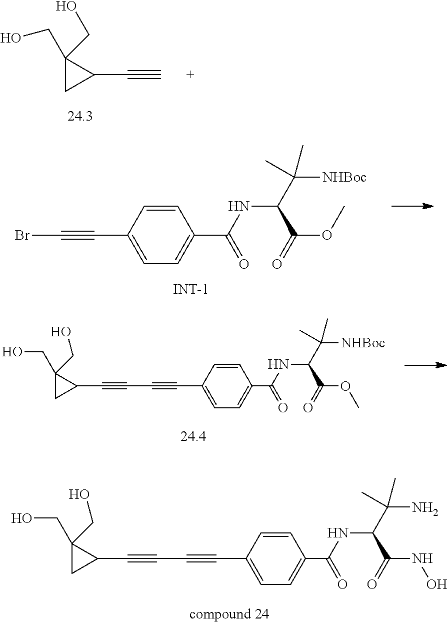

(2-Ethynylcyclopropane-1,1-diyl)dimethanol (24.3) was coupled with (S)-methyl 2-(4-(bromoethynyl)benzamido)-3-((tert-butoxycarbonyl)amino)-3-methylbutanoate (INT-1) using Procedure 1 to give (2S)-methyl 2-(4-((2,2-bis(hydroxymethyl)cyclopropyl)buta-1,3-diyn-1-yl)benzamido)-3-((tert-butoxycarbonyl)amino)-3-methylbutanoate (24.4). Subsequent deprotection of the BOC group using Procedure 2 and hydroxamate formation using Procedure 3 gave after RP-HPLC N—((S)-3-amino-1-(hydroxyamino)-3-methyl-1-oxobutan-2-yl)-4-((2,2-bis(hydroxymethyl)cyclopropyl)buta-1,3-diyn-1-yl)benzamide (24, 64 mg): MS: m/z calcd for C₂₁H₂₅N₃O₅ 399.18. found [M+H]⁺ 400.2.

| | | | Sample |
|---|---|---|---|
| | | Moles | Mass |
| Reactant | MW | (mmol) | (g) |
| hydroxylamine hydrochloride | 33.030 | 0.660 | 0.046 |
| copper(I) chloride | 98.999 | 0.220 | 0.022 |
| (2-ethynylcyclopropane-1,1-diyl)dimethanol | 126.153 | 11 | 1.388 |
| (S)-methyl 2-(4-(bromoethynyl)benzamido)- | 453.327 | 11.03 | 5 |
| 3-((tert-butoxycarbonyl)amino)-3- | | | |
| methylbutanoate (INT-1) | | | |

### 25. N—((S)-3-amino-1-(hydroxyamino)-3-methyl-1-oxobutan-2-yl)-4-((2-(1,2-dihydroxyethyl)cyclopropyl)buta-1,3-diynyl)benzamide (25)

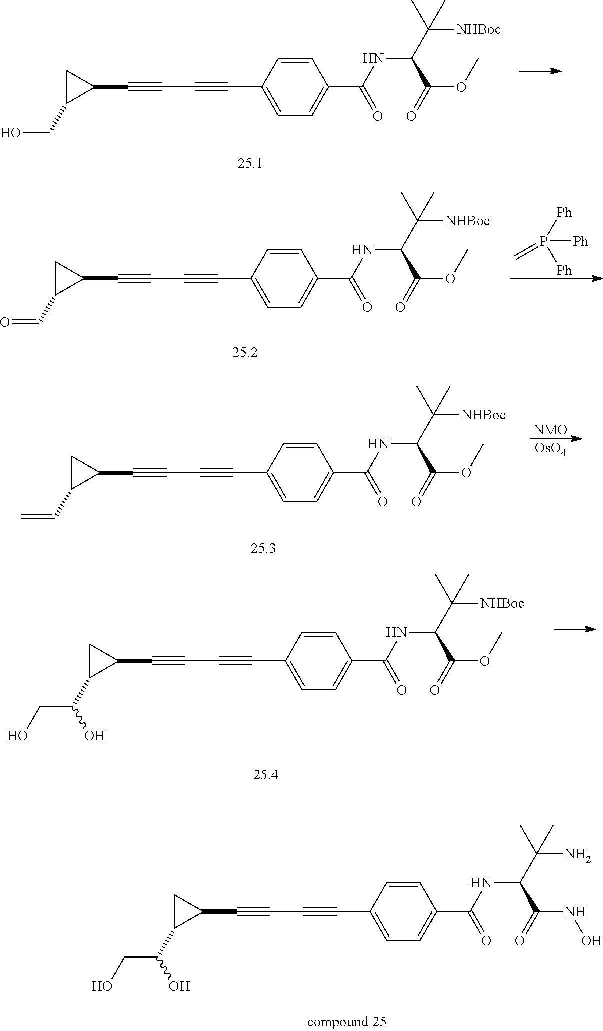

To a stirring solution of compound 25.1 (5 g, 10.67 mmol) (synthesized as described in example C of copending U.S. patent application Ser. No. 13/289,209, incorporated herein by reference) and NaHCO₃ (3.59 g, 42.7 mmol) in CH₂Cl₂ (100 mL) was added Dess-Martin Periodinane (6.79 g, 16.01 mmol) and the reaction mixture was stirred for 2 h. The solids were removed by filtration, water (200 mL) was added and the reaction was stirred for 20 min. The aqueous phase was separated and the organic layer was dried (Na₂SO₄) and concentrated under reduced pressure to give a crude, which was purified by flash chromatography (silica gel/10-40% EtOAc/Hexanes) to afford (S)-methyl 3-((tert-butoxycarbonyl)amino)-2-(4-(((1R,2R)-2-formylcyclopropyl)buta-1,3-diyn-1-yl)benzamido)-3-methylbutanoate (25.2) (4.2 g), MS: m/z calcd for C₂₆H₃₀N₂O₆ 466.21. found [M-Boc+H]⁺ 367.1.

To a stirring solution of potassium tert-butoxide (10.23 g, 18.23 mmol) and bromo(methyl)triphenylphosphorane (6.51 g, 18.23 mmol) in THF (10 mL) was added (2S)-methyl 3-((tert-butoxycarbonyl)amino)-2-(4-((2-formylcyclopropyl)buta-1,3-diyn-1-yl)benzamido)-3-methylbutanoate (25.2) (5.67 g, 12.15 mmol) and the reaction was stirred for 1 h. The reaction was concentrated under reduced pressure and the crude was purified by flash chromatography (silica gel/10-40% EtOAc in hexanes) to yield the desired product 25.3 (3.8 g): MS: m/z calcd for C₂₇H₃₂N₂O₅ 464.23. found [M+H]⁺ 465.0.

To a stirring solution of (2S)-methyl 3-((tert-butoxycarbonyl)amino)-3-methyl-2-(4-((2-vinylcyclopropyl)buta-1,3-diyn-1-yl)benzamido)butanoate (25.3) (2.3 g, 4.95 mmol) and NMO (1.392 g, 5.94 mmol) in acetone (20 mL) was added Osmium tetroxide (0.388 mL, 0.050 mmol) and the reaction was stirred overnight. Excess acetone was removed under reduced pressure and the reaction was extracted with ethyl acetate (3×200 mL), dried and concentrated under reduced pressure to give the desired diol (S)-methyl 3-((tert-butoxycarbonyl)amino)-2-(4-(((1R,2R)-2-(1,2-dihydroxyethyl)cyclopropyl)buta-1,3-diyn-1-yl)benzamido)-3-methylbutanoate (25.4) (2.1 g), which was used in the next step without any further purification. MS: m/z calcd for C₂₇H₃₂N₂O₇ 498.24. found [M+H]⁺ 499.1.

Starting from (S)-methyl 3-((tert-butoxycarbonyl)amino)-2-(4-(((1R,2R)-2-(1,2-dihydroxyethyl)cyclopropyl)buta-1,3-diyn-1-yl)benzamido)-3-methylbutanoate (2.1 g), the Boc group was removed using Procedure 2, followed by hydroxamate formation using Procedure 3 (50% aqueous hydroxylamine, 6 mL) to yield, after RP HPLC purification, N—((S)-3-amino-1-(hydroxyamino)-3-methyl-1-oxobutan-2-yl)-4-(((1R,2R)-2-(1,2-dihydroxyethyl)cyclopropyl)buta-1,3-diyn-1-yl)benzamide as a mixture of two diasteromers (25, 0.61 g). MS: m/z calcd for C₂₁H₂₅N₃O₅ 399.18. found [M+H]⁺400.1.

### 26. N—((S)-3-amino-1-(hydroxyamino)-3-methyl-1-oxobutan-2-yl)-4-(5-hydroxy-5-(2-(hydroxymethyl)cyclopropyl)penta-1,3-diynyl)benzamide (26)

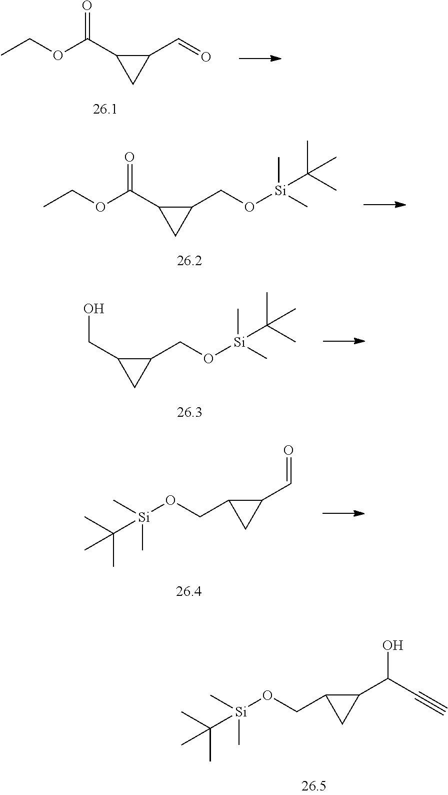

To a stirring solution of ethyl 2-formylcyclopropanecarboxylate (26.1) (10 g, 70.3 mmol) in THF (25 mL) was added sodium borohydride (1.331 g, 35.2 mmol) and the reaction was stirred for 2 hr. Water (10 mL) was added and the product was extracted with ethyl acetate (2×100 mL) to afford the corresponding alcohol (9.2 g, 63.8 mmol), which was treated with 1H-imidazole (5.21 g, 77 mmol), and tert-butylchlorodimethylsilane (11.54 g, 77 mmol) in DCM (20 mL) for 18 hr. The salts were removed by filtration and the solvent was removed under reduced pressure to yield the desired ester (26.2), which was dissolved in THF (25 mL) and treated with LiAlH₄ (32 mL, 2 M, 63.8 mmol) at room temperature for 2 hr. The reaction mixture was quenched with saturated sodium sulfate (20 mL), solids were removed by filtration and the filtrate was dried (Na₂SO₄), filtered and concentrated under reduced pressure to afford compound 26.3 (11.8 g).

To a stirring solution of (2-(((tert-butyldimethylsilyl)oxy)methyl)cyclopropyl)methanol (26.3) (5.5 g, 25.4 mmol) in DCM (100 mL) was added Dess-Martin Periodinane (15.09 g, 35.6 mmol), followed by NaHCO₃ (2.135 g, 25.4 mmol) and the reaction mixture was stirred for 18 hr. The solids were removed by filtration, water (200 mL) was added, and the reaction was extracted with DCM (2×100 mL), dried (Na₂SO₄), filtered and concentrated under reduced pressure to yield, after filtration through a silica gel pad, 2-(((tert-butyldimethylsilyl)oxy)methyl)cyclopropanecarbaldehyde (26.4) (4.81 g) as a liquid.

To a stirring solution of ethynyltrimethylsilane (1.558 g, 15.86 mmol) in THF (25 mL) at −78° C. was added n-Buli (7.61 mL, 19.03 mmol) dropwise over 20 min and the reaction was stirred for 2 hr. 2-(((tert-butyldimethylsilyl)oxy)methyl)cyclopropanecarbaldehyde (26.4) (3.4 g, 15.86 mmol) in THF (5 mL) was added dropwise and stirring was continued for 1 hr. The reaction mixture was slowly quenched with ammonium hydroxide (12.35 mL) and stirred for 18 hr. Excess solvent was removed under reduced pressure and the product was extracted with ethyl acetate (2×100 mL), dried (Na₂SO₄), filtered and concentrated under reduced pressure to afford compound 26.5 (3.22 g).

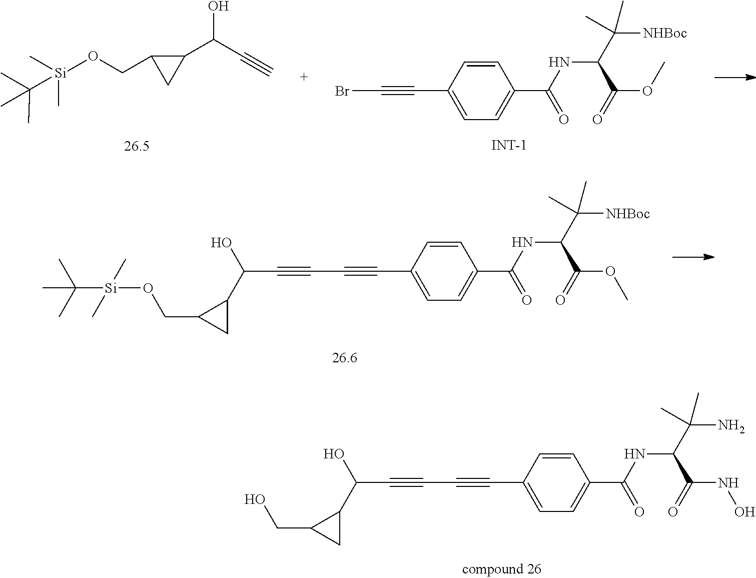

1-(2-(((tert-butyldimethylsilyl)oxy)methyl)cyclopropyl)prop-2-yn-1-ol (26.5) was coupled with (S)-methyl 2-(4-(bromoethynyl)benzamido)-3-((tert-butoxycarbonyl)amino)-3-methylbutanoate (INT-1) using Procedure 1. Subsequent deprotection of BOC and TBDMS group using Procedure 2 (TFA, 5.5 mL) and hydroxamate formation using Procedure 3 (50% aqueous NH₂OH, 3.66 mL) gave, after purification by RP-HPLC, N—((S)-3-amino-1-(hydroxyamino)-3-methyl-1-oxobutan-2-yl)-4-(5-hydroxy-5-(2-(hydroxymethyl)cyclopropyl)penta-1,3-diyn-1-yl)benzamide (26, 140 mgs), MS: m/z calcd for C₂₁H₂₅N₃O₅ 399.18. found [M+H]⁺400.1.

| | | | | Sample |
|---|---|---|---|---|
| | | | Moles | Mass |
| Reactant | MW | Eq | (mmol) | (g) |
| hydroxylamine hydrochloride | 33.030 | 0.06 | 0.799 | 0.055 |
| copper(I) chloride | 98.999 | 0.02 | 0.266 | 0.026 |
| 1-(2-(((tert- | 240.414 | 1.000 | 13.31 | 3.2 |
| butyldimethylsilyl)oxy)methyl)- | | | | |
| cyclopropyl)prop-2-yn-1-ol | | | | |
| (S)-methyl 2-(4- | 453.327 | 1.000 | 13.31 | 6.03 |
| (bromoethynyl)benzamido)-3-((tert- | | | | |
| butoxycarbonyl)amino)-3- | | | | |
| methylbutanoate (INT-1) | | | | |

### 27. N—((S)-3-amino-1-(hydroxyamino)-3-methyl-1-oxobutan-2-yl)-4-(((1S,3R)-1-hydroxy-3-(hydroxymethyl)cyclobutyl)buta-1,3-diynyl)benzamide (27)

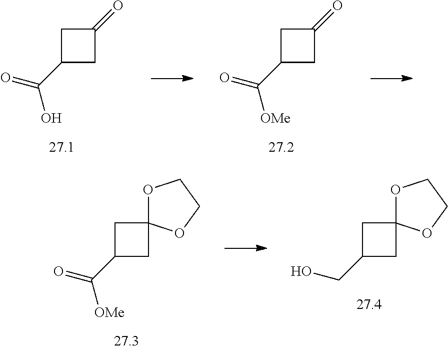 [SMILES: O=C1CC(C(=O)O)C1 ; COC(=O)C1CC(=O)C1 ; COC(=O)C1CC2(C1)OCCO2 ; OCC1CC2(C1)OCCO2]

To a stirring solution of 3-oxocyclobutanecarboxylic acid (27.1) (15 g, 131 mmol) in dichloromethane (70 mL) was added methanol (10.66 mL, 263 mmol), followed by the dropwise addition of a solution of N,N-dimethylpyridin-4-amine (12.85 g, 105 mmol) and N,N′-methanediylidenebis(propan-2-amine) (22.39 mL, 145 mmol) in dichloromethane (20 mL) and the reaction mixture was stirred for 3 days at room temperature.

The dicyclohexylurea precipitate (16.94 g; 117.5 mmole) was removed by filtration, dichloromethane was added and the organic layer was washed with 0.5M HCl, saturated NaHCO₃, saturated NaCl, dried over sodium sulfate, filtered and concentrated under reduced pressure to give a crude (TLC: EtOAc/hex 1:1 Rf 0.57), which was purified by flash chromatography (silica gel/25-50% ethyl acetate/hexanes) to yield the desired product 27.2 (16.49 g). ¹H NMR (400 MHz, DMSO-d₆) δ 3.66 (s, 3H), 3.28 (m, 5H).

To a stirring solution of methyl 3-oxocyclobutanecarboxylate (27.2) (16.49 g, 129 mmol) in benzene (150 mL) was added ethane-1,2-diol (10.77 mL, 193 mmol) followed by 4-methylbenzenesulfonic acid (0.887 g, 5.15 mmol). The flask was fitted with a Dean Stark trap and the reaction was heated to reflux for 3 hours until no more water was being collected. The reaction was then cooled to room temperature, ethyl acetate (100 mL) was added, and the organic layer was washed with 5% NaHCO₃, saturated NaCl, dried over sodium sulfate, filtered and concentrated under reduced pressure to yield a crude (19.13 g, TLC ethyl acetate/hexanes 1:4 Rf: 0.33), which was purified by flash chromatography (silica gel/30% ethyl acetate/hexanes) to yield the desired compound 27.3 (14.26 g). ¹H NMR indicated presence of the desired product, some transesterification of methyl ester to hydroxyethyl ester was also present. Reduction of both esters gave desired product in the next step.

To a stirring solution of lithium aluminum hydride (3.14 g, 83 mmol) in anhydrous diethyl ether (80 mL) was added dropwise a solution of methyl 5,8-dioxaspiro[3.4]octane-2-carboxylate (27.3) (14.26 g, 83 mmol) in anhydrous diethyl ether (80 mL) under a nitrogen atmosphere at a rate such that gentle reflux was achieved, and the reaction was then stirred for 3 hr at room temperature. The reaction was cooled to 0° C. and quenched with 10% aqueous Rochelle's salt (100 mL). The product was extracted into ethyl acetate, and the combined extracts were washed with water, saturated sodium chloride, dried over sodium sulfate, filtered and concentrated under reduced pressure to yield the desired product 27.4. ¹H NMR (400 MHz, DMSO-d₆) δ 4.51 (t, 1H), 3.76 (m 4H), 3.35 (m, 2H), 2.20 (m, 2H), 2.05 (m, 1H), 1.98 (m, 2H).

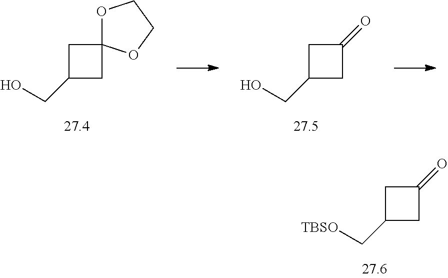 [SMILES: OCC1CC2(C1)OCCO2 ; O=C1CC(CO)C1 ; CC(C)(C)[Si](C)(C)OCC1CC(=O)C1]

To a stirring solution of 5,8-dioxaspiro[3.4]octan-2-ylmethanol (27.4) (10.5 g, 72.8 mmol) in tetrahydrofuran (80 mL) was added water (13.12 g, 728 mmol) and 1M aqueous hydrogen chloride (7.28 mL, 7.28 mmol) and the reaction was heated to 55° C. for 2 hr. The reaction was cooled to 0° C. and neutralized with saturated NaHCO₃ to pH 7. The reaction was extracted with ethyl acetate and the combined extracts were washed with water, dried over sodium sulfate, filtered and concentrated under reduced pressure to give 27.5 (5.58 g) as a colorless liquid, which was carried through to the next step without further purification: TLC: Rf EtOAc 0.32; ¹H NMR (400 MHz, DMSO-d₆:) δ 4.77 (t, 1H), 3.50 (t, 2H), 2.97 (m 2H), 2.75 (m, 2H), 2.45 (m 1H).

To a stirring solution of 3-(hydroxymethyl)cyclobutanone (27.5) (5.58 g, 55.7 mmol) in dimethylformamide (60 mL) was added tert-butylchlorodimethylsilane (9.24 g, 61.3 mmol) followed by 1H-imidazole (5.69 g, 84 mmol) and the reaction was stirred at room temperature overnight. The reaction mixture was partitioned between water (300 mL) and EtOAc (300 mL), and the aqueous phase was extracted with ethyl acetate (3×80 mL). The combined extracts were washed with water, saturated NaCl (3×50 mL), dried over sodium sulfate, filtered and concentrated under reduced pressure to give a crude, which was purified by flash chromatography (silica gel/10-30% EtOAc/hexanes) to yield the desired product 27.6 (8.89 g); TLC: EtOAc/hex 1:10, Rf: 0.75; ¹H NMR (400 MHz, DMSO-d₆) δ 3.74 (d, J=5.2 Hz, 2H), 3.03 (m, 2H), 2.90 (m, 2H), 2.52 (m, 1H), 0.88 (s, 9H), 0.05 (s 6H).

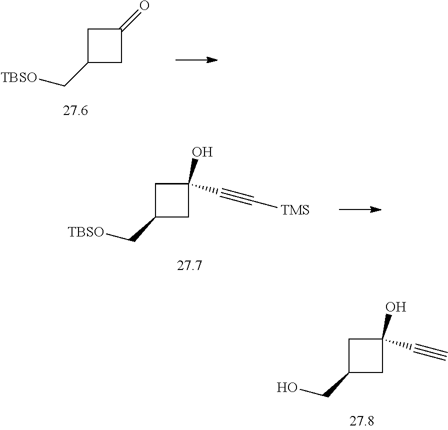

To a stirring solution of ethynyltrimethylsilane (3.48 mL, 24.63 mmol) in anhydrous tetrahydrofuran (40 mL) at −78° C. under a nitrogen atmosphere was added n-butyllithium (9.85 mL, 24.63 mmol, 2.5M hexane) dropwise over 30 minutes, and the reaction was stirred for 1 hour at −78° C. A solution of 3-(((tert-butyldimethylsilyl)oxy)methyl)cyclobutanone (27.6) (4.40 g, 20.52 mmol) in tetrahydrofuran (12 mL) was added dropwise over 30 min, and stirring was continued for an additional 1.5 hr. The reaction was allowed to warm to room temperature with stirring for 1 hour, and was then stored at 4° C. overnight. The reaction mixture was quenched with water (100 mL) and saturated ammonium chloride (10 mL), and was extracted into diethyl ether. The aqueous layer was separated and extracted with diethyl ether, the combined organic extracts were washed with water, saturated NaCl, dried over sodium sulfate, filtered and concentrated under reduced pressure to yield a crude, which was purified by flash chromatography (silica gel/10-30% EtOAc/hexanes) to yield the desired product 27.7 (4.64 g): TLC 1:4 EtOAc/hex Rf 0.62; ¹H NMR (400 MHz, DMSO-d₆) δ 5.70 (s, 1H), 5.52 (d, J=5.52 Hz, 2H), 2.23 (m, 2H), 2.10 (m, 1H), 1.85 (m, 2H), 0.88 (s, 9H), 0.12 (s, 9H), 0.01 (s, 6H).

To a stirring solution of 3-(((tert-butyldimethylsilyl)oxy)methyl)-1-((trimethylsilyl)ethynyl)cyclobutanol (27.7) (4.60 g, 14.72 mmol) in tetrahydrofuran (50 mL) at 0° C. was added tetrabutylammonium fluoride (1M in THF. 35.3 mL, 35.3 mmol) and the reaction was stirred at room temperature for 2 hours. The reaction mixture was poured into ice water (100 mL) and extracted with diethyl ether. The combined extracts were washed with water, saturated NaCl, dried over sodium sulfate, filtered and concentrated under reduced pressure to yield a crude, which was purified by flash chromatography (silica gel/50-100% EtOAc/hexanes) to yield the desired product 27.8 (840 mg): TLC EtOAc Rf: 0.5; ¹H NMR (400 MHz, DMSO-d₆) δ 5.68 (s, 1H), 4.49 (t, 1H), 3.34 (m, 2H), 3.28 (s, 1H), 2.28 (m, 2H), 2.09, (m, 1H), 1.96 (m 2H).

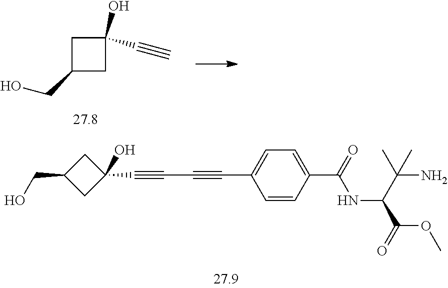

To a stirring solution of hydroxylamine hydrochloride (27.8 mg, 0.400 mmol) and copper(I) chloride (13.18 mg, 0.133 mmol) in 30% aqueous n-butylamine (5 mL) at 0° C. was added a solution of (1S,3S)-1-ethynyl-3-(hydroxymethyl)cyclobutanol (27.8) (840 mg, 6.66 mmol) in 30% n-butylamine/water solution (10 mL), followed by the dropwise addition over 10 min of a solution of (S)-methyl 3-amino-2-(4-(bromoethynyl)benzamido)-3-methylbutanoate hydrochloride (INT-2) (2595 mg, 6.66 mmol) and hydroxylamine hydrochloride (27.8 mg, 0.400 mmol) in 30% n-butylamine/water (15 mL) and MeOH (5 mL) and the reaction was stirred for 2 hours at 0° C. The reaction mixture was extracted with ethyl acetate, and the combined extracts were washed with water, dried over sodium sulfate, filtered and concentrated under reduced pressure to yield a crude, which was purified by flash chromatography (silica gel/0-5% MeOH/DCM), to yield the desired product 27.9 (650 mg) as a yellow foam. MS: m/z calcd for C₂₂H₂₆N₂O₅ 398.18. found [M+H]⁺ 399.2.

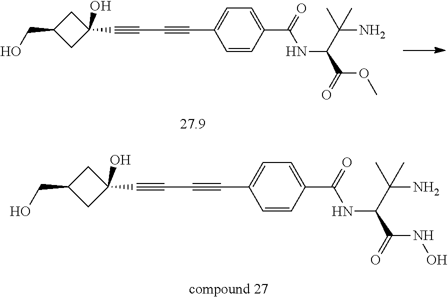 [SMILES: COC(=O)[C@@H](NC(=O)c1ccc(C#CC#C[C@]2(O)C[C@@H](CO)C2)cc1)C(C)(C)N ; CC(C)(N)[C@H](NC(=O)c1ccc(C#CC#C[C@]2(O)C[C@@H](CO)C2)cc1)C(=O)NO]

To a stirring solution of (S)-methyl 3-amino-2-(4-(((1s,3R)-1-hydroxy-3-(hydroxymethyl)cyclobutyl)buta-1,3-diyn-1-yl)benzamido)-3-methylbutanoate (27.9) (0.650 g, 1.631 mmol) in isopropanol (8 mL) at 0° C. was added 50% hydroxylamine/water (1.616 g, 24.47 mmol) and the reaction was stirred at 0° C. for 4 days. The reaction mixture was acidified at 0° C. with AcOH to pH 6, and was concentrated under reduced pressure to approximately 8 mL, which was purified by RP HPLC to yield N—((S)-3-amino-1-(hydroxyamino)-3-methyl-1-oxobutan-2-yl)-4-(((1S,3R)-1-hydroxy-3-(hydroxymethyl)cyclobutyl)buta-1,3-diyn-1-yl)benzamide (27) (295 mg) as its acetate salt: ¹H NMR (400 MHz, DMSO-d₆) δ 8.2 (br s, 1H), 7.88 (d, J=7.6 Hz, 2H), 7.66 (d, J=8 Hz, 2H), 6.07 (br s, 1H), 4.6 (br s, 1H), 4.29 (s, 1H), 3.37 (d, J=5.6 Hz, 2H), 2.37 (m, 2H), 2.14 (m, 1H), 1.99 (t, 2H), 1.94 (s, acetate CH3), 1.09 (s, 3H), 1.02 (s, 3H). m/z calcd for C₂₁H₂₅N₃O₅ 399.18. found [M+H]⁺ 400.1

### 28. N—((S)-3-amino-1-(hydroxyamino)-3-methyl-1-oxobutan-2-yl)-4-(5-((1S,3S)-1-hydroxy-3-(hydroxymethyl)cyclobutyl)penta-1,3-diyn-1-yl)benzamide (28)

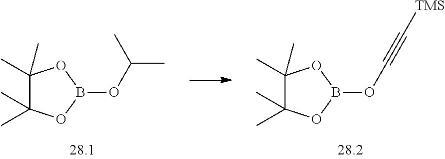 [SMILES: CC(C)OB1OC(C)(C)C(C)(C)O1 ; CC1(C)OB(OC#C[Si](C)(C)C)OC1(C)C]

To a stirring solution of trimethyl(prop-1-yn-1-yl)silane (15.53 mL, 105 mmol) in anhydrous tetrahydrofuran (75 mL) at −25° C. was added dropwise n⁻ butyllithium (40.4 mL, 101 mmol) and the reaction was stirred for 1 hour. The resulting solution was then added via cannula to a solution of 2-isopropoxy-4,4,5,5-tetramethyl-1,3,2-dioxaborolane (28.1) (20.06 mL, 98 mmol) and magnesium chloride (9.34 g, 98 mmol) in anhydrous tetrahydrofuran (75 mL) at −25° C. and the reaction was stirred for 3 hours. The reaction was then cooled to −30° C. and acetyl chloride (7.86 mL, 110 mmol) in MTBE (10 mL) was added and the reaction was stirred for 1 hr, it was then warmed to rt. The reaction was then concentrated under reduced pressure to a volume of approximately 150 mL, MTBE (100 mL) was added and the reaction was concentrated under reduced pressure. Hexanes (350 mL) was added, and the resulting solids were removed by filtration; the filtrate was concentrated under reduced pressure to yield the desired product 28.2 (23.8 g): ¹H NMR (400 MHz, DMSO-d₆) δ 1.82 (s, 2H), 1.20 (s, 12H), 0.08 (s, 9H).

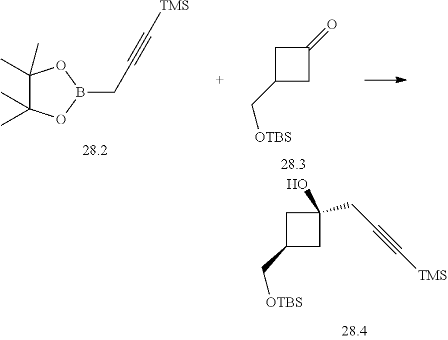 [SMILES: CC1(C)OB(CC#C[Si](C)(C)C)OC1(C)C ; CC(C)(C)[Si](C)(C)OCC1CC(=O)C1 ; CC(C)(C)[Si](C)(C)OC[C@H]1C[C@](O)(CC#C[Si](C)(C)C)C1]

To a stirring solution of 3-(((tert-butyldimethylsilyl)oxy)methyl)cyclobutanone (28.3) (4.3 g, 20.06 mmol) in anhydrous THF (20 mL) at room temperature was added a solution of trimethyl(3-(4,4,5,5-tetramethyl-1,3,2-dioxaborolan-2-yl)prop-1-yn-1-yl)silane (28.2) (5.02 g, 21.06 mmol) in THF (20 mL) under a nitrogen atmosphere followed by diethylzinc (3.65 mL, 4.01 mmol) and the reaction was stirred for 1 hr. Water (30 mL) was added, followed by aqueous HCl (6 M, 1 mL), and the reaction was stirred for 15 min. The reaction was extracted with EtOAc, and the combined extracts were washed with water, dried over sodium sulfate, filtered and concentrated under reduced pressure to give a clear oil (7.62 g), which was purified by flash chromatography (silica gel/30-55% EtOAc/hexanes) to yield the desired product 28.4 (5.20 g): TLC EtOAc/hex 1:1 Rf 0.215; ¹H NMR (400 MHz, DMSO-d₆) δ 5.02 (s 1H), 3.51 (d J=6 Hz, 2H), 2.30 (s, 1H), 2.05 (m, 2H), 1.98 (m 1H), 1.65 (m, 2H), 0.845 (s, 9H), 0.09 (s, 9H), 0.00 (s, 6H).

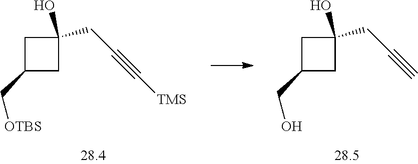

To a stirring solution of (1s,3r)-3-(((tert-butyldimethylsilyl)oxy)methyl)-1-(3-(trimethylsilyl)prop-2-yn-1-yl)cyclobutanol (28.4) (5.2 g) in anhydrous tetrahydrofuran (35 mL) at 0° C. was added tetrabutylammonium fluoride (38 mL, 1 M THF), and the reaction was allowed to warm to room temperature and stirred for 1 hour. Diethyl ether (50 mL) and water (50 mL) were added, and the reaction was stirred for 5 min, it was then extracted into diethyl ether. The combined organic extracts were washed with saturated NaCl, dried over sodium sulfate, filtered and concentrated under reduced pressure to give a crude, which was purified by flash chromatography (silica gel/50-100% EtOAc/hexanes) to yield the desired product 28.5 (790 mg): TLC EtOAc Rf 0.30; ¹H NMR (400 MHz, DMSO-d₆) δ 5.02 (s, 1H), 4.45 (t, J=5.2 Hz), 3.32 (m, 2H), 2.71 (m, 1H), 2.33 (d, J=2.8 Hz, 2H), 2.04 (m, 2H), 1.90 (m, 1H), 1.66 (m, 2H).

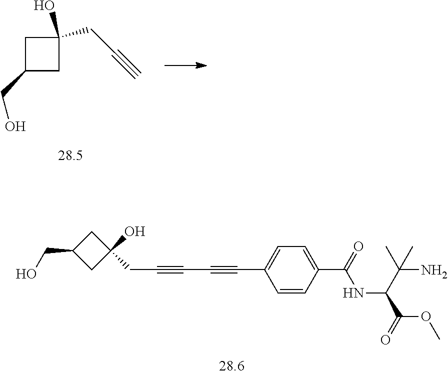

To a stirring solution of hydroxylamine hydrochloride (23.50 mg, 0.338 mmol) and copper(I) chloride (11.16 mg, 0.113 mmol) in 30% aqueous n-butylamine (5 mL) at 0° C. was added a solution of (1s,3r)-3-(hydroxymethyl)-1-(prop-2-yn-1-yl)cyclobutanol (28.5) (790 mg, 5.64 mmol) in 30% n-butylamine/water (10 mL), followed by the addition over 10 min of a solution of (S)-methyl 3-amino-2-(4-(bromoethynyl)benzamido)-3-methylbutanoate hydrochloride (14-1) (2196 mg, 5.64 mmol) and hydroxylamine hydrochloride (23.50 mg, 0.338 mmol) in MeOH (5 mL) and 30% n-butylamine/water (10 mL), and the reaction was stirred for 2 hr at 0° C. The reaction mixture was extracted with ethyl acetate, and the combined extracts were washed with water, dried over sodium sulfate, filtered and concentrated under reduced pressure to give a crude, which was purified by flash chromatography (silica gel/0-5% MeOH/DCM) to yield the desired product 28.6 (1.18 g) as a yellow foam: MS: m/z calcd for C₂₃H₂₈N₂O₅ 412.20. found [M+H]⁺ 413.2.

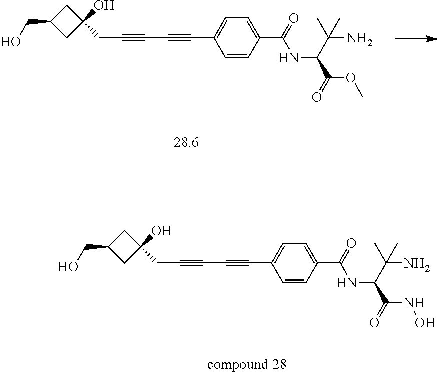 [SMILES: COC(=O)[C@@H](NC(=O)c1ccc(C#CC#CC[C@]2(O)C[C@@H](CO)C2)cc1)C(C)(C)N ; CC(C)(N)[C@H](NC(=O)c1ccc(C#CC#CC[C@]2(O)C[C@@H](CO)C2)cc1)C(=O)NO]

To a stirring solution of (S)-methyl 3-amino-2-(4-(5-(1-hydroxy-3-(hydroxymethyl)cyclobutyl)penta-1,3-diyn-1-yl)benzamido)-3-methylbutanoate (28.6) (1.18 g, 2.86 mmol) in IPA (10 mL) at 0° C. was added 50% aqueous hydroxylamine (2.83 mL, 42.9 mmol) and the reaction was stirred at 0° C. for 3 days. The pH was adjusted to 6 with acetic acid at 0° C., then the mixture was concentrated under reduced pressure to yield a crude, which was purified on a 2-inch column by RP HPLC to yield N—((S)-3-amino-1-(hydroxyamino)-3-methyl-1-oxobutan-2-yl)-4-(5-((1s,3S)-1-hydroxy-3-(hydroxymethyl)cyclobutyl)penta-1,3-diyn-1-yl)benzamide (28) (561 mg) as the partial acetate salt. MS: m/z calcd for C₂₂H₂₇N₃O₅ 413.20. found [M+H]⁺ 414.2; ¹H NMR (400 MHz, DMSO-d₆) δ 8.20 (br s 1H), 7.86 (d, J=8.0 Hz, 2H)), 7.64 (d, J=7.6 Hz, 2H), 5.23 (br s, 1H), 4.29 (s, 1H), 3.35 (d, J=6 Hz, 2H), 2.642 (s, 1H), 2.08 (m 2H), 1.942 (m, 1H), 1.89 (s acetate CH3), 1.759 (t, 2H), 1.097 (s, 3H), 1.017 (s, 3H).

### 29. N—((S)-3-amino-1-(hydroxyamino)-3-methyl-1-oxobutan-2-yl)-4-(((1R,3S)-3-hydroxy-3-(hydroxymethyl)cyclobutyl)buta-1,3-diynyl)benzamide (29)

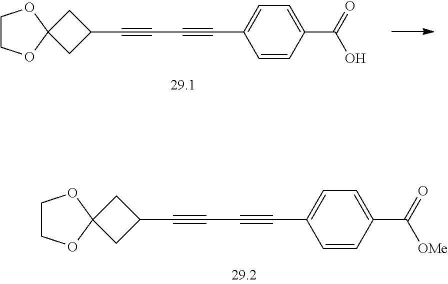 [SMILES: O=C(O)c1ccc(C#CC#CC2CC3(C2)OCCO3)cc1 ; COC(=O)c1ccc(C#CC#CC2CC3(C2)OCCO3)cc1]

To a stirring suspension of compound 29.1 (PCT publication no. WO 2008/154642) (16.8 g, 59.5 mmol) in DOM (60 mL) was added methanol (4.82 mL, 119 mmol), followed by a solution of N,N′-diisopropylcarbodiimide (10.14 mL, 65.5 mmol) and DMAP (5.82 g, 47.6 mmol) in DCM (30 mL), and the reaction was stirred for 24 hours. The salts were removed by filtration and washed with DCM. The organic layer was washed with 1M citric acid, NaHCO₃, brine, dried over Na₂SO₄, filtered and concentrated under reduced pressure to yield compound 29.2 (18.25 g, 61.6 mmol, 103%). MS: m/z calcd for C₁₈H₁₆O₄ 296.10. found [M+H]⁺ 297.1. ¹H NMR: (400 MHz, DMSO-d₆) δ 7.92 (d, 2H), 7.66 (d, 2H), 3.82 (s, 4H), 3.8 (s, 3H), 3.0-3.12 (m, 1H), 2.58-2.7 (m, 2H), 2.28-2.43 (m, 2H).

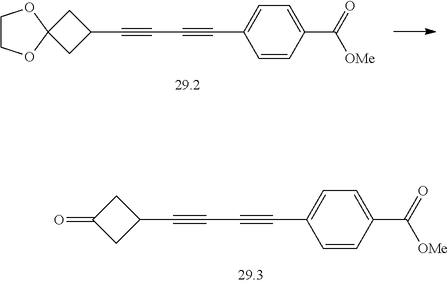 [SMILES: COC(=O)c1ccc(C#CC#CC2CC3(C2)OCCO3)cc1 ; COC(=O)c1ccc(C#CC#CC2CC(=O)C2)cc1]

To a stirring suspension of compound 29.2 (18.25 g, 61.6 mmol) in DCM (205 mL) was added TFA (199 mL, 2587 mmol) and the reaction was stirred for 24 hours. The reaction was diluted with DCM (100 mL), washed with water (250 mL), NaHCO₃ (200 mL), and brine (200 mL), dried over Na₂SO₄, filtered, and concentrated under reduced pressure to give a white solid, which was purified by flash chromatography (silica gel, 0-25% EtOAc/hexanes) to yield compound 29.3 (9.56 g, 37.9 mmol, 61.5%). MS: m/z calcd for C₁₆H₁₂O₃ 252.08. found [M+H]⁺ 253.0. ¹H NMR (400 MHz, DMSO-d₆) δ 7.95 (d, 2H), 7.65 (d, 2H), 3.82 (s, 3H), 3.45-3.55 (m, 3H), 3.2-3.4 (m, 2H).

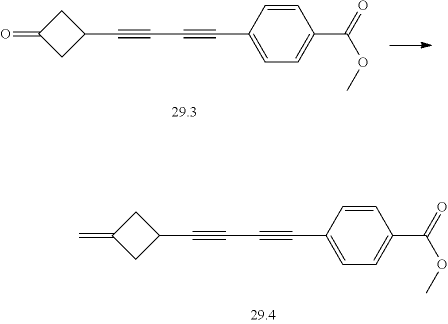 [SMILES: COC(=O)c1ccc(C#CC#CC2CC(=O)C2)cc1 ; C=C1CC(C#CC#Cc2ccc(C(=O)OC)cc2)C1]

To a stirring solution of methyltriphenylphosphonium bromide (2.124 g, 5.95 mmol) in THF (10.01 mL) was added potassium tert-butoxide (20% in THF, 2.67 g, 4.76 mmol) and the reaction was stirred for 2 hours. The resulting orange suspension was cooled to 0° C. A solution of compound 29.3 (1 g, 3.96 mmol) in THF (10.01 mL) was then added and the reaction was stirred for 18 hours. The reaction was concentrated under reduced pressure to give a crude, which was purified by flash chromatography (silica gel, 0-10% EtOAc/hexanes) to yield compound 29.4 (0.2 g, 0.799 mmol, 20%). MS: m/z calcd for C₁₇H₁₄O₂ 250.10. found [M+H]⁺ 251.6. ¹H NMR (400 MHz, DMSO-d₆) δ 7.92 (d, 2H), 7.63 (d, 2H), 4.76-4.83 (m, 2H), 3.83 (s, 3H), 3.23-3.41 (m, 1H), 2.97-3.1 (m, 2H), 2.71-2.86 (m, 2H).

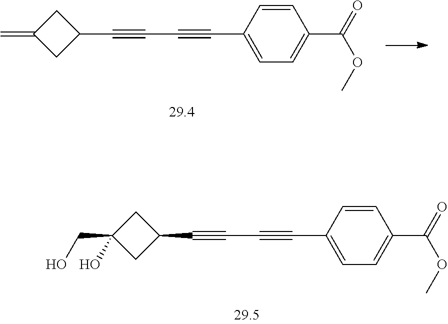 [SMILES: C=C1CC(C#CC#Cc2ccc(C(=O)OC)cc2)C1 ; COC(=O)c1ccc(C#CC#C[C@H]2C[C@@](O)(CO)C2)cc1]

To a stirring solution of compound 29.4 (0.2 g, 0.799 mmol) in water (0.749 mL) and acetone (0.996 mL) was added quinuclidine (0.888 mg, 7.99 μmol) followed by NMO (50% in water, 0.206 mL, 0.879 mmol) and Osmium tetroxide (4% in water, 0.051 mL, 7.99 μmol) and the reaction was stirred for 18 hours and was then heated at 40° C. for 3 days. The reaction was diluted with ethyl acetate, washed with water and brine, dried over Na₂SO₄, filtered, and concentrated under reduced pressure to give an orange oil, which was purified by flash chromatography (silica gel, 0-50% EtOAc/hexanes) to yield compound 29.5 (0.1 g, 0.352 mmol, 44%). MS: m/z calcd for C₁₇H₁₆O₄ 284.10. found [M+H]⁺ 285.7.

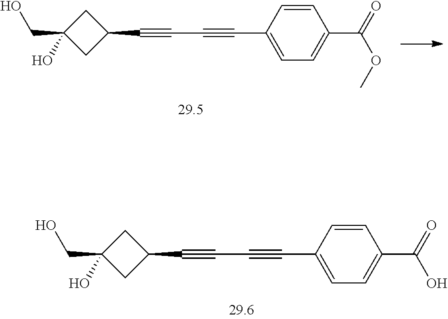 [SMILES: COC(=O)c1ccc(C#CC#C[C@H]2C[C@@](O)(CO)C2)cc1 ; O=C(O)c1ccc(C#CC#C[C@H]2C[C@@](O)(CO)C2)cc1]

To a stirring solution of 29.5 (100 mg, 0.352 mmol) in methanol was added LiOH (528 ul, 0.528 mmol) and the reaction was allowed to stir for 3 hours at room temperature. The reaction was quenched by the addition of HCl (500 μL, 0.5 M, 0.25 mmol). The methanol was removed under reduced pressure and the product was extracted with 2-methyl-THF (10 mL). The resulting organic layer was washed with brine (10 mL) and dried over Na₂SO₄, filtered and concentrated under reduced pressure to provide compound 29.6 (88.5 mg, 0.327 mmol, 93% yield) as a white solid, which was used in the next step without further purification. MS: m/z calcd for C₁₆H₁₄O₄ 270.09. found [M+H]⁺ found 271.1.

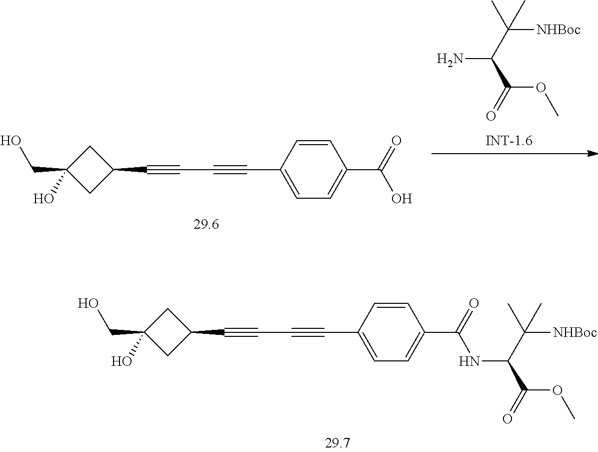 [SMILES: COC(=O)[C@@H](N)C(C)(C)NC(=O)OC(C)(C)C ; O=C(O)c1ccc(C#CC#C[C@H]2C[C@@](O)(CO)C2)cc1 ; COC(=O)[C@@H](NC(=O)c1ccc(C#CC#C[C@H]2C[C@@](O)(CO)C2)cc1)C(C)(C)NC(=O)OC(C)(C)C]

To a stirring solution of compound 29.6 (88 mg, 0.326 mmol) in DMF (1.63 mL) at 0° C. was added a solution of INT-1.6 (80 mg, 0.326 mmol) in DMF (1.63 mL), followed by DIPEA (57 μL) and a solution of HATU (124 mg, 0.326 mmol) in DMF (1.63 mL). After 60 minutes, the reaction was diluted with EtOAc (15 mL) and washed with brine (1×35 mL, 1×15 mL), saturated NaHCO₃ (10 mL), brine (10 mL) and dried over Na₂SO₄. The organic layer was concentrated under reduced pressure to give 29.7 (190 mg, 0.381 mmol, 117%) as a sticky solid, which was used in the next step without further purification. MS m/z calcd for C₂₇H₃₄N₂O₇ 498.24. found [M+H]⁺ 499.3.

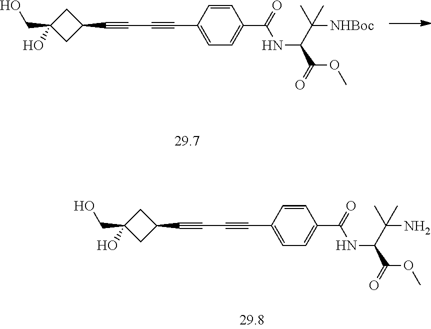 [SMILES: COC(=O)[C@@H](NC(=O)c1ccc(C#CC#C[C@H]2C[C@@](O)(CO)C2)cc1)C(C)(C)NC(=O)OC(C)(C)C ; COC(=O)[C@@H](NC(=O)c1ccc(C#CC#C[C@H]2C[C@@](O)(CO)C2)cc1)C(C)(C)N]

To a stirring solution of 29.7 (190 mg) in DCM (634 μL) at 0° C. was added TFA (1.27 mL) and the reaction was allowed to warm to room temperature and stir for an additional 30 minutes. Volatiles were removed under reduced pressure and the resulting thick oil was diluted with isopropanol (50 mL) and concentrated under reduced pressure to give compound 29.8, which was used in the next step without purification. MS m/z calcd for C₂₂H₂₆N₂O₅ 399.18. found [M+H]⁺ 399.1.

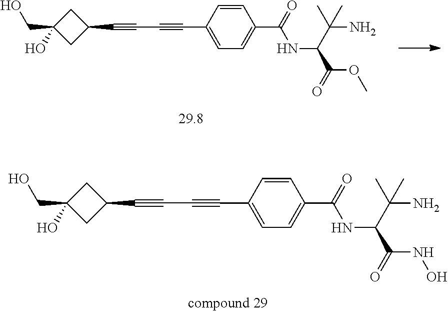 [SMILES: COC(=O)[C@@H](NC(=O)c1ccc(C#CC#C[C@H]2C[C@@](O)(CO)C2)cc1)C(C)(C)N ; CC(C)(N)[C@H](NC(=O)c1ccc(C#CC#C[C@H]2C[C@@](O)(CO)C2)cc1)C(=O)NO]

To a stirring solution of 29.8 (100 mg) in IPA (4.4 mL) at 0° C. was added hydroxylamine (296 μL, 5.02 mmol) and the reaction was allowed to stir at 0-4° C. for 18 hours. The reaction was neutralized with the addition of AcOH (280 μL, 5.0 mmol) and the resulting solution was concentrated under reduced pressure. The residue was dissolved in water (5 mL) and purified by RP HPLC to provide N—((S)-3-amino-1-(hydroxyamino)-3-methyl-1-oxobutan-2-yl)-4-(((1R,3S)-3-hydroxy-3-(hydroxymethyl)cyclobutyl)buta-1,3-diynyl)benzamide (29) (88 mg, 0.192 mmol, 76%) as the acetate salt. MS m/z calcd for C₂₁H₂₅N₃O₅ 400.18. found [M+H]⁺ 400.2; ¹H NMR (400 MHz, DMSO-d₆) δ 8.33 (s, 1H), 7.83 (d, 2H), 7.58 (d, 2H), 4.5-5.3 (bs, 2H), 4.44 (s, 1H), 3.05-4.0 (bs, 4H), 3.24 (d, 2H), 2.72 (m, 1H), 2.40 (t, 1H), 2.11-2.16 (m, 2H), 1.97 (t, 1H), 1.10 and 1.16 (2 s, 6H).

### 30. N—((S)-3-amino-1-(hydroxyamino)-3-methyl-1-oxobutan-2-yl)-4-(((1S,2S,3S)-3-hydroxy-2-(hydroxymethyl)cyclobutyl)buta-1,3-diynyl)benzamide (30A) and N—((S)-3-amino-1-(hydroxyamino)-3-methyl-1-oxobutan-2-yl)-4-(((1S,2S,3R)-3-hydroxy-2-(hydroxymethyl)cyclobutyl)buta-1,3-diynyl)benzamide (30B)

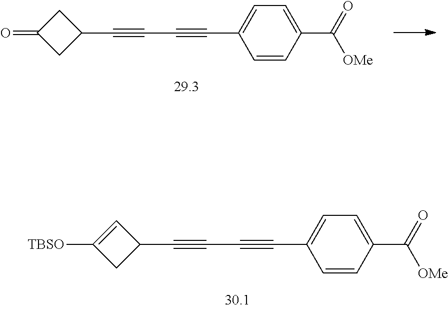 [SMILES: COC(=O)c1ccc(C#CC#CC2CC(=O)C2)cc1 ; COC(=O)c1ccc(C#CC#CC2C=C(O[Si](C)(C)C(C)(C)C)C2)cc1]

To a stirring solution of compound 29.3 (1 g, 3.96 mmol) in THF (17.24 mL) at −78° C. was added dropwise TBSOOTf (1.367 mL, 5.95 mmol), followed by the rapid addition of LiHMDS (19.82 mL, 19.82 mmol), and the reaction was stirred for 1 hour. The reaction was quenched with NH₄Cl, and extracted with ethyl acetate. The organic layer was washed with brine, dried over Na₂SO₄, filtered, and concentrated under reduced pressure to yield compound 30.1 (0.783 g, 2.136 mmol, 54%). MS: m/z calcd for C₂₂H₂₆O₃Si 366.17. found [M+H]⁺ 367.8. ¹H NMR (400 MHz, DMSO-d₆) δ 7.71 (d, 2H), 7.45 (d, 2H), 4.6 (s, 1H), 3.69 (s, 3H), 2.82 (dd, 1H), 2.42 (d, 2H), 0.73 (s, 9H), 0.05 (s, 6H).

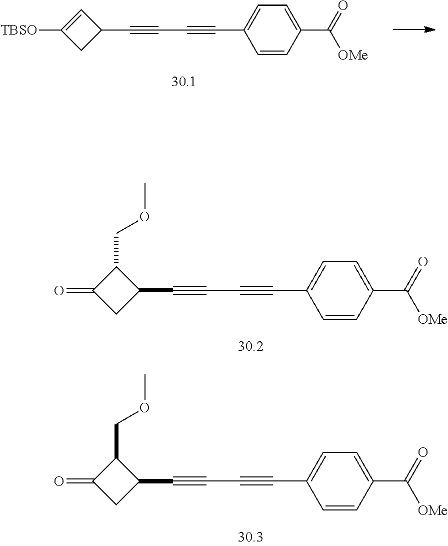 [SMILES: COC(=O)c1ccc(C#CC#CC2C=C(O[Si](C)(C)C(C)(C)C)C2)cc1 ; COC[C@H]1C(=O)C[C@@H]1C#CC#Cc1ccc(C(=O)OC)cc1 ; COC[C@@H]1C(=O)C[C@@H]1C#CC#Cc1ccc(C(=O)OC)cc1]

To a stirring solution of compound 30.1 (0.783 g, 2.136 mmol) in DCM (6.42 mL) at −40° C. was added zinc bromide (0.048 g, 0.214 mmol), followed by chloromethyl methyl ether (0.243 mL, 3.20 mmol) and the reaction was allowed to slowly warm to room temperature and stirred for 2 hours. The reaction was diluted with DCM, washed with water and brine, dried over Na₂SO₄, filtered, and concentrated under reduced pressure to give an orange oil, which was purified by RP-HPLC to yield compound 30.2 (0.147 g, 0.497 mmol, 23%) and 30.3 (0.342 g, 1.16 mmol, 54%). MS for methyl 4-(((trans)-2-(methoxymethyl)-3-oxocyclobutyl)buta-1,3-diyn-1-yl)benzoate: m/z calcd for C₁₈H₁₆O₄ 296.10. found [M+H]⁺ 297.0.

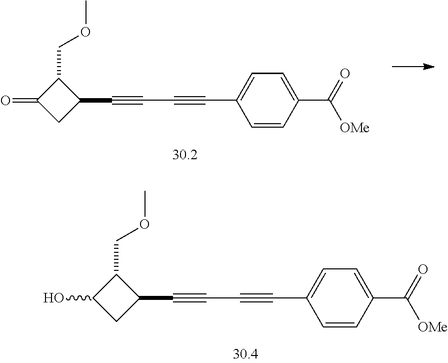 [SMILES: COC[C@H]1C(=O)C[C@@H]1C#CC#Cc1ccc(C(=O)OC)cc1 ; COC[C@H]1C(O)C[C@@H]1C#CC#Cc1ccc(C(=O)OC)cc1]

To a stirring solution of compound 30.2 (0.150 g, 0.506 mmol) in MeOH (5.06 mL) at 0° C. was added NaBH₄ (0.192 g, 5.06 mmol) and the reaction was stirred for 20 minutes. The reaction was quenched with water, extracted with ethyl acetate, washed with brine, dried over Na₂SO₄, filtered, and concentrated under reduced pressure to yield compound 30.4 as a mixture of isomers (0.241 g, 0.808 mmol, 160%). MS: m/z calcd for C₁₈H₁₈O₄ 298.12. found [M+H]⁺ 299.1.

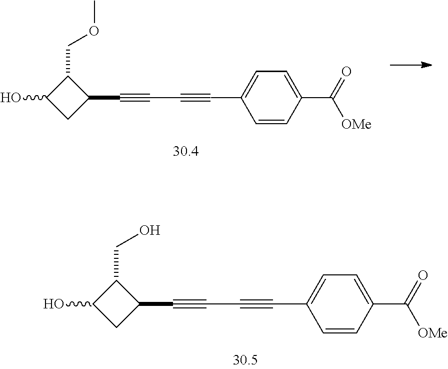 [SMILES: COC[C@H]1C(O)C[C@@H]1C#CC#Cc1ccc(C(=O)OC)cc1 ; COC(=O)c1ccc(C#CC#C[C@H]2CC(O)[C@@H]2CO)cc1]

To a stirring solution of compound 304 (0.240 g, 0.804 mmol) in DCM (12.01 mL) at −78° C. was added BBR₃ (1M in DCM, 1.61 mL, 1.61 mmol) and the reaction was stirred with warming to room temperature over 2 hours. The reaction was quenched with NaHCO₃, extracted with ethyl acetate, washed with brine, dried over Na₂SO₄, filtered, and concentrated under reduced pressure to yield compound 30.5 as a mixture of isomers (0.240 g, 0.804 mmol, 105%). MS: m/z calcd for C₁₇H₁₆O₄ 284.10. found [M+H]⁺ 285.0.

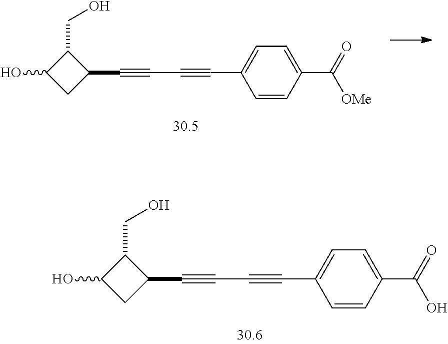 [SMILES: COC(=O)c1ccc(C#CC#C[C@H]2CC(O)[C@@H]2CO)cc1 ; O=C(O)c1ccc(C#CC#C[C@H]2CC(O)[C@@H]2CO)cc1]

To a stirring solution of compound 30.5 (0.240 g, 0.844 mmol) in THF (4.49 mL) and water (4.49 mL) was added LiOH (1M in water, 1.266 mL, 1.266 mmol) and the reaction was stirred for 3 hours. The reaction was diluted with water (50 mL), and washed with ethyl acetate (50 mL). The product rich aqueous layer was acidified with concentrated HCl (0.208 mL, 2.53 mmol), and extracted with ethyl acetate (2×50 mL). The combined organic layers were washed with brine, dried over Na₂SO₄, filtered, and concentrated under reduced pressure to yield compound 30.6 as a mixture of isomers (0.175 g, 0.844 mmol, 77%). MS: m/z calcd for C₁₆H₁₄O₄ 270.09. found [M+H]⁺ 271.0.

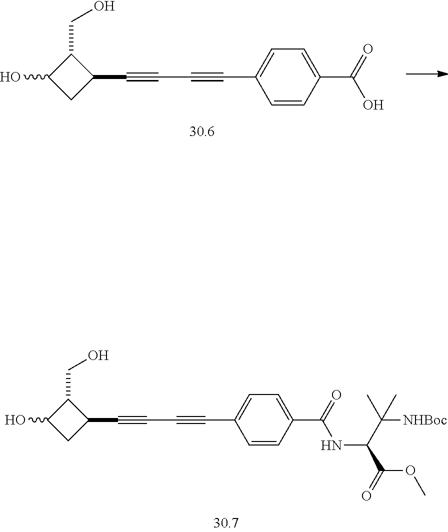

To a stirring solution of methyl 2-amino-3-((tert-butoxycarbonyl)amino)-3-methylbutanoate (0.239 g, 0.971 mmol) in ethyl acetate (2 mL) was added a solution of K₂CO₃ (0.403 g, 2.91 mmol) in water (2 mL) and the reaction was stirred for 1 hour. The two layers were separated and the organic layer was concentrated under reduced pressure to a clear oil.

To a stirring solution of compound 30.6 (0.175 g, 0.647 mmol) in DMF (6.47 mL) was added HATU (0.271 g, 0.712 mmol), followed by DIPEA (0.170 mL, 0.971 mmol) and methyl 2-amino-3-((tert-butoxycarbonyl)amino)-3-methylbutanoate (0.239 g, 0.971 mmol) and the reaction was stirred for 1 hour. The reaction was diluted with ethyl acetate, washed with 1M citric acid, NaHCO₃ and brine, dried over Na₂SO₄, filtered, and concentrated under reduced pressure to yield compound 30.7 as a mixture of isomers (0.323 g, 0.647 mmol, 100%). MS: m/z calcd for C₂₇H₃₄N₂O₇ 498.24. found [M+H]⁺ 499.3.

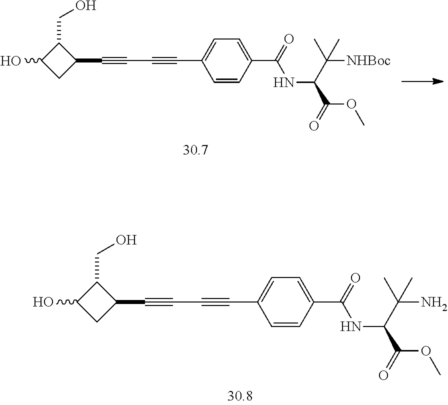 [SMILES: COC(=O)[C@@H](NC(=O)c1ccc(C#CC#C[C@H]2CC(O)[C@@H]2CO)cc1)C(C)(C)NC(=O)OC(C)(C)C ; COC(=O)[C@@H](NC(=O)c1ccc(C#CC#C[C@H]2CC(O)[C@@H]2CO)cc1)C(C)(C)N]

Compound 30.7 (0.323 g, 0.648 mmol) was dissolved in DCM (4.32 mL) and TFA (8.64 mL) and the reaction was stirred for 30 minutes. The reaction was azeotroped with IPA (3×75 mL) and the resulting liquid was basified with K₂CO₃ until basic. This solution was then diluted with ethyl acetate, washed with brine, dried over Na₂SO₄, filtered, and concentrated under reduced pressure to yield compound 30.8 as a mixture of isomers (0.258 g, 0.648 mmol, 100%). MS: m/z calcd for C₂₂H₂₆N₂O₅ 398.18. found [M+H]⁺ 399.2.

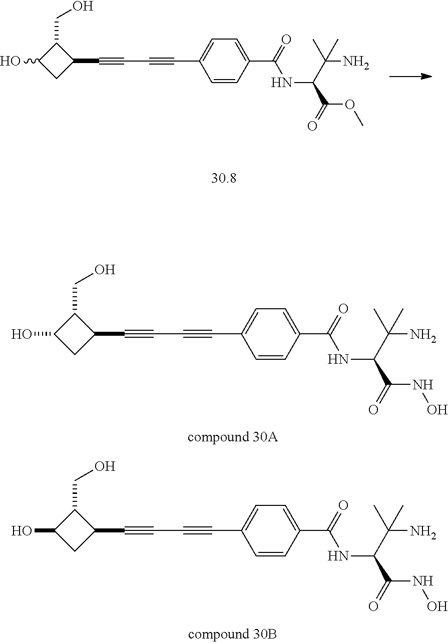

To a stirring solution of compound 30.8 (0.260 g, 0.653 mmol) in IPA (5 mL) was added hydroxylamine (50% solution in water) (3.23 mL, 48.9 mmol) and the reaction was stirred for 18 hours. The reaction was then concentrated under reduced pressure to a thick oil, which was purified by RP-HPLC to yield N—((S)-3-amino-1-(hydroxyamino)-3-methyl-1-oxobutan-2-yl)-4-(((1S,2S,3S)-3-hydroxy-2-(hydroxymethyl)cyclobutyl)buta-1,3-diynyl)benzamide (30A) (0.108 g, 0.257 mmol, 39%) and N—((S)-3-amino-1-(hydroxyamino)-3-methyl-1-oxobutan-2-yl)-4-(((1S,2S,3R)-3-hydroxy-2-(hydroxymethyl)cyclobutyl)buta-1,3-diynyl)benzamide (30B) (0.0094 g, 0.022 mmol, 4%), as a mixture of diasteromers. MS for N—((S)-3-amino-1-(hydroxyamino)-3-methyl-1-oxobutan-2-yl)-4-(((1S,2S,3S)-3-hydroxy-2-(hydroxymethyl)cyclobutyl)buta-1,3-diynyl)benzamide (30A): m/z calcd for C₂₁H₂₅N₃O₅ 399.18. found [M+H]+400.7. MS for N—((S)-3-amino-1-(hydroxyamino)-3-methyl-1-oxobutan-2-yl)-4-(((1S,2S,3R)-3-hydroxy-2-(hydroxymethyl)cyclobutyl)buta-1,3-diynyl)benzamide (30B): m/z calcd for C₂₁H₂₅N₃O₅ 399.18. found [M+H]⁺ 400.7.

### 31. N—((S)-3-amino-1-(hydroxyamino)-3-methyl-1-oxobutan-2-yl)-4-(((1S,2R,3R)-3-hydroxy-2-(hydroxymethyl)cyclobutyl)buta-1,3-diynyl)benzamide (31A) and N—((S)-3-amino-1-(hydroxyamino)-3-methyl-1-oxobutan-2-yl)-4-(((1S,2R,3S)-3-hydroxy-2-(hydroxymethyl)cyclobutyl)buta-1,3-diynyl)benzamide (31B)

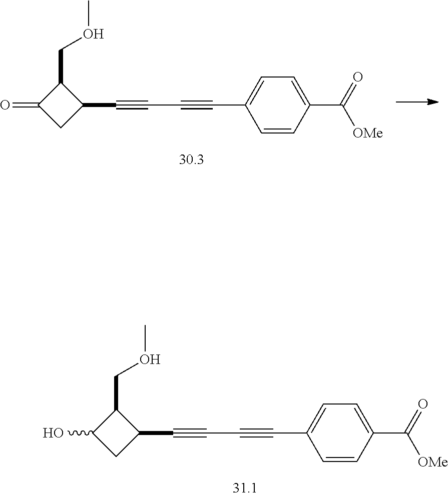

To a stirring solution of methyl 4-(((cis)-2-(methoxymethyl)-3-oxocyclobutyl)buta-1,3-diyn-1-yl)benzoate (30.3, prepared as described in Example 30) (0.340 g, 1.147 mmol) in MeOH (11.47 mL) at 0° C. was added NaBH₄ (0.434 g, 11.47 mmol) and the reaction was stirred for 20 minutes. The reaction was quenched with water, extracted with ethyl acetate, washed with brine, dried over Na₂SO₄, filtered, and concentrated under reduced pressure to yield compound 31.1 as a mixture of isomers (0.362 g, 1.21 mmol, 106%) MS: m/z calcd for C₁₈H₁₈O₄ 298.12. found [M+H]⁺ 299.2.

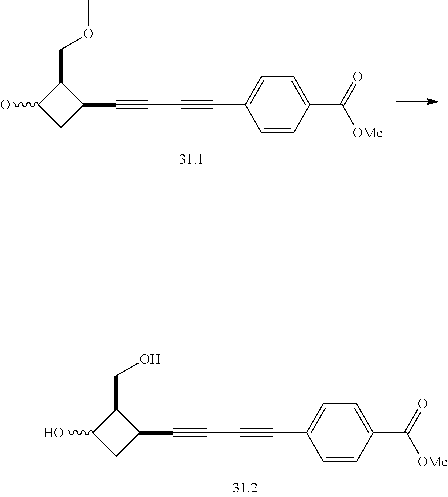

To a stirring solution of compound 31.1 (0.362 g, 1.213 mmol) in DCM (18.11 mL) at −78° C. was added BBR₃ (1M in DCM) (2.426 mL, 2.426 mmol) and the reaction was stirred at room temperature for 1 hour. The reaction was quenched with NaHCO₃, extracted with ethyl acetate, washed with brine, dried over Na₂SO₄, filtered, and concentrated under reduced pressure to yield compound 31.2 as a mixture of isomers (0.480 g, 1.69 mmol, 139%) MS: m/z calcd for C₁₇H₁₆O₄ 284.10. found [M+H]⁺285.0.

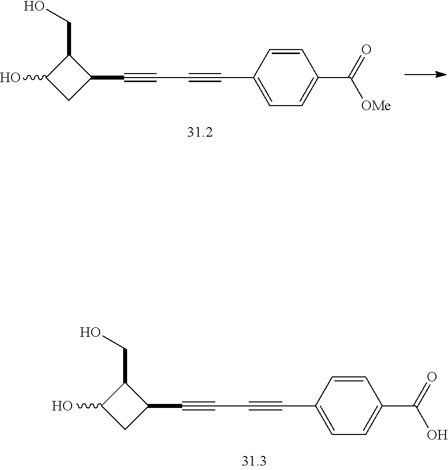

To a stirring solution of compound 31.2 (0.480 g, 1.688 mmol) in THF (8.98 mL) and water (8.98 mL) was added LiOH (1M in water, 5.06 mL, 5.06 mmol) and the reaction was stirred for 5 hours. The reaction was diluted with water (50 mL), washed with ethyl acetate (50 mL), acidified with concentrated HCl (0.555 mL, 6.75 mmol), extracted with ethyl acetate (2×50 mL), and the organic layer was washed with brine, dried over Na₂SO₄, filtered, and concentrated under reduced pressure to yield compound 31.3 as a mixture of isomers (0.254 g, 0.94 mmol, 56%) MS: m/z calcd for C₁₆H₁₄O₄ 270.09. found [M+H]⁺ 271.7.

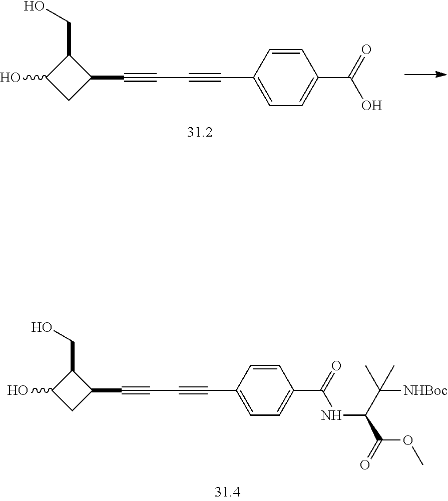

To a stirring solution of methyl 2-amino-3-((tert-butoxycarbonyl)amino)-3-methylbutanoate (0.346 g, 1.404 mmol) in ethyl acetate (3 mL) was added a solution of K₂CO₃ (0.582 g, 4.21 mmol) in water (3 mL) and the reaction was stirred for 1 hour. The layers were separated and the organic layer was concentrated under reduced pressure to a clear oil.

To a stirring solution of compound 31.3 (0.253 g, 0.936 mmol) in DMF (9.36 mL) was added HATU (0.392 g, 1.030 mmol) followed by DIPEA (0.245 mL, 1.404 mmol) and methyl 2-amino-3-((tert-butoxycarbonyl)amino)-3-methylbutanoate (0.346 g, 1.404 mmol) and the reaction was stirred for 1 hour. The reaction was diluted with ethyl acetate, washed with 1M citric acid, NaHCO₃ and brine, dried over Na₂SO₄, filtered, and concentrated under reduced pressure to yield compound 31.4 as a mixture of isomers (0.467 g, 0.94 mmol, 100%) MS: m/z calcd for C₂₇H₃₄N₂O₇ 498.24. found [M+H]⁺ 499.1.

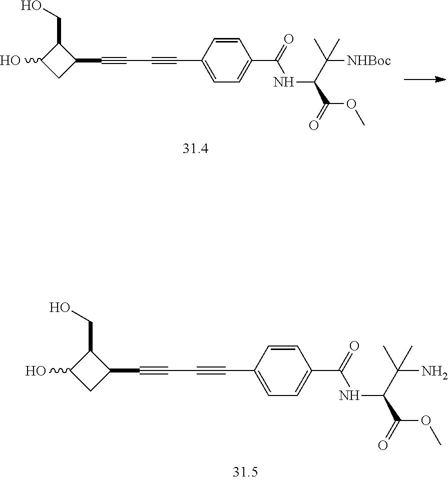

In a 150 mL pear flask compound 31.4 (0.467 g, 0.937 mmol) was dissolved in DCM (6.24 mL) and TFA (12.49 mL) to give a brown solution. The reaction was stirred for 30 minutes, and was then azeotroped with IPA (3×75 mL). The remaining liquid was quenched by adding solid K₂CO₃ until basic. This solution was then diluted with ethyl acetate, washed with brine, dried over Na₂SO₄, filtered, and concentrated under reduced pressure to yield compound 31.5 as a mixture of isomers (0.373 g, 0.94 mmol, 100%) MS: m/z calcd for C₂₂H₂₆N₂O₅ 398.18. found [M+H]⁺ 399.2.

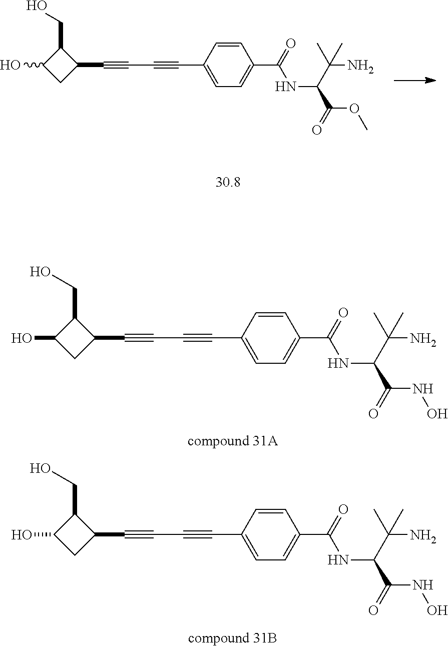

To a stirring solution of compound 31.5 (0.373 g, 0.936 mmol) in IPA (5 mL) was added hydroxylamine (50% solution in water, 4.63 g, 70.2 mmol) and the reaction was stirred for 18 hours. The reaction was then concentrated under reduced pressure to a thick oil, which was purified by RP-HPLC to yield N—((S)-3-amino-1-(hydroxyamino)-3-methyl-1-oxobutan-2-yl)-4-(((1S,2R,3R)-3-hydroxy-2-(hydroxymethyl)cyclobutyl)buta-1,3-diynyl)benzamide (31A) (0.023 g, 0.055 mmol, 6%) and N—((S)-3-amino-1-(hydroxyamino)-3-methyl-1-oxobutan-2-yl)-4-(((1S,2R,3S)-3-hydroxy-2-(hydroxymethyl)cyclobutyl)buta-1,3-diynyl)benzamide (31B) (0.0011 g, 0.003 mmol, 3%), as a mixture of diasteromers. MS for N—((S)-3-amino-1-(hydroxyamino)-3-methyl-1-oxobutan-2-yl)-4-(((1S,2R,3R)-3-hydroxy-2-(hydroxymethyl)cyclobutyl)buta-1,3-diynyl)benzamide (31A): m/z calcd for C₂₁H₂₅N₃O₅ 399.18. found [M+H]⁺ 400.7. MS for N—((S)-3-amino-1-(hydroxyamino)-3-methyl-1-oxobutan-2-yl)-4-(((1S,2R,3S)-3-hydroxy-2-(hydroxymethyl)cyclobutyl)buta-1,3-diynyl)benzamide (31B): m/z calcd for C₂₁H₂₅N₃O₅ 399.18. found [M+H]⁺ 400.7.

### 32. N—((S)-3-amino-1-(hydroxyamino)-3-methyl-1-oxobutan-2-yl)-4-(((1S,3R,4S)-3,4-dihydroxycyclopentyl)buta-1,3-diynyl)benzamide (32)

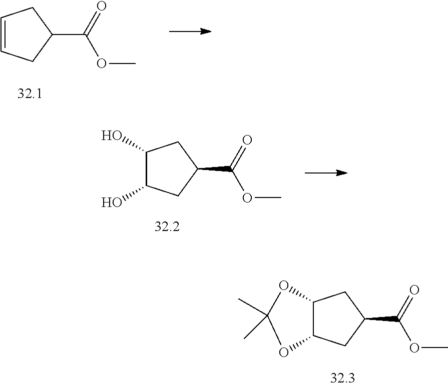

Methyl cyclopent-3-enecarboxylate (32.1) (25 g, 198 mmol) and quinuclidine (0.220 g, 1.982 mmol) were dissolved in water (186 mL) and acetone (247 mL) to give a yellow solution. NMO (50% in water, 51.1 mL, 218 mmol) was added followed by osmium tetroxide (4% in water, 12.60 mL, 1.982 mmol) and the reaction was stirred for 24 hours. The reaction mixture was concentrated under reduced pressure, azeotroped with acetonitrile (2×100 mL), and dried under vacuum for 18 hours to yield a crude oil, which was purified by flash chromatography (silica gel, 0-100% EtOAc/hexanes) to yield compound 32.2 (25.1 g, 157 mmol, 79%). ¹H NMR (400 MHz, DMSO-d₆) δ 4.42-4.48 (m, 2H), 3.8-3.88 (m, 2H), 3.55 (s, 3H), 2.89-3.02 (m, 1H), 1.67-1.87 (m, 4H).

To a stirring solution of compound 32.2 (25 g, 156 mmol) in dimethoxypropane (249 mL) was added Ts-OH (1.989 g, 10.46 mmol) and the reaction was stirred for 1 hour. The reaction was diluted with ethyl acetate, washed with NaHCO₃ and brine, dried over Na₂SO₄, filtered, and concentrated under reduced pressure to yield compound 32.3 (28.5 g, 142 mmol, 91%). ¹H NMR (400 MHz, DMSO-d₆) δ 4.54-4.63 (m, 2H), 3.58 (s, 3H), 2.77-2.9 (m, 1H), 1.87-1.95 (m, 2H), 1.53-1.67 (m, 2H), 1.3 (s, 3H), 1.18 (s, 3H).

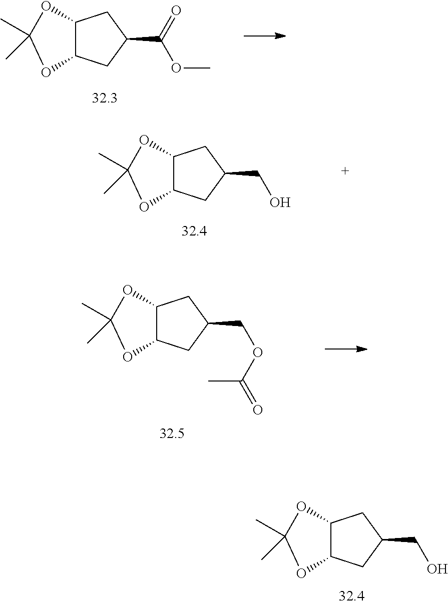

To a stirring solution of compound 32.3 (26.57 g, 133 mmol) in THF (266 mL) at 0° C. was added DIBAL-H (1M in hexane, 398 mL, 398 mmol) and the reaction was stirred for 2 hours. The reaction was quenched by the addition of ethyl acetate (65.0 mL, 663 mmol). Solvents were removed under reduced pressure to yield a brown oil, which was dissolved in ethyl acetate, washed with 1M citric acid, NaHCO₃, brine, dried over Na₂SO₄, filtered, and concentrated under reduced pressure to yield a mixture of compounds 32.4 and 32.5, which was dissolved in DMA (377 mL) and treated with methyl amine (40% in water, 322 mL, 4152 mmol) for 18 hours. The reaction mixture was then diluted with ethyl acetate, washed with NH₄Cl, brine, dried over Na₂SO₄, filtered, and concentrated under reduced pressure to yield compound 32.4 (11.21 g, 65 mmol, 49%). ¹H NMR (400 MHz, DMSO-d₆) δ 4.49-4.59 (m, 2H), 3.96 (d, 1H), 3.35 (d, 2H), 2.09-2.2 (m, 1H), 1.62-1.81 (m, 4H), 1.29 (s, 3H), 1.6 (s, 3H).

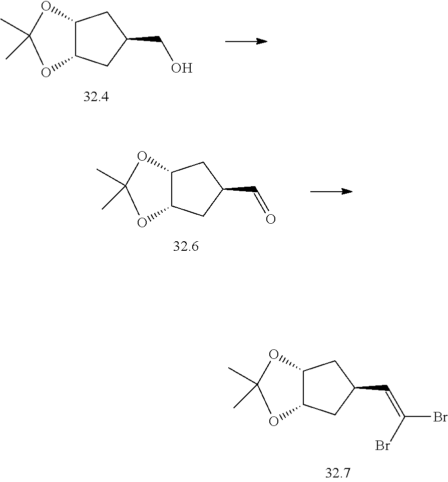

To a stirring solution of compound 32.4 (11.2 g, 65.0 mmol) in DCM (203 mL) was added DIPEA (45.4 mL, 260 mmol), followed by a solution of sulfur trioxide-pyridine (20.83 g, 260 mmol) in DMSO (203 mL). The reaction mixture was stirred for 3 hours, and was then diluted with DCM (200 mL) and washed with 1M citric acid, NaHCO₃, and brine. The organic layer containing 32.6 was then used in the next reaction as is. MS: m/z calcd for C₉H₁₄O₃ 170.1. found [M+H]⁺ 171.4.

To a stirring solution of carbon tetrabromide (3.90 g, 11.75 mmol) in DCM (10 mL, 10 vol.) at −20° C. under a nitrogen atmosphere was added dropwise a solution of triphenylphosphine (6.16 g, 23.50 mmol) in DCM (10 mL) and the reaction was stirred for 30 minutes. It was then cooled to −78° C. and a solution of compound 32.6 (1 g, 5.88 mmol) in DCM (20 mL) was added dropwise and the reaction was stirred for 30 minutes and was then allowed to warm to room temperature and stirred for 18 hours. Solvent removal under reduced pressure gave a brown foam, which was triturated with DCM and the resulting solids were removed by filtration. The organic layer was purified by flash chromatography chromatography (silica gel, 0-20% MTBE/hexanes) to yield compound 32.7 (0.424 g, 1.3 mmol, 22%). ¹H NMR (400 MHz, DMSO-d₆) δ 6.43-6.52 (m, 1H), 4.5-4.6 (m, 2H), 2.73-2.91 (m, 1H), 1.72-1.83 (m, 2H), 1.32-1.43 (m, 2H), 1.3 (s, 3H), 1.18 (s, 3H).

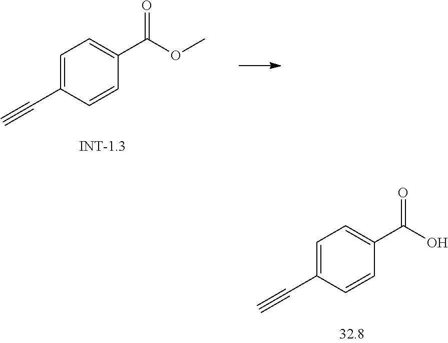

To a stirring solution of methyl 4-ethynylbenzoate (INT-1.3) (20 g, 125 mmol) in THF (100 mL), water (80 mL), and MeOH (60.1 mL) at 0° C., was added LiOH (9.57 g, 400 mmol) and the reaction was stirred for 18 hours. The reaction was pH adjusted to 1 using HCl (41.6 mL, 499 mmol) and was stirred for 2 hours. The resulting solids were removed by filtration, and dried under reduced pressure to yield 4-ethynylbenzoic acid (32.8) (16.55 g, 113 mmol, 91%). MS: m/z calcd for C₉H₆O₂ 146.0. found [M+H]⁺ 147.0.

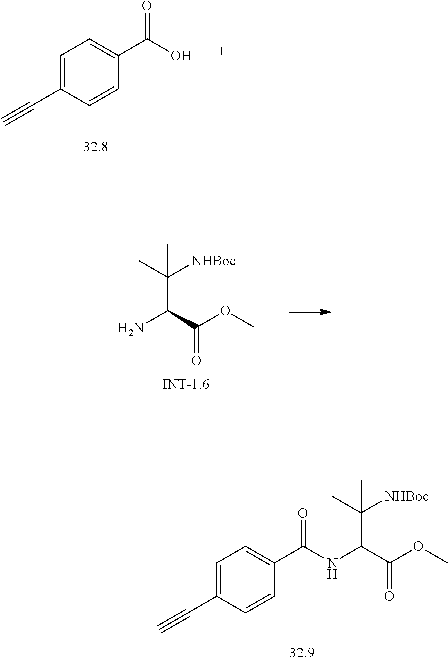 [SMILES: C#Cc1ccc(C(=O)O)cc1 ; COC(=O)[C@@H](N)C(C)(C)NC(=O)OC(C)(C)C ; C#Cc1ccc(C(=O)NC(C(=O)OC)C(C)(C)NC(=O)OC(C)(C)C)cc1]

To a stirring solution of (S)-methyl 2-amino-3-(tert-butoxycarbonylamino)-3-methylbutanoate oxalic acid (INT-1.6; synthesized as described in WO 2008/154642, at page 240 et seq) (98 g, 291 mmol) in ethyl acetate (200 mL) was added a solution of K₂CO₃ (72.3 g, 523 mmol) in water (200 mL) and the reaction was stirred for 1 hour. The reaction mixture was diluted with brine, and partitioned. The aqueous layer was extracted with ethyl acetate and the organic layers were combined and concentrated under reduced pressure to a clear oil. The oil and compound 32.8 (17 g, 116 mmol) were suspended in THF (332 mL) and TEA (48.6 mL, 349 mmol) was added, followed by HATU (53.1 g, 140 mmol) and the reaction was stirred for 45 minutes. Additional (S)-methyl 2-amino-3-(tert-butoxycarbonylamino)-3-methylbutanoate oxalic acid (INT-1.6) (15 g), HATU (25 g), and triethylamine (50 mL) were added and the reaction was stirred for 18 hours. The reaction mixture was diluted with ethyl acetate, washed with 1M citric acid, NaHCO₃, brine, dried over Na₂SO₄, filtered, and concentrated under reduced pressure to a brown foam, which was purified by flash chromatography (silica gel) to yield compound 32.9 (32.16 g, 86 mmol, 74%). MS: m/z calcd for C₂₀H₂₆N₂O₅ 374.18. found [M+H]⁺ 375.5.

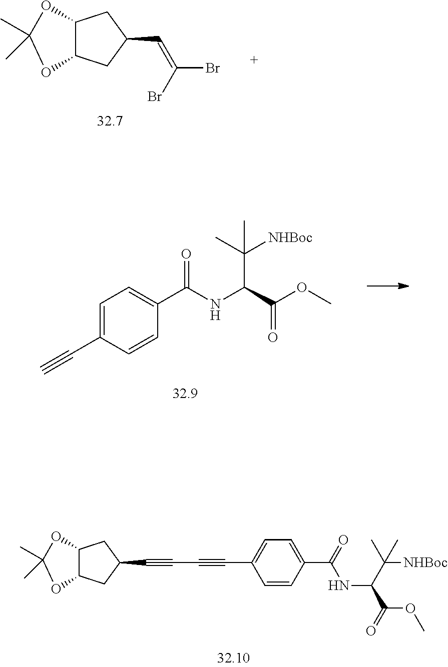

To a stirring solution of compound 32.9 (19.54 mL, 3.07 mmol, 0.157M in DMF) was added compound 32.7 (0.5 g, 1.534 mmol) followed by triethylamine (0.641 mL, 4.60 mmol) and Pd₂(dba)₃ (0.140 g, 0.153 mmol). Tris(4-methoxyphenyl)phosphine (0.216 g, 0.613 mmol) was added and the reaction was heated at 40° C. for 18 hours. Additional triethylamine (0.641 mL, 4.60 mmol), tris(4-methoxyphenyl)phosphine (0.216 g, 0.613 mmol), and Pd₂(dba)₃ (0.140 g, 0.153 mmol) were added and the reaction was stirred at room temperature for 48 hours. The reaction was diluted with ethyl acetate, washed with 1M citric acid, NaHCO₃, brine, dried over Na₂SO₄, filtered, and concentrated under reduced pressure to give a brown oil, which was purified by flash chromatography (silica gel, 0-50% EtOAc/hexanes) to yield compound 32.10 (0.356 g, 0.661 mmol, 43%). MS: m/z calcd for C₃₀H₃₈N₂O₇ 538.27. found [M+H]⁺ 539.3. ¹H NMR (400 MHz, DMSO-d₆) δ 8.77 (s, 1H), 7.8 (d, 2H), 7.6 (d, 2H), 6.81 (s, 1H), 4.77-4.88 (m, 1H), 4.5-4.61 (m, 2H), 3.6 (s, 3H), 2.82-3.02 (m, 1H), 1.93-2.09 (m, 2H), 1.52-1.68 (m, 2H), 1.35 (s, 9H), 1.3 (s, 6H), 1.18 (s, 6H).

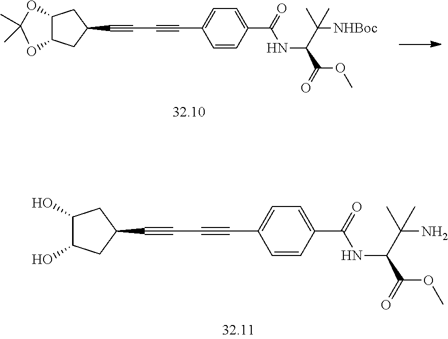

To a stirring solution of compound 32.10 (2.4 g, 4.46 mmol) in DCM (26.2 mL) was added TFA (25.7 mL, 334 mmol) and the reaction was stirred for 72 hours. conc. HCl (5 mL) was added and the reaction was stirred for 2 hours. The reaction was concentrated under reduced pressure to give a brown liquid which was concentrated under reduced pressure with DCM (2×100 mL). The resulting oil was diluted with DCM, washed with NaHCO₃, brine, dried over Na₂SO₄, filtered, and concentrated under reduced pressure to yield compound 32.11 (1.98 g, 4.96 mmol, 111%). MS: m/z calcd for C₂₂H₂₆N₂O₅ 398.18. found [M+H]⁺ 399.2.

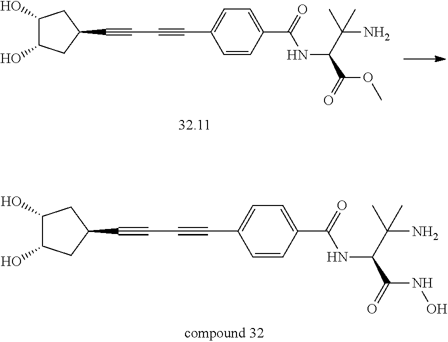 [SMILES: COC(=O)[C@@H](NC(=O)c1ccc(C#CC#C[C@@H]2C[C@@H](O)[C@@H](O)C2)cc1)C(C)(C)N ; CC(C)(N)[C@H](NC(=O)c1ccc(C#CC#C[C@@H]2C[C@@H](O)[C@@H](O)C2)cc1)C(=O)NO]

### 33. N—((S)-3-amino-1-(hydroxyamino)-3-methyl-1-oxobutan-2-yl)-4-(((1S,4R)-4-hydroxy-4-(hydroxymethyl)cyclohexyl)buta-1,3-diynyl)benzamide (33A) and N—((S)-3-amino-1-(hydroxyamino)-3-methyl-1-oxobutan-2-yl)-4-(((1R,4S)-4-hydroxy-4-(hydroxymethyl)cyclohexyl)buta-1,3-diynyl)benzamide (33B)

To a stirring solution of compound 32.11 (2.4 g, 6.02 mmol) in IPA (48.2 mL) was added hydroxylamine (50% in water, 48.0 g, 345 mmol) and the reaction was stirred for 18 hours. The reaction was concentrated under reduced pressure and purified by RP-HPLC to yield N—((S)-3-amino-1-(hydroxyamino)-3-methyl-1-oxobutan-2-yl)-4-(((1S,3R,4S)-3,4-dihydroxycyclopentyl)buta-1,3-diynyl)benzamide (32, 1.14 g, 2.85 mmol, 47%). MS: m/z calcd for C₂₁H₂₅N₃O₅ 399.18. found [M+H]⁺ 400.3. ¹H NMR (400 MHz, DMSO-d₆) δ 7.84 (d, 2H), 7.6 (d, 2H), 4.29 (s, 1H), 3.85-3.98 (m, 2H), 3.09-3.2 (m, 1H), 1.83-1.98 (m, 2H), 1.65-1.81 (m, 2H), 1.08 (s, 3H), 1 (s, 3H).

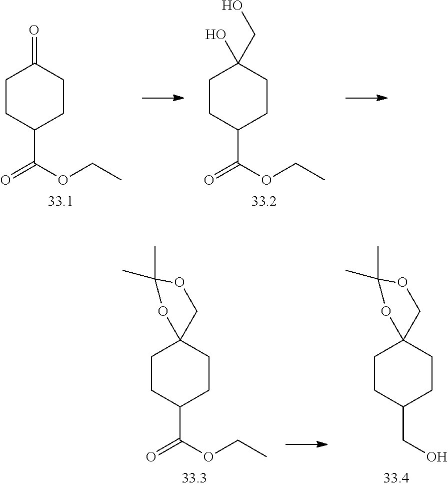 [SMILES: CCOC(=O)C1CCC(=O)CC1 ; CCOC(=O)C1CCC(O)(CO)CC1 ; CCOC(=O)C1CCC2(CC1)COC(C)(C)O2 ; CC1(C)OCC2(CCC(CO)CC2)O1]

Vanadium(III) chloride THF complex (12.08 g, 32.3 mmol) and zinc (4.80 g, 73.4 mmol) were dissolved in DCM (36.7 mL) to give an orange solution, which was stirred for 15 minutes or until green. Paraformaldehyde (8.82 g, 294 mmol) was added to the reaction, followed by a solution of ketone 33.1 (2.358 mL, 14.69 mmol) in DCM (36.7 mL) and the reaction mixture was stirred for 48 hours. The reaction was diluted with DCM (40 mL), quenched with 10% Rochelle's salt (40 mL) and the resulting solution was stirred for 30 minutes. The salts were removed by filtration and washed with DCM. The filtrate was washed with brine and concentrated under reduced pressure to give a white foam, which was purified by flash chromatography (silica gel, 0-5% DCM/methanol) to yield compound 33.2 as a mixture of isomers (1.54 g, 7.61 mmol, 52%). ¹H NMR (400 MHz, DMSO-d₆) δ 4.49 (t, 1H), 4.38 (t, 1H), 3.99-4.05 (m, 4H), 3.98 (s, 1H), 3.9 (s, 1H), 3.18 (d, 2H), 3.11 (d, 2H), 2.36-2.43 (m, 1H), 2.09-2.18 (m, 1H), 1.2-1.8 (m, 16H), 1.15 (t, 6H).

To a stirring solution of compound 33.2 (1.54 g, 7.61 mmol) in dimethoxypropane (12.09 mL) was added Ts-OH (0.970 g, 5.10 mmol) and the reaction was stirred for 5 hours. The reaction was diluted with ethyl acetate, washed with NaHCO₃, brine, dried over Na₂SO₄, filtered, and concentrated under reduced pressure to yield compound 33.3 as a mixture of isomers (1.51 g, 6.23 mmol, 82%), which was carried through to the next step without further purification. ¹H NMR (400 MHz, DMSO-d₆) δ 4.02 (t, 4H), 3.72 (s, 2H), 3.64 (s, 2H), 3.18 (s, 2H), 3.11 (s, 2H), 2.22-2.35 (m, 2H), 1.78-1.87 (m, 2H), 1.3-1.73 (m, 16H), 1.26 (s, 3H), 1.24 (s, 3H), 1.15 (t, 6H).

To a stirring solution of compound 33.3 (1.51 g, 6.23 mmol) in DCM (12.46 mL) at −40° C. was added DIBAL-H (1M in hexane, 15.58 mL, 15.58 mmol) and the reaction was stirred for 2 hours with warming to rt. The reaction was cooled to −40° C. and then quenched with aqueous 10% potassium sodium tartrate and stirred for 1 hour. The reaction mixture was diluted with DCM, washed with brine, dried over Na₂SO₄, filtered and concentrated under reduced pressure to yield compound 33.4 (1.3 g), which was carried through to the next step without further purification.

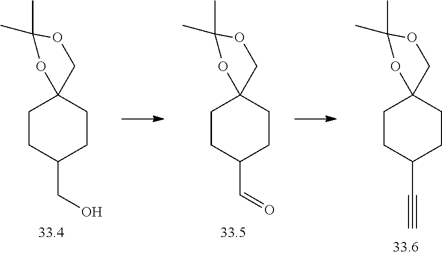

To a stirring solution of compound 33.4 (1.3 g, 6.49 mmol) in DCM (32.5 mL), was added DIPEA (12.47 mL, 71.4 mmol), followed by a solution of sulfur trioxide-pyridine (4.68 g, 58.4 mmol) in DMSO (32.5 mL) and the reaction was stirred for 18 hours. The reaction was diluted with ethyl acetate and washed with 1M citric acid, NaHCO₃, brine, dried over Na₂SO₄, filtered and concentrated under reduced pressure to yield compound 33.5 as a mixture of isomers (1.4 g, 7.06 mmol), which was carried through to the next step without further purification. ¹H NMR (400 MHz, DMSO-d₆) δ 9.55 (s, 2H), 3.67 (d, 4H), 2.20-2.35 (m, 1H), 2.0-2.18 (m, 1H), 1.3-1.9 (m, 16H), 1.24 (s, 12H).

To a stirring solution of dimethyl (2-oxoheptyl)phosphonate (0.210 mL, 1.009 mmol) in ACN (0.841 mL) was added Cs₂CO₃ (0.657 g, 2.018 mmol), followed by benzenesulfonyl azide (0.185 g, 1.009 mmol) and the reaction was stirred at room temperature for 2 hours. Then a solution of compound 33.5 (0.1 g, 0.504 mmol) in ACN/MeOH (0.841 mL/0.168 mL) was added and the reaction was stirred for 18 hours. The reaction mixture was concentrated under reduced pressure to yield an orange oil, which was dissolved in ethyl acetate, washed with 1M citric acid, NaHCO₃, and brine, dried over Na₂SO₄, filtered, and concentrated under reduced pressure to yield compound 33.6 as a mixture of isomers (0.1 g), which was carried through to the next step without further purification. ¹H NMR (400 MHz, DMSO-d₆) δ 3.72 (s, 2H), 3.14 (s, 2H), 2.85 (s, 2H), 2.39-2.47 (m, 1H), 2.28-2.38 (m, 1H), 1.35-1.88 (m, 16H), 1.25 (s, 12H).

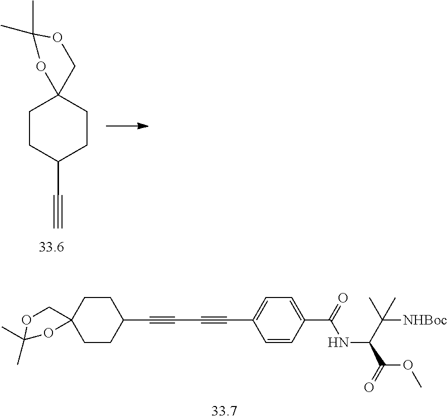

To a stirring solution of compound 33.6 (0.1 g, 0.515 mmol) in 30% aqueous n-butylamine (0.790 mL) at 0° C. was added copper(I) chloride (1.019 mg, 10.29 μmol) followed by hydroxylamine hydrochloride (2.146 mg, 0.031 mmol). In a separate flask (S)-methyl 2-(4-(bromoethynyl)benzamido)-3-((tert-butoxycarbonyl)amino)-3-methylbutanoate (INT-1, 0.233 g, 0.515 mmol) was dissolved in 30% aqueous n-butylamine (0.564 mL) to give an orange solution, to which was added hydroxylamine hydrochloride (2.146 mg, 0.031 mmol) and this solution was added dropwise to the reaction mixture. The reaction was stirred for 4 hours and was then diluted with ethyl acetate, washed with 1M citric acid, NaHCO₃, brine, dried over Na₂SO₄, filtered, and concentrated under reduced pressure to yield compound 33.7 as a mixture of isomers (0.292 g, 0.515 mmol, 100%). MS: m/z calcd for C₃₂H₄₂N₂O₇ 566.30. found [M+H]⁺ 567.4.

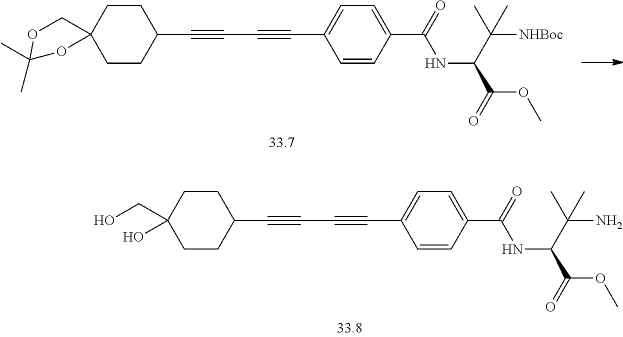

To a stirring solution of compound 33.7 (0.292 g, 0.515 mmol) in DCM (4 mL) was added TFA (6 mL) and the reaction was stirred for 18 hours. The reaction was concentrated under reduced pressure with DCM (30 mL) three times to yield a crude, which was dried under reduced pressure to yield compound 33.8 (0.220 g, 0.515 mmol, 100%). MS: m/z calcd for C₂₄H₃₀N₂O₅ 426.22. found [M+H]⁺ 427.4.

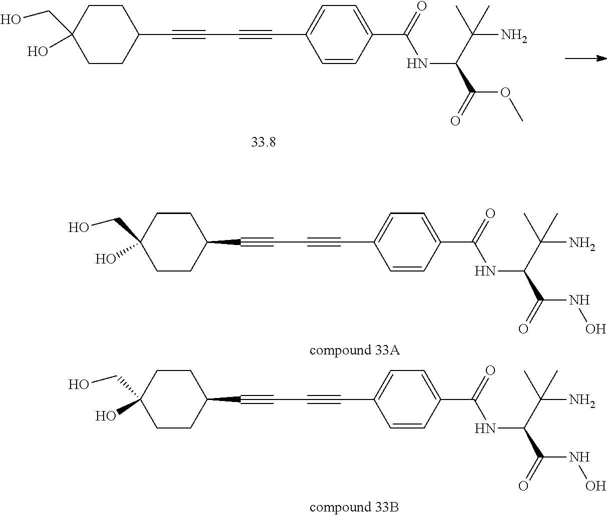

### 34. (S)—N-(3-hydroxy-1-(hydroxyamino)-3-methyl-1-oxobutan-2-yl)-4-(6-hydroxyhexa-1,3-diynyl)benzamide (34)

To a stirring solution of compound 33.8 (0.220 g, 0.388 mmol) in IPA (3.11 mL) was added hydroxylamine (50% in water) (3.09 mL, 22.25 mmol) and the reaction was stirred for 18 hours. The reaction was concentrated under reduced pressure to give a solution, which was acidified to pH 6 with acetic acid and purified by RP-HPLC to yield N—((S)-3-amino-1-(hydroxyamino)-3-methyl-1-oxobutan-2-yl)-4-(((1S,4R)-4-hydroxy-4-(hydroxymethyl)cyclohexyl)buta-1,3-diynyl)benzamide (33A) (0.0047 g, 0.0010 mmol, 2.7%). MS: m/z calcd for C₂₃H₂₉N₃O₅ 427.21. found [M+H]⁺428.4 and N—((S)-3-amino-1-(hydroxyamino)-3-methyl-1-oxobutan-2-yl)-4-(((1R,4S)-4-hydroxy-4-(hydroxymethyl)cyclohexyl)buta-1,3-diynyl)benzamide (33B) (0.0041 g, 0.008 mmol, 2%). MS: m/z calcd for C₂₃H₂₉N₃O₅ 427.21. found [M+H]⁺ 428.4.

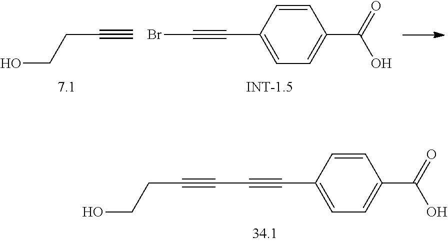

To a stirring solution of but-3-yn-1-ol (7.1) (10 mL, 132 mmol) in 30% aqueous n-butylamine (70.1 mL) at 0° C. was added copper(I) chloride (0.262 g, 2.64 mmol) and hydroxylamine hydrochloride (0.551 g, 7.93 mmol).

In a separate flask, 4-(bromoethynyl)benzoic acid (INT-1.5) (29.7 g, 132 mmol) was dissolved in 30% aqueous n-butylamine (150 mL), then hydroxylamine hydrochloride (0.551 g, 7.93 mmol) was added, and the resulting solution was added dropwise to the initial reaction. After 1 hr, the reaction was washed with MTBE (2×200 mL), cooled to 0° C., diluted with MeTHF (400 mL), and acidified to pH 1 with concentrated HCl. This solution was then filtered through Celite, and the layers were separated. The aqueous layer was extracted with MeTHF (200 mL), and the combined organic layers were washed with 2M HCl (2×200 mL), water (200 mL), brine (200 mL), dried over Na₂SO₄, filtered, and concentrated under reduced pressure to yield compound 34.1 (19.87 g, 93 mmol, 70%). MS: m/z calcd for C₁₃H₁₀O₃ 214.06. found [M+Na]⁺ 237.0.

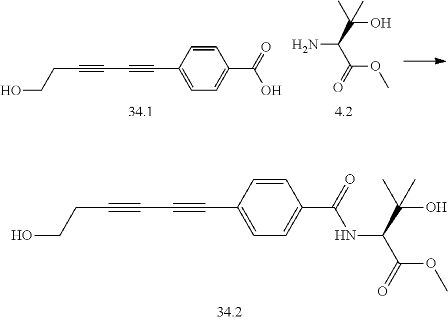

To a stirring solution of (S)-methyl 2-amino-3-hydroxy-3-methylbutanoate (4.2) (5.15 g, 35.0 mmol) in DMF (190 mL) was added K₂CO₃ (14.52 g, 105 mmol) and the reaction was stirred for 1 hour. Compound 34.1 (5 g, 23.34 mmol) and DIPEA (6.11 mL, 35.0 mmol) were added, followed by HATU (9.76 g, 25.7 mmol) and the reaction was stirred for 1 hour. The reaction was diluted with ethyl acetate, washed with 1M citric acid, NaHCO₃ and brine, dried over Na₂SO₄, filtered, and concentrated under reduced pressure to yield compound 34.2 (8 g, 23.3 mmol, 100%). MS: m/z calcd for C₁₉H₂₁NO₅ 343.14. found [M+H]⁺ 344.1.

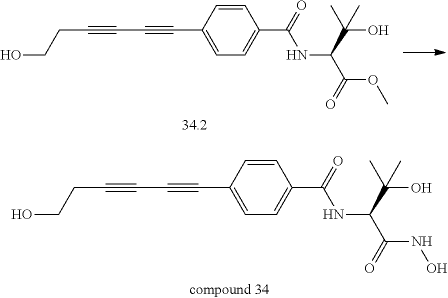 [SMILES: COC(=O)[C@@H](NC(=O)c1ccc(C#CC#CCCO)cc1)C(C)(C)O ; CC(C)(O)[C@H](NC(=O)c1ccc(C#CC#CCCO)cc1)C(=O)NO]

To a stirring solution of compound 34.2 (8 g, 23.30 mmol) in IPA (165 mL) was added hydroxylamine (50% solution in water) (185 mL, 2796 mmol) and the reaction was stirred for 18 hours. The reaction was concentrated under reduced pressure to a thick oil, which was diluted with water and acetic acid and purified by RP HPLC to yield ((3.59 g, 9.9 mmol, 43%). MS: m/z calcd for C₁₈H₂₀N₂O₅ 344.14. found [M+H]⁺ 345.0.

### 35. N-((2S,3R)-3-amino-1-(hydroxyamino)-1-oxobutan-2-yl)-4-(6-hydroxyhexa-1,3-diyn-1-yl)benzamide (35)

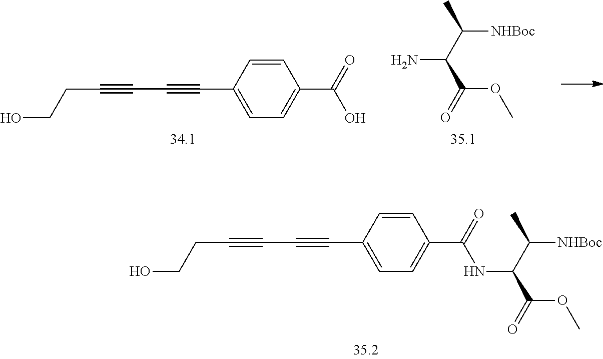 [SMILES: O=C(O)c1ccc(C#CC#CCCO)cc1 ; COC(=O)[C@@H](N)[C@@H](C)NC(=O)OC(C)(C)C ; COC(=O)[C@@H](NC(=O)c1ccc(C#CC#CCCO)cc1)[C@@H](C)NC(=O)OC(C)(C)C]

To a stirring solution of (2S,3R)-methyl 2-amino-3-((tert-butoxycarbonyl)amino)butanoate (35.1, synthesized as described in WO 2008/154642 at page 236 et seq) (8.13 g, 35.0 mmol) in DMF (190 mL) were added compound 34.1 (5 g, 23.34 mmol) and DIPEA (6.11 mL, 35.0 mmol), followed by HATU (9.76 g, 25.7 mmol) and the reaction was stirred for 1 hour. The reaction was diluted with ethyl acetate, washed with 1M citric acid, NaHCO₃ and brine, dried over Na₂SO₄, filtered, and concentrated under reduced pressure to yield compound 35.2 (10.87 g, 25.4 mmol, 109%). MS: m/z calcd for C₂₃H₂₈N₂O₆ 428.19. found [M+H]⁺ 429.7.

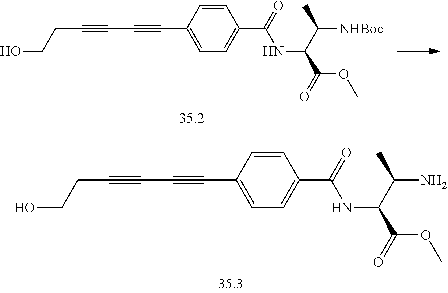 [SMILES: COC(=O)[C@@H](NC(=O)c1ccc(C#CC#CCCO)cc1)[C@@H](C)NC(=O)OC(C)(C)C ; COC(=O)[C@@H](NC(=O)c1ccc(C#CC#CCCO)cc1)[C@@H](C)N]

To a stirring solution of compound 35.2 (10 g, 23.34 mmol) in DCM (78 mL) was added TFA (156 mL) and the reaction was stirred for 1 hour. The reaction was azeotroped with IPA (3×75 mL) and then concentrated under reduced pressure to yield compound 35.3 (7.66 g, 23.34 mmol, 100%). MS: m/z calcd for C₁₈H₂₀N₂O₄ 328.14. found [M+H]⁺ 329.1.

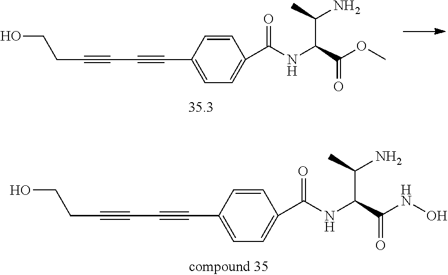 [SMILES: COC(=O)[C@@H](NC(=O)c1ccc(C#CC#CCCO)cc1)[C@@H](C)N ; C[C@@H](N)[C@H](NC(=O)c1ccc(C#CC#CCCO)cc1)C(=O)NO]

To a stirring solution of compound 35.3 (8 g, 24.36 mmol) in IPA (122 mL) was added hydroxylamine (50% solution in water) (96 mL, 1462 mmol), and the reaction was stirred for 18 hours. The reaction was then concentrated under reduced pressure to a thick oil, which was diluted with water and acetic acid, then purified by RP HPLC to yield (2.2 g, 6.35 mmol, 26%). MS: m/z calcd for C₁₇H₁₉N₃O₄329.14. found [M+H]⁺ 330.1.

### 36. N—((S)-3-amino-1-(hydroxyamino)-3-methyl-1-oxobutan-2-yl)-4-((S)-6,7-dihydroxyhepta-1,3-diyn-1-yl)benzamide (36)

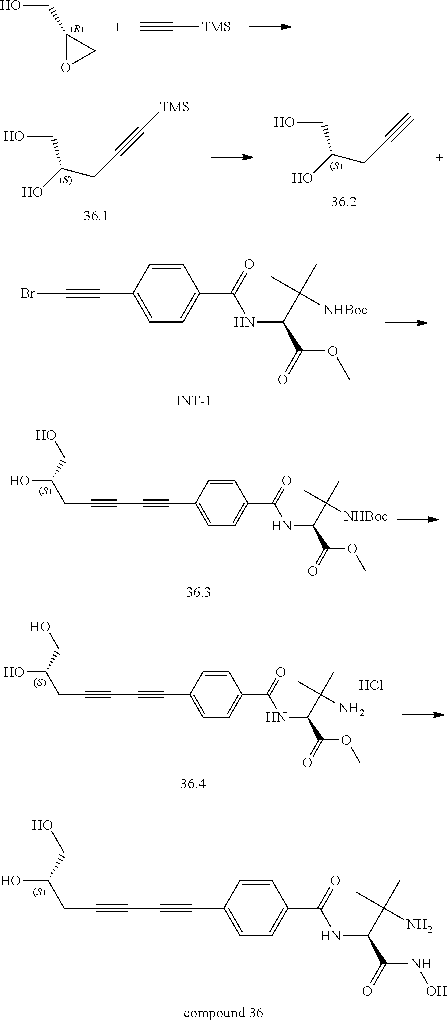

### (S)-5-(trimethylsilyl)pent-4-yne-1,2-diol (36.1)

| Reagent | MW | Eq. | mol | g, mL | |
|---|---|---|---|---|---|
| (R)-oxiran-2-ylmethanol | 74.08 | 1 | 47.4 mmol | 3.51 | g |
| Ethynyltrimethylsilane | 98.22 | 3 | 142 mmol | 20 | mL |
| n-Buli | 64.09 | 2.9 | 137 mmol | 51 | Ml |
| BF₃•Et₂O | 141.93 | 2.9 | 137 mmol | 16.9 | mL |
| THF | | | | | |

To a stirring solution of ethynyltrimethylsilane (20 mL, 142 mmol) in dry THF (200 mL) at −65° C., was added dropwise n-Buli (2.5 M in hexane, 51 mL, 137 mmol). After 20 min BF₃.Et₂O (16.9 mL, 137 mmol) was added and the reaction was stirred for 20 min. (R)-oxiran-2-ylmethanol (3.51 g, 47.4 mmol) was added, and the reaction mixture was allowed to warm to room temperature before NaHCO₃ (saturated. aq) wad added. The solvent was removed under reduced pressure, and the residue was extracted with ethyl acetate (3×100 mL). The combined organic layers were dried and concentrated under reduced pressure to yield compound 36.1 as a yellow oil (6 g), which was used in the next step without further purification.

### (S)-pent-4-yne-1,2-diol (36.2)

| Reagent | MW | Eq. | mmol | g, mL | |
|---|---|---|---|---|---|
| Compound 36.1 | 172.30 | 1 | 17.4 | 3 | g |
| K₂CO₃ | 138.12 | 2.5 | 43.5 | 6.02 | g |
| MeOH | | | | 30 | mL |
| THF | | | | 3 | mL |

To a stirring solution of compound 36.1 (3 g, 17.4 mmoL) in MeOH (30 mL) and THF (3 mL), was added K₂CO₃ (6.02 g, 43.5 mmoL), and the mixture was stirred at rt for 16 hours. The solvent was removed under reduced pressure, and the residue was diluted with water (100 mL), and extracted with ethyl acetate (3×50 mL). The combined organic layers were dried, concentrated under reduced pressure to give a crude, which was purified by flash chromatography (silica gel/Petroleum ether:ethyl acetate 10:1 to 2:1) to yield compound 36.2 as a colorless oil (1.5 g, 42%).

### (S)-methyl 3-(tert-butoxycarbonylamino)-2-(4-((S)-6,7-dihydroxyhepta-1,3-diynyl)benzamido)-3-methylbutanoate (36.3)

| Reagent | MW | Eq. | mmol | g, mL | |
|---|---|---|---|---|---|
| Compound 36.2 | 100.12 | 1.1 | 10 | 1 | g |
| INT-1 | 453.88 | 1 | 9 | 4.9 | g |
| CuCl | 190.45 | 0.02 | 0.18 | 17.57 | mg |
| hydroxylamine hydrochloride | 70.19 | 0.06 | 0.54 | 45.4 | mg |
| butan-1-amine | 101.19 | 23 | 27 | 18.32 | g |
| MeOH | | | | 20 | mL |
| THF | | | | 10 | mL |
| H₂O | | | | 20 | g |

To a stirring solution of CuCl (17.57 mg, 0.18 mmoL) and hydroxylamine hydrochloride (45.4 mg, 0.54 mmol) in 23% butan-1-amine (aqueous) at 0° C. was added a solution of compound 36.2 (1 g, 10 mmol) in 23% butan-1-amine (aqueous). A solution of INT-1 (4.9 g, 9 mmol) in 23% butan-1-amine (12.2 g, 120 mmol), MeOH (20 mL), and THF (10 mL) was then added, and the reaction progress was monitored by TLC. The mixture was diluted with water (100 mL), and extracted with ethyl acetate (3×20 mL). The combined organic layers were dried, concentrated under reduced pressure to give a crude, which was purified by flash chromatography (silica gel/petroleum ether:ethyl acetate 10:1 to 5:1) to yield compound 36.3 as a solid (2.5 g, 60%). MS: m/z calcd for C₂₅H₃₂N₂O₇ 472.2. found [M+H]⁺ 473.

### (S)-methyl 3-amino-2-(4-((S)-6,7-dihydroxyhepta-1,3-diynyl)benzamido)-3-methylbutanoate hydrochloride (36.4)

| Reagent | MW | Eq. | mmol | g, mL | |
|---|---|---|---|---|---|
| Compound 36.3 | 472.53 | 1 | 0.2 | 100 | mg |
| MeOH•HCl | | 5 | 1 | 0.08 | mL |
| MeOH | | | | 2 | mL |

To a stirring solution of compound 36.3 (100 mg, 0.2 mmol) in MeOH (2 mL) was added MeOH.HCl (0.08 mL) and the reaction progress was followed by TLC. The reaction mixture was diluted with Et₂O (50 mL), and filtered to collect the desired product 36.4 as a white solid (68 mg, 95%). ¹H NMR (400 MHz, DMSO-d₆) δ:9.07 (d, J=8.0 Hz, 1H), 8.40 (s, 3H), 8.00 (d, J=8.0 Hz, 2H), 7.66 (d, J=8.0 Hz, 2H), 4.88 (d, J=8 Hz, 1H), 3.72 (s, 3H), 3.5 (m, 1H), 3.38 (m, 3H), 2.64 (m, 1H), 2.49 (m, 1H), 1.41 (d, J=2 Hz, 6H).

### N—((S)-3-amino-1-(hydroxyamino)-3-methyl-1-oxobutan-2-yl)-4-((S)-6,7-dihydroxyhepta-1,3-diyn-1-yl)benzamide (36)

To a stirring solution of compound 36.4 (5 g, 13.43 mmol) in isopropyl alcohol (57.6 ml) was added hydroxylamine (50% solution in water, 53.2 ml, 806 mmol) and the reaction was stirred for 18 hours. The reaction mixture was then concentrated under reduced pressure to a thick oil, which was diluted with acetic acid (70 mL) and purified by RP HPLC (0.1% AcOH in H₂O/ACN) to yield 36 (3.77 g, 71.4%). MS: m/z calcd for C₁₉H₂₃N₃O₅ 373.2. found [M+H]⁺ 374.2.

### 37. N—((S)-3-amino-1-(hydroxyamino)-3-methyl-1-oxobutan-2-yl)-4-((R)-6,7-dihydroxyhepta-1,3-diyn-1-yl)benzamide (37)

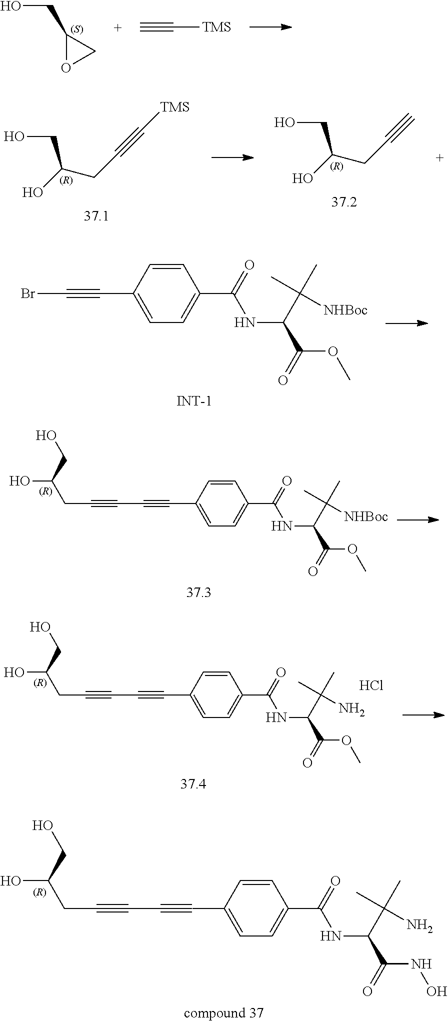

### (R)-5-(trimethylsilyl)pent-4-yne-1,2-diol (37.1)

| Reagent | MW | Eq. | mol | g, mL | |
|---|---|---|---|---|---|
| (S)-oxiran-2-ylmethanol | 74.08 | 1 | 47.4 mmol | 3.51 | g |
| Ethynyltrimethylsilane | 98.22 | 3 | 142 mmol | 20 | mL |
| n-Buli | 64.09 | 2.9 | 137 mmol | 51 | mL |
| BF₃•Et₂O | 141.93 | 2.9 | 137 mmol | 16.9 | mL |
| THF | | | | 200 | mL |

To a stirring solution of ethynyltrimethylsilane (20 mL, 142 mmol) in dry THF (200 mL), cooled to −65° C. was added n-Buli (2.5 M in hexane, 51 mL, 137 mmol) dropwise. After 20 min, BF₃.Et₂O (16.9 mL, 137 mmol) was added and the reaction was stirred for 20 min. (S)-oxiran-2-ylmethanol (3.51 g, 47.4 mmol) was added, and the reaction was allowed to warm to room temperature before addition of NaHCO₃ (saturated. aq). The solvent was removed under reduced pressure, and the residue was extracted with EA (3×100 mL). The combined organic layers were dried and concentrated to yield compound 37.1 as a yellow oil (6 g), which was used in the next reaction without further purification.

### (R)-pent-4-yne-1,2-diol (37.2)

| Reagent | MW | Eq. | mmol | g, mL | |
|---|---|---|---|---|---|
| Compound 37.1 | 172.30 | 1 | 17.4 | 3 | g |
| K₂CO₃ | 138.12 | 2.5 | 43.5 | 6.02 | g |
| MeOH | | | | 30 | mL |
| THF | | | | 3 | mL |

To a stirring solution of compound 37.1 (3 g, 17.4 mmoL) in MeOH (30 mL) and THF (3 mL), was added K₂CO₃ (6.02 g, 43.5 mmoL) and the reaction mixture was stirred at it for 16 hours. The solvent was removed under reduced pressure, the residue was diluted with water (100 mL), and extracted with EA (3×50 mL). The organic layer was dried and purified by flash chromatography (silica gel/PE:EA 10:1 to 2:1) to give compound 37.2 as a colorless oil (1.5 g, 42%)¹H NMR (400 MHz, CDCl₃) δ 2.06-2.07 (m, 1H), 2.43-2.45 (m, 2H), 3.58-3.63 (m, 1H), 3.74-3.77 (m, 1H), 3.88-3.91 (m, 1H).

### (S)-methyl-3-(tert-butoxycarbonylamino)-2-(4-((R)-6,7-dihydroxyhepta-1,3-diyny)benzamido)-3-methylbutanoate (37.3)

| Reagent | MW | Eq. | mmol | g, mL | |
|---|---|---|---|---|---|
| Compound 37.2 | 100.12 | 1.1 | 10 | 1 | g |
| INT-1 | 453.88 | 1 | 9 | 4.9 | g |
| CuCl | 190.45 | 0.02 | 0.18 | 17.57 | mg |
| hydroxylamine hydrochloride | 70.19 | 0.06 | 0.54 | 45.4 | mg |
| butan-1-amine | 101.19 | 23 | 27 | 18.32 | g |
| MeOH | | | | 20 | mL |
| THF | | | | 10 | mL |
| H₂O | | | | 20 | g |

To a stirring solution of CuCl (17.57 mg, 0.18 mmoL) and hydroxylamine hydrochloride (45.4 mg, 0.54 mmol) in 23% butan-1-amine (aq) at 0° C. was added compound 37.2 (1 g, 10 mmol) in 23% butan-1-amine (aq). A solution of INT-1 (4.9 g, 9 mmol) in butan-1-amine (12.2 g, 120 mmol), MeOH (20 mL), and THF (10 mL) was added, and the reaction progress was followed by TLC. The reaction mixture was diluted with water (100 mL), and extracted with EA (3×20 mL). The combined organic layers were dried and purified by flash chromatography (silica gel/PE:EA 10:1 to 5:1) to yield compound 37.3 as a solid (2.5 g, 60%). MS: m/z calcd for C₂₅H₃₂N₂O₇ 472.2. found [M+H]⁺ 473.

### (S)-methyl 3-amino-2-(4-((R)-6,7-dihydroxyhepta-1,3-diynyl)benzamido)-3-methylbutanoate hydrochloride (37.4)

| Reagent | MW | Eq. | mmol | g, mL | |
|---|---|---|---|---|---|
| Compound 37.3 | 472.53 | 1 | 0.2 | 100 | mg |
| MeOH•HCl | | 5 | 1 | 0.08 | mL |
| MeOH | | | | 2 | mL |

To a stirring solution of compound 37.3 (100 mg, 0.2 mmol) in MeOH (2 mL) was added MeOH.HCl (0.08 mL), and the reaction progress was monitored by TLC. The reaction mixture was diluted with Et₂O (50 mL), and filtered to collect compound 37.4 HCl salt as a white solid (71 mg, 96%). ¹H NMR (400 MHz, DMSO-d₆) δ 9.09 (t, J=4.0 Hz, 1H), 8.45 (s, 3H), 8.01 (d, J=8.0 Hz, 2H), 7.66 (d, J=8.0 Hz, 2H), 5.11 (s, 1H), 4.87 (d, J=8 Hz, 1H), 4.77 (s, 1H), 3.72 (s, 3H), 3.67 (s, 1H), 3.4 (m, 1H), 2.64 (m, 1H), 2.49 (m, 1H), 1.41 (d, J=2 Hz, 6H).

### N—((S)-3-amino-1-(hydroxyamino)-3-methyl-1-oxobutan-2-yl)-4-((R)-6,7-dihydroxyhepta-1,3-diyn-1-yl)benzamide, Acetate (37)

To a stirring solution of compound 37.4 (5 g, 13.43 mmol) in isopropyl alcohol (57.6 ml) was added hydroxylamine (50% solution in water, 53.2 ml, 806 mmol) and the reaction was stirred for 18 hours. The reaction mixture was then concentrated under reduced pressure to a thick oil, which was diluted with acetic acid (70 mL) and purified by RP HPLC (0.1% AcOH in H₂O/ACN) to yield 37 as its acetate salt (3.22 g, 61%). MS: m/z calcd for C₁₉H₂₃N₃O₅373.2. found [M+H]⁺ 374.1.

### 38. N-((2S,3R)-3-hydroxy-1-(hydroxyamino)-1-oxobutan-2-yl)-4-(6-hydroxyhexa-1,3-diyn-1-yl)benzamide (38)

### (2S,3R)-methyl 3-hydroxy-2-(4-(6-hydroxyhexa-1,3-diyn-1-yl)benzamido)butanoate (38.2)

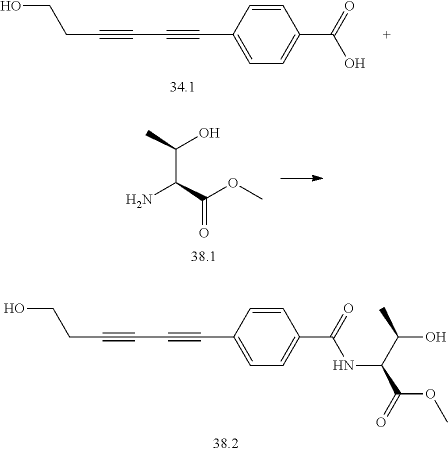

| | | | | | Sample | |
|---|---|---|---|---|---|---|
| | | | Density | Moles | Mass | Vol |
| Reactant | MW | Eq | (g/ml) | (mmol) | (g) | (ml) |
| Compound 34.1 | 214.217 | 1.000 | | 14.00 | 3 | |
| (2S,3R)-methyl 2- | 133.146 | 1.5 | | 21.01 | 3.56 | |
| amino-3- | | | | | | |
| hydroxybutanoate, | | | | | | |
| HCI | | | | | | |
| HATU | 235.265 | 1.1 | | 15.40 | 5.86 | |
| DIPEA | 129.243 | 1.5 | 0.74 | 21.01 | 2.71 | 3.67 |
| K₂CO₃ | 60.010 | 4.5 | | 63.0 | 8.71 | |

To a stirring solution of (2S,3R)-methyl 2-amino-3-hydroxybutanoate (3.56 g, 21.01 mmol) in DMF (114 mL) was added K₂CO₃ (8.71 g, 63.0 mmol) and the reaction was stirred for 1 hour. Compound 34.1 (3 g, 14.00 mmol) and DIPEA (3.67 ml, 21.01 mmol) were added, followed by HATU (5.86 g, 15.40 mmol) and the reaction was stirred for 1 hour. The reaction mixture was diluted with ethyl acetate, washed with 1M citric acid, NaHCO₃ and brine, dried over Na₂SO₄, filtered, and concentrated under reduced pressure to yield 38.2 as a brown oil, which was carried through to the next step without further purification. MS: m/z calcd for C₁₈H₁₉NO₅ 329.1. found [M+H]⁺330.1.

### N-((2S,3R)-3-hydroxy-1-(hydroxyamino)-1-oxobutan-2-yl)-4-(6-hydroxyhexa-1,3-diyn-1-yl)benzamide (38)

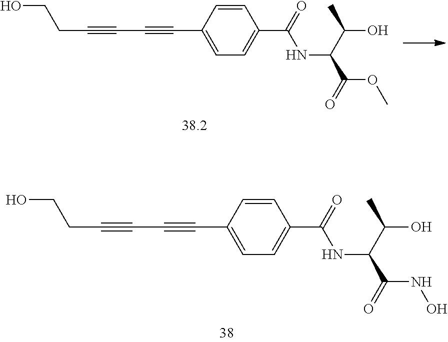 [SMILES: COC(=O)[C@@H](NC(=O)c1ccc(C#CC#CCCO)cc1)[C@@H](C)O ; C[C@@H](O)[C@H](NC(=O)c1ccc(C#CC#CCCO)cc1)C(=O)NO]

To a stirring solution of compound 38.2 (4.61 g, 14.00 mmol) in IPA (60.1 mL) was added hydroxylamine (50% solution in water, 55.4 ml, 840 mmol) and the reaction was stirred for 18 hours. The reaction was then concentrated under reduced pressure to a thick oil, which was diluted with acetic acid (70 mL) and purified by RP HPLC (0.1% AcOH in H₂O/ACN) to yield 38 (1.87 g, 38.4%). MS: m/z calcd for C₁₇H₁₈N₂O₅330.1. found [M+H]⁺ 331.3.

### 39. N—((S)-3-amino-1-(hydroxyamino)-3-methyl-1-oxobutan-2-yl)-4-(6-hydroxy-5-methylhexa-1,3-diyn-1-yl)benzamide

### 2-methylbut-3-yn-1-ol (39.1)

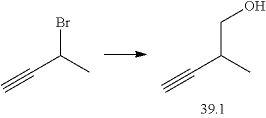

| | | | Moles | | |
|---|---|---|---|---|---|
| Reactant | MW | Eq | (mmol) | Sample Mass | |
| 3-bromobut-1-yne | 132.987 | 1 | 26.8 | 3.56 | g |
| Aluminium | 27 | .75 | 20.08 | 0.542 | g |
| mercury (II) chloride | 271.496 | .001 | 0.027 | 7.27 | mg |
| paraformaldehyde | 30.026 | 2 | 53.5 | 1.608 | g |

To a stirring white suspension of aluminium (0.542 g, 20.08 mmol) in THF (5.95 ml) was added 3-bromobut-1-yne (3.56 g, 26.8 mmol), followed by mercury (II) chloride (7.27 mg, 0.027 mmol) and the reaction was heated at 50° C. for 15 minutes. Then paraformaldehyde (1.608 g, 53.5 mmol) was added portionwise keeping the temperature below 60° C. After all the paraformaldehyde was added the reaction was heated at 60° C. for 2 hours. The reaction was cooled to room temperature, poured into 3M H₂SO₄, extracted with MTBE, dried over Na₂SO₄, filtered, and concentrated under reduced pressure to yield compound 39.1 as a brown oil, which was carried through to the next step without further purification.

### 4-(6-hydroxy-5-methylhexa-1,3-diyn-1-yl)benzoic acid (39.2)

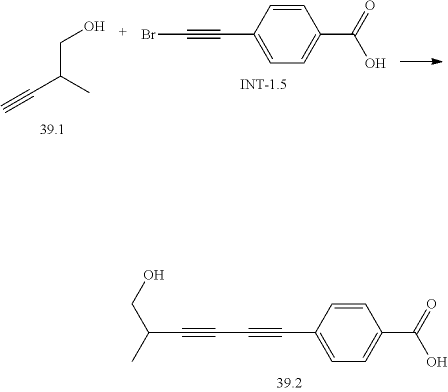

| | | | Moles | Sample |
|---|---|---|---|---|
| Reactant | MW | Eq | (mmol) | Mass (g) |
| Compound 39.1 | 84.116 | 1.000 | 27.3 | 2.3 |
| 4-(bromoethynyl)benzoic acid | 225.039 | .6 | 16.41 | 3.69 |
| copper chloride | 98.999 | .02 | 0.547 | 0.054 |
| hydroxylamine hydrochloride | 33.030 | .06 | 1.641 | 0.114 |

To a stirring solution of compound 39.1 (2.3 g, 27.3 mmol) in 30% aqueous n-butylamine (14.50 ml) at 0° C. were added copper chloride (0.054 g, 0.547 mmol) and hydroxylamine hydrochloride (0.114 g, 1.641 mmol). In a separate flask 4-(bromoethynyl)benzoic acid (3.69 g, 16.41 mmol) was dissolved in 30% aqueous n-butylamine (31.1 ml). Then hydroxylamine hydrochloride (0.114 g, 1.641 mmol) was added, and the resulting solution was added dropwise to the previously prepared solution of 39.1, and the reaction mixture was stirred for 1 hr. The reaction was diluted with MeTHF (40 mL), and was then acidified to pH 1 with concentrated HCl. This solution was then filtered through Celite, and the layers were separated. The aqueous layer was extracted again with MeTHF (40 mL), and the organic layers were combined. The organic layers were washed with 2M HCl (2×40 mL) and brine (40 mL), dried over Na₂SO₄, filtered, and concentrated under reduced pressure to yield compound 39.2 (2.41 g, 38.6%) as a white solid.

### Methyl 3-((tert-butoxycarbonyl)amino)-2-(4-(6-hydroxy-5-methylhexa-1,3-diyn-1-yl)benzamido)-3-methylbutanoate (39.3)

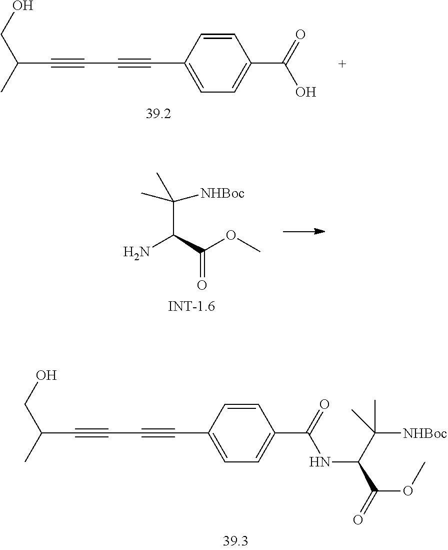 [SMILES: CC(C#CC#Cc1ccc(C(=O)O)cc1)CO ; COC(=O)[C@@H](N)C(C)(C)NC(=O)OC(C)(C)C ; COC(=O)[C@@H](NC(=O)c1ccc(C#CC#CC(C)CO)cc1)C(C)(C)NC(=O)OC(C)(C)C]

| | | | | | Sample | |
|---|---|---|---|---|---|---|
| | | | Density | Moles | Mass | Vol |
| Reactant | MW | Eq | (g/ml) | (mmol) | (g) | (ml) |
| Compound 39.2 | 228.243 | 1.000 | | 11.57 | 2.64 | |
| methyl 2-amino-3-((tert- | 246.303 | 1.5 | | 17.35 | 4.27 | |
| butoxycarbonyl)amino)-3- | | | | | | |
| methylbutanoate | | | | | | |
| HATU | 235.265 | 1.1 | | 12.72 | 4.84 | |
| DIPEA | 129.243 | 1.5 | 0.74 | 17.35 | 2.242 | 3.03 |
| K₂CO₃ | 60.010 | 4.5 | | 52.0 | 7.19 | |

To a stirring solution of methyl 2-amino-3-((tert-butoxycarbonyl)amino)-3-methylbutanoate (4.27 g, 17.35 mmol) in DMF (94 ml) was added K₂CO₃ (7.19 g, 52.0 mmol) and the reaction was stirred for 1 hour. Then compound 39.2 (2.64 g, 11.57 mmol) and DIPEA (3.03 ml, 17.35 mmol) were added, followed by HATU (4.84 g, 12.72 mmol) and the reaction was stirred for 1 hour. The reaction mixture was diluted with ethyl acetate, washed with 1M citric acid, NaHCO₃ and brine, dried over Na₂SO₄, filtered, and concentrated under reduced pressure to yield compound 39.3 as a brown oil, which was carried through to the next step without further purification. MS: m/z calcd for C₂₅H₃₂N₂O₆ 456.2. found [M+H]⁺ 457.3.

### Methyl 3-amino-2-(4-(6-hydroxy-5-methylhexa-1,3-diyn-1-yl)benzamido)-3-methylbutanoate (39.4)

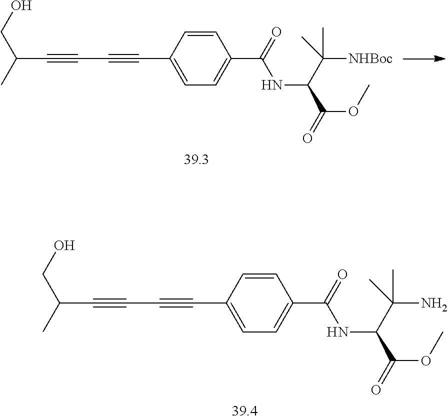 [SMILES: COC(=O)[C@@H](NC(=O)c1ccc(C#CC#CC(C)CO)cc1)C(C)(C)NC(=O)OC(C)(C)C ; COC(=O)[C@@H](NC(=O)c1ccc(C#CC#CC(C)CO)cc1)C(C)(C)N]

In a 50 mL flask compound 39.3 (5.3 g, 11.61 mmol) was dissolved in DCM (38.7 ml) and TFA (77 ml) and the reaction was stirred for 1 hour. The reaction was then azeotroped with DCM (3×75 mL) and then concentrated under reduced pressure to yield compound 39.4 as a brown oil, which was carried through to the next step without further purification.

### N—((S)-3-amino-1-(hydroxyamino)-3-methyl-1-oxobutan-2-yl)-4-(6-hydroxy-5-methylhexa-1,3-diyn-1-yl)benzamide (39)

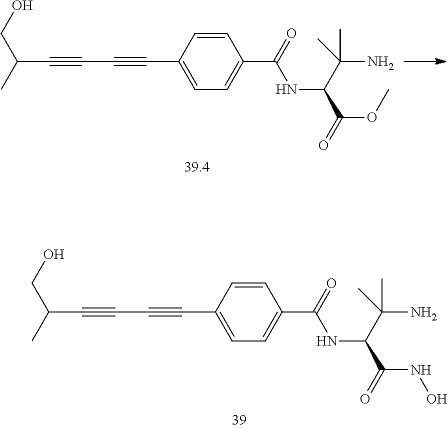 [SMILES: COC(=O)[C@@H](NC(=O)c1ccc(C#CC#CC(C)CO)cc1)C(C)(C)N ; CC(C#CC#Cc1ccc(C(=O)N[C@H](C(=O)NO)C(C)(C)N)cc1)CO]

| | | | | | Sample | | |
|---|---|---|---|---|---|---|---|
| | | | Density | Moles | Mass | Vol | % Wt |
| Reactant | MW | Eq | (g/ml) | (mmol) | (g) | (ml) | (%) |
| Compound 39.4 | 356.416 | 1.000 | | 11.62 | 4.14 | | |
| hydroxylamine (50% | 33.030 | 60 | 1 | 697 | 46.0 | 46.0 | 50 |
| solution in water) | | | | | | | |

To a stirring solution of compound 39.4 (4.14 g, 11.62 mmol) in IPA (49.9 mL) was added hydroxylamine (50% solution in water, 46.0 ml, 697 mmol) and the reaction was stirred for 18 hours. The reaction mixture was then concentrated under reduced pressure to a thick oil. which was diluted with acetic acid (70 mL) and purified by RP HPLC (0.1% AcOH in H₂O/ACN) to yield 39 (1.28 g, 29.3%). MS: m/z calcd for C₁₉H₂₃N₃O₄357.2. found [M+H]⁺ 358.1.

### 40. N—((S)-3-hydroxy-1-(hydroxyamino)-3-methyl-1-oxobutan-2-yl)-4-((S)-5-hydroxyhexa-1,3-diyn-1-yl)benzamide (40)

### (S)-4-(5-hydroxyhexa-1,3-diyn-1-yl)benzoic acid (40.1)

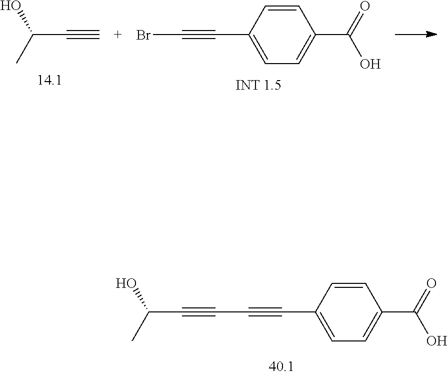

| | | | Moles | Sample |
|---|---|---|---|---|
| Reactant | MW | Eq | (mmol) | Mass (g) |
| (S)-but-3-yn-2-ol | 70.090 | 1.000 | 27.1 | 1.9 |
| 4-(bromoethynyl)benzoic acid | 225.039 | 1.000 | 27.1 | 6.10 |
| Copper(I) chloride | 98.999 | .02 | 0.542 | 0.054 |
| Hydroxylamine hydrochloride | 33.030 | .06 | 1.626 | 0.113 |

To a stirring solution of (S)-but-3-yn-2-ol (1.9 g, 27.1 mmol) in 30% aqueous butylamine (14.38 ml) at 0° C. were added copper(I) chloride (0.054 g, 0.542 mmol) and hydroxylamine hydrochloride (0.113 g, 1.626 mmol). In a separate flask 4-(bromoethynyl)benzoic acid (6.10 g, 27.1 mmol) was dissolved in 30% aqueous butylamine (30.8 ml) to give an orange solution. Then hydroxylamine hydrochloride (0.113 g, 1.626 mmol) was added and the resulting solution was added dropwise to the previously prepared solution of (S)-but-3-yn-2-ol and the reaction was stirred for 1 hr. The reaction mixture was then washed with MTBE (2×200 mL), and recooled to 0° C. The reaction was diluted with MeTHF (400 mL), and then acidified to pH 1 with concentrated HCl. This solution was then filtered through Celite, and the layers were separated. The aqueous layer was extracted with MeTHF (200 mL), and the organic layers were combined. The organic layers were washed with 2M HCl (2×200 mL), water (200 mL), and brine (200 mL), dried over Na₂SO₄, filtered, and concentrated under reduced pressure to yield compound 40.1 as a white solid (4.88 g, 84%). ¹H NMR (400 MHz, DMSO-d₆) δ 13.2 (s, 1H), 7.9 (s, 1H), 7.6 (s, 1H), 5.6 (s, 1H), 4.5 (m, 1H), 1.3 (d, 3H).

### (S)-methyl 3-hydroxy-2-(4-((S)-5-hydroxyhexa-1,3-diyn-1-yl)benzamido)-3methylbutanoate (40.2)

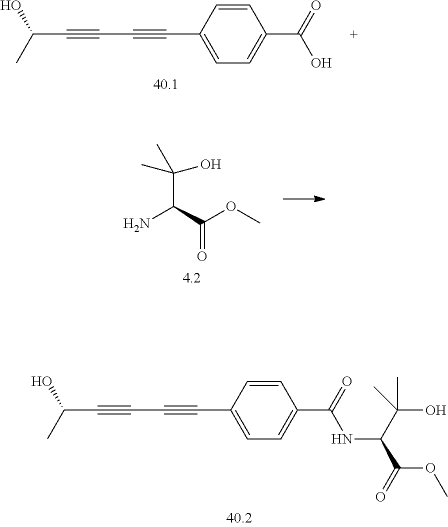

| | | | | | Sample | |
|---|---|---|---|---|---|---|
| | | | Density | Moles | Mass | Vol |
| Reactant | MW | Eq | (g/ml) | (mmol) | (g) | (ml) |
| Compound 40.1 | 214.217 | 1.000 | | 14.00 | 3 | |
| methyl 2-amino-3- | 147.172 | 1.5 | | 21.01 | 3.09 | |
| hydroxy-3- | | | | | | |
| methylbutanoate | | | | | | |
| HATU | 235.265 | 1.1 | | 15.40 | 5.86 | |
| DIPEA | 129.243 | 1.5 | 0.74 | 21.01 | 2.71 | 3.67 |
| K₂CO₃ | 60.010 | 4.5 | | 63.0 | 8.71 | |

To a stirring solution of methyl 2-amino-3-hydroxy-3-methylbutanoate (3.09 g, 21.01 mmol) in DMF (114 mL) was added K₂CO₃ (8.71 g, 63.0 mmol) and the reaction was stirred for 1 hour. Compound 40.1 (3 g, 14.00 mmol) and DIPEA (3.67 ml, 21.01 mmol) were then added, followed by HATU (5.86 g, 15.40 mmol) and the reaction mixture was stirred for 1 hour. The reaction was then diluted with ethyl acetate, washed with 1M citric acid, NaHCO₃ and brine, dried over Na₂SO₄, filtered, and concentrated under reduced pressure to yield compound 40.2 as a brown oil (4.23 g, 88%), which was carried through to the next step without further purification. MS: m/z calcd for C₁₉H₂₁NO₅343.1. found [M+H]⁺ 344.1

### N—((S)-3-hydroxy-1-(hydroxyamino)-3-methyl-1-oxobutan-2-yl)-4-((S)-5-hydroxyhexa-1,3-diyn-1-yl)benzamide (40)

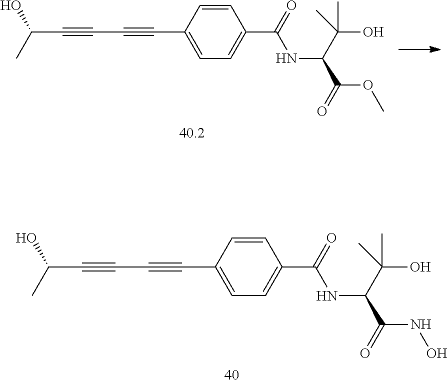 [SMILES: COC(=O)[C@@H](NC(=O)c1ccc(C#CC#C[C@H](C)O)cc1)C(C)(C)O ; C[C@H](O)C#CC#Cc1ccc(C(=O)N[C@H](C(=O)NO)C(C)(C)O)cc1]

To a stirring solution of compound 40.2 (4.8 g, 13.98 mmol) in IPA (60.0 mL) was added hydroxylamine (50% solution in water, 111 ml, 1677 mmol) and the reaction was stirred for 18 hours. The reaction was then concentrated under reduced pressure to a thick oil, which was diluted with acetic acid (70 mL) and purified by RP HPLC (0.1% AcOH in H₂O/ACN) to yield 40 (1.4 g, 27.6%). MS: m/z calcd for C₁₈H₂₀N₂O₅344.1. found [M+H]⁺ 345.2.

### 41. N-((1S,3R)-3-amino-1-(hydroxyamino)-1-oxobutan-2-yl)-4-((S)-5-hydroxyhexa-1,3-diyn-1-yl)benzamide (41)

### (2S,3R)-methyl 3-((tert-butoxycarbonyl)amino)-2-(4-((S)-5-hydroxyhexa-1,3-diyn-1-yl)benzamido)butanoate (41.1)

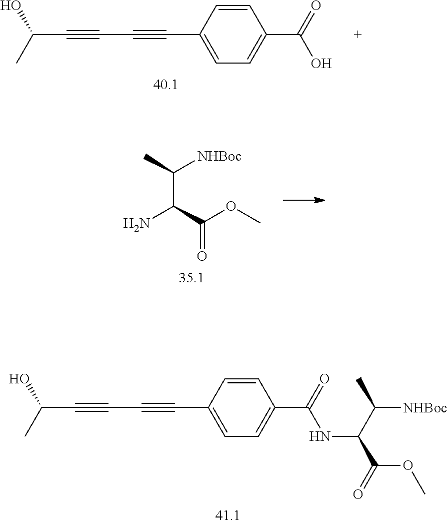 [SMILES: C[C@H](O)C#CC#Cc1ccc(C(=O)O)cc1 ; COC(=O)[C@@H](N)[C@@H](C)NC(=O)OC(C)(C)C ; COC(=O)[C@@H](NC(=O)c1ccc(C#CC#C[C@H](C)O)cc1)[C@@H](C)NC(=O)OC(C)(C)C]

| | | | | | Sample | |
|---|---|---|---|---|---|---|
| | | | Density | Moles | Mass | Vol |
| Reactant | MW | Eq | (g/ml) | (mmol) | (g) | (ml) |
| Compound 40.1 | 214.217 | 1.000 | | 14.00 | 3 | |
| (2S,3R)-methyl 2-amino-3-((tert- | 232.277 | 1.5 | | 21.01 | 4.88 | |
| butoxycarbonyl)amino)butanoate | | | | | | |
| HATU | 235.265 | 1.1 | | 15.40 | 5.86 | |
| DIPEA | 129.243 | 1.5 | 0.74 | 21.01 | 2.71 | 3.67 |
| K₂CO₃ | 60.010 | 4.5 | | 63.0 | 8.71 | |

To a stirring solution of (2S,3R)-methyl 2-amino-3-((tert-butoxycarbonyl)amino)butanoate (4.88 g, 21.01 mmol) in DMF (114 ml) was added K₂CO₃ (8.71 g, 63.0 mmol) and the reaction was stirred for 1 hour. Compound 40.1 (3 g, 14.00 mmol) and DIPEA (3.67 ml, 21.01 mmol) were added, followed by HATU (5.86 g, 15.40 mmol) and the reaction was stirred for 1 hour. The reaction was diluted with ethyl acetate, washed with 1M citric acid, NaHCO₃ and brine, dried over Na₂SO₄, filtered, and concentrated under reduced pressure to yield compound 41.1 as a brown oil (6.17 g), which was carried through to the next step without further purification. MS: m/z calcd for C₂₃H₂₈N₂O₆ 428.2. found [M+Na]⁺ 451.3.

### (2S,3R)-methyl 3-amino-2-(4-((S)-5-hydroxyhexa-1,3-diyn-1-yl)benzamido)butanoate (41.2)

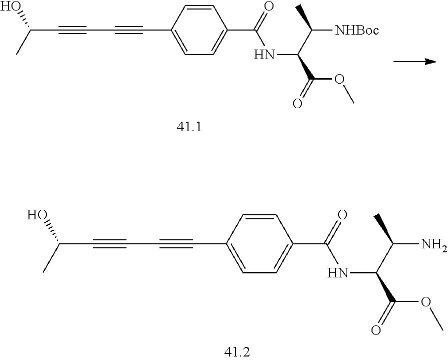 [SMILES: COC(=O)[C@@H](NC(=O)c1ccc(C#CC#C[C@H](C)O)cc1)[C@@H](C)NC(=O)OC(C)(C)C ; COC(=O)[C@@H](NC(=O)c1ccc(C#CC#C[C@H](C)O)cc1)[C@@H](C)N]

To a stirring solution of compound 41.1 (6 g, 14.00 mmol) in DCM (46.7 mL) was added TFA (93 mL) and the reaction was stirred for 1 hour. The reaction was azeotroped with DCM (3×75 mL) and then concentrated under reduced pressure to yield compound 41.2 as a brown oil, which was carried through to the next step without further purification. MS: m/z calcd for C₁₈H₂₀N₂O₄328.1. found [M+H]⁺ 329.2.

### N-((2S,3R)-3-amino-1-(hydroxyamino)-1-oxobutan-2-yl)-4-((S)-5-hydroxyhexa-1,3-diyn-1-yl)benzamide (41)

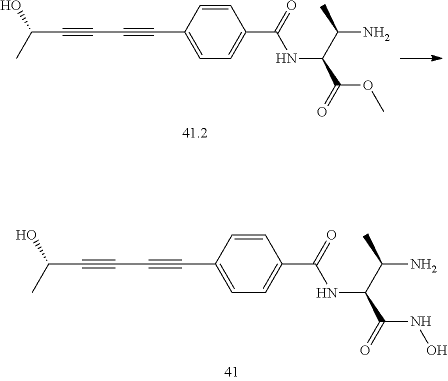 [SMILES: COC(=O)[C@@H](NC(=O)c1ccc(C#CC#C[C@H](C)O)cc1)[C@@H](C)N ; C[C@H](O)C#CC#Cc1ccc(C(=O)N[C@H](C(=O)NO)[C@@H](C)N)cc1]

To a stirring solution of compound 41.2 (4.6 g, 14.01 mmol) in IPA (60.1 mL) was added hydroxylamine (50% solution in water, 55.5 mL, 841 mmol) and the reaction was stirred for 18 hours. The reaction mixture was then concentrated under reduced pressure to a thick oil, which was diluted with acetic acid (70 mL) and purified by RP HPLC (0.1% AcOH in H₂O/ACN) to yield 41 (1.47 g, 30.3%). MS: m/z calcd for C₁₇H₁₉N₃O₄329.1. found [M+H]⁺ 330.2.

### 42. N-((1S,3R)-3-hydroxy-1-(hydroxyamino)-1-oxobutan-2-yl)-4-((S)-5-hydroxyhexa-1,3-diyn-1-yl)benzamide (42)

### (2S,3R)-methyl 3-hydroxy-2-(4-((S)-5-hydroxyhexa-1,3-diyn-1-yl)benzamido)butanoate (42.1)

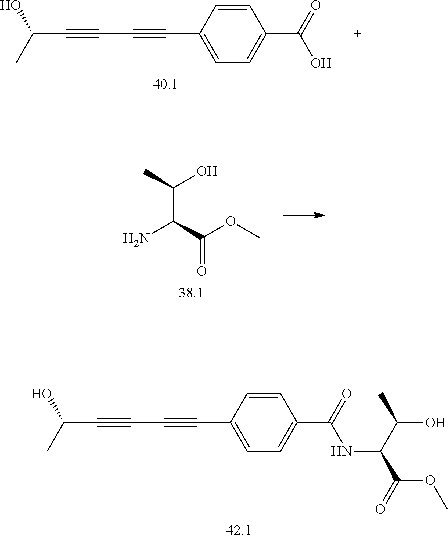

| | | | | | Sample | |
|---|---|---|---|---|---|---|
| | | | Density | Moles | Mass | Vol |
| Reactant | MW | Eq | (g/ml) | (mmol) | (g) | (ml) |
| Compound 40.1 | 214.217 | 1.000 | | 14.00 | 3 | |
| (2S,3R)-methyl 2- | 133.146 | 1.5 | | 21.01 | 3.56 | |
| amino-3- | | | | | | |
| hydroxybutanoate, | | | | | | |
| HCI | | | | | | |
| HATU | 235.265 | 1.1 | | 15.40 | 5.86 | |
| DIPEA | 129.243 | 1.5 | 0.74 | 21.01 | 2.71 | 3.67 |
| K₂CO₃ | 60.010 | 4.5 | | 63.0 | 8.71 | |

To a stirring solution of (2S,3R)-methyl 2-amino-3-hydroxybutanoate (3.56 g, 21.01 mmol) in DMF (114 mL) was added K₂CO₃ (8.71 g, 63.0 mmol) and the reaction was stirred for 1 hour. Then compound 40.1 (3 g, 14.00 mmol) and DIPEA (3.67 ml, 21.01 mmol) were added, followed by HATU (5.86 g, 15.40 mmol) and the reaction was stirred for 1 hour. The reaction was then diluted with ethyl acetate, washed with 1M citric acid, NaHCO₃ and brine, dried over Na₂SO₄, filtered, and concentrated under reduced pressure to yield compound 42.1 as a brown oil (3.05 g, 66.1%), which was carried through to the next step without further purification. MS: m/z calcd for C₁₈H₁₉NO₅329.1. found [M+H]⁺ 330.2.

### N-((2S,3R)-3-hydroxy-1-(hydroxyamino)-1-oxobutan-2-yl)-4-((S)-5-hydroxyhexa-1,3-diyn-1-yl)benzamide (42)

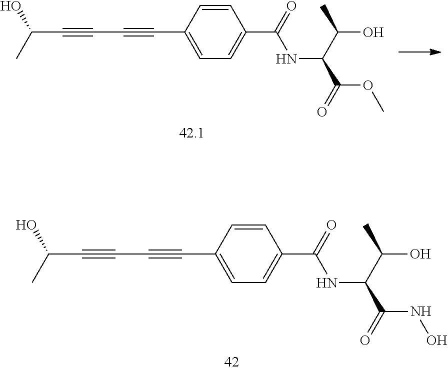 [SMILES: COC(=O)[C@@H](NC(=O)c1ccc(C#CC#C[C@H](C)O)cc1)[C@@H](C)O ; C[C@H](O)C#CC#Cc1ccc(C(=O)N[C@H](C(=O)NO)[C@@H](C)O)cc1]

To a stirring solution of compound 42.1 (4.61 g, 14.00 mmol) was dissolved in IPA (60.1 mL) was added hydroxylamine (50% solution in water, 55.4 mL, 840 mmol) and the reaction was stirred for 5 hours. The reaction was then concentrated under reduced pressure to a thick oil, which was diluted with acetic acid (120 mL) and purified by RP HPLC (0.1% AcOH in H₂O/ACN) to yield 42 (1.5 g, 30.8%). MS: m/z calcd for C₁₇H₁₈N₂O₅330.1. found [M+H]⁺ 331.0.

### 43. 4-((S)-6,7-dihydroxyhepta-1,3-diyn-1-yl)-N—((S)-1-(hydroxyamino)-3-methyl-3-(methylamino)-1-oxobutan-2-yl)benzamide (43)

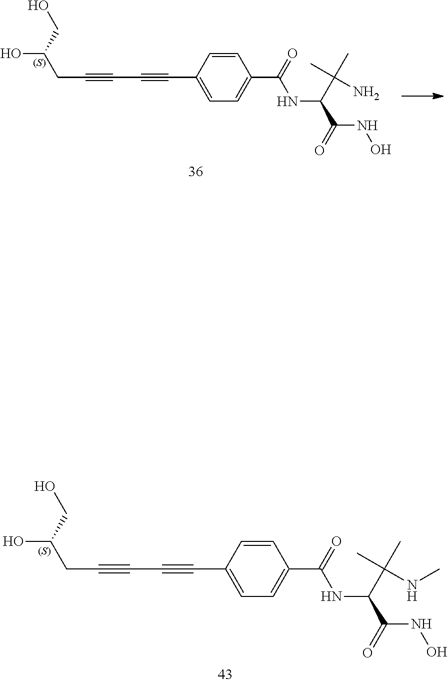

| | | | | Sample | | | % |
|---|---|---|---|---|---|---|---|
| | | | Moles | Mass | Vol | Density | Wt |
| Reactant | MW | Eq | (mmol) | (g) | (ml) | (g/ml) | (%) |
| SODIUM | 39.853 | 1.000 | 57.3 | 3.6 | | | |
| CYANOBOROHYDRIDE | | | | | | | |
| DIPEA | 129.243 | 0.200 | 11.45 | 1.480 | 2 | 0.74 | |
| FORMALDEHYDE | 30.026 | 0.469 | 26.9 | 2.180 | 2 | 1.09 | 37 |
| N-((S)-3-amino-1- | 373.403 | 0.093 | 5.36 | 2 | | | |
| (hydroxyamino)-3-methyl-1- | | | | | | | |
| oxobutan-2-yl)-4((S)-6,7- | | | | | | | |
| dihydroxyhepta-1,3-diyn-1- | | | | | | | |
| yl)benzamide | | | | | | | |

To a stirring solution of N—((S)-3-amino-1-(hydroxyamino)-3-methyl-1-oxobutan-2-yl)-4-((S)-6,7-dihydroxyhepta-1,3-diyn-1-yl)benzamide (2 g, 5.36 mmol) in THF was added DIPEA (3 mL) and the reaction was stirred for 1 h. Formaldehyde (37% aqueous, 2 mL, 26.9 mmol) was added and the reaction was stirred for 24 hr. n-Butylamine (2 mL) was then added and the reaction was stirred for 2 hr. Solvent evaporation under reduced pressure gave a crude, which was redissolved in THF (30 mL) and AcOH was added (12 mL) followed by sodium cyanoborohydride (3.6 g) and the reaction was stirred for 20 min. Water (10 mL) was added and THF was removed under reduced pressure and the residue was purified by RP HPLC (2″, 0.1% AcOH in water/ACN) to yield compound 43 (1.13 g).

### 44. 4-((R)-6,7-dihydroxyhepta-1,3-diyn-1-yl)-N—((S)-1-(hydroxyamino)-3-methyl-3-(methylamino)-1-oxobutan-2-yl)benzamide (44)

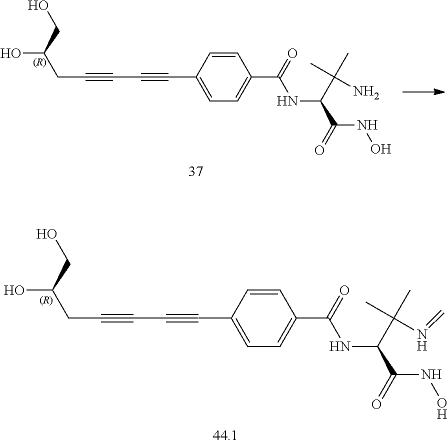

| | | | | | Sample | | |
|---|---|---|---|---|---|---|---|
| | | | Density | Moles | Mass | Vol | % Wt |
| Reactant | MW | Eq | (g/ml) | (mmol) | (g) | (ml) | (%) |
| Compound 37 | 373.403 | 1.000 | | 4.61 | 2 | | |
| DIPEA | 129.243 | 2.5 | 0.74 | 11.54 | 1.491 | 2.015 | |
| formaldehyde | 30.026 | 5 | 1.09 | 23.07 | 1.872 | 1.718 | 37 |

To a stirring solution of compound 37 (2 g, 4.61 mmol) in THF (46.1 mL) was added DIPEA (2.015 ml, 11.54 mmol) and the reaction was stirred for 1 hour. Next formaldehyde (1.718 ml, 23.07 mmol) was added and the reaction was stirred for 18 hours. The reaction was quenched with n-butylamine (3 mL) and stirred for 3 hours; it was then was concentrated under reduced pressure to yield compound 44.1, which was carried through to the next step without further purification. MS: m/z calcd for C₂₀H₂₃N₃O₅385.2. found [M+H]⁺ 386.3.

### 4-((R)-6,7-dihydroxyhepta-1,3-diyn-1-yl)-N—((S)-1-(hydroxyamino)-3-methyl-3-(methylamino)-1-oxobutan-2-yl)benzamide (44)

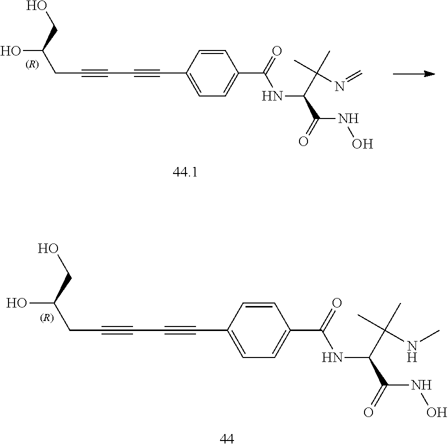

| | | | Moles | Sample |
|---|---|---|---|---|
| Reactant | MW | Eq | (mmol) | Mass (g) |
| Compound 44.1 | 385.414 | 1.000 | 4.04 | 1.8 |
| Sodium cyanoborohydride | 39.853 | 15 | 60.6 | 3.81 |

To a stirring solution of compound 44.1 (1.8 g, 4.04 mmol) in THF (29.9 mL) was added acetic acid (12 mL) followed by sodium cyanoborohydride (3.81 g, 60.6 mmol) and the reaction was stirred for 1 hour. The reaction was then quenched with water (10 mL) and THF was removed under reduced pressure. The resulting aqueous solution was purified by RP HPLC (2″, 0.1% AcOH in H₂O/ACN) to yield compound 44 (0.72 g, 43.9%). MS: m/z calcd for C₂₀H₂₅N₃O₅387.2. found [M+H]⁺ 388.3.

### 45. N-((2S)-3-amino-1-(hydroxyamino)-1-oxobutan-2-yl)-4-(5,6-dihydroxyhexa-1,3-diyn-1-yl)benzamide (45)

### (2S,3R)-methyl 2-(4-(bromoethynyl)benzamido)-3-((tert-butoxycarbonyl)amino) butanoate (45.1)

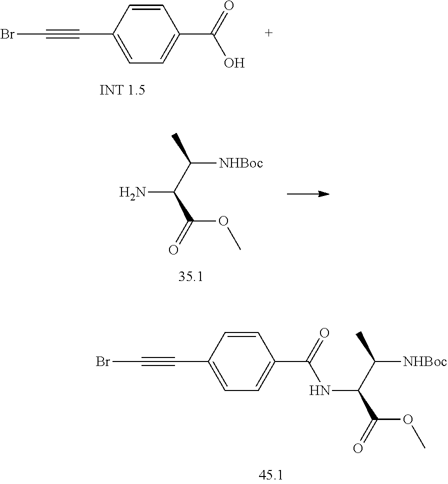 [SMILES: O=C(O)c1ccc(C#CBr)cc1 ; COC(=O)[C@@H](N)[C@@H](C)NC(=O)OC(C)(C)C ; COC(=O)[C@@H](NC(=O)c1ccc(C#CBr)cc1)[C@@H](C)NC(=O)OC(C)(C)C]

| | | | | Sample | | | |
|---|---|---|---|---|---|---|---|
| | | | Moles | Mass | Vol | Molarity | Density |
| Reactant | MW | Eq | (mmol) | (g) | (ml) | (molar) | (g/ml) |
| 4-(bromoethynyl)benzoic | 225.039 | 1.000 | 96 | 21.7 | | | |
| acid | | | | | | | |
| HATU | 235.27 | 1.1 | 106 | 40.3 | | | |
| TEA | 101.19 | 2.5 | 241 | 24.39 | 33.6 | | 0.73 |
| CITRIC ACID | 192.12 | 2 | 193 | | 193 | 1 | |

To a stirring solution of 4-(bromoethynyl)benzoic acid (21.7 g, 96 mmol) in acetonitrile (321 mL) was added compound 35.1 (25.8 g, 96 mmol) followed by TEA (33.6 mL, 241 mmol) and the reaction was cooled to 0° C. HATU (40.3 g, 106 mmol) was added and the reaction was stirred while warming to rt for 1 hr. The reaction was concentrated under reduced pressure to approximately 15 mL, and was diluted with EtOAc (75 mL), and washed with 1 M citric acid (25 mL). The organic layer was washed with saturated aqueous NaHCO3/water (45 mL/20 mL), brine (40 mL), and concentrated under reduced pressure to yield a crude, which was purified by flash chromatography (silica gel/10-40% EtOAc in hexanes) to yield compound 45.1 (35 g, 83%). MS: m/z calcd for C₁₉H₂₃BrN₂O₅ 438.07 & 440.07. found [M+H]⁺ 439.0 & 441.0.

### N-((2S,3R)-3-amino-1-(hydroxyamino)-1-oxobutan-2-yl)-4-(5,6-dihydroxyhexa-1,3-diyn-1-yl)benzamide (45)

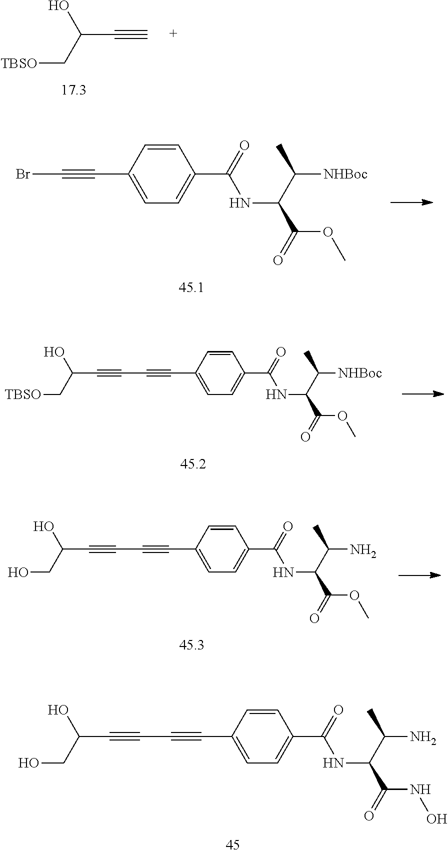 [SMILES: C#CC(O)CO[Si](C)(C)C(C)(C)C ; COC(=O)[C@@H](NC(=O)c1ccc(C#CBr)cc1)[C@@H](C)NC(=O)OC(C)(C)C ; COC(=O)[C@@H](NC(=O)c1ccc(C#CC#CC(O)CO[Si](C)(C)C(C)(C)C)cc1)[C@@H](C)NC(=O)OC(C)(C)C ; COC(=O)[C@@H](NC(=O)c1ccc(C#CC#CC(O)CO)cc1)[C@@H](C)N ; C[C@@H](N)[C@H](NC(=O)c1ccc(C#CC#CC(O)CO)cc1)C(=O)NO]

| | | | Moles | Sample |
|---|---|---|---|---|
| Reactant | MW | Eq | (mmol) | Mass (g) |
| hydroxylamine hydrochloride | 33.030 | 0.06 | 0.898 | 0.062 |
| copper(I) chloride | 98.999 | 0.02 | 0.299 | 0.030 |
| 1-((tert-butyldimethylsilyl)oxy)but- | 200.350 | 1.000 | 14.97 | 3.00 |
| 3-yn-2-ol | | | | |
| (2S)-methyl 2-(4- | 439.300 | 1.000 | 14.97 | 6.58 |
| (bromoethynyl)benzamido)-3- | | | | |
| ((tert- | | | | |
| butoxycarbonyl)amino)butanoate | | | | |

To a stirring solution of hydroxylamine hydrochloride (0.062 g, 0.898 mmol) and copper(I) chloride (0.030 g, 0.299 mmol) in 30% n-butylamine (aq, 70 mL) was added a solution of 1-((tert-butyldimethylsilyl)oxy)but-3-yn-2-ol (3.00 g, 14.97 mmol) in 30% n-butylamine (aq, 20 mL), followed by a solution of (2S)-methyl 2-(4-(bromoethynyl)benzamido)-3-((tert-butoxycarbonyl)amino)butanoate (6.58 g, 14.97 mmol) and hydroxylamine hydrochloride (62 mg) in 30% n-butylamine (aq, 20 mL) and THF (20 mL) and the reaction was stirred for 2 hr. The reaction mixture was extracted with EtOAc (2×300 mL), and the combined organic layers were dried over Na₂SO₄, filtered and concentrated under reduced pressure to yield compound 45.2 (MS: m/z calcd for C₂₉H₄₂N₂O₇Si 558.3. found [M+Na]⁺ 581.3), which was reacted with TFA (20 mL) for 2 hr. The reaction mixture was concentrated under reduced pressure, and the residue was diluted with water (100 mL) and neutralized with K₂CO₃ (solid) till pH 9. The aqueous layer was extracted with EtOAc (3×200 mL), and the combined organic layers were dried over Na₂SO₄, filtered and concentrated under reduced pressure to yield compound 45.3. To a stirring solution of compound 45.3 in IPA (25 mL) was added hydroxylamine (50% aqueous, 25 mL) and the reaction was stirred at rt for 3 hr. The reaction mixture was concentrated under reduced pressure, was diluted with AcOH (30 mL) and was purified by RPLC (6″, 0.1% AcOH in water and ACN) to yield compound 45.

### 46. N—((S)-3-amino-1-(hydroxyamino)-3-methyl-1-oxobutan-2-yl)-4-(5,7-dihydroxyhepta-1,3-diyn-1-yl)benzamide (46)

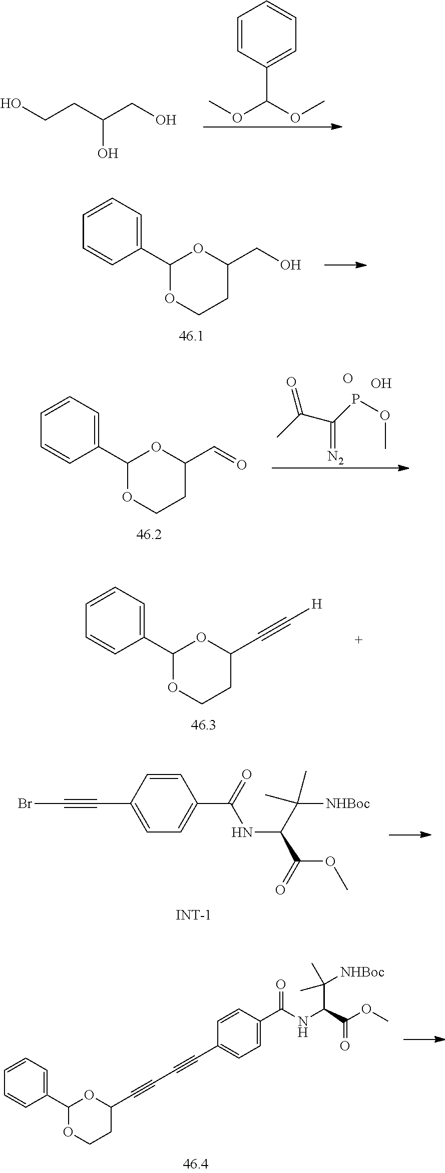

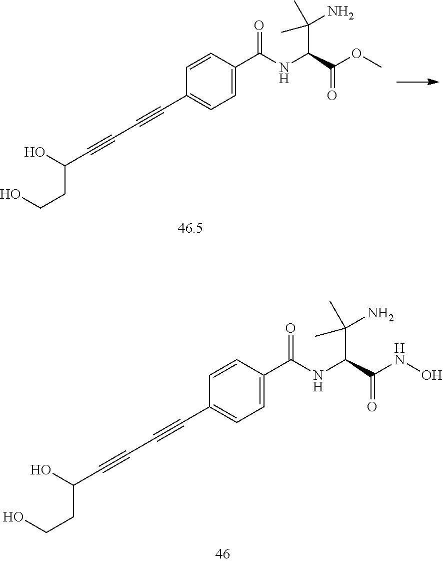 [SMILES: COC(=O)[C@@H](NC(=O)c1ccc(C#CC#CC(O)CCO)cc1)C(C)(C)N ; CC(C)(N)[C@H](NC(=O)c1ccc(C#CC#CC(O)CCO)cc1)C(=O)NO]

### (2-phenyl-1,3-dioxan-4-yl)methanol (46.1)

| Reagent | MW | Eq. | mmol | g, mL | |
|---|---|---|---|---|---|
| Butane-1,2,4-triol | 106.12 | 1 | 14.9 | 1.58 | g |
| Dimethoxymethyl-benzene | 152.19 | 1.07 | 15.9 | 2.64 | g |
| CSA | 232 | 0.05 | 0.75 | 174 | mg |
| TEA | 101.19 | 0.105 | 1.57 | 144 | mg |
| DCM | | | | 50 | mL |

Butane-1,2,4-triol (1.58 g, 14.9 mmol) and dimethoxymethyl-benzene (2.64 g, 15.9 mmol) in dry DCM (50 mL) were stirred at rt in the presence of CSA (174 mg, 0.75 mmol) for 16 hours. TEA (144 mg, 1.57 mmol) was then added and the solvents were removed under reduced pressure to yield compound 46.1 (82%) as a colorless oil, which was carried through to the next step without further purification. ¹H NMR (400 MHz, CDCl₃) δ 1.91-1.95 (m, 1H), 2.04-2.14 (m, 1H), 3.66-3.85 (m, 3H), 4.11-4.41 (m, 2H), 5.45-5.94 (m, 1H), 7.35-7.40 (m, 3H), 7.45-7.51 (m, 2H). MS: m/z calcd for C₁₁H₁₁O₃194.1. found [M+H]⁺ 195.1.

### 2-phenyl-1,3-dioxane-4-carbaldehyde (46.2)

| Reagent | MW | Eq. | mmol | g, mL | |
|---|---|---|---|---|---|
| Compound 46.1 | 194.2 | 1 | 8.5 | 1.65 | g |
| Oxalyl chloride | 126.8 | 1.08 | 9.18 | 0.8 | mL |
| DMSO | 78 | 2.32 | 19.72 | 1.4 | mL |
| TEA | 101.19 | 4.73 | 40.2 | 5.6 | mL |
| DCM | | | | 40 | mL |

To a stirring solution of dry DMSO (1.4 mL, 19.72 mmol) in DCM (10 mL) at −60° C. under argon was added dropwise a solution of oxalyl chloride (0.8 mL, 9.18 mmol) in DCM (20 mL) and the reaction mixture was stirred for 12 min. A solution of compound 46.1 (1.65 g, 8.5 mmol) in DCM (10 mL) was then added dropwise and the reaction mixture was stirred for 30 min. TEA (5.6 mL, 40.2 mmol) was then added and the reaction was stirred for 5 min. The cooling bath was removed, and water was added, and the mixture was allowed to warm up to rt. The phases were separated, the aqueous phase was extracted with DCM, and the combined organic layers were washed with saturated ammonium chloride solution, water, dried, filtered and concentrated under reduced pressure to yield aldehyde 46.2 (1.4 g), which was carried through to the next step without further purification. ¹H NMR (400 MHz, CDCl₃) δ 1.79-1.84 (m, 1H), 1.95-2.01 (m, 1H), 3.80-4.00 (m, 1H), 4.30-4.38 (m, 2H), 5.56-5.61 (m, 1H), 7.35-7.40 (m, 3H), 7.45-7.49 (m, 2H), 9.73 (s, 1H).

### 4-ethynyl-2-phenyl-1,3-dioxane (46.3)

| Reagent | MW | Eq. | mmol | g, mL | |
|---|---|---|---|---|---|
| Compound 46.2 | 192.21 | 1 | 7.28 | 1.4 | g |
| Bestmann reagent | 192.11 | 1.5 | 10.9 | 2.1 | g |
| K₂CO₃ | 138.46 | 2 | 14.56 | 2 | g |
| CH₃OH/Et₂O (2:1) | | | | 15 | mL |

To a stirring solution of compound 46.2 (1.4 g, 7.28 mmoL) in CH₃OH/Et₂O (15 mL, 2:1) were added Bestmann reagent (2.1 g, 10.9 mmoL) and K₂CO₃ (2 g, 14.56 mmol) and the reaction mixture was stirred for 5 hours. The reaction mixture was diluted with water (30 mL), extracted with PE (3×60 mL), and the combined organic layers were dried and concentrated under reduced pressure to give a residue, which was purified by flash chromatography (silica gel/PE:Et₂O 5:1 to 1:1) to yield compound 46.3 as a colorless oil (563 mg). ¹H NMR (400 MHz, CDCl₃) δ 1.76-1.81 (m, 1H), 2.25-2.30 (m, 1H), 2.54 (m, 1H), 3.94-4.00 (m, 1H), 4.26-4.30 (m, 1H), 4.64-4.69 (m, 1H), 5.51 (s, 1H), 7.33-7.38 (m, 3H), 7.49-7.51 (m, 2H).

### (2S)-methyl-3-(tert-butoxycarbonylamino)-3-methyl-2-(4-((2-phenyl-1,3-dioxan-4-yl)buta-1,3-diynyl)benzamido)butanoate (46.4)

| Reagent | MW | Eq. | mmol | g, mL | |
|---|---|---|---|---|---|
| Compound 46.3 | 188.22 | 1 | 2.85 | 563 | mg |
| INT-1 | 453.33 | 1.1 | 3.135 | 1.43 | g |
| Pd(PPh₃)₂Cl₂ | 701.9 | 0.05 | 0.143 | 100 | mg |
| CuI | 190.23 | 0.1 | 0.285 | 54.2 | mg |
| TEA | 101.19 | 3 | 8.55 | 1.24 | ml |
| THF | | | | 20 | mL |

To a stirring solution of compound 46.3 (563 mg, 2.85 mmol), Pd(PPh₃)₂Cl₂ (100 mg, 0.143 mmol), CuI (54.2 mg, 0.285 mmol), and TEA (1.24 mL, 8.55 mmol) in THF (20 mL) under Argon was added INT-1 (1.43 g, 3.135 mmol) and the reaction mixture was stirred for 5 hours. The solvent was removed under reduced pressure, the residue was diluted with water (50 mL), extracted with DCM (2×100 mL). The combined organic layers were dried and concentrated under reduced pressure to give a crude oil, which was purified by flash chromatography (silica gel/PE:EA 5:1 to 3:1) to yield compound 46.4 (640 mg, 40%) as a yellow solid.

### (2S)-methyl 3-amino-2-(4-(5,7-dihydroxyhepta-1,3-diynyl)benzamido)-3-methylbutanoate (46.5)

| Reagent | MW | Eq. | mmol | g, mL |
|---|---|---|---|---|
| Compound 46.4 | 560.64 | 1 | 1.14 | 640 mg |
| HCl(g) | 36.5 | | | |
| CH₃OH | | | | 5 mL |

Compound 46.4 (640 mg, 1.14 mmoL) was dissolved in CH₃OH (5 mL) and was treated with dry HCl(g) for 10 mins, and the progress of the reaction was followed by LCMS. The solvent was removed under reduced pressure to yield compound 46.5 (110 mg) as a yellow solid. MS: m/z calcd for C₂₀H₂₄N₂O₅ 372.2. found [M+H]⁺ 373; ¹H NMR (400 MHz, DMSO-d₆) δ 8.42 (s, 1H), 7.88 (d, J=8 Hz, 2H), 7.67 (d, J=8.1 Hz, 2H), 5.7 (s, 2H), 4.59 (s, 1H), 4.55-4.52 (m, 1H), 4.38 (s, 1H), 3.64 (s, 3.55-3.49 (m, 3H), 1.85-1.71 (m, 2H), 1.12 (d, J=7.6 Hz, 1H)

### N—((S)-3-amino-1-(hydroxyamino)-3-methyl-1-oxobutan-2-yl)-4-(5,7-dihydroxyhepta-1,3-diyn-1-yl)benzamide (46)

To a stirring solution of compound 46.5 (110 mg, 0.295 mmol) in IPA (0.3 ml) was added 50% hydroxylamine in water (0.390 ml, 5.91 mmol) at 0° C. and the reaction was stirred for 20 hr. The reaction was concentrated under reduced pressure and acidified with AcOH (0.4 mL) and then purified by RP HPLC (1″, 0.1% AcOH in water/ACN) to yield N—((S)-3-amino-1-(hydroxyamino)-3-methyl-1-oxobutan-2-yl)-4-(5,7-dihydroxyhepta-1,3-diyn-1-yl)benzamide 46 (55 mg, 0.143 mmol, 48.6%). MS: m/z calcd for C₁₉H₂₃N₃O₅ 373.2. found [M+H]⁺ 374.2.

### 47. N—((S)-3-amino-1-(hydroxyamino)-3-methyl-1-oxobutan-2-yl)-4-(6-hydroxy-5-methoxyhexa-1,3-diyn-1-yl)benzamide (47)

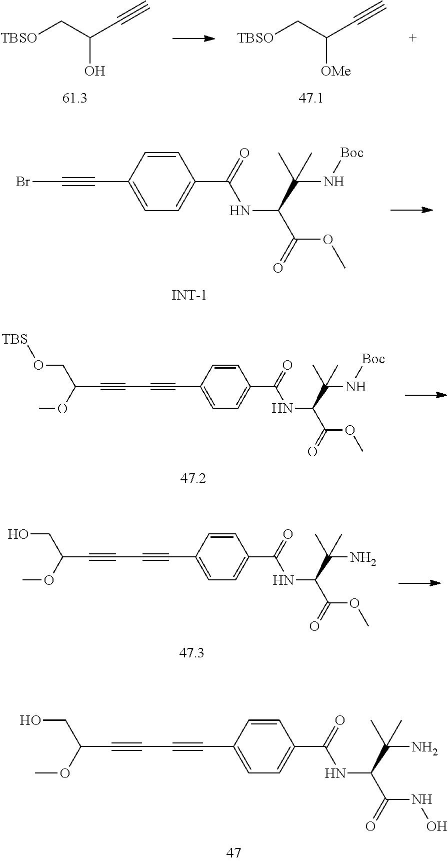

### 1-((tert-butyldimethylsilyl)oxy)but-3-yn-2-ol (47.1)

| Reagent | MW | Eq. | mmol | g, mL | |
|---|---|---|---|---|---|
| Compound 61.3 | 272.53 | 1.0 | 10.3 | 2.80 | g |
| NaH | 24 (60%) | 1.5 | 15.4 | 0.671 | g |
| MeI | 141.94 | 1.1 | 12.4 | 0.77 | mL |
| THF | | | | 60 | mL |

To a stirring solution of NaH (0.617 g, 15.4 mmol) (60% in oil) in THF (60 mL) was added 1-(tert-butyldimethylsilyloxy)-4-(trimethylsilyl)but-3-yn-2-ol (2.80 g, 10.3 mmol) and the reaction was stirred at rt for 40 minutes. MeI (0.77 mL, 12.4 mmol) was added dropwise, and the reaction mixture was stirred at rt for 2 hr. The reaction mixture was poured into ice water and extracted with EtOAc. The organic layer was washed with brine, dried over anhydrous Na₂SO₄, filtered and concentrated under reduced pressure to yield a crude, which was purified by flash chromatography (silica gel/PE/EA 50/1) to give tert-butyl(2-methoxybut-3-ynyloxy)dimethylsilane (2.0 g, 90.9%) as colorless oil. ¹H NMR (400 MHz, CDCl₃) δ 4.03-4.00 (m, 1H), 3.78-3.76 (m, 2H), 3.45 (s, 3H), 2.43 (d, J=2.5 Hz, 1H), 0.90 (s, 9H), 0.085 (s, 3H), 0.083 (s, 3H).

### (2S)-methyl 3-(tert-butoxycarbonylamino)-2-(4-(6-(tert-butyldimethylsilyloxy)-5-methoxyhexa-1,3-diynyl)benzamido)-3-methylbutanoate (47.2)

| Reagent | MW | Eq. | mmol | g, mL | |
|---|---|---|---|---|---|
| Compound 47.1 | 214.4 | 1.0 | 1.40 | 300 | mg |
| INT-1 | 453.3 | 1.0 | 1.40 | 634 | mg |
| PdCl₂(Ph₃)₂ | 701.9 | 0.1 | 0.12 | 84 | mg |
| CuI | 190.5 | 0.05 | 0.06 | 12 | mg |
| Et₃N | 101 | 3.0 | 4.2 | 0.6 | mL |
| THF | | | | 10 | mL |

To a stirring solution of compound INT-1 (634 mg, 1.40 mmol), PdCl₂(Ph₃)₂ (84 mg, 0.12 mmol), and CuI (12 mg, 0.06 mmol) in THF (8 mL) was added compound 47.1 (150 mg, 1.19 mmol) in THF (5 mL), followed by Et₃N (0.6 mL, 4.2 mmol) and the reaction mixture was stirred at room temperature overnight. Water was then added, and the resulting mixture was extracted with EtOAc, washed with brine, concentrated and purified by flash chromatography (silica gel/PE:EA 1/1) to yield compound 47.2 (300 mg, 37%) as a pale yellow solid. MS: m/z calcd for C₃₁H₄₆N₂O₇Si 586.3. found [M+Na]⁺ 609.

### (2S)-methyl 3-amino-2-(4-(6-hydroxy-5-methoxyhexa-1,3-diynyl)benzamido)-3-methylbutanoate (47.3)

| Reagent | MW | Eq. | mmol | g, mL |
|---|---|---|---|---|
| Compound 47.2 | 586 | 1.0 | 0.41 | 240 mg |
| HCl (5M in MeOH) | | 36.5 | 15 | 3 mL |
| MeOH | | | | 2 mL |

To a stirring solution of compound 47.2 (240 mg, 0.41 mmol) in MeOH (2 mL) was added HCl (5 M in MeOH, 3 mL, 15 mmol) and the reaction was stirred for 2 h. NaHCO₃ (1.0 g) was added, and the reaction mixture was filtered. The filtrate was concentrated under reduced pressure and purified by flash chromatography to yield compound 47.3 as a white solid (100 mg, 66%). MS: m/z calcd for C₂₀H₂₄N₂O₅ 372.2. found [M+H]⁺ 373; ¹H NMR (400 MHz, DMSO-d₆) δ 8.43 (br s, 1H), 7.89 (d, J=8.4 Hz, 2H), 7.70 (d, J=8.1 Hz, 2H), 5.18 (t, J=6.0 Hz, 1H), 4.38 (s, 1H), 4.22 (t, J=5.7 Hz, 1H), 3.65 (t, J=5.8 Hz, 2H), 3.36 (s, 3H), 1.87 (br s, 2H), 1.13 (s, 3H), 1.12 (s, 3H).

### N—((S)-3-amino-1-(hydroxyamino)-3-methyl-1-oxobutan-2-yl)-4-(6-hydroxy-5-methoxyhexa-1,3-diyn-1-yl)benzamide (47)

To a stirring solution of compound 47.3 (110 mg, 0.295 mmol) in IPA (0.3 ml) was added 50% hydroxylamine in water (0.390 mL, 5.91 mmol) at 0° C. and the reaction was stirred for 20 hr. The reaction mixture was concentrated under reduced pressure and acidified with AcOH (0.4 mL) and then purified by RP HPLC (1″, 0.1% AcOH in water/ACN) to yield N—((S)-3-amino-1-(hydroxyamino)-3-methyl-1-oxobutan-2-yl)-4-(6-hydroxy-5-methoxyhexa-1,3-diyn-1-yl)benzamide (54 mg, 0.144 mmol, 48.7%). MS: m/z calcd for C₁₉H₂₃N₃O₅ 373.2. found [M+H]⁺ 374.0.

### 48. N-((2S,3R)-1-(hydroxyamino)-3-(methylamino)-1-oxobutan-2-yl)-4-(6-hydroxyhexa-1,3-diyn-1-yl)benzamide (48)

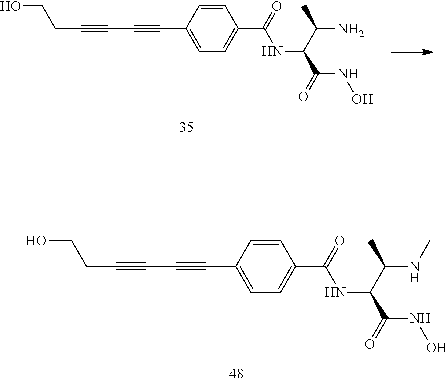 [SMILES: C[C@@H](N)[C@H](NC(=O)c1ccc(C#CC#CCCO)cc1)C(=O)NO ; CN[C@H](C)[C@H](NC(=O)c1ccc(C#CC#CCCO)cc1)C(=O)NO]

| | | | | | Sample | | |
|---|---|---|---|---|---|---|---|
| | Reg | | | Moles | Mass | Vol | Density |
| Reactant | Number | MW | Eq | (mmol) | (mg) | (ml) | (g/ml) |
| N-((2S,3R)-3-amino-1- | | 329.350 | 1.000 | 0.243 | 80 | | |
| (hydroxyamino)-1- | | | | | | | |
| oxobutan-2-yl)-4-(6- | | | | | | | |
| hydroxyhexa-1,3-diyn-1- | | | | | | | |
| yl)benzamide | | | | | | | |
| paraformaldehyde | | 30.026 | 0.9 | 0.219 | 6.56 | | |
| Sodium cyanoborohydride | | 39.853 | 3 | 0.729 | 45.8 | | |
| TFA | | 114.023 | 5 | 1.215 | 138 | 0.094 | 1.48 |
| DIPEA | | 129.243 | 2 | 0.486 | 62.8 | 0.085 | 0.74 |

To a stirring solution of N-((2S,3R)-3-amino-1-(hydroxyamino)-1-oxobutan-2-yl)-4-(6-hydroxyhexa-1,3-diyn-1-yl)benzamide (80 mg, 0.243 mmol) in DMF (2 mL) was added paraformaldehyde (6.56 mg, 0.219 mmol) followed by DIPEA (0.085 mL, 0.486 mmol) and methanol (1 mL) and the reaction mixture was stirred overnight. Additionally, 37% formaldehyde in water (10 μL) was then added. Sodium cyanoborohydride (45.8 mg, 0.729 mmol) was added, followed by TFA (0.094 mL, 1.215 mmol)—After 2 h the reaction was complete and the crude was purified by RP HPLC (1″, 0.1% TFA in water/ACN) to yield N-((2S,3R)-1-(hydroxyamino)-3-(methylamino)-1-oxobutan-2-yl)-4-(6-hydroxyhexa-1,3-diyn-1-yl)benzamide (42.4 mg, 0.089 mmol, 36.5% yield). MS: m/z calcd for C₁₈H₂₁N₃O₄343.1. found [M+H]⁺ 344.0.

### 49. N-((2S,3R)-1-(hydroxyamino)-3-(methylamino)-1-oxobutan-2-yl)-4-((S)-5-hydroxyhexa-1,3-diyn-1-yl)benzamide (49)

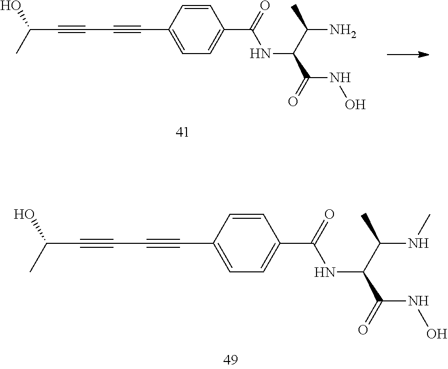 [SMILES: C[C@H](O)C#CC#Cc1ccc(C(=O)N[C@H](C(=O)NO)[C@@H](C)N)cc1 ; CN[C@H](C)[C@H](NC(=O)c1ccc(C#CC#C[C@H](C)O)cc1)C(=O)NO]

Sample

| | | | Moles | Mass | | Density | % |
|---|---|---|---|---|---|---|---|
| Reactant | MW | Eq | (mmol) | (mg) | Vol | (g/ml) | Wt (%) |
| N-((2S,3R)-3-amino-1- | 329.350 | 1.000 | 0.128 | 42 | | | |
| (hydroxyamino)-1- | | | | | | | |
| oxobutan-2-yl)-4-((S)-5- | | | | | | | |
| hydroxyhexa-1,3-diyn-1- | | | | | | | |
| yl)benzamide | | | | | | | |
| 37% formaldehyde in water | 30.026 | 0.5 | 0.064 | 5.17 | 4.75 | 1.09 | 37 |
| | | | | | μL | | |
| Sodium cyanoborohydride | 39.853 | 3 | 0.383 | 24.04 | | | |
| TFA | 114.023 | 3 | 0.383 | 43.6 | 0.029 | 1.48 | |
| | | | | | ml | | |

N-((2S,3R)-3-amino-1-(hydroxyamino)-1-oxobutan-2-yl)-4-((S)-5-hydroxyhexa-1,3-diyn-1-yl)benzamide (42 mg, 0.128 mmol) was dissolved in DMF (1 mL) and 37% formaldehyde in water (4.75 μL, 0.064 mmol) was added and the reaction was stirred overnight. Methanol (1 ml) was added followed by sodium cyanoborohydride (24.04 mg, 0.383 mmol) and TFA (0.029 ml, 0.383 mmol). After 1 h, the reaction was complete, and the reaction mixture was purified by RP HPLC (1″, 0.1% TFA in water/ACN) to yield N-((2S,3R)-1-(hydroxyamino)-3-(methylamino)-1-oxobutan-2-yl)-4-((S)-5-hydroxyhexa-1,3-diyn-1-yl)benzamide (8 mg, 0.017 mmol, 13.4%). MS: m/z calcd for C₁₈H₂₁N₃O₄343.1. found [M+H]⁺ 344.0.

### 50. N—((S)-3-amino-1-(hydroxyamino)-3-methyl-1-oxobutan-2-yl)-4-(5,6-dihydroxy-5-methylhexa-1,3-diyn-1-yl)benzamide (50)

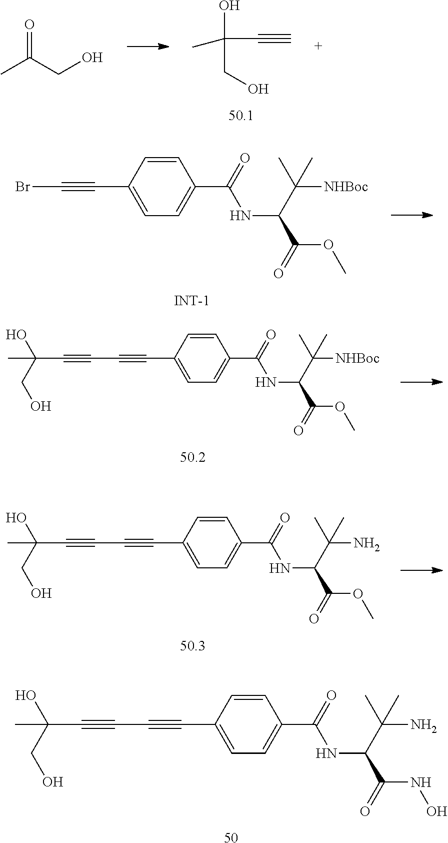

### 2-methylbut-3-yne-1,2-diol (74.1)

| Reagent | MW | Eq. | mmol | g, mL |
|---|---|---|---|---|
| 1-hydroxypropan-2-one | 74.08 | 1.0 | 4 | 296 mg |
| Ethynylmagnesium bromide | 129.24 | 1.0 | 4 | 8 mL |
| THF | | | | 10 mL |

To a stirring solution of 1-hydroxypropan-2-one (296 mg, 4 mmol) in THF (10 mL) was added ethynylmagnesium bromide (8 mL, 4 mmol) and the reaction was stirred at rt for 12 hr. After filtration and evaporation of the solvent under reduced pressure, the crude was purified by flash chromatography (silica gel) to yield compound 50.1 (150 mg, 38%) as a yellow oil. ¹H NMR (400 MHz, CDCl₃) δ 1.47 (s, 3H), 2.5 (s, 1H), 3.51 (d, J=11.2 Hz, 1H), 3.68 (d, J=10.8 Hz, 1H).

### (S)-methyl 3-(tert-butoxycarbonylamino)-2-(4-(5,6-dihydroxy-5-methylhexa-1,3-diynyl)benzamido)-3-methylbutanoate (50.2)

| Reagent | MW | Eq. | mmol | g, mL | |
|---|---|---|---|---|---|
| Compound 50.1 | 150 | 1.0 | 1.50 | 150 | mg |
| INT-1 | 453 | 1.0 | 1.50 | 680 | mg |
| Pd(PPh₃)₂Cl₂ | 702 | 0.05 | 0.075 | 52.7 | mg |
| CuI | 190 | 0.03 | 0.045 | 9 | mg |
| TEA | 101 | 2 | | 202 | mg |
| THF | | | 2 | 15 | mL |

To a stirring solution of compound 50.1 (150 mg, 1.5 mmol) in THF (15 mL) were added INT-1 (680 mg, 1.5 mmol), Pd(PPh₃)₂Cl₂ (52.7 mg, 0.075 mmol), CuI (9 mg, 0.045 mmol), and TEA (202 mg, 2 mmol) at rt under a N₂ atmosphere, and the reaction was stirred for 12 h. Solvent removal under reduced pressure gave a residue, which was purified by flash chromatography (silica gel/PE:EA 1:2) to yield compound 50.2 (300 mg, 64%) as a yellow oil.

### (S)-methyl 3-amino-2-(4-(5,6-dihydroxy-5-methylhexa-1,3-diynyl)benzamido)-3-methylbutanoate (50.3)

To a stirring solution of compound 50.2 (300 mg, 0.64 mmol) in DCM was added HCl (1N in MeOH, 10 mL) and the reaction was stirred for 12 hr. Solvent removal under reduced pressure gave a crude, which was purified by reverse column (CH₃CN:H₂O=30:70) to yield compound 50.3 (123 mg, 52%) as a yellow solid. ¹H NMR (400 MHz, DMSO-d₆) δ 8.41 (s, 3H), 7.88 (d, J=8.8 Hz, 2H), 7.67 (d, J=8.0 Hz, 2H), 5.63 (s, 1H), 5.12 (t, J=6.0 Hz, 1H), 4.38 (s, 1H), 3.65 (s, 3H), 3.39-3.43 (m, 1H), 3.32-3.45 (m, 1H), 1.77-1.89 (m, 2H), 1.36 (s, 3H), 1.12 (d, J=7.2 Hz, 6H).

### N—((S)-3-amino-1-(hydroxyamino)-3-methyl-1-oxobutan-2-yl)-4-(5,6-dihydroxy-5-methylhexa-1,3-diyn-1-yl)benzamide (50)

To a stirring solution of compound 50.3 (40 mg, 0.107 mmol) in IPA (0.3 mL) at 0° C. was added hydroxylamine (50% aqueous, 0.3 mL) and the reaction was stirred overnight. The reaction was acidified with AcOH (0.3 mL), diluted with water (0.7 mL), and purified by RP HPLC (1″, 0.1% AcOH in water/ACN) to yield compound 50 (6.8 mg, 16.9%). MS: m/z calcd for C₁₉H₂₃N₃O₅ 373.2. found [M+H]⁺ 374.2.

### 51. 4-(5,6-dihydroxyhexa-1,3-diyn-1-yl)-N—((S)-1-(hydroxyamino)-3-methyl-3-(methylamino)-1-oxobutan-2-yl)benzamide (51)

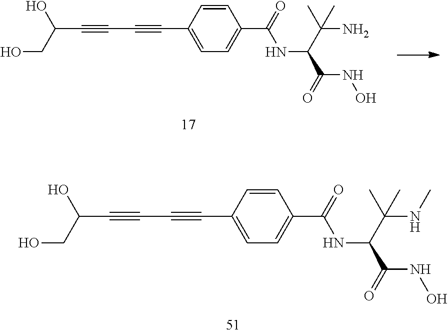 [SMILES: CC(C)(N)[C@H](NC(=O)c1ccc(C#CC#CC(O)CO)cc1)C(=O)NO ; CNC(C)(C)[C@H](NC(=O)c1ccc(C#CC#CC(O)CO)cc1)C(=O)NO]

| | | | | Sample | | | % |
|---|---|---|---|---|---|---|---|
| | | | Moles | Mass | Vol | | Wt |
| Reactant | MW | Eq | (mmol) | (g) | (ml) | Density | (%) |
| N-((S)-3-amino-1- | 359.376 | 1.000 | 3.06 | 1.1 | | | |
| (hydroxyamino)-3-methyl- | | | | | | | |
| 1-oxobutan-2-yl)-4-(5,6- | | | | | | | |
| dihydroxyhexa-1,3-diyn-1- | | | | | | | |
| yl)benzamide | | | | | | | |
| ethanamine | 45.084 | 9 | 27.5 | 1.774 | 1.774 | 1 g/ml | 70 |
| formaldehyde | 30.026 | 9 | 27.5 | 2.363 | 2.363 | 1 | 35 |
| AcOH | 60.052 | 5 | 15.30 | 0.919 | 0.876 | 1.049 | |
| | | | | | | g/ml | |
| sodium cyanoborohydride | 39.853 | 20 | 61.2 | 3.85 | | | |

To a stirring solution of compound 17 (1.1 g, 3.06 mmol) in THF (5 mL) and MeOH (5 mL) was added formaldehyde (2.36 mL, 27.5 mmol) and the reaction was stirred overnight. Ethylamine (70% in water, 2.4 mL) was added, and the reaction mixture was concentrated under reduced pressure to give a residue, which was dissolved THF (5 mL). AcOH (1.6 mL) was added, followed by sodium cyanoborohydride (3.85 g, 61.2 mmol). The reaction was exothermic, and additional THF (10 mL) and MeOH (10 mL) were added. Additional AcOH (4 mL) was added, and the reaction mixture was stirred overnight. Solvent evaporation under reduced pressure gave a crude, which was purified by RP HPLC (2″, 0.1% AcOH in water/ACN) to yield compound 51 (281 mg, 24.6%). MS: m/z calcd for C₁₉H₂₃N₃O₅ 373.2. found [M+H]⁺ 374.3.

### 52. N—((S)-3-amino-1-(hydroxyamino)-3-methyl-1-oxobutan-2-yl)-4-((E)-6,7-dihydroxyhept-3-en-1-yn-1-yl)benzamide (52)

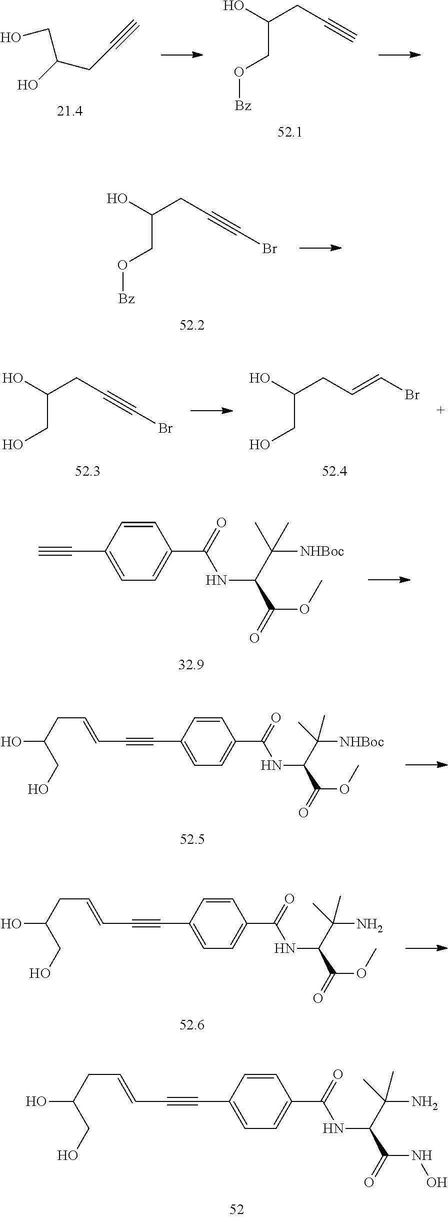

### 2-hydroxypent-4-ynyl benzoate (52.1)

| Reagent | MW | Eqiv. | Mmol | Amount | |
|---|---|---|---|---|---|
| Compound | 100.12 | 1.0 | 1.0 | 1.0 | g |
| 21.4 | | | | | |
| PhCOCl | 140.57 | 1.1 | 1.1 | 1.54 | g |
| DIPEA | 129.25 | 2.0 | 2.0 | 2.58 | g |
| THF | | | | 25 | mL |

To a stirring solution of compound 21.4 (1.0 g, 1.0 mmol) and DIPEA (2.58 g, 2.0 mmol) in anhydrous THF (20 mL) was added a solution of benzoyl chloride (1.54 g, 1.1 mmol) in THF (5 mL) dropwise and the reaction mixture was stirred for 12 h. The reaction was diluted with water (100 mL) and extracted with EA (3×100 mL). The combined organic layers were washed with brine (200 mL), dried (Na₂SO₄), and filtered. The filtrate was concentrated under reduced pressure to give a crude, which was purified by flash chromatography (silica gel/PE/EA: 20/1 to 5/1) to yield compound 52.1 (686 mg, 33%). MS: m/z calcd for C₁₂H₁₂O₃204.1. found [M+H]⁺ 205.1.

### 5-bromo-2-hydroxypent-4-ynyl benzoate (52.2)

| Reagent | MW | Eqiv. | Mmol | Amount |
|---|---|---|---|---|
| Compound | 204.22 | 1.0 | 3.36 | 686 mg |
| 52.1 | | | | |
| NBS | 177.98 | 1.1 | 3.70 | 658 mg |
| AgNO₃ | 169.87 | 0.1 (w/w) | | 69 mg |
| Acetone | | | | 10 mL |

To a stirring solution of 2-hydroxypent-4-ynyl benzoate (686 mg, 3.36 mmol) and AgNO₃ (69 mg, 10% (w/w)) in acetone (10 mL) was added NBS (658 mg, 3.70 mmol) in portions. The mixture was stirred for 1 h, it was then diluted with ether (20 mL), and filtered. The filtrate was concentrated under reduced pressure to give a crude, which was purified by flash chromatography (silica gel/PE/EA: 20/1 to 5/1) to yield compound 52.2 (790 mg, 83%). MS: m/z calcd for C₁₂H₁₁BrO₃ 282.0. found [M+H]⁺ 282.9.

### 5-bromopent-4-yne-1,2-diol (52.3)

| Reagent | MW | Eqiv. | Mmol | Amount |
|---|---|---|---|---|
| Compound | 283.12 | 1.0 | 2.79 | 790 mg |
| 52.2 | | | | |
| K₂CO₃ | 138.21 | 1.5 | 4.19 | 578 mg |
| MeOH | | | | 3 mL |

To a stirring solution of 5-bromo-2-hydroxypent-4-ynyl benzoate (790 mg, 2.79 mmol) in MeOH (3 mL) was added K₂CO₃ (53 g, 500 mmol) and the reaction was stirred for 12 hr. It was concentrated under reduced pressure to give a crude, which was purified by flash chromatography (silica gel/PE/EA: 10/1 to 1/3) to give compound 52.3 (245 mg, 49%). ¹H NMR (400 MHz, CDCl₃) δ 1.87 (s, 1H), 2.31 (d, J=3.2 Hz, 1H), 2.40-2.42 (m, 2H), 3.51-3.55 (m, 1H), 3.67-3.70 (m, 1H), 3.82-3.84 (m, 1H).

### (E)-5-bromopent-4-ene-1,2-diol (52.4)

| Reagent | MW | Eqiv. | Mmol | Amount |
|---|---|---|---|---|
| Compound | 179.01 | 1.0 | 1.37 | 245 mg |
| 52.3 | | | | |
| AlCl₃ | 133.34 | 1.05 | 1.44 | 192 mg |
| LiAlH₄ | 37.95 | 2.0 | 2.74 | 104 mg |
| Et₂O | | | | 1 mL |

To a suspension of LiAlH₄ (104 mg, 2.74 mmol) in dry Et₂O (1.0 mL) was added a solution of AlCl₃ (192 mg, 1.44 mmol) in Et₂O (1.0 mL) dropwise at −5° C., followed by a solution of 5-bromopent-4-yne-1,2-diol (245 mg, 1.37 mmol) in Et₂O (1.0 mL). The mixture was warmed to rt and then refluxed for 3 hr. The reaction mixture was cooled to −5° C., water (0.1 mL) was carefully added, followed by diethyl ether (5 mL). NaOH (15%, 0.1 mL) was carefully added, followed by water (0.2 mL) and ether (5 mL). The reaction was dried over (MgSO₄), filtered, and concentrated under reduced pressure to yield compound 52.4 (250 mg), which was carried through to the next step without further purification.

### (2S)-methyl-3-(tert-butoxycarbonylamino)-2-(4-((E)-6,7-dihydroxyhept-3-en-1-ynyl)benzamido)-3-methylbutanoate (52.5)

| Reagent | MW | Eqiv. | Mmol | Amount |
|---|---|---|---|---|
| Compound | 181.03 | 1.0 | 1.37 | 248 mg |
| 52.4 | | | | |
| Compound | 374.43 | 1.2 | 1.64 | 616 mg |
| 32.9 | | | | |
| CuI | 190.45 | 0.1 | 0.137 | 26 mg |
| Pd(PPh₂)₂Cl₂ | 547.69 | 0.03 | 0.041 | 23 mg |
| TEA | | | | 10 mL |

A mixture of (E)-5-bromopent-4-ene-1,2-diol (248 mg, 1.37 mmol), (S)-methyl 3-(tert-butoxycarbonylamino)-2-(4-ethynylbenzamido)-3-methylbutanoate (616 mg, 1.64 mmol), CuI (26 mg, 0.137 mmol), Pd(PPh₃)₂Cl₂ (23 mg, 0.041 mmol) and TEA (10 mL) in THF was stirred at 90° C. under nitrogen for 30 min. The reaction mixture was concentrated under reduced pressure to a crude, which was purified by flash chromatography (silica gel/PE/EA: 5/1 to 1/2) to yield compound 52.5 (27 mg, 4% for 2 steps). MS: m/z calcd for C₂₅H₃₄N₂O₇474.2. found [M+H]⁺ 475.2

### (2S)-methyl-3-amino-2-(4-((E)-6,7-dihydroxyhept-3-en-1-ynyl)benzamido)-3-methylbutanoate (52.6)

| Reagent | MW | Mmol | Amount |
|---|---|---|---|
| compound 52.5 | 474.55 | 0.057 | 27 mg |
| TFA | | | 0.2 mL |
| DCM | | | 1 mL |

To a stirring solution of (2S)-methyl 3-(tert-butoxycarbonylamino)-2-(4-((E)-6,7-dihydroxyhept-3-en-1-ynyl)benzamido)-3-methylbutanoate (27 mg, 0.057 mmol) in DCM (1 mL) was added TFA (0.2 mL) and the reaction mixture was stirred for 4 hr. Solvent evaporation under reduced pressure gave a crude, which was purified by RP-HPLC (0.01% TFA in water and ACN) to give compound 52.6 as TFA salt (9 mg, 32%). MS: m/z calcd for C₂₀H₂₆N₂O₅ 374.2. found [M+H]⁺ 375.2; ¹H NMR (400 MHz, DMSO-d₆) δ 1.12 (d, J=7.2 Hz, 6H), 1.92 (bs, 1H), 2.12-2.20 (m, 1H), 2.33-2.40 (m, 1H), 3.25-3.26 (m, 2H), 3.5 (s, 1H), 3.64 (s, 3H), 4.37 (s, 1H), 4.61 (s, 1H), 4.72 (d, J=4.4 Hz, 1H), 75.84 (d, J=15.6 Hz, 1H), 6.30-6.38 (m, 1H), 7.53 (d, J=7.6 Hz, 2 H), 7.86 (d, J=8.4 Hz, 2H), 8.37 (s, 1H). ¹⁹F NMR (376 MHz, DMSO-d₆) δ 73.46 (s, 3H).

### N—((S)-3-amino-1-(hydroxyamino)-3-methyl-1-oxobutan-2-yl)-4-((E)-6,7-dihydroxyhept-3-en-1-yn-1-yl)benzamide (52)

To a stirring solution of compound 52.6 in IPA (0.5 ml) was added hydroxylamine (50% aqueous, 0.3 ml) and the reaction was stirred for 26 hr. Solvent evaporation under reduced pressure gave a crude, which was acidified with AcOH and purified by RP HPLC to yield compound 52. MS: m/z calcd for C₁₉H₂₆N₃O₅ 375.2. found [M+H]⁺ 376.3.

### 53. N—((S)-3-amino-1-(hydroxyamino)-3-methyl-1-oxobutan-2-yl)-4-((5S,6S)-5,7-dihydroxy-6-methylhepta-1,3-diyn-1-yl)benzamide (53)

### (S)-methyl 3-((tert-butyldimethylsilyl)oxy)-2-methylpropanoate (53.1)

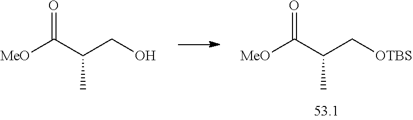

| | | | | Sample |
|---|---|---|---|---|
| | | | Moles | Mass |
| Reactant | MW | Eq | (mmol) | (g) |
| (S)-methyl 3-hydroxy-2- | 118.131 | 1.000 | 85 | 10 |
| methylpropanoate | | | | |
| 1H-imidazole | 68.077 | 1.2 | 102 | 6.92 |
| tert-butylchlorodimethylsilane | 150.722 | 1.1 | 93 | 14.03 |

To a stirring solution of (S)-methyl 3-hydroxy-2-methylpropanoate (10 g, 85 mmol) in DCM (250 mL) was added 1H-imidazole (6.92 g, 102 mmol) followed by tert-butylchlorodimethylsilane (14.03 g, 93 mmol) and the reaction was stirred overnight at room temperature. The reaction mixture was filtered and the filtrate was concentrated to give an oil, which was purified by flash chromatography (silica gel/10% EtOAc/hexanes) to yield compound 53.1 (17.31 g, 88%) as a clear liquid. TLC 1:10 EtOAc/hexanes Rf: 0.56; ¹H NMR (400 MHz, DMSO-d₆) δ 3.67 (d, J=6 Hz, 2H), 3.58 (s, 3H), 2.60 (m, 1H), 1.04 (d, J=6.8 Hz, 3H), 0.84 (s, 9H), 0.01 (s, 6H).

### (S)-3-((tert-butyldimethylsilyl)oxy)-N-methoxy-N,2-dimethylpropanamide (53.2)

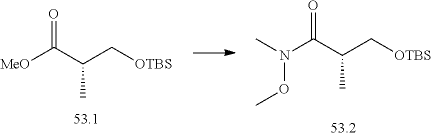 [SMILES: COC(=O)[C@@H](C)CO[Si](C)(C)C(C)(C)C ; CON(C)C(=O)[C@@H](C)CO[Si](C)(C)C(C)(C)C]

| | | | | Sample | | |
|---|---|---|---|---|---|---|
| | | | Moles | Mass | Vol | Molarity |
| Reactant | MW | Eq | (mmol) | (g) | (ml) | (molar) |
| (S)-methyl 3-((tert- | 232.392 | 1.000 | 74.4 | 17.3 | | |
| butyldimethylsilyl)oxy)-2- | | | | | | |
| methylpropanoate | | | | | | |
| N,O-dimethylhydroxylamine | 61.083 | 1.50 | 112 | 10.89 | | |
| hydrochloride | | | | | | |
| isopropylmagnesium chloride | 102.846 | 3 | 223 | | 112 | 2 |

To a stirring solution of (S)-methyl 3-((tert-butyldimethylsilyl)oxy)-2-methylpropanoate (17.3 g, 74.4 mmol) in anhydrous THF (200 mL) was added N,O-dimethylhydroxylamine hydrochloride (10.89 g, 112 mmol) and the reaction was cooled to −20° C. Isopropylmagnesium chloride (112 ml, 223 mmol) was then added dropwise over 30 min while maintaining the temperature below −20° C., and the reaction was stirred for 2.5 hours. The reaction was warmed to 0° C. and quenched with saturated NH₄Cl (100 mL). Diethyl ether (100 mL) was added, and the layers were separated. The aqueous layer was extracted with Et₂O (2×100 mL), the combined organic layers were washed with water, brine, dried over sodium sulfate, filtered and concentrated under reduced pressure to yield compound 53.2 (18.9 g), which was carried through to the next step without further purification. TLC 1:10 EtOAc/hex product Rf: 0.16. ¹H NMR (400 MHz, DMSO-d₆) δ 3.94 (s and t, 4H), 3.50 (t, 1H), 3.10 (s+m, 4H), 0.95 (d, 3H), 0.83 (s, 9H), 0.0 (s, 6H).

### (S)-5-((tert-butyldimethylsilyl)oxy)-4-methylpent-1-yn-3-one (53.3)

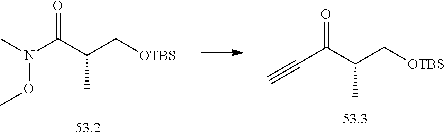 [SMILES: CON(C)C(=O)[C@@H](C)CO[Si](C)(C)C(C)(C)C ; C#CC(=O)[C@@H](C)CO[Si](C)(C)C(C)(C)C]

| | | | | Sample | | |
|---|---|---|---|---|---|---|
| | | | Moles | Mass | Vol | Molarity |
| Reactant | MW | Eq | (mmol) | (g) | (ml) | (molar) |
| (S)-3-((tert- | 261.433 | 1.000 | 72.3 | 18.89 | | |
| butyldimethylsilyl)oxy)-N- | | | | | | |
| methoxy-N,2- | | | | | | |
| dimethylpropanannide | | | | | | |
| ethynylmagnesium | 129.238 | 1.5 | 108 | | 217 | 0.5 |
| bromide | | | | | | |

To a stirring solution of (S)-3-((tert-butyldimethylsilyl)oxy)-N-methoxy-N,2-dimethylpropanamide (18.89 g, 72.3 mmol) in anhydrous THF (150 mL) at 0° C. was added dropwise over 1 hr ethynylmagnesium bromide (217 ml, 108 mmol) and the reaction was stirred for 2 hr at 0° C. The reaction mixture was poured into ice water (200 mL), and diethyl ether (100 mL) was added. Separation of the layers was aided by the addition of saturated NH₄Cl (30 mL), and the aqueous layer was extracted with Et₂O (3×75 mL). The combined organic layers were washed with water, brine, dried over sodium sulfate, filtered and concentrated under reduced pressure to an amber oil, which was purified by flash chromatography (silica gel/10-25% EtOAc/hex) to yield compound 53.3 (5.98 g, 36.6%). TLC 1:4 EtOAc/hex Rf 0.70. ¹H NMR (400 MHz, CDCl₃) δ 3.86 (m, 1H), 3.83 (m, 1H), 3.21 (s, 1H), 2.8 (m, 1H), 1.18 (d, J=7.2 Hz, 3H), 0.87 (s, 9H), 0.04 (s, 6H).

### (3S,4S)-5-((tert-butyldimethylsilyl)oxy)-4-methylpent-1-yn-3-ol (53.4)

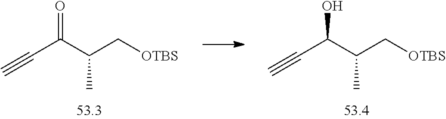

| | | | | Sam- | | Mo- |
|---|---|---|---|---|---|---|
| | | | | ple | | larity |
| | | | Moles | Mass | Vol | (mo- |
| Reactant | MW | Eq | (mmol) | (g) | (ml) | lar) |
| (S)-5-((tert-butyl- | 226.387 | 1.000 | 13.25 | 3.00 | | |
| dimethylsilyl)oxy)-4- | | | | | | |
| methylpent-1-yn-3-one | | | | | | |
| (S)-1-methyl-3,3- | 277.169 | 1.90 | 25.2 | 6.98 | | |
| diphenylhexahydro | | | | | | |
| pyrrolo[1,2-c][1,3,2]- | | | | | | |
| oxazaborole | | | | | | |
| Borane dimethyl sulfide | 62.134 | 5 | 66.3 | | 33.1 | 2 |

To a stirring solution of (S)-5-((tert-butyldimethylsilyl)oxy)-4-methylpent-1-yn-3-one (3.00 g, 13.25 mmol) in anhydrous THF (65 mL), under a nitrogen atmosphere at −30° C. was added (S)-1-methyl-3,3-diphenylhexahydropyrrolo[1,2-c][1,3,2]oxazaborole (6.98 g, 25.2 mmol, 2M in toluene), followed by the dropwise addition over 10 min of borane dimethyl sulfide (33.1 ml, 66.3 mmol) and the reaction was stirred for 2 hours at −30° C. The reaction was quenched by the slow addition of cold ethanol (50 mL). The reaction was then warmed to rt, poured into water (100 mL), and Et₂O (200 mL) was added. The layers were separated and the aqueous layer was extracted with Et₂O. The combined organic layers were washed with brine, dried over sodium sulfate, filtered and concentrated under reduced pressure to give a crude, which was purified by flash chromatography (silica gel/10-20% EtOAc/hexanes) to yield compound 53.4 (2.10 g, 9.19 mmol). TLC 1:4 EtOAc/hexanes Rf 0.53 ¹H NMR (400 MHz, CDCl₃) δ 4.40 (m, 1H), 3.93 (dd, 1H), 3.60 (t, 1H), 3.48 (m, 1H), 2.45 (s, 1H), 1.95 (m, 1H), 1.04 (d, 3H), 0.90 (s, 9H), 0.08 (s, 6H).

### (2S,3S)-2-methylpent-4-yne-1,3-diol (53.5)

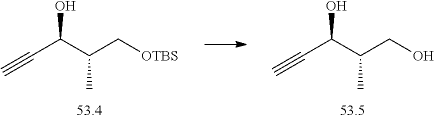

To a stirring solution of (3S,4S)-5-((tert-butyldimethylsilyl)oxy)-4-methylpent-1-yn-3-ol (2 g, 8.76 mmol) in anhydrous THF (40 mL) at 0° C. was added TBAF (1M in THF, 9.63 mL), and the progress of the reaction was followed by TLC. After 2 hours, the reaction was poured into ice water (100 mL), and Et₂O (100 mL) was added. The aqueous layer was extracted with Et₂O, and the combined organic layers were washed with water, brine, and concentrated under reduced pressure to give a crude, which was purified by flash chromatography (silica gel/50-100 EtOAc/hex) to yield compound 53.5 (0.6 g, 5.26 mmol). TLC 1:1 EtOAc/hex Rf 0.1. ¹H NMR (400 MHz, CDCl₃) δ 4.40 (m. 1H), 3.85 (m, 1H), 3.65 (m, 1H), 2.85 (bs, 1H), 2.51 (s, 1H), 2.25 (bs, 1H), 2.0 (m, 1H), 1.05 (d, 3H).

### (S)-methyl 3-amino-2-(4-((5S,6S)-5,7-dihydroxy-6-methylhepta-1,3-diyn-1-yl)benzamido)-3-methylbutanoate (53.6)

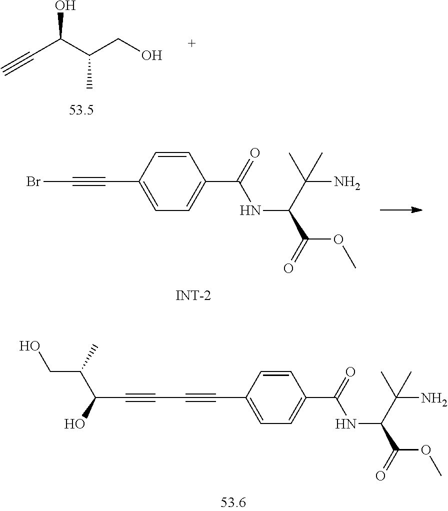

| | | | Moles | Sample Mass |
|---|---|---|---|---|
| Reactant | MW | Eq | (mmol) | (mg) |
| copper(I) chloride | 98.999 | 0.02 | 0.105 | 10.41 |
| butan-1-amine | 73.137 | 23 | 121 | 8842 |
| (2S,3S)-2-methylpent-4- | 114.142 | 1.000 | 5.26 | 600 |
| yne-1,3-diol | | | | |
| hydroxylamine | 69.491 | 0.06 | 0.315 | 21.92 |
| hydrochloride | | | | |
| (S)-methyl 3-amino-2-(4- | 353.211 | 1.000 | 5.26 | 2048 |
| (bromoethynyl)benzamido)- | | | | |
| 3-methylbutanoate | | | | |
| hydrochloride | | | | |

To a stirring solution of hydroxylamine hydrochloride (21.92 mg, 0.315 mmol) and copper(I) chloride (10.41 mg, 0.105 mmol) in 30% n-butylamine/water (5 mL) at 0° C. was added a solution of (2S,3S)-2-methylpent-4-yne-1,3-diol (600 mg, 5.26 mmol) in 30% n-butylamine/water (15 mL), followed by the dropwise addition over 20 min of a solution of (S)-methyl 3-amino-2-(4-(bromoethynyl)benzamido)-3-methylbutanoate hydrochloride (2048 mg, 5.26 mmol) in 30% n-butylamine/water (15 mL) and MeOH (25 mL), and hydroxylamine hydrochloride (21.92 mg, 0.315 mmol), and the reaction was stirred for 2 hr at 0° C. After solvent evaporation under reduced pressure, the aqueous layer was extracted with EtOAc, and the combined organic layers were washed with water, dried over sodium sulfate and concentrated under reduced pressure to give a crude, which was purified by flash chromatography (silica gel/5-10% MeOH/DCM) to yield compound 53.6 (1.07 g, 52.7%). MS: m/z calcd for C₂₁H₂₆N₂O₅386.2. found [M+H]⁺ 387.3.

### N—((S)-3-amino-1-(hydroxyamino)-3-methyl-1-oxobutan-2-yl)-4-((5S,6S)-5,7-dihydroxy-6-methylhepta-1,3-diyn-1-yl)benzamide (53)

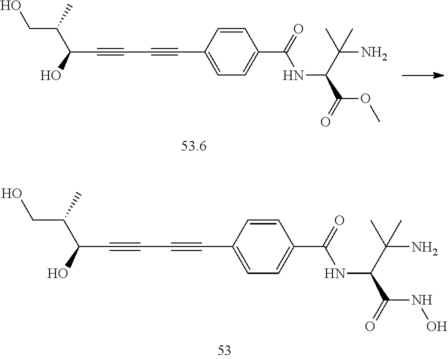 [SMILES: COC(=O)[C@@H](NC(=O)c1ccc(C#CC#C[C@@H](O)[C@@H](C)CO)cc1)C(C)(C)N ; C[C@@H](CO)[C@H](O)C#CC#Cc1ccc(C(=O)N[C@H](C(=O)NO)C(C)(C)N)cc1]

To a stirring solution of (S)-methyl 3-amino-2-(4-((5S,6S)-5,7-dihydroxy-6-methylhepta-1,3-diyn-1-yl)benzamido)-3-methylbutanoate (1.07 g, 2.77 mmol) in isopropanol (7 mL) at 0° C. was added hydroxylamine (50% aq, 3.66 ml, 55.4 mmol) and the reaction was stirred at 0° C. for 3 days. IPA was removed under reduced pressure, and the reaction mixture was acidified with AcOH (5 mL), diluted with water (4 mL) and purified by RP HPLC (2″, 0.1% TFA in water/ACN), to yield compound 53 (162 mg, 15.1%). MS: m/z calcd for C₂₀H₂₅N₃O₅387.2. found [M+H]⁺ 388.2.

### 54. N—((S)-3-amino-1-(hydroxyamino)-3-methyl-1-oxobutan-2-yl)-4-((S)-5,7-dihydroxyhepta-1,3-diyn-1-yl)benzamide (54)

### 3-((tert-butyldimethylsilyl)oxy)-N-methoxy-N-methylpropanamide (54.1)

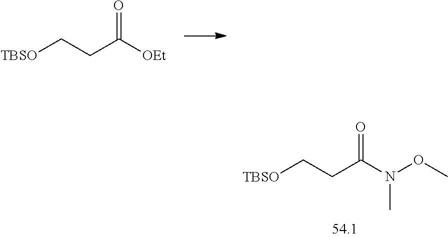 [SMILES: CCOC(=O)CCO[Si](C)(C)C(C)(C)C ; CON(C)C(=O)CCO[Si](C)(C)C(C)(C)C]

| | | | | Sam- | | Mo- |
|---|---|---|---|---|---|---|
| | | | | ple | | larity |
| | | | Moles | Mass | Vol | (mo- |
| Reactant | MW | Eq | (mmol) | (g) | (ml) | lar) |
| ethyl 3-((tert- | 232.392 | 1.000 | 53.4 | 12.4 | | |
| butyldimethylsilyl)- | | | | | | |
| oxy)propanoate | | | | | | |
| N,O-dimethylhydroxyl- | 61.083 | 1.5 | 80 | 7.81 | | |
| amine hydrochloride | | | | | | |
| isopropylmagnesium | 102.846 | 3 | 160 | | 80 | 2 |
| chloride | | | | | | |

To a stirring solution of ethyl 3-((tert-butyldimethylsilyl)oxy)propanoate (12.4 g, 53.4 mmol) in anhydrous THF (1300 mL) was added N, 0-dimethylhydroxylamine hydrochloride (7.81 g, 80 mmol) and the reaction was cooled to −20° C. Isopropylmagnesium chloride (80 ml, 160 mmol) was then added dropwise under a nitrogen atmosphere and the reaction was stirred for 1.5 hr. The reaction was quenched with saturated NH₄Cl, extracted into diethyl ether, and the combined organic layers were washed with water, brine, dried over sodium sulfate, filtered and concentrated under reduced pressure to yield compound 54.1 (12.3 g, 93%), which was carried through to the next step without further purification. ¹H NMR (400 MHz, CDCl₃) δ 3.94 (t, 7.2 Hz, 2H), 3.70 (s, 3H), 3.18 (t, 3H), 2.67 (t, 2H), 0.88 (s, 9H), 0.06 (6H).

### 5-((tert-butyldimethylsilyl)oxy)pent-1-yn-3-one (54.2)

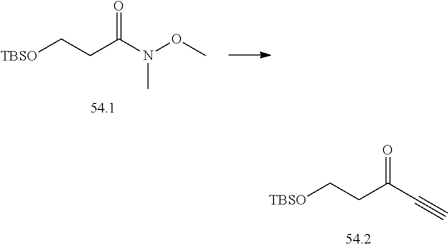 [SMILES: CON(C)C(=O)CCO[Si](C)(C)C(C)(C)C ; C#CC(=O)CCO[Si](C)(C)C(C)(C)C]

| | | | | Sam- | | Mo- |
|---|---|---|---|---|---|---|
| | | | | ple | | larity |
| | | | Moles | Mass | Vol | (mo- |
| Reactant | MW | Eq | (mmol) | (g) | (ml) | lar) |
| 3-((tert- | 247.407 | 1.000 | 49.7 | 12.30 | | |
| butyldimethylsilyl)oxy)- | | | | | | |
| N-methoxy-N- | | | | | | |
| methylpropanamide | | | | | | |
| ethynylmagnesium | 129.238 | 1.5 | 74.6 | | 149 | 0.50 |
| bromide | | | | | | |

To a stirring solution of 3-((tert-butyldimethylsilyl)oxy)-N-methoxy-N-methylpropanamide (12.30 g, 49.7 mmol) in anhydrous. THF (100 mL) at 0° C. under a nitrogen atmosphere was added ethynylmagnesium bromide (149 ml, 74.6 mmol) dropwise over 1 hour, and the reaction was stirred at 0° C. for 20 hr. The reaction mixture was slowly quenched with ice water (100 mL), and saturated aqueous NH₄Cl (100 mL). Et₂O (200 mL) was then added and the layers were separated. The aqueous layer was extracted with Et₂O, and the combined organic layers were washed with water, brine, dried over sodium sulfate, filtered and concentrated under reduced pressure to give a crude, which was purified by flash chromatography (silica gel/10-20% EtOAC/hexanes) to yield compound 54.2 (5.65 g, 53.3%). TLC 1:1 EtOAc/hex Rf 0.75 TLC 1:4 EtOAc/hex Rf 0.62. ¹H NMR (400 MHz, CDCl₃) δ 3.99 (t, 6 Hz, 3H), 3.22 (s, 1H), 2.78 (t, 6 Hz, 3H), 0.88 (s, 9H), 0.06 (s, 6H).

### (S)-5-((tert-butyldimethylsilyl)oxy)pent-1-yn-3-ol (54.3)

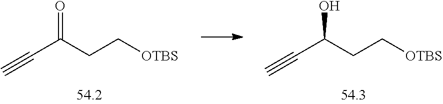

| | | | | Sam- | | Mo- |
|---|---|---|---|---|---|---|
| | | | | ple | | larity |
| | | | Moles | Mass | Vol | (mo- |
| Reactant | MW | Eq | (mmol) | (g) | (ml) | lar) |
| 5-((tert-butyl- | 212.361 | 1.000 | 14.03 | 2.98 | | |
| dimethylsilyl)oxy)- | | | | | | |
| pent-1-yn-3-one | | | | | | |
| (S)-1-methyl-3,3- | 277.169 | 1.85 | 26.0 | | 26.0 | 1 |
| diphenylhexahydro- | | | | | | |
| pyrrolo[1,2-c][1,3,2]- | | | | | | |
| oxazaborole | | | | | | |
| Borane dimethyl sulfide | 62.134 | 5 | 70.2 | | 35.1 | 2 |

To a stirring solution of 5-((tert-butyldimethylsilyl)oxy)pent-1-yn-3-one (2.98 g, 14.03 mmol) in THF (65 mL), under a nitrogen atmosphere at −30° C. was added (S)-1-methyl-3,3-diphenylhexahydropyrrolo[1,2-c][1,3,2]oxazaborole (26.0 ml, 26.0 mmol, 2M in toluene) followed by the dropwise addition of borane dimethyl sulfide (35.1 ml, 70.2 mmol) and the reaction was stirred for 2 hours at −30° C. The reaction was quenched by the dropwise addition of cold ethanol (50 mL). The reaction mixture was warmed to room temperature, and poured into water (100 mL). Et₂O (200 mL) was added, and the layers were separated. The aqueous layer was extracted with Et₂O, and the combined layers were washed with brine, dried over sodium sulfate, filtered and concentrated under reduced pressure to give a crude, which was purified by flash chromatography (silica gel/10-20% EtOAc/hexanes) to yield compound 54.3 (2.51 g, 11.71 mmol). TLC 1:4 EtOAc/hexanes Rf 0.48. ¹H NMR (400 MHz, CDCl₃) δ 4.6 (m, 1H), 4.05 (m, 1H), 3.84 (m, 1H), 3.44 (d, 6 Hz, 1H), 2.46 (dd, 1H), 2.0 (m, 1H), 1.87 (m, 1H), 0.90 (s, 9H), 0.08 (s, 6H).

### (S)-pent-4-yne-1,3-diol (54.4)

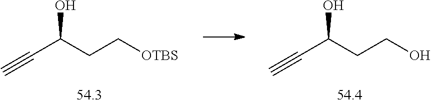

| | | | | Sam- | | Mo- |
|---|---|---|---|---|---|---|
| | | | | ple | | larity |
| | | | Moles | Mass | Vol | (mo- |
| Reactant | MW | Eq | (mmol) | (g) | (ml) | lar) |
| (S)-5-((tert- | 214.377 | 1.000 | 10.26 | 2.2 | | |
| butyldimethylsilyl)- | | | | | | |
| oxy)pent-1-yn-3-ol | | | | | | |
| TBAF | | 1.10 | 11.29 | | 11.29 | 1 |

To a stirring solution of (S)-5-((tert-butyldimethylsilyl)oxy)pent-1-yn-3-ol (2.2 g, 10.26 mmol) in anhydrous THF (40 mL) at 0° C. was added dropwise tetrabutylammonium fluoride (1M in THF, 11.29 mL), and the reaction was allowed to warm to rt for 2 hours. The reaction mixture was poured into ice water (100 mL), and Et₂O (100 mL) was added. The aqueous layer was extracted with Et₂O, and the combined layers were washed with water, brine, concentrated under reduced pressure to give a crude, which was purified by flash chromatography (silica gel/40-95% EtOAc/hex) to yield compound 54.4 (220 mg, 21.4%). TLC 1:1 EtOAc/hex Rf 0.10 ¹H NMR (400 MHz, CDCl₃) δ 4.66 (m, 1H), 4.03 (m, 1H), 3.88 (m, 1H), 2.51 (dd, 1H), 2.0 (m, 4H).

### (S)-methyl 3-amino-2-(4-((S)-5,7-dihydroxyhepta-1,3-diyn-1-yl)benzamido)-3-methylbutanoate (54.5)

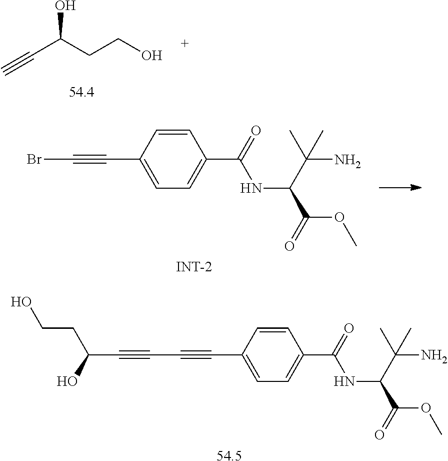

| | | | | Sample |
|---|---|---|---|---|
| | | | Moles | Mass |
| Reactant | MW | Eq | (mmol) | (mg) |
| copper(I) chloride | 98.999 | 0.02 | 0.044 | 4.35 |
| butan-1-amine | 73.137 | 23 | 50.5 | 3696 |
| (S)-pent-4-yne-1,3-diol | 100.116 | 1.000 | 2.197 | 220 |
| hydroxylamine hydrochloride | 69.491 | 0.06 | 0.132 | 9.16 |
| (S)-methyl 3-amino-2-(4- | 353.211 | 1.000 | 2.197 | 856 |
| (bromoethynyl)benzamido)-3- | | | | |
| methylbutanoate hydrochloride | | | | |

To a stirring solution of hydroxylamine hydrochloride (9.16 mg, 0.132 mmol) and copper(I) chloride (4.35 mg, 0.044 mmol) in 30% n-butylamine/water (3 mL) at 0° C. was added a solution containing (S)-pent-4-yne-1,3-diol (220 mg, 2.197 mmol) in 30% n-butylamine/water (8 mL), followed by dropwise addition over 20 min of a solution of (S)-methyl 3-amino-2-(4-(bromoethynyl)benzamido)-3-methylbutanoate hydrochloride (856 mg, 2.197 mmol) and hydroxylamine hydrochloride (9.16 mg, 0.132 mmol) in 30% n-butylamine/water (8 mL) and MeOH (12 mL), and the reaction was stirred for 2 hr at 0° C. After solvent evaporation under reduced pressure, the aqueous layer was extracted with EtOAc, and the combined organic layers were washed with water, dried over sodium sulfate, filtered and concentrated under reduced pressure to give a crude, which was purified by flash chromatography (silica gel/0-10% MeOH/DCM) to yield compound 54.5 (0.23 g, 28.1%). MS: m/z calcd for C₂₀H₂₄N₂O₅ 372.2. found [M+H]⁺ 373.2.

### N—((S)-3-amino-1-(hydroxyamino)-3-methyl-1-oxobutan-2-yl)-4-((S)-5,7-dihydroxyhepta-1,3-diyn-1-yl)benzamide (54)

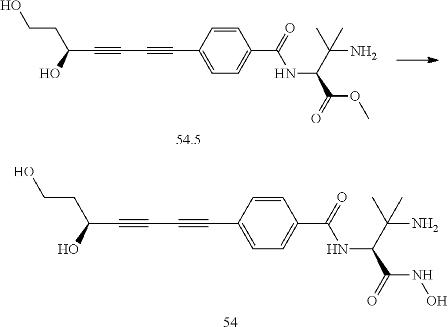 [SMILES: COC(=O)[C@@H](NC(=O)c1ccc(C#CC#C[C@@H](O)CCO)cc1)C(C)(C)N ; "54.5" ; CC(C)(N)[C@H](NC(=O)c1ccc(C#CC#C[C@@H](O)CCO)cc1)C(=O)NO ; "54"]

To a stirring solution of (S)-methyl 3-amino-2-(4-((S)-5,7-dihydroxyhepta-1,3-diyn-1-yl)benzamido)-3-methylbutanoate (231 mg, 0.620 mmol) in IPA (4 mL) at 0° C. was added hydroxylamine (820 μL, 12.41 mmol) and the mixture was stirred at 0° C. for 4 days. The mixture was acidified with acetic acid, diluted with water and purified by RP HPLC (2″, 0.1% AcOH in water/ACN) to yield compound 54. MS: m/z calcd for C₁₉H₂₃N₃O₅373.2. found [M+H]⁺ 374.1.

### 55. (S,E)-N-(3-amino-1-(hydroxyamino)-3-methyl-1-oxobutan-2-yl)-4-(6-hydroxyhex-3-en-1-yn-1-yl)benzamide (55)

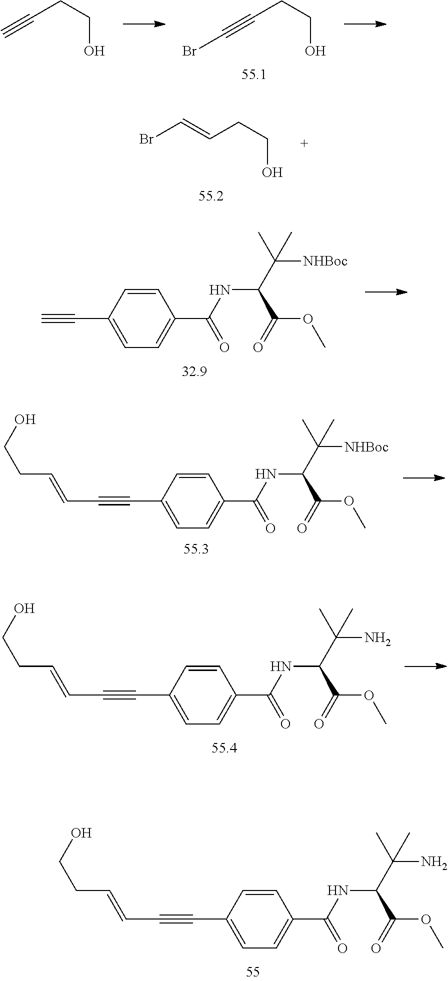

### 4-bromobut-3-yn-1-ol (55.1)

| Reagent | MW | Eq. | mmol | g, mL | |
|---|---|---|---|---|---|
| But-3-yn-1-ol | 70.09 | 1.0 | 57.07 | 4.0 | g |
| NBS | 178.68 | 1.2 | 68.48 | 12.2 | g |
| AgNO₃ | 169.87 | 0.05 | 2.85 | 484 | mg |
| Acetone | | | | 200 | mL |

To a stirring solution of but-3-yn-1-ol (4.0 g, 57.07 mmol) and NBS (12.2 g, 68.48 mmol) in acetone (200 mL) was added AgNO₃ (484 mg, 2.85 mmol) and the mixture was stirred at 25° C. for 1 hr. After filtration and evaporation of solvent under reduced pressure, the crude was purified by flash chromatography (silica gel) to give compound 55.1 (8.0 g, 95%) as yellow oil. ¹H NMR (400 MHz, CDCl₃) δ 3.72-3.76 (m, 2H), 2.48-2.51 (m, 2H), 1.90 (brs, 1H).

### (E)-4-bromobut-3-en-1-ol (55.2)

| Reagent | MW | Eq. | mmol | g, mL | |
|---|---|---|---|---|---|
| Compound 55.1 | 148.99 | 1.0 | 33.60 | 5.0 | g |
| AlCl₃ | 133.34 | 1.5 | 50.40 | 6.7 | g |
| LiAlH₄ | 37.95 | 2.0 | 67.20 | 2.6 | g |
| Diethyl ether | | | | 200 | mL |

To a stirring solution of LiAlH₄ (2.6 g, 67.2 mmol) in diethyl ether (200 mL) was added AlCl₃ (6.7 g, 50.4 mmol) at −5° C. under a N₂ atmosphere, and the reaction mixture was stirred for 10 minutes. Compound 55.1 (5.0 g, 33.6 mmol) was added dropwise at −5° C. and the reaction mixture was refluxed for 2.5 hr. The reaction mixture was cooled to 0° C., diluted with diethyl ether (200 mL), and quenched with 2N HCl. The layers were separated, and the aqueous layer was extracted with diethyl ether (3×80 mL). The combined organic layers were washed with brine, dried over Na₂SO₄ and filtered. The solvent was removed under reduced pressure to give 55.2 (4.5 g, 89%) as a yellow oil, which was carried through to the next step without further purification.

(S,E)-methyl 3-(tert-butoxycarbonylamino)-2-(4-(6-hydroxyhex-3-en-1-ynyl)benzamido)-3-methylbutanoate (55.3)

| Reagent | MW | Eq. | mmol | g, mL | |
|---|---|---|---|---|---|
| Compound 32.9 | 374.43 | 1.0 | 8.5 | 3.2 | g |
| Compound 55.2 | 151.00 | 2.0 | 17.0 | 2.6 | g |
| Pd(PPh₃)₂Cl₂ | 698.24 | 0.05 | 0.4 | 279 | mg |
| CuI | 189.47 | 0.1 | 0.8 | 152 | mg |
| TEA | 101.19 | 2.0 | 17.0 | 1.7 | g |
| THF | | | | 40 | mL |

To a stirring solution of compound 32.9 (3.2 g, 8.5 mmol), Pd(PPh₃)₂Cl₂ (279 m g, 0.4 mmol), CuI (152 mg, 0.8 mmol), and TEA (1.7 g, 17.0 mmol) in THF (40 mL), was added compound 55.2 (2.6 g, 17.0 mmol) and the reaction mixture was stirred under an Ar atmosphere at rt overnight. After solvent evaporation under reduced pressure, the residue was diluted with water (30 mL), and extracted with DCM (3×80 mL). The combined organic layers were dried and concentrated under reduced pressure to give a crude, which was purified by flash chromatography (silica gel/PE:EA: 2:1) to yield compound 55.3 (1.1 g, 30%) as a yellow oil. MS: m/z calcd for C₂₄H₃₂N₂O₆ 444.2. found [M+H]⁺ 445.1.

### (S,E)-3-(4-(6-hydroxyhex-3-en-1-ynyl)benzamido)-4-methoxy-2-methyl-4-oxobutan-2-aminium 2,2,2-trifluoroacetate (55.4)

| | | | | mg, | |
|---|---|---|---|---|---|
| Reagent | MW | Eq. | mmol | mL | |
| Compound 55.3 | 444.52 | 1 | 2.47 | 1.1 | g |
| TFA | 114.02 | | | 6 | mL |
| CH₂Cl₂ | | | | 10 | mL |

To a stirring solution of 55.3 (1.1 g, 2.47 mmoL) in CH₂Cl₂ (10 mL) was added TFA (6 mL) at 0° C. and the reaction was stirred for 1 hr. The solvent was removed under reduced pressure to give a crude, which was purified by RP HPLC to yield compound 55.4 (380 mg, 45%) as a yellow solid. MS: m/z calcd for C₁₉H₂₄N₂O₄ 344.2. found [M+H]⁺ 345.2; ¹H NMR (400 MHz, DMSO-d₆) δ 8.89 (d, J=9.2 Hz, 1H), 8.12 (s, 3H), 7.92 (d, J=8.8 Hz, 2H), 7.56 (d, J=8.8 Hz, 2H), 6.28-6.36 (m, 1H), 5.85-5.90 (m, 1H), 4.94 (d, J=8.8 Hz, 1H), 4.66 (t, J=5.2 Hz, 1H), 3.72 (s, 3H), 3.49 (dd, J=4.5 Hz, 11.2 Hz, 2H), 2.30-2.35 (m, 2H), 1.37 (d, J=6.8 Hz, 6H).

### (S,E)-N-(3-amino-1-(hydroxyamino)-3-methyl-1-oxobutan-2-yl)-4-(6-hydroxyhex-3-en-1-yn-1-yl)benzamide, Acetate (55)

To a stirring solution of compound 55.4 (1.58 g, 4.59 mmol) in IPA (4.5 ml) was added hydroxylamine (50% aqueous, 6.06 ml, 92 mmol) at 0° C. and the reaction was stirred for 18 hr. The reaction was concentrated under reduced pressure, acidified with AcOH (6 mL), and purified by RP HPLC (2″, 0.1% AcOH in water/ACN) to yield (S,E)-N-(3-amino-1-(hydroxyamino)-3-methyl-1-oxobutan-2-yl)-4-(6-hydroxyhex-3-en-1-yn-1-yl)benzamide (1.077 g, 2.63 mmol, 57.3%). MS: m/z calcd for C₁₈H₂₃N₃O₄ 345.2. found [M+H]⁺ 346.3.

### 56. N-((2S,3R)-1-(hydroxyamino)-3-(methylamino)-1-oxobutan-2-yl)-4-(5-hydroxypenta-1,3-diyn-1-yl)benzamide (56)

### (2S,3R)-methyl 3-amino-2-(4-(5-hydroxypenta-1,3-diyn-1-yl)benzamido)butanoate (56.2)

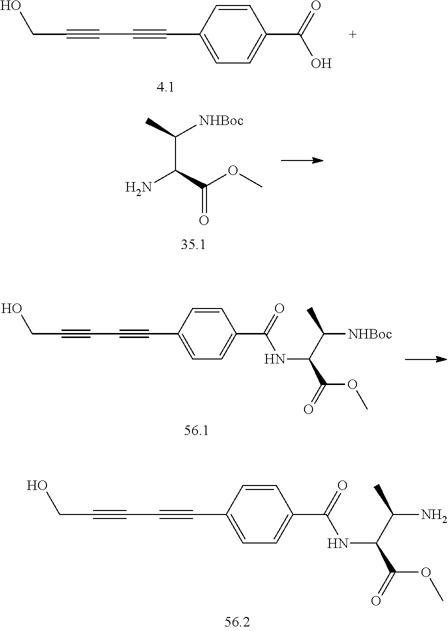 [SMILES: O=C(O)c1ccc(C#CC#CCO)cc1 ; COC(=O)[C@@H](N)[C@@H](C)NC(=O)OC(C)(C)C ; COC(=O)[C@@H](NC(=O)c1ccc(C#CC#CCO)cc1)[C@@H](C)NC(=O)OC(C)(C)C ; COC(=O)[C@@H](NC(=O)c1ccc(C#CC#CCO)cc1)[C@@H](C)N]

| | | | | Sam- | | Den- |
|---|---|---|---|---|---|---|
| | | | | ple | | sity |
| | | | Moles | Mass | Vol | (g/ |
| Reactant | MW | Eq | (mmol) | (mg) | (ul) | ml) |
| 4-(5-hydroxypenta-1,3- | 200.190 | 1.000 | 0.999 | 200 | | |
| diyn-1-yl)benzoic acid | | | | | | |
| (2S,3R)-methyl 2-amino-3- | 232.277 | 1.1 | 1.099 | 255 | | |
| ((tert-butoxycarbonyl)amino)- | | | | | | |
| butanoate | | | | | | |
| DIPEA | 129.243 | 1.05 | 1.049 | 136 | 183 | 0.74 |
| HATU | 235.265 | 1.05 | 1.049 | 399 | | |

To a stirring solution of compound 4.1 (200 mg, 0.999 mmol) in THF (3.3 mL) was added HATU (400 mg, 1.05 mmol), followed by DIPEA (183 μL, 1.05 mmol) and (2S,3R)-methyl 2-amino-3-((tert-butoxycarbonyl)amino)butanoate (255 mg, 1.099 mmol) and the reaction was stirred for 1 hr. Additional DIPEA (183 μL) was added and the reaction was stirred for 3 hr. The mixture was then cooled to 0° C. and TFA (6 mL) was added, and the reaction was stirred for 1 min. TFA was removed under reduced pressure and the solution was basified by the addition of conc NH₄OH (10 mL) and DIPEA (500 μl). The mixture was purified by RP HPLC (2″, 0.25 mM NH₄OH in water, 100% ACN) to yield compound 56.2 (221 mg, 70.4%). MS: m/z calcd for C₁₇H₁₈N₂O₄ 314.13. found [M+H]⁺ 315.1.

### N-((2S,3R)-3-amino-1-(hydroxyamino)-1-oxobutan-2-yl)-4-(5-hydroxypenta-1,3-diyn-1-yl)benzamide (56.3)

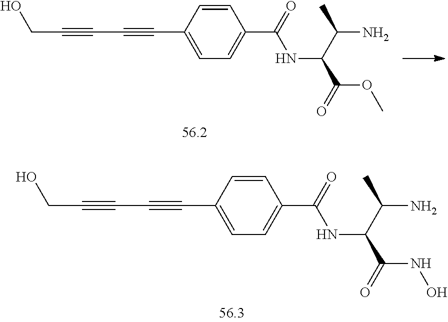 [SMILES: COC(=O)[C@@H](NC(=O)c1ccc(C#CC#CCO)cc1)[C@@H](C)N ; C[C@@H](N)[C@H](NC(=O)c1ccc(C#CC#CCO)cc1)C(=O)NO]

| | | | | Sam- | | | |
|---|---|---|---|---|---|---|---|
| | | | | ple | | Den- | % |
| | | | Moles | Mass | Vol | sity | Wt |
| Reactant | MW | Eq | (mmol) | (mg) | (ml) | (g/ml) | (%) |
| (2S,3R)-methyl | 314.336 | 1.000 | 0.703 | 221 | | | |
| 3-amino-2-(4-(5- | | | | | | | |
| hydroxypenta-1,3- | | | | | | | |
| diyn-1-yl) | | | | | | | |
| benzamido)- | | | | | | | |
| butanoate | | | | | | | |
| Hydroxylamine | 33.030 | 20 | 14.06 | 929 | 0.829 | 1.12 | 50 |
| AcOH | 60.052 | 22 | 15.47 | 929 | 0.885 | 1.049 | |

To a stirring solution of compound 56.2 (221 mg, 0.70 mmol) in IPA (829 μL) cooled in an ice bath was added hydroxylamine (50% aq, 829 μL) and the reaction was stirred overnight. The reaction was acidified with AcOH (88 5 μl), diluted with water (5 mL) and purified by RP HPLC (2″, 0.1% TFA in water/ACN) to yield compound 56.3. MS: m/z calcd for C₁₆H₁₇N₃O₄ 315.12. found [M+H]⁺ 316.1.

### N-((2S,3R)-1-(hydroxyamino)-3-(methylamino)-1-oxobutan-2-yl)-4-(5-hydroxypenta-1,3-diyn-1-yl)benzamide (56)

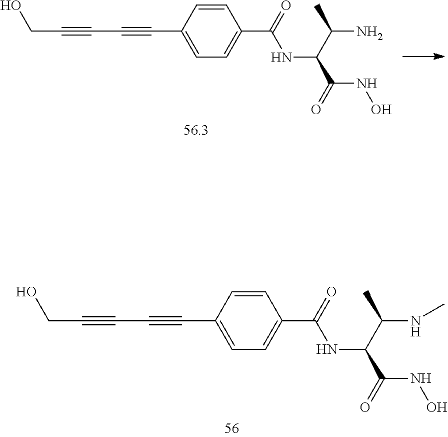 [SMILES: C[C@@H](N)[C@H](NC(=O)c1ccc(C#CC#CCO)cc1)C(=O)NO ; CN[C@H](C)[C@H](NC(=O)c1ccc(C#CC#CCO)cc1)C(=O)NO]

| | | | | Sam- | | Den- | |
|---|---|---|---|---|---|---|---|
| | | | | ple | | sity | % |
| | | | Moles | Mass | Vol | (g/ | Wt |
| Reactant | MW | Eq | (mmol) | (mg) | (ul) | ml) | (%) |
| N-((2S,3R)-3-amino-1- | 315.324 | 1.000 | 0.133 | 57 | | | |
| (hydroxyamino)-1- | | | | | | | |
| oxobutan-2-yl)-4-(5- | | | | | | | |
| hydroxypenta-1,3-diyn- | | | | | | | |
| 1-yl)benzamide, TFA | | | | | | | |
| TEA | 101.190 | 2 | 0.266 | 26.9 | 37.0 | 0.726 | |
| formaldehyde | 30.026 | .8 | 0.107 | 8.72 | 8 | 1.09 | 37 |
| butylamine | 73.137 | 1.000 | 0.133 | 9.71 | 13.12 | .74 | |
| Sodium | 39.853 | 3 | 0.398 | 25.03 | | | |
| cyanoborohydride | | | | | | | |
| TFA | 114.023 | 6 | 0.797 | 91 | 61.4 | 1.48 | |

To a stirring solution of compound 56.3 (57 mg, 0.133 mmol) in DMF (221 μL) and MeOH (221 μL) was added TEA (37 μL), followed by water (663 μL) and the slurry was stirred overnight. The mixture was filtered and to the filtrate was added formaldehyde (8 μL, 37% in water, 0.8 equiv). The reaction was quenched with n-butylamine (13.2 μL, 1 equiv), and the reaction was stirred for 2.5 hr. NaCNBH₃ (25 mg, 3 equiv) was added, followed by TFA (61 μl, 6 equiv) and the reaction was stirred for 20 min. The mixture was purified by RP HPLC (1, 0.1% TFA in water/ACN) to yield compound 56 (3 mg, 5.1%). MS: m/z calcd for C₁₇H₁₉N₃O₄ 329.14. found [M+H]⁺ 330.1.

### 57. N—((S)-3-amino-1-(hydroxyamino)-3-methyl-1-oxobutan-2-yl)-4-((1-hydroxy-3-(hydroxymethyl)cyclopentyl)buta-1,3-diyn-1-yl)benzamide (57)

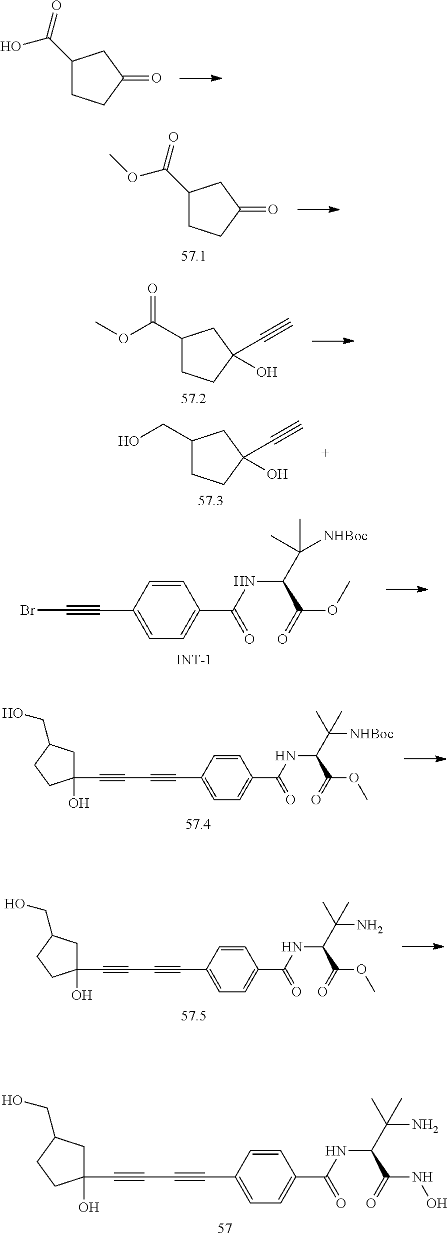

### Methyl 3-oxocyclopentanecarboxylate (57.1)

| Reagent | MW | Eq. | Mmol | g, mL | |
|---|---|---|---|---|---|
| 3-oxocyclopentanecarboxylic acid | 128.13 | 1.0 | 6 | 768 | mg |
| SOCl₂ | | | | 1 | mL |
| MeOH | | | | 10 | mL |

A solution of 3-oxocyclopentanecarboxylic acid (768 mg, 6 mmol) and SOCl₂ (1 mL) in MeOH (10 mL) was stirred at 70° C. for 2 h. The reaction mixture was concentrated under reduced pressure to give a residue, which was dissolved in EtOAc, and washed with saturated. aqueous NaHCO₃. Solvent evaporation under reduced pressure yielded compound 57.1, which was carried through to the next step without further purification.

### Methyl 3-ethynyl-3-hydroxycyclopentanecarboxylate (57.2)

| Reagent | MW | Eq. | Mmol | g, mL | |
|---|---|---|---|---|---|
| Compound 57.1 | 142.15 | 1.0 | 5 | 710 | mg |
| Ethynylmagnesium bromide | 129.24 | 1.0 | 5 | 10 | mL |
| THF | | | | 20 | mL |

A solution of compound 57.1 (710 mg, 5 mmol) and ethynylmagnesium bromide (10 mL, 5 mmol) in THF (20 mL) was stirred at rt for 12 h. After filtration and solvent evaporation under reduced pressure, the crude was purified by flash chromatography (silica gel) to give compound 57.2 (200 mg, 24%) as yellow oil. ¹H NMR (400 MHz, CDCl₃) δ 1.96-2.37 (m, 7H), 2.49 (s, 1H), 3.71 (s, 3H).

### Ethynyl-3-(hydroxymethyl)cyclopentanol (57.3)

| Reagent | MW | Eq. | Mmol | g, mL | |
|---|---|---|---|---|---|
| Compound 57.2 | 168 | 1.0 | 1.2 | 200 | mg |
| LiAlH₄ | 38 | 2.0 | 2.4 | 91 | mg |
| THF | | | | 5 | mL |

To a stirring solution of compound 57.2 (200 mg, 1.2 mmol) in THF (5 mL) was added LiAlH₄ (91 mg, 2.4 mmol) and the reaction mixture was stirred at rt for 12 h. After filtration and solvent evaporation under reduced pressure, the crude was purified by flash chromatography (silica gel) to give compound 57.3 (100 mg, 60%) as a yellow oil. ¹H NMR (400 MHz, CDCl₃) δ 1.70-2.20 (m, 7H), 2.47 (s, 1H), 3.55-3.60 (m, 2H).

### (2S)-methyl 3-(tert-butoxycarbonylamino)-2-(4-((1-hydroxy-3-(hydroxymethyl)cyclopentyl)buta-1,3-diynyl)benzamido)-3-methylbutanoate (57.4)

| Reagent | MW | Eq. | Mmol | g, mL | |
|---|---|---|---|---|---|
| Compound 57.3 | 140 | 1.0 | 0.71 | 100 | mg |
| INT-1 | 453 | 1.0 | 0.71 | 322 | mg |
| Pd(PPh₃)₂Cl₂ | 702 | 0.05 | 0.035 | 25 | mg |
| CuI | 190 | 0.03 | 0.021 | 4 | mg |
| TEA | 101 | 2 | | 142 | mg |
| THF | | | 1.42 | 10 | mL |

To a stirring solution of compound 57.3 (100 mg, 0.71 mmol) in THF (10 mL) were added INT-1 (322 mg, 0.71 mmol), Pd(PPh₃)₂Cl₂ (25 mg, 0.035 mmol), CuI (4 mg, 0.021 mmol), and TEA (142 mg, 1.42 mmol) at rt under a N₂ atmosphere, and the reaction was stirred for 12 hr. Solvent removal under reduced pressure yielded a residue, which was purified by flash chromatography (silica gel/PE:EA: 2:1) to give compound 57.4 (145 mg, 40%) as a white solid. ¹H NMR (400 MHz, DMSO-d₆) δ 1.34-1.37 (m, 3H), 1.44-1.53 (m, 1H), 1.63-1.67 (m, 1H), 1.75-1.90 (m, 3H), 2.03-2.09 (m, 1H), 2.15-2.22 (m, 1H), 3.33 (s, 2H), 3.63 (s, 1H), 4.54 (t, J=3.6 Hz, 1H), 4.86 (s, 1H), 5.65 (s, 1H), 6.82 (s, 1H), 7.68 (d, J=7.2 Hz, 2H), 7.85 (d, J=7.6 Hz, 2H), 8.80 (s, 1H).

### (2S)-methyl 3-amino-2-(4-((1-hydroxy-3-(hydroxymethyl)cyclopentyl)buta-1,3-diyn-1-yl)benzamido)-3-methylbutanoate 2,2,2-trifluoroacetate (57.5)

To a stirring solution of compound 57.4 (30 mg, 0.059 mmol) in methanol (1 mL) was added conc HCl (500 μL) and the reaction was stirred for 4 hr. The reaction was diluted with water and purified by RP HPLC (1″, 0.1% TFA in water/ACN) to yield compound 57.5. MS: m/z calcd for C₂₃H₂₈N₂O₅ 412.20. found [M+H]⁺ 413.2.

### N—((S)-3-amino-1-(hydroxyamino)-3-methyl-1-oxobutan-2-yl)-4-((1-hydroxy-3-(hydroxymethyl)cyclopentyl)buta-1,3-diyn-1-yl)benzamide (57)

To a stirring solution of compound 57.5 in IPA (500 μL) was added 50% hydroxylamine (1 mL) and the reaction was stirred at 0° C. for 4 hr and at it overnight. The reaction mixture was acidified with acetic acid, diluted with water and purified by RP HPLC (1″, 0.1% TFA in water/ACN) to yield compound 57 (17.6 mg). MS: m/z calcd for C₂₂H₂₇N₃O₅ 413.2. found [M+H]⁺ 414.0

### 58. N—((S)-3-amino-1-(hydroxyamino)-3-methyl-1-oxobutan-2-yl)-4-((E)-5,6-dihydroxyhex-3-en-1-yn-1-yl)benzamide (58)

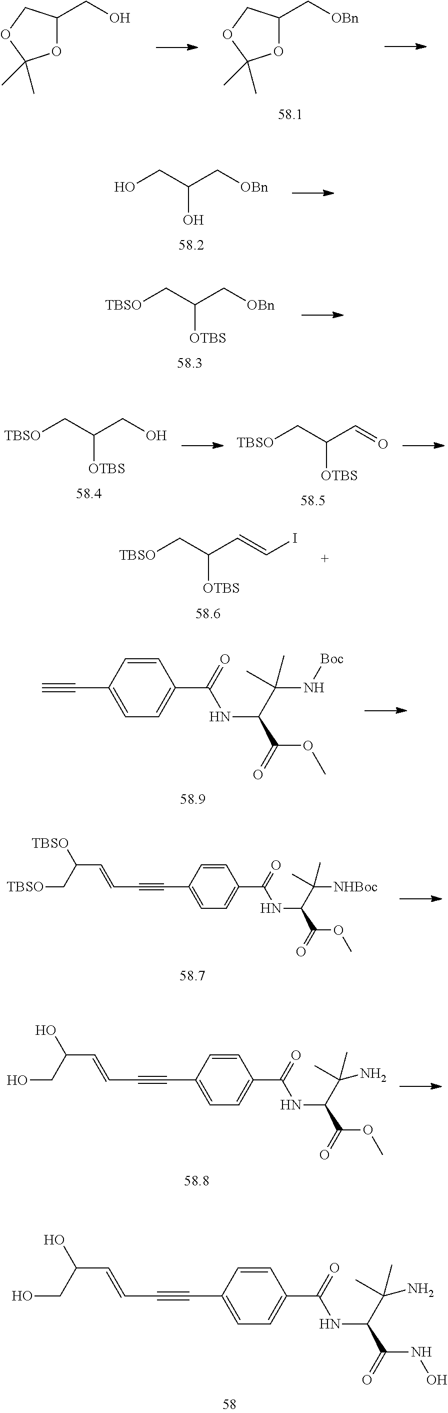

### 4-(benzyloxymethyl)-2,2-dimethyl-1,3-dioxolane (58.1)

| Reagent | MW | Eq. | Mmol | g, mL | |
|---|---|---|---|---|---|
| (2,2-dimethyl-1,3-dioxolan- | 132.16 | 1.0 | 15.1 | 2.0 | g |
| 4yl)methanol | | | | | |
| NaH | 24 (60%) | 1.2 | 18.1 | 0.73 | g |
| BnBr | 171.03 | 1.1 | 16.6 | 2.84 | g |
| DMF | | | | 50 | mL |

To a stirring solution of (2,2-dimethyl-1,3-dioxolan-4yl)methanol (2.0 g, 15.1 mmol) in DMF (50 mL) was added NaH (0.73 g, 18.1 mmol)(60% in oil) and the resulting mixture was stirred at 0° C. for 1 hr. BnBr (2.84 g, 16.6 mmol) was added dropwise, and the resulting mixture was stirred at 0° C. for 2 hr. The reaction mixture was poured into ice water (500 mL), extracted with EtOAc, washed with brine, dried over Na₂SO₄, filtered and concentrated under reduced pressure to give a crude, which was purified by flash chromatography (silica gel) to give compound 58.1 (3.0 g, 89%) as a colorless oil. MS: m/z calcd for C₁₃H₁₈O₃222.1. found [M+H]⁺ 223.

### 3-(benzyloxy)propane-1,2-diol (58.2)

| Reagent | MW | Eq. | mmol | g, mL | |
|---|---|---|---|---|---|
| Compound 58.1 | 222.28 | 1.0 | 13.5 | 3.0 | g |
| HCl (2M in MeOH) | | 5.0 | 67.5 | 33.7 | mL |
| MeOH | | | | 10 | mL |

Compound 58.1 (3.0 g, 13.5 mmol) was dissolved in MeOH (10 mL), and HCl (33.7 mL, 67.5 mmol, 2 M in MeOH) was added, and the reaction mixture was stirred at room temperature for 4 hr. The reaction mixture was diluted with EtOAc (100 mL) and NaHCO₃ (3 g) was added, followed by H₂O (100 mL). The reaction mixture was extracted with EtOAc, washed with brine, dried over Na₂SO₄, filtered and concentrated under reduced pressure to give a crude, which was purified by flash chromatography (silica gel/PE/EA:2/1) to yield compound 58.2 (2.2 g, 90%) as a colorless oil. MS: m/z calcd for C₁₀H₁₄O₃182.1. found [M+Na]⁺ 205.

### 5-(benzyloxymethyl)-2,2,3,3,8,8,9,9-octamethyl-4,7-dioxa-3,8-disiladecane (58.3)

| Reagent | MW | Eq. | Mmol | g, mL | |
|---|---|---|---|---|---|
| Compound 58.2 | 182.2 | 1.0 | 5.49 | 1.0 | g |
| TBSCI | 150.7 | 2.5 | 13.72 | 2.07 | g |
| Imidazole | 68 | 3.0 | 16.5 | 1.12 | g |
| DMAP | 122 | 0.1 | 0.55 | 67 | mg |
| DMF | | | | 5 | mL |

To a stirring solution of 58.2 (1.0 g, 5.49 mmol) in DMF (5 mL) were added TBSCl (2.07 g, 13.72 mmol, imidazole (1.12 g, 16.5 mmol), and DMAP (67 mg, 0.55 mmol) and the resulting mixture was stirred at it overnight. The reaction mixture was poured into water (60 mL), and extracted with EtOAc. The combined organic layers were washed with brine, dried over Na₂SO₄, filtered and concentrated under reduced pressure to give a crude, which was purified by flash chromatography (silica gel/PE/EA 100:1) to give compound 58.3 (1.80 g, 80%) as a colorless oil. MS: m/z calcd for C₂₂H₄₂O₃Si₂ 410.3. found [M+Na]⁺ 433.

### 2,3-bis(tert-butyldimethylsilyloxy)propan-1-ol (58.4)

| Reagent | MW | Eq. | Mmol | g, mL | |
|---|---|---|---|---|---|
| Compound 58.3 | 410.7 | 1.0 | 2.19 | 0.90 | g |
| Pd—C | | 0.1 | | | |
| EtOAc | | | | 5 | mL |

To a stirring solution of compound 58.3 (0.90 g, 2.19 mmol) in EtOAc (5 mL) was added Pd—C (100 mg), and the reaction mixture was stirred under H₂ atmosphere for 4 h. The reaction mixture was filtered, concentrated under reduced pressure to give a crude, which was purified by flash chromatography (silica gel/PE/EA 4/1) to yield compound 58.4 (0.784 g, 100%) as a colorless oil. ¹H NMR (400 MHz, CDCl₃) δ 3.72-3.68 (m, 1H), 3.62-3.47 (m, 5H), 0.828 (s, 9H), 0.826 (s, 9H), 0.030 (s, 3H), 0.024 (s, 3H), −0.001 (s, 3H), −0.004 (s, 3H).

### 2,2,3,3,8,8,9,9-octamethyl-4,7-dioxa-3,8-disiladecane-5-carbaldehyde (58.5)

| Reagent | MW | Eq. | Mmol | g, mL | |
|---|---|---|---|---|---|
| Compound 58.4 | 320 | 1.0 | 2.44 | 0.781 | g |
| (COCl)₂ | 126.93 | 2.0 | 4.88 | 0.46 | mL |
| DMSO | 78.12 | 4.0 | 9.76 | 0.69 | mL |
| Et₃N | 101 | 5.0 | 12.2 | 1.76 | mL |
| CH₂Cl₂ | | | | 24 | mL |

Anhydrous DMSO (0.69 mL, 9.76 mmol) was added dropwise to a stirring solution of (COCl)₂ (0.46 mL, 4.88 mmol) in anhydrous CH₂Cl₂ (20 mL) at −78° C. under a N₂ atmosphere. The reaction mixture was stirred for 30 min and then a solution of alcohol 58.4 (0.781 g, 2.44 mmol) in anhydrous CH₂Cl₂ (2 mL) was added. Stirring was continued at −78° C. for another 30 min, then Et₃N (1.76 mL, 12.2 mmol) was added. The mixture was stirred at −78° C. for 5 min and then at room temperature for 25 min. The reaction was quenched with aqueous saturated. NH₄Cl and was extracted with CH₂Cl₂. The combined organic layers were washed with brine and dried over anhydrous Na₂SO₄. Solvent removal under reduced pressure gave a crude, which was purified by flash chromatography (silica gel PE/EA 100/1) to yield compound 58.5 (0.76 g, 98%) as a colorless oil. ¹H NMR (400 MHz, CDCl₃) δ 9.60 (s, 1H), 4.00 (t, J=5.4 Hz, 1H), 3.74 (d, J=5.5 Hz, 2H), 0.86 (s, 9H), 0.82 (s, 9H), 0.04 (s, 3H), 0.03 (s, 3H), 0.00 (s, 3H), −0.003 (s, 3H).

### (E)-5-(2-iodovinyl)-2,2,3,3,8,8,9,9-octamethyl-4,7-dioxa-3,8-disiladecane (58.6)

| Reagent | MW | Eq. | Mmol | g, mL | |
|---|---|---|---|---|---|
| Compound 58.5 | 318 | 1.0 | 2.39 | 0.76 | g |
| CrCl₂ | 122.9 | 5.0 | 11.95 | 1.47 | g |
| CHI₃ | 393.7 | 2.0 | 4.78 | 1.88 | g |
| THF | | | | 20 | mL |

To a solution of CrCl₂ (1.47 g, 0.76 mmol) in THF (15 mL) was added CHI₃ (1.88 g, 4.78 mmol) in THF (5 mL) dropwise at 0° C., then compound 58.5 (0.76 g, 2.39 mmol) in THF (5 mL) was added and the reaction mixture was stirred at 0° C. for 2 hr and then at room temperature for 1 h. The reaction mixture was added to ice water, and extracted with EtOAc. The organic layer was washed with aqueous Na₂S₂O₃, brine, then concentrated under reduced pressure to give a residue, which was purified by flash chromatography (silica gel/PE/EA 50:1) to yield compound 58.6 (0.6 g, 57%) as a yellow oil. MS: m/z calcd for C₁₆H₃₅IO₂Si₂ 442.1. found [M+Na]⁺ 465.

### (2S,E)-methyl 2-(4-(5,6-bis(tert-butyldimethylsilyloxy)hex-3-en-1-ynyl)benzamido)-3-(tert-butoxycarbonylamino)-3-methylbutanoate (58.7)

| Reagent | MW | Eq. | mmol | g, mL | |
|---|---|---|---|---|---|
| Compound 58.6 | 442.52 | 1.0 | 1.35 | 0.60 | g |
| Compound 32.9 | 374.4 | 1.0 | 1.35 | 0.508 | g |
| PdCl₂(Ph₃)₂ | 701.9 | 0.1 | 0.135 | 95 | mg |
| CuI | 190.5 | 0.1 | 0.135 | 26 | mg |
| Et₃N | 101 | 3.0 | 4.35 | 0.6 | mL |
| THF | | | | 10 | mL |

To a solution of compound 32.9 (508 mg, 1.35 mmol), PdCl₂(Ph₃)₂ (95 mg, 0.135 mmol), and CuI (26 mg, 0.135 mmol) in THF (8 mL) was added compound 58.6 (600 mg, 1.35 mmol) in THF (5 mL), followed by Et₃N (0.6 mL, 4.35 mmol) and the reaction mixture was stirred at room temperature overnight. Water was added, and the resulting mixture was extracted with EtOAc. The organic layer was washed with brine, concentrated under reduced pressure to give a crude, which was purified by flash chromatography (silica gel/PE/EA: 10/1) to yield compound 58.7 (700 mg, 75%) as a pale yellow oil. MS: m/z calcd for C₃₆H₆₀N₂O₇Si₂ 688.4. found [M+Na]⁺ 711.3; ¹H NMR (400 MHz, CDCl₃) δ 8.99 (br s, 1H), 7.80 (d, J=8.3 Hz, 2H), 7.44 (d, J=8.3 Hz, 2H), 6.27 (dd, J=5.8, 4.7 Hz, 1H), 5.92 (dd, J=15.9, 1.6 Hz, 1H), 4.68-4.64 (m, 2H), 4.24-4.19 (m, 1H), 3.67 (s, 3H), 3.50 (dd, J=10.0, 6.5 Hz, 1H), 3.42 (dd, J=9.9, 6.1 Hz, 1H), 1.44 (s, 3H), 1.41 (s, 3H), 1.38 (s, 9H), 0.86 (s, 9H), 0.84 (s, 9H), 0.03 (s, 3H), 0.02 (s, 3H), 0.00 (s, 6H).

### (2S,E)-methyl 3-amino-2-(4-(5,6-dihydroxyhex-3-en-1-ynyl)benzamido)-3-methylbutanoate (58.8)

| Reagent | MW | Eq. | mmol | g, mL | |
|---|---|---|---|---|---|
| Compound 58.7 | 689 | 1.0 | 0.87 | 0.60 | g |
| HCl (1M in MeOH) | | 11.5 | 10 | 10 | mL |
| MeOH | | | | 2 | mL |

To a solution of compound 58.7 (600 mg, 0.87 mmol) in MeOH (2 mL) was added HCl (10 mL, 10 mmol, 1 M in MeOH), and the reaction mixture was stirred at room temperature for 2 hr. NaHCO₃ (1.2 g) was added and the mixture was filtered. The filtrate was concentrated under reduced pressure to give a residue, which was purified by flash chromatography to yield 58.8 (280 mg, 89%) as a white solid. MS: m/z calcd for C₁₉H₂₄N₂O₅ 360.2. found [M+H]⁺ 361; ¹H NMR (400 MHz, DMSO-d₆) δ 8.85 (br s, 1H), 7.87 (d, J=8.6 Hz, 2H), 7.55 (d, J=8.6 Hz, 2H), 6.37 (dd, J=11.1, 4.7 Hz, 1H), 5.99 (dd, J=15.9, 1.8 Hz, 1H), 5.13 (d, J=4.8 Hz, 1H), 4.74 (t, J=5.8 Hz, 1H), 4.38 (s, 1H), 4.12-4.10 (m, 1H), 3.65 (s, 3H), 1.79 (br s, 2H), 1.13 (s, 3H), 1.12 (s, 3H).

### N—((S)-3-amino-1-(hydroxyamino)-3-methyl-1-oxobutan-2-yl)-4-((E)-5,6-dihydroxyhex-3-en-1-yn-1-yl)benzamide (58)

To a stirring solution of compound 58.8 in IPA (500 μL) was added 50% aqueous hydroxylamine (1 mL) and the reaction was stirred overnight. The reaction mixture was acidified with acetic acid, diluted with water and purified by RP HPLC (1″, 0.1% TFA in water/ACN) to yield compound 58 (38 mg). MS: m/z calcd for C₁₈H₂₃N₃O₅ 361.2. found [M+H]⁺ 362.0.

### 59. N-((2S,3R)-3-amino-4,4,4-trifluoro-1-(hydroxyamino)-1-oxobutan-2-yl)-4-((S)-5-hydroxyhexa-1,3-diyn-1-yl)benzamide (59)

### (2S,3R)-ethyl 3-((S)-1,1-dimethylethylsulfinamido)-2-((diphenylmethylene)amino)-4,4,4-trifluorobutanoate (59.1)

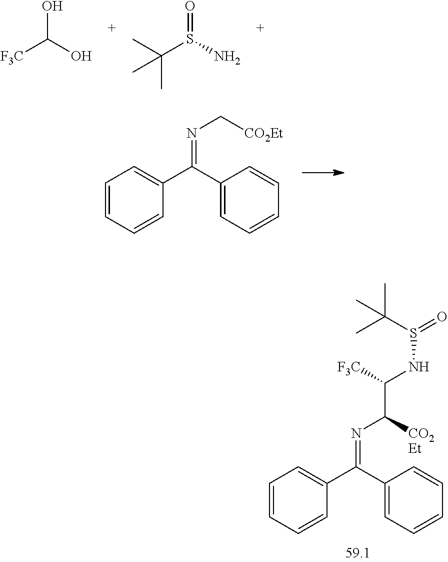

| | | | | Sam- | | |
|---|---|---|---|---|---|---|
| | | | | ple | | % |
| | | | Moles | Mass | Vol | Wt |
| Reactant | MW | Eq | (mmol) | (g) | (mL) | (%) |
| 2,2,2-trifluoroethane-1,1- | 116.039 | 1.000 | 43.3 | 6.7 | 6.70 | 75 |
| diol, 75% in water | | | | | | |
| (S)-2-methylpropane-2- | 121.201 | 1.1 | 47.6 | 5.77 | | |
| sulfinamide | | | | | | |
| ethyl 2- | 267.322 | 1.7 | 73.6 | 19.68 | | |
| ((diphenylmethylene)amino)- | | | | | | |
| acetate | | | | | | |
| LiHMDS, 1 M in THF | 167.326 | 1.7 | 73.6 | | 73.6 | |

Molecular sieves (3 A, 25 g) were heated under vacuum; they were allowed to cool and were suspended in toluene (80 mL). (S)-2-Methylpropane-2-sulfinamide (5.77 g, 47.6 mmol) was added, followed by 2,2,2-trifluoroethane-1,1-diol (6.7 mL, 43.3 mmol) and the reaction was heated to 40° C. for 4 hr. The reaction mixture was cooled to rt and filtered. To a stirring solution of ethyl 2-((diphenylmethylene)amino)acetate (19.7 g, 73.6 mmol) in THF (625 ml) at −78° C. was added LiHMDS (1 M, 73.6 mL, 73.6 mmol) and the solution was stirred for 30 min. The toluene solution prepared previously was then slowly added and the reaction mixture was stirred at −78° C. for 30 min. Saturated aqueous NH₄Cl (225 mL) was added and the reaction was allowed to warm to rt. The aqueous layer was separated and extracted with MTBE. The combined organic layers were washed with brine, dried over magnesium sulfate and concentrated under reduced pressure to give a crude, which was purified by flash chromatography (silica gel, 0-100% EtOAc in hexanes) to yield compound 59.1 (4.1 g, 8.75 mmol, 20.2%). MS: m/z calcd for C₂₃H₂₇F₃N₂O₃S 468.17. found [M+H]⁺ 469.2.

### (2S,3R)-ethyl 2-amino-3-((S)-1,1-dimethylethylsulfinamido)-4,4,4-trifluorobutanoate (59.2)

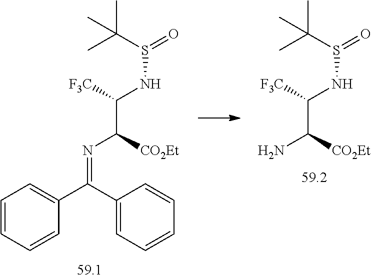

| | | | | Sam- | | |
|---|---|---|---|---|---|---|
| | | | | ple | | Den- |
| | | | Moles | Mass | Vol | sity |
| Reactant | MW | Eq | (mmol) | (g) | (mL) | (g/ml) |
| (2S,3R)-ethyl 3-((S)-1,1- | 468.532 | 1.000 | 8.75 | 4.1 | | |
| dimethylethylsulfin- | | | | | | |
| amido)-2- | | | | | | |
| ((diphenylmethylene)- | | | | | | |
| amino)-4,4,4- | | | | | | |
| trifluorobutanoate | | | | | | |
| TFA | 114.023 | 3 | 26.3 | 2.99 | 2.023 | 1.48 |

To a stirring solution of compound 59.1 (4.1 g, 8.75 mmol) in THF (20 mL) was added water (20 mL) followed by TFA (2 mL, 26.3 mmol) and the reaction was stirred for 20 min. The reaction mixture was partitioned between water and MTBE. The aqueous layer was washed with MTBE, was basified with saturated aqueous NaHCO₃, extracted with MTBE (3×) and EtOAc. The initial organic phase was diluted with hexanes and extracted with water. This aqueous phase was also basified with saturated aqueous NaHCO₃ and extracted with EtOAc. The combined organic layers were washed with brine, dried over sodium sulfate, and concentrated under reduced pressure to yield compound 59.2 (880 mg, 33%), which was carried through to the next step without further purification. MS: m/z calcd for C₁₀H₁₉F₃N₂O₃S 304.11. found [M+H]⁺ 305.2.

### (2S,3R)-ethyl 3-((S)-1,1-dimethylethylsulfinamido)-4,4,4-trifluoro-2-(4-((S)-5-hydroxyhexa-1,3-diyn-1-yl)benzamido)butanoate (59.3)

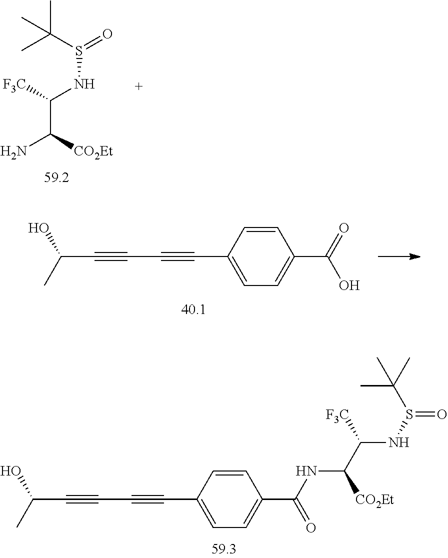

| | | | | Sam- | | |
|---|---|---|---|---|---|---|
| | | | | ple | | Den- |
| | | | Moles | Mass | Vol | sity |
| Reactant | MW | Eq | (mmol) | (mg) | (mL) | (g/ml) |
| (S)-4-(5-hydroxyhexa-1,3- | 214.217 | 1 | 2.79 | 598 | | |
| diyn-1-yl)benzoic acid | | | | | | |
| (2S,3R)-ethyl 2-amino-3- | 304.330 | 1.000 | 2.79 | 850 | | |
| ((S)-1,1-dimethylethyl- | | | | | | |
| sulfinamido)-4,4,4- | | | | | | |
| trifluorobutanoate | | | | | | |
| HATU | 235.265 | 1.3 | 3.63 | 1381 | | |
| DIPEA | 129.243 | 2.5 | 6.98 | 902 | 1.220 | 0.74 |

To a stirring solution of compound 59.2 (850 mg, 2.79 mmol) in DMF (9 mL) at 0° C. was added compound 40.1 (598 mg, 2.79 mmol), followed by DIPEA (1.22 mL, 6.98 mmol) and HATU (1.38 g, 3.63 mmol) and the reaction was allowed to warm to rt and stirred for 1 hr. The reaction was quenched with water and MTBE. The reaction was partitioned and the organic layer was washed with 1M citric acid, saturated sodium bicarbonate, saturated sodium chloride, dried over magnesium sulfate and concentrated under reduced pressure to give a residue, which was purified by RP HPLC (2″, 0.1% AcOH in water/ACN) to yield compound 59.3. MS: m/z calcd for C₂₃H₂₇F₃N₂O₅S 500.16. found [M+H]⁺ 500.2.

### (2S,3R)-ethyl 3-amino-4,4,4-trifluoro-2-(4-((S)-5-hydroxyhexa-1,3-diyn-1-yl)benzamido)butanoate (59.4)

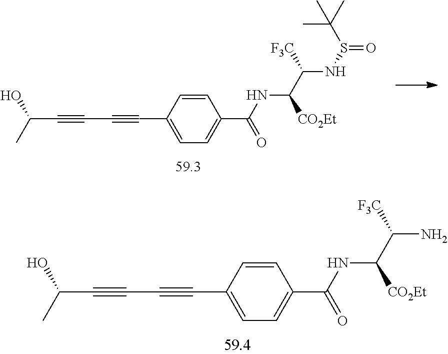

To a stirring solution of compound 59.3 in THF (1 ml) was added 2M HCl (200 ul) followed by conc HCl (200 ul) and the reaction was stirred for 2 days at 5-10° C. The reaction was partitioned between saturated sodium bicarbonate and ethyl acetate. The organic layer was washed with saturated sodium chloride, dried over magnesium sulfate and concentrated under reduced pressure to give a residue, which was purified by RP HPLC (2″, 0.1% AcOH in water/ACN) to yield compound 59.4. MS: m/z calcd for C₁₉H₁₉F₃N₂O₄396.13. found [M+H]⁺ 397.1.

### N-((2S,3R)-3-amino-4,4,4-trifluoro-1-(hydroxyamino)-1-oxobutan-2-yl)-4-((S)-5-hydroxyhexa-1,3-diyn-1-yl)benzamide (59)

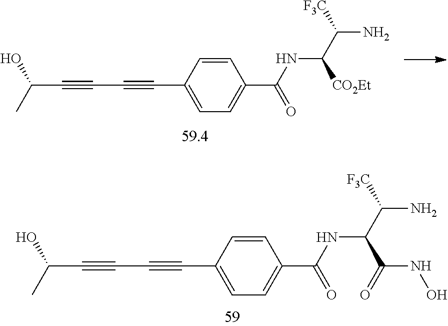 [SMILES: CCOC(=O)[C@@H](NC(=O)c1ccc(C#CC#C[C@H](C)O)cc1)[C@@H](N)C(F)(F)F ; C[C@H](O)C#CC#Cc1ccc(C(=O)N[C@H](C(=O)NO)[C@@H](N)C(F)(F)F)cc1]

Compound 59.4 was treated with IPA/50% aqueous hydroxylamine at 0° C. overnight. The reaction mixture was diluted with water, acidified with AcOH and purified by RP HPLC (2″, 0.1% AcOH in water/ACN) to yield compound 59. MS: m/z calcd for C₁₇H₁₆F₃N₃O₄383.1. found [M+H]⁺ 384.0.

### 60. 4-(5,6-dihydroxyhexa-1,3-diyn-1-yl)-N—((S)-3-hydroxy-1-(hydroxyamino)-3-methyl-1-oxobutan-2-yl)benzamide (60)

### (S)-methyl 2-(4-(bromoethynyl)benzamido)-3-hydroxy-3-methylbutanoate (60.1)

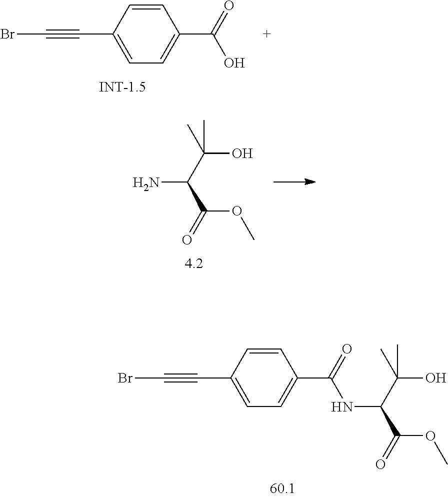

| | | | | | Sample | |
|---|---|---|---|---|---|---|
| | | | Density | Moles | Mass | Vol |
| Reactant | MW | Eq | (g/ml) | (mmol) | (g) | (mL) |
| INT-1.5 | 225.04 | 1.003 | | 13.33 | 3 | |
| (R)-methyl 2-amino-3- | 147.17 | 1 | | 13.29 | 2.441 | |
| hydroxy-3- | | | | | | |
| methylbutanoate, HCl | | | | | | |
| HATU | 235.27 | 1.2 | | 15.95 | 6.06 | |
| TEA | 101.19 | 3 | 0.73 | 39.9 | 4.03 | 5.56 |

To a stirring solution of (R)-methyl 2-amino-3-hydroxy-3-methylbutanoate, HCl (2.441 g, 13.29 mmol) in acetonitrile (25 mL) was added INT-1.5 (3 g, 13.33 mmol) followed by TEA (5.56 mL, 39.9 mmol). HATU (6.06 g, 15.95 mmol) was then added portionwise over 20 minutes, and the reaction was stirred for 3 hours. The reaction mixture was concentrated under reduced pressure to a residue, which was diluted with ethyl acetate (30 mL), and filtered. The filtrate was washed with 1M citric acid (2×30 mL), saturated. aqueous NaHCO₃ (4×30 mL), 1M NaOH (30 mL), and brine (30 mL). The organic layer was concentrated under reduced pressure to yield compound 60.1 (4.49 g, 95%) as a brown solid, which was carried through to the next step without further purification.

### (2S)-methyl-2-(4-(5,6-dihydroxyhexa-1,3-diyn-1-yl)benzamido)-3-hydroxy-3-methylbutanoate (60.3)

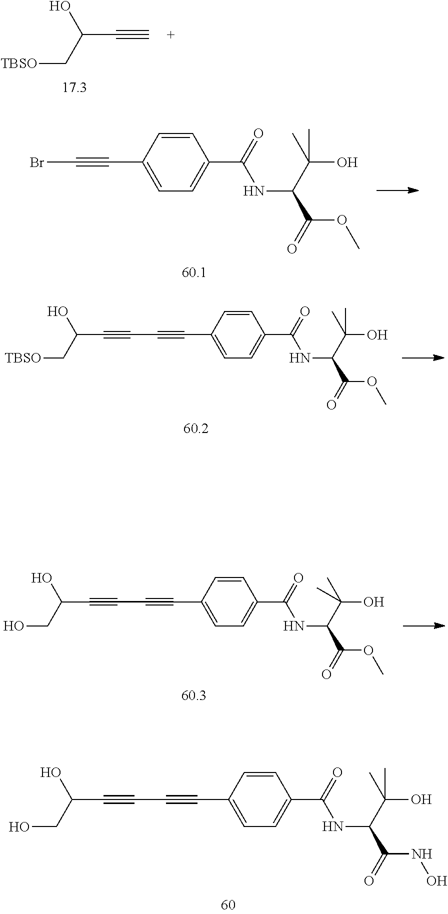 [SMILES: C#CC(O)CO[Si](C)(C)C(C)(C)C ; COC(=O)[C@@H](NC(=O)c1ccc(C#CBr)cc1)C(C)(C)O ; COC(=O)[C@@H](NC(=O)c1ccc(C#CC#CC(O)CO[Si](C)(C)C(C)(C)C)cc1)C(C)(C)O ; COC(=O)[C@@H](NC(=O)c1ccc(C#CC#CC(O)CO)cc1)C(C)(C)O ; CC(C)(O)[C@H](NC(=O)c1ccc(C#CC#CC(O)CO)cc1)C(=O)NO]

| | | | Moles | Sample |
|---|---|---|---|---|
| Reactant | MW | Eq | (mmol) | Mass (g) |
| hydroxylamine hydrochloride | 33.030 | 0.06 | 0.898 | 0.062 |
| copper(I) chloride | 98.999 | 0.02 | 0.299 | 0.030 |
| 1-((tert-butyldimethylsilyl)oxy)but- | 200.350 | 1.000 | 14.97 | 3.00 |
| 3-yn-2-ol | | | | |
| (S)-methyl 2-(4- | 354.196 | 1.000 | 14.97 | 5.30 |
| (bromoethynyl)benzamido)-3- | | | | |
| hydroxy-3-methylbutanoate | | | | |

To a stirring solution of hydroxylamine hydrochloride (0.062 g, 0.898 mmol) and copper(I) chloride (0.030 g, 0.299 mmol) in 30% n-butylamine/water (60 mL) at 0° C. was added a solution of 1-((tert-butyldimethylsilyl)oxy)but-3-yn-2-ol (3.00 g, 14.97 mmol) in 30% n-butylamine/water (10 mL), followed by a solution of (S)-methyl 2-(4-(bromoethynyl)benzamido)-3-hydroxy-3-methylbutanoate (5.30 g, 14.97 mmol) in 30% n-butylamine/water (20 mL) and THF (20 mL) and the reaction was stirred for 1 hr. The reaction mixture was extracted with EtOAc, and concentrated under reduced pressure to yield compound 60.2 (5.92 g, MS: m/z calcd for C₂₅H₃₅NO₆Si 473.2. found [M+Na]⁺ 496.2), which was treated with TFA (20 mL) for 15 min. Excess TFA was removed under reduced pressure, water (100 mL) was added and the mixture was extracted with EtOAc, dried over Na₂SO₄ and concentrated under reduced pressure to yield compound 60.3 (4.67 g).

### 4-(5,6-dihydroxyhexa-1,3-diyn-1-yl)-N—((S)-3-hydroxy-1-(hydroxyamino)-3-methyl-1-oxobutan-2-yl)benzamide (60)

To a stirring solution of compound 60.3 (4.67 g) in IPA (50 mL) was added hydroxylamine (50% aqueous, 50 mL) and the reaction was stirred for 3 days. The solvent was removed under reduced pressure and the mixture was acidified with AcOH and purified by RP HPLC (6″, 0.1% AcOH in water/ACN) to yield compound 60 (665 mg). MS: m/z calcd for C₁₈H₂₀N₂O₆ 360.1. found [M+H]⁺ 361.1.

### 61. N—((S)-3-amino-1-(hydroxyamino)-3-methyl-1-oxobutan-2-yl)-4-((S)-5,6-dihydroxyhexa-1,3-diyn-1-yl)benzamide (61)

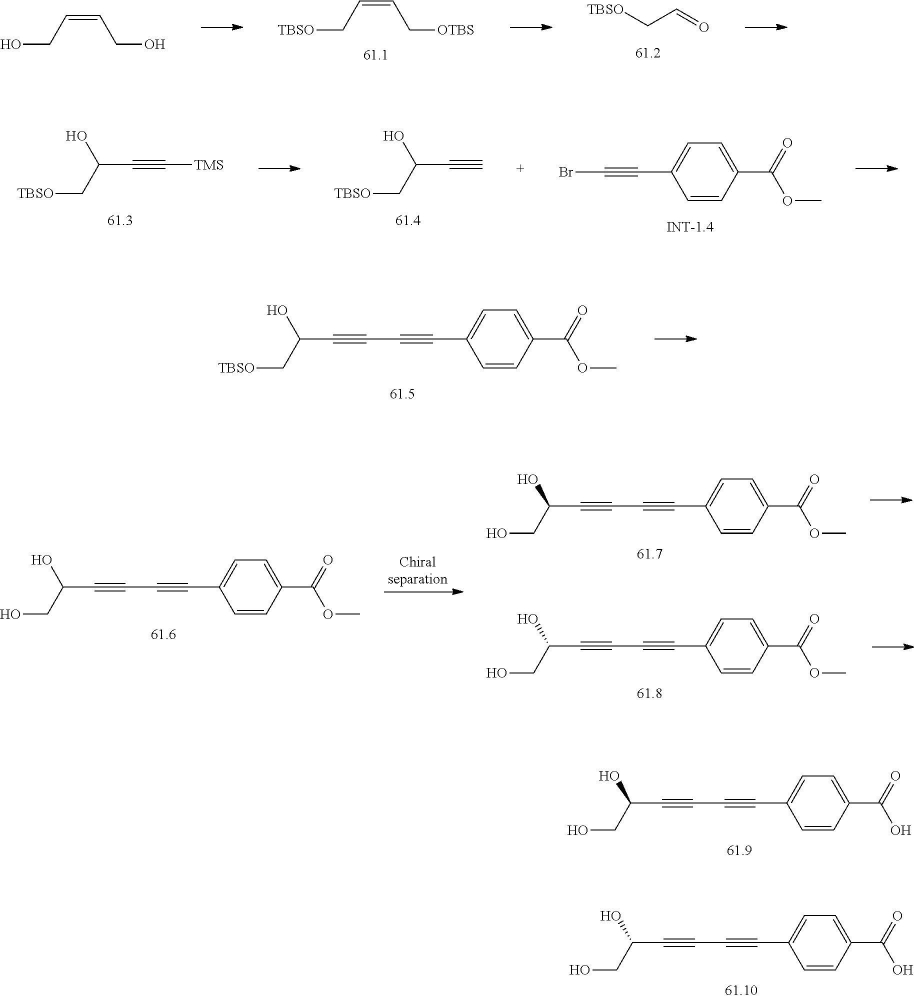

### (Z)-2,2,3,3,10,10,11,11-octamethyl-4,9-dioxa-3,10-disiladodec-6-ene (61.1)

| Reagent | MW | Eq. | mmol | g, mL | |
|---|---|---|---|---|---|
| But-2-ene-1,4-diol | 88.11 | 1 | 110 | 10 | g |
| TBSCl | 98.22 | 2.2 | 250 | 36.7 | g |
| 1H-imidazole | 64.09 | 1.5 | 170 | 11.6 | g |
| DMF | | | | 200 | mL |
| THF | | | | 50 | mL |

To a stirring solution of but-2-ene-1,4-diol (10 g, 110 mmol) and 1H-imidazole (11.6 g, 170 mmol) in dry DMF (200 mL) at 0° C. was added a solution of TBSCl (36.7 g, 250 mmol) in dry THF (50 mL) and the reaction was allowed to warm to rt. The progress of the reaction was followed by TLC. THF was removed under reduced pressure, and the resulting residue was diluted with water (1 L) and extracted with EA (3×200 mL). The combined organic layers were washed with 5% NaOH (aq), dried and concentrated under reduced pressure to give 61.1 as a yellow oil, which was carried through to the next step without further purification. ¹H NMR (400 MHz, CDCl₃) δ 0.06 (s, 12H), 0.89 (s, 24H), 4.22 (d, J=2 Hz, 1H), 5.55 (t, J=3.6 Hz, 1H) 2-(tert-butyldimethylsilyloxy)acetaldehyde (61.2)

| Reagent | MW | Eq. | mmol | g, mL | |
|---|---|---|---|---|---|
| Compound 61.1 | 316.63 | 1 | 100 | 30 | g |
| PPh₃ | 279 | 1.1 | 110 | 30.69 | g |
| DCM | | | | 300 | mL |

A solution of compound 61.1 (30 g, 100 mmol) in DCM (300 mL) was treated with ozone at −78° C. until the solution turned lightly blue. PPh₃ (30.69 g, 110 mmol) was added and the mixture was stirred at rt for 12 h. The reaction mixture was concentrated under reduced pressure to give a crude which was distilled under reduced pressure (bp 62° C. at 10 mbar) to yield compound 61.2 (30 g, 90%) as a colorless oil. ¹H NMR (400 MHz, CDCl₃) δ 0.10 (s, 6H), 0.92 (s, 24H), 4.21 (d, J=0.4 Hz, 2H), 9.69 (s, 1H) 1-(tert-butyldimethylsilyloxy)-4-(trimethylsilyl)but-3-yn-2-ol (61.3)

| Reagent | MW | Eq. | mmol | g, mL | |
|---|---|---|---|---|---|
| Compound 61.2 | 174.31 | 1 | 172 | 30 | g |
| Ethynyltrimethylsilane | 98.22 | 1.1 | 189 | 18.59 | g |
| n-Buli | 64.06 | 1.2 | 200 | 80 | mL |
| THF | | | | 300 | mL |

To a stirring solution of ethynyltrimethylsilane (18.59 g, 189.0 mmoL) in dry THF (300 mL) at −78° C. was added n-Buli (80 mL, 200.0 mmoL) and the mixture was stirred for 30 min. Compound 61.2 (30 g, 172 mmol) was added dropwise and the reaction progress was followed by TLC. The reaction mixture was diluted with water (300 mL), and extracted with EA (3×100 mL). The combined organic layers were washed with saturated aqueous NaHCO₃, dried and concentrated under reduced pressure to yield compound 61.3 (42.0 g) as a red oil, which was used in the next reaction without further purification. MS: m/z calcd for C₁₃H₂₈O₂Si₂ 272.2. found [M+Na]⁺ 295.1.

### 1-(tert-butyldimethylsilyloxy)but-3-yn-2-ol (61.4)

| Reagent | MW | Eq. | Mmol | g, mL | |
|---|---|---|---|---|---|
| Compound 61.3 | 272.53 | 1 | 73.3 | 20 | g |
| K₂CO₃ | 138.12 | 2.5 | 183.25 | 25.34 | g |
| MeOH | | | | 50 | mL |
| THF | | | | 5 | mL |

To a stirring solution of compound 61.3 (20 g, 73.3 mmoL) in MeOH (50 mL) and THF (5 mL) was added K₂CO₃ (2534 g, 183.25 mmoL) and the mixture was stirred for 16 hours. The reaction mixture was concentrated under reduced pressure to give a residue, which was diluted with water (100 mL), and extracted with EA (3×150 mL). The combined organic layers were dried and concentrated under reduced pressure to give a crude, which was purified by flash chromatography (silica gel/PE:EA 10:1 to 2:1) to yield compound 61.4 (12 g) as a colorless oil.

### Methyl 4-(6-(tert-butyldimethylsilyloxy)-5-hydroxyhexa-1,3-diynyl)benzoate (61.5)

| Reagent | MW | Eq. | mmol | g, mL | |
|---|---|---|---|---|---|
| Compound 61.4 | 200.35 | 1.1 | 10 | 2 | g |
| INT-1 | 239 | 1 | 9 | 2.7 | g |
| CuCl | 99.00 | 0.02 | 0.02 | 20 | mg |
| hydroxylamine hydrochloride | 69.49 | 0.06 | 0.06 | 45.4 | mg |
| butan-1-amine | 73.14 | 23 | 23 | 16.8 | g |
| MeOH | | | | 10 | mL |
| THF | | | | 5 | mL |
| H₂O | | | | 20 | g |

To a stirring solution of CuCl (20 mg, 0.02 mmoL) and hydroxylamine hydrochloride (45.4 mg, 0.06 mmol) in 23% butan-1-amine (aq) at 0° C. was added a solution of compound 61.4 (2 g, 10 mmol) in 23% butan-1-amine (aq). A solution of INT-1 (2.7 g, 9 mmol) in butan-1-amine (12.2 g, 120 mmol), MeOH (10 mL), and THF (5 mL) was added and the reaction progress was followed by TLC. The reaction mixture was diluted with water (100 mL), and extracted with EA (3×20 mL). The combined organic layers were dried, concentrated under reduced pressure to give a crude, which was purified by flash chromatography (silica gel/PE:EA 10:1 to 5:1) to give compound 61.5 (2.5 g, 60%) as a solid. MS: m/z calcd for C₂₀H₂₆O₄Si 358.2. found [M+H]⁺ 359.

### Methyl 4-(5,6-dihydroxyhexa-1,3-diynyl)benzoate (61.6)

| Reagent | MW | Eq. | Mmol | g, mL | |
|---|---|---|---|---|---|
| Compound 61.5 | 358.5 | 1 | 5.57 | 2 | g |
| MeOH•HCl | | 5 | 27.8 | 5 | mL |
| MeOH | | | | 20 | mL |

To a stirring solution of compound 61.6 (2 g, 5.57 mmol) in MeOH (20 mL) was added MeOH.HCl (0.08 mL) and the reaction progress was followed by TLC. The solvent was removed under reduced pressure, and the desired product 61.6 was collected as a white solid (1.3 g, 95%).

### (S)-methyl 4-(5,6-dihydroxyhexa-1,3-diynyl)benzoate (61.7) and (R)-methyl dihydroxyhexa-1,3-diynyl)benzoate (61.8)

Chiral separation (column AD-H 30×250 mm, 35° C., mobile phase CO₂/MeOH (65/35), 60 g/min) of compound 61.6 yielded compound 61.7 (0.6 g) and compound 61.8 (0.5 g).

### (S)-4-(5,6-dihydroxyhexa-1,3-diynyl)benzoic acid (61.9)

| Reagent | MW | Eq. | Mmol | g, mL | |
|---|---|---|---|---|---|
| Compound 61.7 | 244 | 1 | 4 | 0.488 | g |
| NaOH(a•q) | 56 | 3 | 12 | 0.336 | g |
| MeOH | | | | 20 | mL |
| H₂O | | | | 4 | mL |

To a stirring solution of compound 61.7 (0.488 g, 4.0 mmol) in MeOH (20 mL) and H₂O (4 mL) was added NaOH (0.336 g, 12.0 mmol) and the resulting mixture was refluxed for 2 h. After cooling to room temperature, 2N HCl was added to adjust the pH to 3. The reaction was filtered and the target compound 61.9 was collected as a white solid (414 mg, 90%). ¹H NMR (400 MHz, DMSO-d₆) δ 13.273 (s, 1H), 7.94 (d, J=8.0 Hz, 2H), 7.67 (d, J=8.0 Hz, 2H), 5.79 (s, 1H), 5.12 (s, 1H), 4.37 (s, 1H), 3.46 (s, 2H).

### (R)-4-(5,6-dihydroxyhexa-1,3-diynyl)benzoic acid (61.10)

| Reagent | MW | Eq. | mmol | g, mL |
|---|---|---|---|---|
| Compound 61.8 | 244 | 1 | 4 | 0.488 |
| NaOH(a•q) | 56 | 3 | 12 | 0.336 |
| MeOH | | | | 20 mL |

To a stirring solution of compound 61.8 (0.488 g, 4.0 mmol) in MeOH (20 mL) and H₂O (4 mL) was added NaOH (0.336 g, 12.0 mmol) and the resulting mixture was refluxed for 2 hr. After cooling to room temperature, 2N HCl was added to adjust the pH to 3. The reaction was filtered and the target compound 61.10 was collected as a white solid (400 mg, 82%). ¹H NMR (400 MHz, DMSO-d₆) δ 13.25 (s, 1H), 7.94 (d, J=8.0 Hz, 2H), 7.67 (d, J=8.0 Hz, 2H), 5.79 (s, 1H), 5.12 (s, 1H), 4.37 (s, 1H), 3.46 (s, 2H).

### (S)-methyl 3-((tert-butoxycarbonyl)amino)-2-(4-((S)-5,6-dihydroxyhexa-1,3-diyn-1-yl)benzamido)-3-methylbutanoate (61.11)

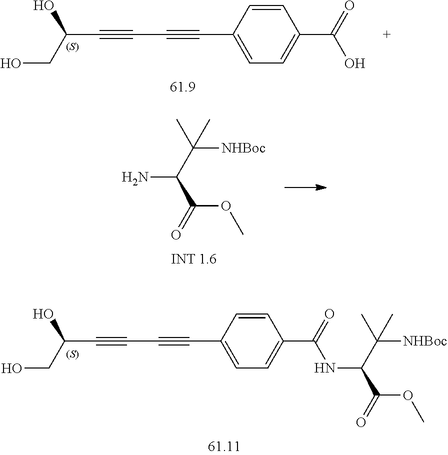

| | | | | Sam- | | |
|---|---|---|---|---|---|---|
| | | | | ple | | Den- |
| | | | Moles | Mass | Vol | sity |
| Reactant | MW | Eq | (mmol) | (g) | (ml) | (g/ml) |
| diisopropylethylamine | 101.190 | 1.3 | 24.85 | 2.51 | 3.54 | 0.71 |
| 2-(3H-[1,2,3]triazolo[4,5- | 235.265 | 1.2 | 22.93 | 8.72 | | |
| b]pyridin-3-yl)-1,1,3,3- | | | | | | |
| tetramethylisouronium | | | | | | |
| hexafluorophosphate(V) | | | | | | |
| (S)-4-(5,6-dihydroxyhexa- | 230.216 | 1.000 | 19.11 | 4.4 | | |
| 1,3-diyn-1-yl)benzoic acid | | | | | | |
| (S)-methyl 2-amino-3-((tert- | 246.303 | 1.08 | 20.64 | 5.08 | | |
| butoxycarbonyl)amino)-3- | | | | | | |
| methylbutanoate | | | | | | |

To a stirring solution of (S)-methyl 2-amino-3-((tert-butoxycarbonyl)amino)-3-methylbutanoate (5.08 g, 20.64 mmol) in DMF (100 mL) at 0° C. was added DIPEA (3.54 mL, 24.85 mmol), followed by compound 61.9 (4.4 g, 19.11 mmol) and HATU (8.72 g, 22.93 mmol) and the reaction was allowed to stir for 3 hr. The mixture was diluted with EtOAc, washed with saturated aq NH₄Cl, saturated bicarbonate, brine, dried over Na₂SO₄, filtered and concentrated under reduced pressure to yield compound 61.11, which was carried through to the next step without further purification. MS: m/z calcd for C₂₄H₃₀N₂O₇458.2. found [M+H]⁺ 459.2.

### (S)-methyl 3-amino-2-(4-((S)-5,6-dihydroxyhexa-1,3-diyn-1-yl)benzamido)-3-methylbutanoate (61.12)

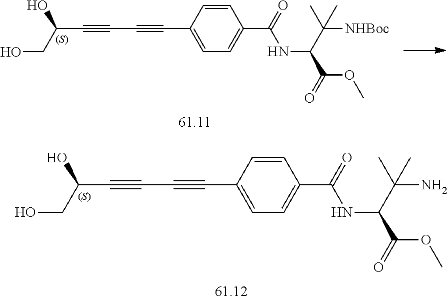

To a stirring solution of compound 61.11 (19.11 mmol) in DCM (30 mL) at 0° C. was added TFA (40 ml) and the reaction was stirred for 4 hr. The reaction mixture was concentrated under reduced pressure to give a crude, which was azeotroped with IPA twice. The resulting residue was partitioned between EtOAc and saturated aqueous NaHCO₃. The aqueous layer was extracted with MeTHF (3×), and the combined organic layers were dried over Na₂SO₄, filtered and concentrated under reduced pressure to yield 61.12, which was carried through to the next step without further purification. MS: m/z calcd for C₁₉H₂₂N₂O₅358.1. found [M+H]⁺ 359.1.

### N—((S)-3-amino-1-(hydroxyamino)-3-methyl-1-oxobutan-2-yl)-4-((S)-5,6-dihydroxyhexa-1,3-diyn-1-yl)benzamide, Acetate (61)

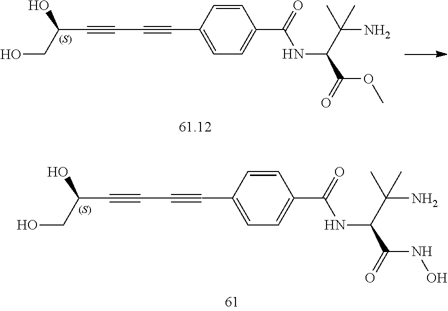

To a stirring solution of compound 61.12 (19.11 mmol) in IPA (15 mL) at 0° C. was added hydroxylamine (25 mL), and the reaction was stirred overnight. IPA was removed under reduced pressure and the residue was purified by RP HPLC to yield compound 61 (2.12 g, 5.05 mmol). MS: m/z calcd for C₁₈H₂₁N₃O₅ 359.1. found [M+H]⁺ 360.3.

### 62. N—((S)-3-amino-1-(hydroxyamino)-3-methyl-1-oxobutan-2-yl)-4-((R)-5,6-dihydroxyhexa-1,3-diyn-1-yl)benzamide (62)

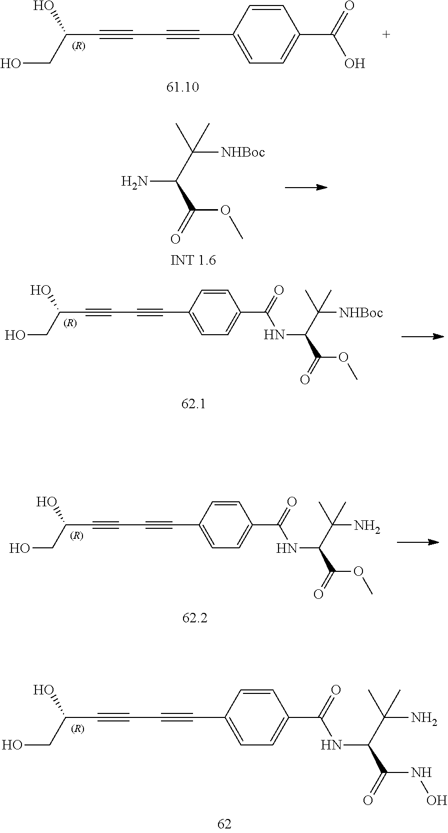

### (S)-methyl 3-(tert-butoxycarbonylamino)-2-(4-((R)-5,6-dihydroxyhexa-1,3-diynyl)benzamido)-3-methylbutanoate (62.1)

| | | | | |
|---|---|---|---|---|
| Reagent | MW | Eq. | mmol | g, mL |

| | | | | | |
|---|---|---|---|---|---|
| Compound 61.10 | 230 | 1 | 1 | 0.23 | g |
| (S)-methyl2-amino-3-((tert- | 246 | 1 | 1 | 0.246 | g |
| butoxycarbonyl)amino)-3- | | | | | |
| methylbutanoate | 380 | 1 | 1 | 0.38 | g |
| HATU | 128 | 4 | 4l | 0.512 | g |
| DIPEA | | | | 20 | mL |
| DMF | | | | | |

To a stirring solution of (S)-methyl2-amino-3-((tert-butoxycarbonyl)amino)-3-methylbutanoate (0.246 g, 1.0 mmol) and HATU (0.38 g, 1.0 mmol) in DMF (20 mL) was added compound 61.10 (0.23 g, 1.0 mmoL) followed by DIPEA (0.512 g, 4.0 mmoL) and the reaction was stirred overnight. Water (100 mL) was added and the solution was extracted with EA (3×20 mL). The combined organic layers were washed with brine, dried over Na₂SO₄, filtered and concentrated under reduced pressure to give a crude, which was purified by flash chromatography (silica gel) to give compound 62.1 as a white solid. MS: m/z calcd for C₂₄H₃₀N₂O₇ 458.2. found [M+H]⁺ 459.

### (S)-methyl 3-amino-2-(4-((R)-5,6-dihydroxyhexa-1,3-diynyl)benzamido)-3-methylbutanoate (62.2)

To a stirring solution of compound 62.1 (0.458 g, 1 mmol) in MeOH (3 mL) was added MeOH.HCl (0.08 mL) and the reaction progress was monitored by TLC. The reaction was diluted with Et₂O (200 mL), filtered and the desired compound 62.2 was collected as a white solid (330 mg, 93%). ¹H NMR (400 MHz, DMSO-d₆) δ 9.03 (d, J=8.0 Hz, 1H), 8.26 (s, 3H), 7.98 (d, J=8.0 Hz, 2H), 7.70 (d, J=8.0 Hz, 2H), 5.77 (d, J=8 Hz, 1H), 5.01 (t, J=8 Hz, 1H), 4.99 (d, J=8 Hz, 1H), 3.72 (s, 3H), 3.45 (m, 2H), 1.39 (d, J=2 Hz, 6H).

### N—((S)-3-amino-1-(hydroxyamino)-3-methyl-1-oxobutan-2-yl)-4-((R)-5,6-dihydroxyhexa-1,3-diyn-1-yl)benzamide (62)

To a stirring solution of compound 62.2 (22 mg, 0.056 mmol) in IPA (500 μL) was added hydroxylamine (50% aq, 500 μL) and the reaction was stirred overnight at 5-10° C. The reaction mixture was acidified with acetic acid, diluted with water and purified by RP HPLC (1″, 0.1% TFA in water/ACN) to yield compound 62 (13.1 mg, 49.7%). MS: m/z calcd for C₁₈H₂₁N₃O₅ 359.1. found [M+H]⁺ 360.1.

### 63. 4-(5,6-dihydroxyhexa-1,3-diyn-1-yl)-N—((S)-3-hydroxy-1-(hydroxyamino)-3-methyl-1-oxobutan-2-yl)benzamide (63)

### (S)-methyl 2-(4-(bromoethynyl)benzamido)-3-hydroxy-3-methylbutanoate (63.1)

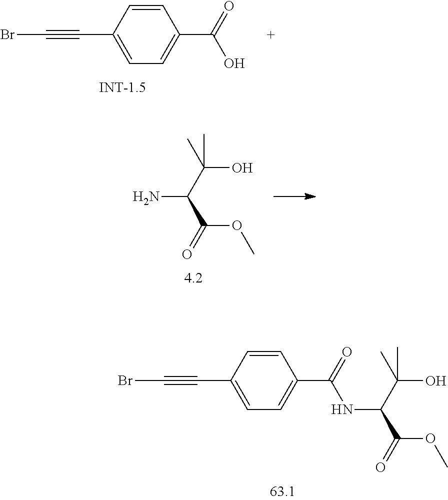

| | | | | | Sample | |
|---|---|---|---|---|---|---|
| | | | Density | Moles | Mass | Vol |
| Reactant | MW | Eq | (g/ml) | (mmol) | (g) | (mL) |
| INT-1.5 | 225.04 | 1.003 | | 13.33 | 3 | |
| (R)-methyl 2-amino-3- | 147.17 | 1 | | 13.29 | 2.441 | |
| hydroxy-3- | | | | | | |
| methylbutanoate, HCl | | | | | | |
| HATU | 235.27 | 1.2 | | 15.95 | 6.06 | |
| TEA | 101.19 | 3 | 0.73 | 39.9 | 4.03 | 5.56 |

To a stirring solution of (R)-methyl 2-amino-3-hydroxy-3-methylbutanoate, HCl (2.441 g, 13.29 mmol) in acetonitrile (25 mL) was added INT-1.5 (3 g, 13.33 mmol) followed by TEA (5.56 mL, 39.9 mmol). HATU (6.06 g, 15.95 mmol) was then added portionwise over 20 minutes, and the reaction was stirred for 3 hours. The reaction mixture was concentrated under reduced pressure to a residue, which was diluted with ethyl acetate (30 mL), and filtered. The filtrate was washed with 1M citric acid (2×30 mL), saturated. aqueous NaHCO₃ (4×30 mL), 1M NaOH (30 mL), and brine (30 mL). The organic layer was concentrated under reduced pressure to yield compound 63.1 (4.49 g, 95%) as a brown solid, which was carried through to the next step without further purification.

### (2S)-methyl-2-(4-(5,6-dihydroxyhexa-1,3-diyn-1-yl)benzamido)-3-hydroxy-3-methylbutanoate (63.3)

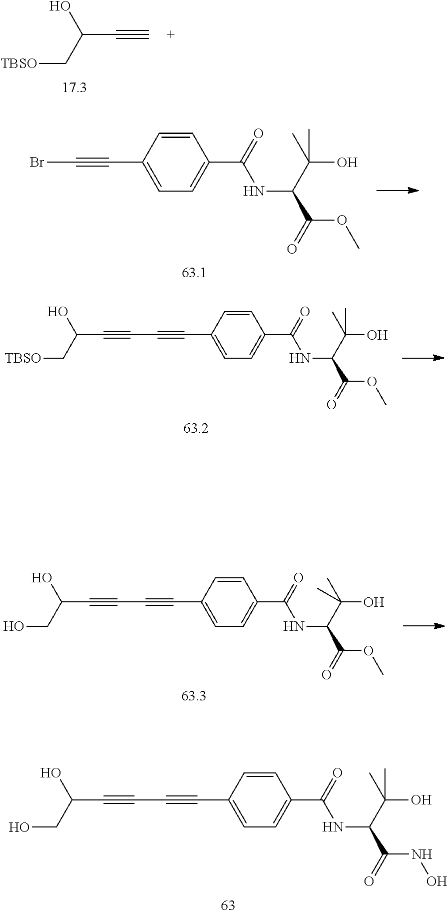 [SMILES: C#CC(O)CO[Si](C)(C)C(C)(C)C ; COC(=O)[C@@H](NC(=O)c1ccc(C#CBr)cc1)C(C)(C)O ; COC(=O)[C@@H](NC(=O)c1ccc(C#CC#CC(O)CO[Si](C)(C)C(C)(C)C)cc1)C(C)(C)O ; COC(=O)[C@@H](NC(=O)c1ccc(C#CC#CC(O)CO)cc1)C(C)(C)O ; CC(C)(O)[C@H](NC(=O)c1ccc(C#CC#CC(O)CO)cc1)C(=O)NO]

| | | | | Sample |
|---|---|---|---|---|
| | | | Moles | Mass |
| Reactant | MW | Eq | (mmol) | (g) |
| hydroxylamine hydrochloride | 33.030 | 0.06 | 0.898 | 0.062 |
| copper(I) chloride | 98.999 | 0.02 | 0.299 | 0.030 |
| 1-((tert-butyldimethylsilyl)oxy)but-3-yn- | 200.350 | 1.000 | 14.97 | 3.00 |
| 2-ol | | | | |
| (S)-methyl 2-(4- | 354.196 | 1.000 | 14.97 | 5.30 |
| (bromoethynyl)benzamido)-3-hydroxy- | | | | |
| 3-methylbutanoate | | | | |

To a stirring solution of hydroxylamine hydrochloride (0.062 g, 0.898 mmol) and copper(I) chloride (0.030 g, 0.299 mmol) in 30% n-butylamine/water (60 mL) at 0° C. was added a solution of 1-((tert-butyldimethylsilyl)oxy)but-3-yn-2-ol (3.00 g, 14.97 mmol) in 30% n-butylamine/water (10 mL), followed by a solution of (S)-methyl 2-(4-(bromoethynyl)benzamido)-3-hydroxy-3-methylbutanoate (5.30 g, 14.97 mmol) in 30% n-butylamine/water (20 mL) and THF (20 mL) and the reaction was stirred for 1 hr. The reaction mixture was extracted with EtOAc, and concentrated under reduced pressure to yield compound 63.2 (5.92 g, MS: m/z calcd for C₂₅H₃₅NO₆Si 473.2. found [M+Na]⁺ 496.2), which was treated with TFA (20 mL) for 15 min. Excess TFA was removed under reduced pressure, water (100 mL) was added and the mixture was extracted with EtOAc, dried over Na₂SO₄ and concentrated under reduced pressure to yield compound 63.3 (4.67 g).

### 4-(5,6-dihydroxyhexa-1,3-diyn-1-yl)-N—((S)-3-hydroxy-1-(hydroxyamino)-3-methyl-1-oxobutan-2-yl)benzamide (63)

To a stirring solution of compound 63.3 (4.67 g) in IPA (50 mL) was added hydroxylamine (50% aqueous, 50 mL) and the reaction was stirred for 3 days. The solvent was removed under reduced pressure and the mixture was acidified with AcOH and purified by RP HPLC (6″, 0.1% AcOH in water/ACN) to yield compound 63 (665 mg). MS: m/z calcd for C₁₈H₂₀N₂O₆ 360.1. found [M+H]⁺ 361.1.

B. Antimicrobial Activity

### 1. Bacterial Screens and Cultures

Bacterial isolates were cultivated from −70° C. frozen stocks by overnight passages at 35° C. in ambient air on Mueller-Hinton agar (Beckton Dickinson, Franklin Lakes, N.J.). Clinical isolates tested were obtained from various geographically diverse hospitals in the US and abroad (Focus Diagnostics, Herndon, Va. and JMI, North Liberty, Iowa). Quality control strains were from the American Type Culture Collection (ATCC; Rockville, Md.).

### 2. Susceptibility Testing

Minimum Inhibitory Concentrations (MICs) were determined by the broth microdilution method in accordance with the Clinical and Laboratory Standards Institute (CLSI) guidelines. In brief, organism suspensions were adjusted to a 0.5 McFarland standard to yield a final inoculum between 3×10⁵ and 7×10⁵ colony-forming units (CFU)/mL. Drug dilutions and inocula were made in sterile, cation adjusted Mueller-Hinton Broth (Beckton Dickinson). An inoculum volume of 100 μL was added to wells containing 100 μL of broth with 2-fold serial dilutions of drug. All inoculated microdilution trays were incubated in ambient air at 35° C. for 18-24 h. Following incubation, the lowest concentration of the drug that prevented visible growth (OD600 nm<0.05) was recorded as the MIC. Performance of the assay was monitored by the use of laboratory quality-control strains and levofloxacin, a compound with a defined MIC spectrum, in accordance with CLSI guidelines. Typically, compounds of the present invention have MIC values of 0.03-16 μg/mL. To this end, data for certain representative compounds is shown in Table I below.

TABLE I
Minimum Inhibitory Concentrations (MICs)

| Cmpd | APAE001 | APAE002 | APAE1096 |
|---|---|---|---|
| 1 | A | A | A |
| 2 | A | B | C |
| 3 | A | A | B |
| 4 | A | A | B |
| 5 | A | A | A |
| 6 | A | A | B |
| 7 | A | A | A |
| 8 | B | B | C |
| 9 | A | A | A |
| 10 | A | A | A |
| 11 | A | A | B |
| 12 | B | B | C |
| 13 | A | B | B |
| 14 | A | A | B |
| 15 | A | A | A |
| 16 | A | A | B |
| 17 | B | B | C |
| 18 | B | B | C |
| 19 | A | B | B |
| 20A | B | B | C |
| 20B | B | B | C |
| 21 | B | B | B |
| 22 | A | A | B |
| 23A | B | B | C |
| 23B | B | B | C |
| 24 | B | B | B |
| 25 | A | A | B |
| 26 | A | A | B |
| 27 | A | A | B |
| 28 | B | B | D |
| 29 | A | A | B |
| 30A | A | A | C |
| 30B | A | A | B |
| 31A | B | B | C |
| 31B | A | A | B |
| 32 | A | A | B |
| 33A | B | A | C |
| 33B | A | B | B |
| 34 | A | A | B |
| 35 | A | A | B |
| 36 | A | A | B |
| 37 | B | B | B |
| 38 | B | A | B |
| 39 | A | A | B |
| 40 | B | A | B |
| 41 | A | A | B |
| 42 | B | B | C |
| 43 | A | A | B |
| 44 | A | A | B |
| 45 | B | A | B |
| 46 | A | A | B |
| 47 | A | A | B |
| 48 | A | A | B |
| 49 | A | A | B |
| 50 | A | B | C |
| 51 | A | A | B |
| 52 | B | B | C |
| 53 | B | B | C |
| 54 | A | A | C |
| 55 | A | A | B |
| 56 | A | A | B |
| 57 | A | A | B |
| 58 | B | B | C |
| 59 | B | A | B |
| 60 | B | ND | ND |
| 61 | A | A | B |
| 62 | A | A | B |

MIC Key:
A = MIC's of 1.0 μg/mL or less
B = MIC's of greater than 1.0 μg/mL to 8.0 μg/mL
C = MIC's of greater than 8.0 μg/mL to 16.0 μg/mL
D = MIC's of greater than 16.0 μg/mL
* APAE001 is Pseudomonas aeruginosa ATCC27853; APAE002 is a wildtype lab strain of Pseudomonas aeruginosa known as PAM1020 or PA01. APAE1096 is a clinical isolate, gift of Kurt Munson (FOCUS, Herndon, VA).

C. Protein Binding

The effect of human serum albumin (HSA) and α-1-acid glycoprotein (AGP) from human plasma, which are known to nonspecifically bind to certain antibiotics and thereby reduce their activity (Banéres-Roquet et al. 2009 “Use of a Surface Plasmon Resonance Method to Investigate Antibiotic and Plasma Protein Interactions” Antimicrob. Agents and Chemother. 53(4):1528-1531), was assessed against 3 P. aeruginosa strains (APAE001, APAE002, APAE006). APAE001 is Pseudomonas aeruginosa ATCC27853; APAE002 is a wildtype lab strain of Pseudomonas aeruginosa known as PAM1020 or PA01. APAE006 is a strain of PAM2010/PA01 with efflux deletions: delta mexAB-oprM::CM delta CD-oprJ::Gm delta mexEF-oprN::omega Hg. Lomovskaya, 0 et al. 1999. Use of a Genetic Approach To Evaluate the Consequences of Inhibition of Efflux Pumps in P aeruginosa. Antimicrob. Agents Chemother. 43: 1340-1346.

The standard procedure described in Susceptibility Testing was utilized with one adjustment, specifically 2% HSA and 0.05% AGP were added to the cation adjusted Mueller-Hinton Broth. The MIC for each strain of interest was then assessed in the presence and absence of HSA/AGP. Following MIC determination, the fold change in MIC was determined for each individual strain by dividing the MIC in the presence of HSA/AGP by the MIC in the absence of HSA/AGP. The protein binding Geomean was then calculated using these 3 values. Table II summarizes the results of these measurements.

TABLE II
Protein Binding

| Cmpd | PB |
|---|---|
| 1 | 4.1 |
| 2 | 1.3 |
| 3 | 1.3 |
| 4 | n.d. |
| 5 | 1.6 |
| 6 | 2.6 |
| 7 | 2.5 |
| 8 | 0.9 |
| 9 | 1.5 |
| 10 | 2.2 |
| 11 | 1.5 |
| 12 | 2.9 |
| 13 | 1.4 |
| 14 | 3.4 |
| 15 | 2.3 |
| 16 | 3.6 |
| 17 | 1.5 |
| 18 | 1.6 |
| 19 | 1.0 |
| 20A | 2.0 |
| 20B | 1.0 |
| 21 | 1.9 |
| 22 | 1.6 |
| 23A | 2.3 |
| 23B | 2.0 |
| 24 | 2.5 |
| 25 | 11.5 |
| 26 | 6.4 |
| 27 | 1.8 |
| 28 | 1.3 |
| 29 | 2.9 |
| 30A | 2.5 |
| 30B | 3.2 |
| 31A | 2.5 |
| 31B | 3.2 |
| 32 | 5.5 |
| 33A | 2.6 |
| 33B | 5.1 |
| 34 | 2.8 |
| 35 | 2.0 |
| 36 | 2.3 |
| 37 | 1.4 |
| 38 | 2 |
| 39 | 2.8 |
| 40 | 2.6 |
| 41 | 2 |
| 42 | 2.6 |
| 43 | 1.6 |
| 44 | 2.0 |
| 45 | 2.0 |
| 46 | nd |
| 47 | 2.6 |
| 48 | 2.0 |
| 49 | 2.0 |
| 50 | 1.6 |
| 51 | 1.6 |
| 52 | 1.0 |
| 53 | 2.0 |
| 54 | 2.0 |
| 55 | 2.0 |
| 56 | 2.5 |
| 57 | 1.6 |
| 58 | 1.0 |
| 59 | 3.2 |
| 60 | nd |
| 61 | 1.6 |
| 62 | 2.0 |

It should be understood that the organic compounds according to the invention may exhibit the phenomenon of tautomerism. As the chemical structures within this specification can only represent one of the possible tautomeric forms, it should be understood that the invention encompasses any tautomeric form of the drawn structure.

Furthermore, while particular embodiments of the present invention have been shown and described herein for purposes of illustration, it will be understood, of course, that the invention is not limited thereto since modifications may be made by persons skilled in the art, particularly in light of the foregoing teachings, without deviating from the spirit and scope of the invention. Accordingly, the invention is not limited except as by the appended claims.

All of the U.S. patents, U.S. patent application publications, U.S. patent applications, foreign patents, foreign patent applications and non-patent publications referred to in this specification are incorporated herein by reference, in their entirety to the extent not inconsistent with the present description.

1. A compound of formula I: 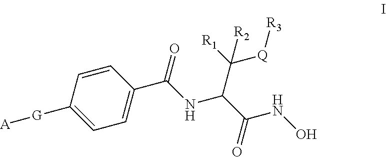
or a stereoisomer, pharmaceutically acceptable salt, or ester thereof, wherein
A is selected from the group consisting of:
(a) substituted C₁-C₂ alkyl, wherein at least one substituent is hydroxy;
(b) substituted C₃-C₅ alkyl, wherein at least two substituents are hydroxy;
(c) substituted cycloalkyl, wherein;
(i) at least one substituent is dihydroxyalkyl; or
(ii) at least two substituents independently are selected from hydroxy and hydroxyalkyl; and
(d) substituted cycloalkylalkyl, wherein at least two substituents independently are selected from hydroxy and hydroxyalkyl and wherein each substitution independently is to either the cyclic portion or alkyl portion of the cycloalkylalkyl;
G is selected from the group consisting of —C≡C—, —CH═CH—C≡C—, —C≡C—CH═CH—, and —C≡C—C≡C—;
Q is O or NR, wherein R is hydrogen or an unsubstituted C₁-C₃ alkyl;
R¹ and R² independently are selected from the group consisting of hydrogen and substituted or unsubstituted C₁-C₃ alkyl, or R¹ and R², together with the carbon atom to which they are attached, form an unsubstituted C₃-C₆ cycloalkyl group or an unsubstituted 4-6 membered heterocyclic group; and
R³ is selected from the group consisting of hydrogen, substituted or unsubstituted C₁-C₆ alkyl, substituted or unsubstituted cycloalkyl, substituted or unsubstituted cycloalkylalkyl, substituted or unsubstituted aryl, substituted or unsubstituted arylalkyl, substituted or unsubstituted heterocyclyl, substituted or unsubstituted heterocyclylalkyl, substituted or unsubstituted heteroaryl, and substituted or unsubstituted heteroarylalkyl.

2. The compound of claim 1, wherein R³ is hydrogen or a substituted or unsubstituted C₁-C₆ alkyl.

3. The compound of claim 1, wherein G is —C≡C—C≡C—.

4. The compound of claim 1, wherein Q is NR.

5. The compound of claim 4, wherein R is hydrogen.

6. The compound of claim 1, wherein R¹ is methyl and R² is methyl.

7. The compound of claim 1, wherein A is substituted C₁-C₂ alkyl, wherein at least one substituent is hydroxy.

8. The compound of claim 7, wherein A is hydroxymethyl.

9. The compound of claim 7, wherein A is hydroxyethyl.

10. The compound of claim 1, wherein A is substituted C₃-C₈ alkyl, wherein at least two substituents are hydroxyl.

11. The compound of claim 1, wherein A is substituted cycloalkyl, wherein at least two substituents independently are selected from hydroxy and hydroxyalkyl.

12. The compound of claim 11, wherein the at least two substituents independently are selected from hydroxy and hydroxymethyl.

13. The compound of claim 1, wherein A is substituted cycloalkylalkyl, wherein at least two substituents are independently selected from hydroxy and hydroxyalkyl, and wherein each substitution independently is to either the cyclic portion or alkyl portion of the cycloalkylalkyl.

14. The compound of claim 13, wherein the at least two substituents independently are selected from hydroxy and hydroxymethyl.

15. The compound of claim 1, wherein said compound is:
(S)—N-(3-amino-1-(hydroxyamino)-3-methyl-1-oxobutan-2-yl)-4-(5-hydroxypenta-1,3-diynyl)benzamide (1);
(S)—N-(3-amino-1-(hydroxyamino)-3-methyl-1-oxobutan-2-yl)-4-(3-hydroxyprop-1-ynyl)benzamide (2);
(S,E)-N-(3-amino-1-(hydroxyamino)-3-methyl-1-oxobutan-2-yl)-4-(5-hydroxypent-3-en-1-ynyl)benzamide (3);
(S)—N-(3-hydroxy-1-(hydroxyamino)-3-methyl-1-oxobutan-2-yl)-4-(5-hydroxypenta-1,3-diynyl)benzamide (4);
(S)—N-(1-(hydroxyamino)-3-methyl-3-(methylamino)-1-oxobutan-2-yl)-4-(5-hydroxypenta-1,3-diynyl)benzamide (5);
(S)—N-(1-(hydroxyamino)-3-(2-hydroxyethylamino)-3-methyl-1-oxobutan-2-yl)-4-(5-hydroxypenta-1,3-diynyl)benzamide (6);
(S)—N-(3-amino-1-(hydroxyamino)-3-methyl-1-oxobutan-2-yl)-4-(6-hydroxyhexa-1,3-diynyl)benzamide (7);
(S)—N-(3-amino-1-(hydroxyamino)-3-methyl-1-oxobutan-2-yl)-4-(4-hydroxybut-1-ynyl)benzamide (8);
(S)—N-(1-(hydroxyamino)-3-methyl-3-(methylamino)-1-oxobutan-2-yl)-4-(6-hydroxyhexa-1,3-diynyl)benzamide (9);
(S)—N-(3-(ethylamino)-1-(hydroxyamino)-3-methyl-1-oxobutan-2-yl)-4-(6-hydroxyhexa-1,3-diynyl)benzamide (10);
(S)—N-(1-(hydroxyamino)-3-(2-hydroxyethylamino)-3-methyl-1-oxobutan-2-yl)-4-(6-hydroxyhexa-1,3-diynyl)benzamide (11);
(S)—N-(1-(hydroxyamino)-3-methyl-3-((5-methylisoxazol-3-yl)methylamino)-1-oxobutan-2-yl)-4-(6-hydroxyhexa-1,3-diynyl)benzamide (12);
(S)—N-(3-((1H-imidazol-4-yl)methylamino)-1-(hydroxyamino)-3-methyl-1-oxobutan-2-yl)-4-(6-hydroxyhexa-1,3-diynyl)benzamide (13);
N—((S)-3-amino-1-(hydroxyamino)-3-methyl-1-oxobutan-2-yl)-4-((S)-5-hydroxyhexa-1,3-diynyl)benzamide (14);
N—((S)-3-amino-1-(hydroxyamino)-3-methyl-1-oxobutan-2-yl)-4-((R)-5-hydroxyhexa-1,3-diynyl)benzamide (15);
N—((S)-1-(hydroxyamino)-3-methyl-3-(methylamino)-1-oxobutan-2-yl)-4-((S)-5-hydroxyhexa-1,3-diynyl)benzamide (16);
N—((S)-3-amino-1-(hydroxyamino)-3-methyl-1-oxobutan-2-yl)-4-(5,6-dihydroxyhexa-1,3-diynyl)benzamide (17);
(S)—N-(3-amino-1-(hydroxyamino)-3-methyl-1-oxobutan-2-yl)-4-(6-hydroxy-5-(hydroxymethyl)hexa-1,3-diynyl)benzamide (18);
N—((S)-3-amino-1-(hydroxyamino)-3-methyl-1-oxobutan-2-yl)-4-(6-chloro-5-hydroxy-5-(hydroxymethyl)hexa-1,3-diynyl)benzamide (19);
N—((S)-3-amino-1-(hydroxyamino)-3-methyl-1-oxobutan-2-yl)-4-((5S,6S)-6,7-dihydroxy-5-methylhepta-1,3-diynyl)benzamide (20A);
N—((S)-3-amino-1-(hydroxyamino)-3-methyl-1-oxobutan-2-yl)-4-((5S,6R)-6,7-dihydroxy-5-methylhepta-1,3-diynyl)benzamide (20B);
N—((S)-3-amino-1-(hydroxyamino)-3-methyl-1-oxobutan-2-yl)-4-(6,7-dihydroxyhepta-1,3-diynyl)benzamide (21);
4-(6,7-dihydroxyhepta-1,3-diynyl)-N—((S)-1-(hydroxyamino)-3-methyl-3-(methylamino)-1-oxobutan-2-yl)benzamide (22);
N—((S)-3-amino-1-(hydroxyamino)-3-methyl-1-oxobutan-2-yl)-4-(((1S,2R,3S)-2,3-bis(hydroxymethyl)cyclopropyl)buta-1,3-diynyl)benzamide (23A);
N—((S)-3-amino-1-(hydroxyamino)-3-methyl-1-oxobutan-2-yl)-4-(((1R,2R,3S)-2,3-bis(hydroxymethyl)cyclopropyl)buta-1,3-diynyl)benzamide (23B);
N—((S)-3-amino-1-(hydroxyamino)-3-methyl-1-oxobutan-2-yl)-4-((R)-2,2-bis(hydroxymethyl)cyclopropyl)buta-1,3-diynyl)benzamide (24);
N—((S)-3-amino-1-(hydroxyamino)-3-methyl-1-oxobutan-2-yl)-4-((2-(1,2-dihydroxyethyl)cyclopropyl)buta-1,3-diynyl)benzamide (25);
N—((S)-3-amino-1-(hydroxyamino)-3-methyl-1-oxobutan-2-yl)-4-(5-hydroxy-5-(2-(hydroxymethyl)cyclopropyl)penta-1,3-diynyl)benzamide (26);
N—((S)-3-amino-1-(hydroxyamino)-3-methyl-1-oxobutan-2-yl)-4-(((1S,3R)-1-hydroxy-3-(hydroxymethyl)cyclobutyl)buta-1,3-diynyl)benzamide (27);
N—((S)-3-amino-1-(hydroxyamino)-3-methyl-1-oxobutan-2-yl)-4-(5-(1-hydroxy-3-(hydroxymethyl)cyclobutyl)penta-1,3-diynyl)benzamide (28);
N—((S)-3-amino-1-(hydroxyamino)-3-methyl-1-oxobutan-2-yl)-4-(((1R,3S)-3-hydroxy-3-(hydroxymethyl)cyclobutyl)buta-1,3-diynyl)benzamide (29);
N—((S)-3-amino-1-(hydroxyamino)-3-methyl-1-oxobutan-2-yl)-4-(((1S,2S,3S)-3-hydroxy-2-(hydroxymethyl)cyclobutyl)buta-1,3-diynyl)benzamide (30A);
N—((S)-3-amino-1-(hydroxyamino)-3-methyl-1-oxobutan-2-yl)-4-(((1S,2S,3R)-3-hydroxy-2-(hydroxymethyl)cyclobutyl)buta-1,3-diynyl)benzamide (30B);
N—((S)-3-amino-1-(hydroxyamino)-3-methyl-1-oxobutan-2-yl)-4-(((1S,2R,3R)-3-hydroxy-2-(hydroxymethyl)cyclobutyl)buta-1,3-diynyl)benzamide (31A);
N—((S)-3-amino-1-(hydroxyamino)-3-methyl-1-oxobutan-2-yl)-4-(((1S,2R,3S)-3-hydroxy-2-(hydroxymethyl)cyclobutyl)buta-1,3-diynyl)benzamide (31B);
N—((S)-3-amino-1-(hydroxyamino)-3-methyl-1-oxobutan-2-yl)-4-(((1S,3R,4S)-3,4-dihydroxycyclopentyl)buta-1,3-diynyl)benzamide (32);
N—((S)-3-amino-1-(hydroxyamino)-3-methyl-1-oxobutan-2-yl)-4-(((1S,4R)-4-hydroxy-4-(hydroxymethyl)cyclohexyl)buta-1,3-diynyl)benzamide (33A);
N—((S)-3-amino-1-(hydroxyamino)-3-methyl-1-oxobutan-2-yl)-4-(((1R,4S)-4-hydroxy-4-(hydroxymethyl)cyclohexyl)buta-1,3-diynyl)benzamide (33B);
(S)—N-(3-hydroxy-1-(hydroxyamino)-3-methyl-1-oxobutan-2-yl)-4-(6-hydroxyhexa-1,3-diynyl)benzamide (34);
N-((2S,3R)-3-amino-1-(hydroxyamino)-1-oxobutan-2-yl)-4-(6-hydroxyhexa-1,3-diyn-1-yl)benzamide (35);
N—((S)-3-amino-1-(hydroxyamino)-3-methyl-1-oxobutan-2-yl)-4-((S)-6,7-dihydroxyhepta-1,3-diyn-1-yl)benzamide (36);
N—((S)-3-amino-1-(hydroxyamino)-3-methyl-1-oxobutan-2-yl)-4-((R)-6,7-dihydroxyhepta-1,3-diyn-1-yl)benzamide (37);
N-((2S,3R)-3-hydroxy-1-(hydroxyamino)-1-oxobutan-2-yl)-4-(6-hydroxyhexa-1,3-diyn-1-yl)benzamide (38);
N—((S)-3-amino-1-(hydroxyamino)-3-methyl-1-oxobutan-2-yl)-4-(6-hydroxy-5-methylhexa-1,3-diyn-1-yl)benzamide (39);
N—((S)-3-hydroxy-1-(hydroxyamino)-3-methyl-1-oxobutan-2-yl)-4-((S)-5-hydroxyhexa-1,3-diyn-1-yl)benzamide (40);
N-((2S,3R)-3-amino-1-(hydroxyamino)-1-oxobutan-2-yl)-4-((S)-5-hydroxyhexa-1,3-diyn-1-yl)benzamide (41);
N-((2S,3R)-3-hydroxy-1-(hydroxyamino)-1-oxobutan-2-yl)-4-((S)-5-hydroxyhexa-1,3-diyn-1-yl)benzamide (42);
4-((S)-6,7-dihydroxyhepta-1,3-diyn-1-yl)-N—((S)-1-(hydroxyamino)-3-methyl-3-(methylamino)-1-oxobutan-2-yl)benzamide (43);
4-((R)-6,7-dihydroxyhepta-1,3-diyn-1-yl)-N—((S)-1-(hydroxyamino)-3-methyl-3-(methylamino)-1-oxobutan-2-yl)benzamide (44);
N-((2S,3R)-3-amino-1-(hydroxyamino)-1-oxobutan-2-yl)-4-(5,6-dihydroxyhexa-1,3-diyn-1-yl)benzamide (45);
N—((S)-3-amino-1-(hydroxyamino)-3-methyl-1-oxobutan-2-yl)-4-(5,7-dihydroxyhepta-1,3-diyn-1-yl)benzamide (46);
N—((S)-3-amino-1-(hydroxyamino)-3-methyl-1-oxobutan-2-yl)-4-(6-hydroxy-5-methoxyhexa-1,3-diyn-1-yl)benzamide (47);
N-((2S,3R)-1-(hydroxyamino)-3-(methylamino)-1-oxobutan-2-yl)-4-(6-hydroxyhexa-1,3-diyn-1-yl)benzamide (48);
N-((2S,3R)-1-(hydroxyamino)-3-(methylamino)-1-oxobutan-2-yl)-4-((S)-5-hydroxyhexa-1,3-diyn-1-yl)benzamide (49);
N—((S)-3-amino-1-(hydroxyamino)-3-methyl-1-oxobutan-2-yl)-4-(5,6-dihydroxy-5-methylhexa-1,3-diyn-1-yl)benzamide (50);
4-(5,6-dihydroxyhexa-1,3-diyn-1-yl)-N—((S)-1-(hydroxyamino)-3-methyl-3-(methylamino)-1-oxobutan-2-yl)benzamide (51);
N—((S)-3-amino-1-(hydroxyamino)-3-methyl-1-oxobutan-2-yl)-4-((E)-6,7-dihydroxyhept-3-en-1-yn-1-yl)benzamide (52);
N—((S)-3-amino-1-(hydroxyamino)-3-methyl-1-oxobutan-2-yl)-4-((5S,6S)-5,7-dihydroxy-6-methylhepta-1,3-diyn-1-yl)benzamide (53);
N—((S)-3-amino-1-(hydroxyamino)-3-methyl-1-oxobutan-2-yl)-4-((S)-5,7-dihydroxyhepta-1,3-diyn-1-yl)benzamide (54);
(S,E)-N-(3-amino-1-(hydroxyamino)-3-methyl-1-oxobutan-2-yl)-4-(6-hydroxyhex-3-en-1-yn-1-yl)benzamide (55);
N-((2S,3R)-1-(hydroxyamino)-3-(methylamino)-1-oxobutan-2-yl)-4-(5-hydroxypenta-1,3-diyn-1-yl)benzamide (56);
N—((S)-3-amino-1-(hydroxyamino)-3-methyl-1-oxobutan-2-yl)-4-((1-hydroxy-3-(hydroxymethyl)cyclopentyl)buta-1,3-diyn-1-yl)benzamide (57);
N—((S)-3-amino-1-(hydroxyamino)-3-methyl-1-oxobutan-2-yl)-4-((E)-5,6-dihydroxyhex-3-en-1-yn-1-yl)benzamide (58);
N-((2S,3R)-3-amino-4,4,4-trifluoro-1-(hydroxyamino)-1-oxobutan-2-yl)-4-((S)-5-hydroxyhexa-1,3-diyn-1-yl)benzamide (59);
4-(5,6-dihydroxyhexa-1,3-diyn-1-yl)-N—((S)-3-hydroxy-1-(hydroxyamino)-3-methyl-1-oxobutan-2-yl)benzamide (60);
N—((S)-3-amino-1-(hydroxyamino)-3-methyl-1-oxobutan-2-yl)-4-((S)-5,6-dihydroxyhexa-1,3-diyn-1-yl)benzamide (61); or
N—((S)-3-amino-1-(hydroxyamino)-3-methyl-1-oxobutan-2-yl)-4-((R)-5,6-dihydroxyhexa-1,3-diyn-1-yl)benzamide (62).

16. A pharmaceutical composition comprising a pharmaceutically acceptable carrier or diluent and a compound of claim 1.

17. A method for treating a patient having a bacterial infection comprising administering to a patient in need thereof a therapeutically effective amount of a compound of claim 1 or a pharmaceutical composition of claim 16.

18. A method according to claim 17, wherein said bacterial infection is a gram-negative bacterial infection.

19. A method according to claim 18, wherein said gram-negative bacterial infection is Pseudomonas aeruginosa, Stenotrophomonas maltophila, Burkholderia cepacia, Alcaligenes xylosoxidans, Enterobacteriaceae, Haemophilus, Franciscellaceae or a Neisseria species.

20. Use of a compound of claim 1 in the preparation of a medicament for treating a gram-negative bacterial infection.
